(19) United States
(12) Patent Application Publication
(10) Pub. No.: US 2022/0348595 A1
(43) Pub. Date: Nov. 3, 2022

(54) COMPOUNDS AND METHODS FOR THE TARGETED DEGRADATION OF ESTROGEN RECEPTORS

(71) Applicants: Endotarget Inc., New Orleans, LA (US); EnhancedBio Inc., Seoul (KR)

(72) Inventors: Guangdi Wang, New Orleans, LA (US); Xianyou Peng, New Orleans, LA (US); Borui Kang, New Orleans, LA (US); HongJoong Kim, Seoul (KR); Hunsoon Jung, Seoul (KR); Hyunjung Choi, Seoul (KR); Bongsu Park, Seoul (KR)

(21) Appl. No.: 17/716,051

(22) Filed: Apr. 8, 2022

Related U.S. Application Data
(60) Provisional application No. 63/302,321, filed on Jan. 24, 2022.
(60) Provisional application No. 63/172,846, filed on Apr. 9, 2021.

(51) Int. Cl. C07F 5/02; C07D 401/14; A61P 35/00

(52) U.S. Cl. CPC C07F 5/025; C07D 401/14; A61P 35/00

(57) ABSTRACT

The present disclosure relates to bifunctional compounds that serve as degraders (and/or inhibitors) of the estrogen receptor (target protein). In the present disclosure, the bifunctional compounds, which contain a target protein (estrogen receptor) binding moiety and a E3 ubiquitin ligase (CRBN) binding moiety, are directed to bind to both estrogen receptor and CRBN, such that the ER is placed in close proximity to the E3 ligase to mediate ubiquitylation of the target protein followed by degradation of the target protein by the proteasome. The present disclosure provides methods for synthesizing the herein disclosed bifunctional compounds, and their pharmacological activities associated with degradation or inhibition of the target protein. Further, the present disclosure teaches the utilization of such compounds in a treatment for proliferative diseases, including cancer, particularly breast cancer, and especially ER+ breast cancer.

### CROSS-REFERENCE TO RELATED APPLICATIONS

This application claims priority to and the benefit of U.S. Provisional Application No. 63/172,846, filed Apr. 9, 2021, and U.S. Provisional Application No. 63/302,321, filed Jan. 24, 2022, the disclosures of which are hereby incorporated by reference herein in their entireties.

### FIELD OF THE INVENTION

The present description relates to bifunctional compounds that perform as modulators of a target protein, e.g., estrogen receptor (ER), which are degraded or inhibited as a result of ubiquitination and subsequent degradation of the ubiquitinated targeted protein by the proteasome. The bifunctional compounds contain one ligand that binds the target protein and another ligand that binds to a specific E3 ubiquitin ligase, which are linked via a linker molecule. In the present disclosure the bifunctional compounds can simultaneously bind estrogen receptor alpha (ERα) (target protein) and a cereblon (CRBN) E3 ubiquitin ligase, which promotes ubiquitination of ER and leads to degradation of ER by the proteasome. While small molecules can easily bind enzymes or receptors in tight and well-defined pockets, protein-protein interactions are difficult to target by small molecules. Since E3 ubiquitin ligases confer substrates specifically for ubiquitination, bifunctional compounds of which one end can bind to E3 ligases and promote ubiquitination of a target protein has become an attractive therapeutic strategy for protein degradation by proteasome.

### BACKGROUND OF THE INVENTION

Estrogen receptor is a member of the nuclear hormone receptor superfamily of transcription factors, and has a great pharmaceutical interest as a target for the treatment of breast cancer, osteoporosis and other endocrine female disorders. Binding of the natural ligand, 17-beta-estradiol, to the ER causes dimerization of the ER, which in turn binds to the estrogen response elements (ERE) in the promoters of the target gene or can interact with other transcription factor complexes like Fos/Jun (AP-1-responsive elements), and influence transcription of genes. The ERα regulates a large number of genes in many different target tissues and plays important roles in the development and progression of breast cancer. Thus, modulation of ER through binding of natural or synthetic ER ligands can have great impacts on the physiology and pathophysiology of the organism. Towards this goal several selective estrogen receptor modulators (SERMs) and selective estrogen receptor down-regulators (SERDs) have been developed, as antiestrogens. In this approach, these antiestrogens that bind to and compete for ER present in the estrogen responsive tissue antagonize ER activation by estrogen withdrawal from the binding site. Even though traditional antiestrogens, such as tamoxifen, vie efficiently for ER binding, their effectiveness is often hampered by partial agonism or acquired resistance to drugs, which results in partial blockade of estrogen-mediated activity. Hence there is an urgent need for other approaches to antagonize the ER.

Thus, there is a need in the art for compositions and methods for modulating specific target proteins, e.g., estrogen receptor (ER), which are degraded or inhibited as a result of ubiquitination and subsequent degradation of the ubiquitinated targeted protein by the proteasome. The present invention addresses this unmet need in the art.

### SUMMARY OF THE INVENTION

The present disclosure relates generally to novel bifunctional compounds and compositions useful for the degradation of a target protein by recruiting the target protein to an E3 ubiquitin ligase for degradation by the endogenous cellular ubiquitin proteasome system (UPS). In particular, the present disclosure furnishes bifunctional compounds, otherwise known as proteolysis targeting chimeric (PROTAC) compounds, which facilitates targeted ubiquitination of target protein (i.e., estrogen receptor (ER)), and then undergo degradation and/or exhibit inhibition of the target protein by the bifunctional compounds disclosed herein. In addition, the description provides the methods of making such compounds and compositions; methods of using such compounds and compositions; pharmaceutical compositions comprising such compounds and compositions; and methods of using such pharmaceutical compositions, for the treatment or amelioration of a disease condition, such as cancer, especially breast cancer.

In an additional aspect, the present disclosure provides a method of ubiquitinating followed by degrading a target protein by bifunctional compounds attached by a chemical linker; therapeutic compositions comprising an effective amount of a compound disclosed herein or salt/solvate form thereof, and its delivery using a pharmaceutically acceptable carrier. In yet another aspect, the therapeutic compositions of a compound or multiple compounds that degrade and/or inhibit the target protein in a patient or subject, such as a human or animal, can be used for treating or ameliorating disease conditions/states, e.g., breast cancer, through modulation of wild-type ER or mutant ER or other variants of ER.

In an embodiment, provided here is a compound of the Formula (I), or a tautomer, stereoisomer, mixture of stereoisomers, pharmaceutically acceptable salt, or solvate thereof:

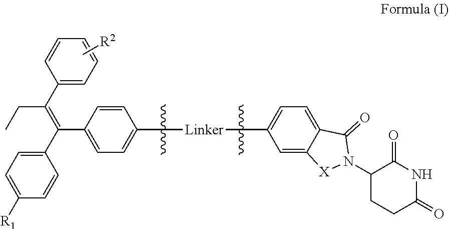

In some embodiments, R¹ and R² are each independently hydrogen, deuterium, halogen, hydroxyl, alkyl, alkoxy,

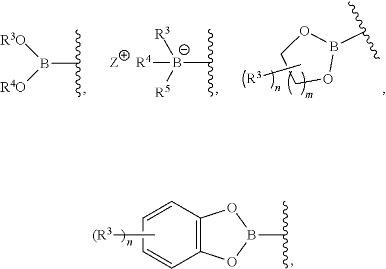

or any combination thereof. In some embodiments, R¹ is

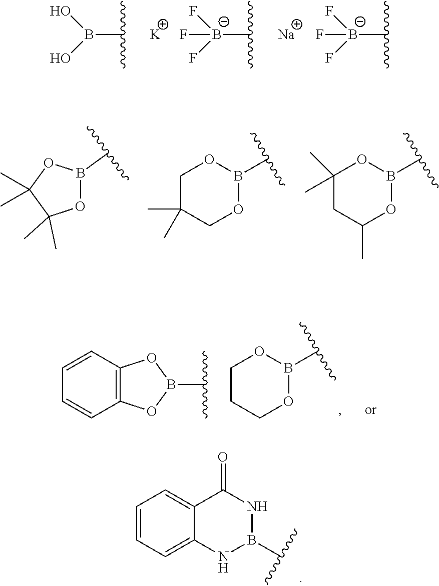

In some embodiments, the R¹ substituent point of attachment is on the substituent boron atom of R¹. In some embodiments, R² is hydrogen, F, Cl, Br, or I.

In some embodiments, X is O, S, C(R³)(R⁴), or C═O. In some embodiments, X is CH₂ or C═O.

In some embodiments, each occurrence of R³, R⁴, and R⁵ is independently hydrogen, deuterium, halogen, hydroxyl, alkyl, cycloalkyl, heterocycloalkyl, alkenyl, cycloalkenyl, alkynyl, cycloalkynyl, aryl, aryl alkyl, heteroaryl, heteroaryl alkyl, or any combination thereof.

In some embodiments, each occurrence of Z is independently Li, Na, or K.

In some embodiments, the linker is an optionally substituted linking moiety. In some embodiments, the linker comprises a branched or unbranched, cyclized or uncyclized, saturated or unsaturated chain of 5 to 16 carbon atoms in length, or any combination thereof; wherein 1 to 6 of the carbon atoms are optionally replaced with a heteroatom. In some embodiments, the heteroatom is independently O, N, and S.

In some embodiments, the linking moiety comprises a branched or linear C₅-C₁₆ alkyl, branched or linear amino-C₅-C₁₆ alkyl, branched or linear C₅-C₁₆ alkoxy, branched or linear thio-C₅-C₁₆ alkyl, C₅-C₁₆ cycloalkyl, amino-C₅-C₁₆ cycloalkyl, hydroxy-C₅-C₁₆ cycloalky, thio-C₅-C₁₆ cycloalkyl, or any combination thereof; wherein 1 to 6 of the carbon atoms are optionally replaced with a heteroatom. In some embodiments, the heteroatom is independently O, N, and S.

In one aspect, the present invention relates, in part, to a composition comprising at least one compound of the present invention. In various embodiments, the composition is a pharmaceutical composition. In one embodiment, the pharmaceutical composition comprises at least one compound of the present invention and a pharmaceutically acceptable carrier.

In one embodiment, the pharmaceutical composition is suitable for enteral administration. In one embodiment, the pharmaceutical composition is suitable for oral administration. In one embodiment, the pharmaceutical composition is suitable for parenteral administration.

In one aspect, the present invention relates, in part, to a pharmaceutical formulation comprising at least one compound of the present invention. In one embodiment, the pharmaceutical formulation comprises at least one compound of the present invention and a pharmaceutically acceptable carrier.

In one embodiment, the pharmaceutical formulation is suitable for enteral administration. In one embodiment, the pharmaceutical formulation is suitable for oral administration. In one embodiment, the pharmaceutical formulation is suitable for parenteral administration.

In one aspect, the present invention relates, in part, to a method of preparing at least one compound, composition, pharmaceutical composition, or pharmaceutical formulation of the present invention.

In one aspect, the present invention relates, in part, to a method of treating a disease or disorder in a subject in need thereof, the method comprising administering at least one compound, composition, pharmaceutical composition, or pharmaceutical formulation of the present invention to the subject. In one embodiment, the disease or disorder is a disease or disorder associated with at least one ER. In some embodiments, the disease or disorder is a breast cancer, all stages of breast cancer, estrogen receptor (ER)-positive breast cancer, invasive breast cancer, or any combination thereof.

In one aspect, the present invention relates, in part, to a method for treating breast cancer in a subject in need thereof, the method comprising administering an effective amount of at least one compound, composition, pharmaceutical composition, or pharmaceutical formulation of the present invention to the subject. In one embodiment, the breast cancer is an ER-positive breast cancer. In one embodiment, the breast cancer is an invasive breast cancer. In one embodiment, the subject expresses a mutant ER-α protein.

In one aspect, the present invention relates, in part, to a method of reducing the level or activity of at least one target protein, the method comprising administering at least one compound, composition, pharmaceutical composition, or pharmaceutical formulation of the present invention.

In another aspect, the present invention relates, in part, to a method of inhibiting at least one target protein, the method comprising administering at least one compound, composition, pharmaceutical composition, or pharmaceutical formulation of the present invention. In one embodiment, the target protein is an estrogen receptor.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

The following detailed description of preferred embodiments of the invention will be better understood when read in conjunction with the appended drawings. It should be understood, however, that the invention is not limited to the precise arrangements and instrumentalities of the embodiments shown in the drawings.

FIG. 1 shows the dose-dependent ERα degradation by exemplary compounds 3, compound 5, and compound 35 of the present disclosure in MCF-7 breast cancer cells.

FIG. 2 shows the dose-dependent ERα degradation by exemplary compound 33, compound 38, and compound 37 of the present disclosure in MCF-7 breast cancer cells.

FIG. 3 shows the dose-dependent ERα degradation by exemplary compound 9, compound 11, and compound 12 of the present disclosure in MCF-7 breast cancer cells.

FIG. 4 shows the dose-dependent ERα degradation by exemplary compound 21, compound 24, and compound 23 of the present disclosure in MCF-7 breast cancer cells.

FIG. 5 shows the dose-dependent ERα degradation by exemplary compound 40 and compound 16 of the present disclosure in MCF-7 breast cancer cells.

FIG. 6 shows the dose-dependent ERα degradation by exemplary compound 18, compound 29, and compound 30 of the present disclosure in MCF-7 breast cancer cells.

FIG. 7 shows the dose-dependent ERα degradation by exemplary compound 31 and compound 32 of the present disclosure in MCF-7 breast cancer cells.

FIG. 8 shows the dose-dependent ERα degradation by exemplary compound 34, compound 26, and compound 25 of the present disclosure in MCF-7 breast cancer cells.

FIG. 9 shows the dose-dependent ERα degradation by exemplary compound 19 and compound 20 of the present disclosure in MCF-7 breast cancer cells.

FIG. 10 shows the dose-dependent ERα degradation by exemplary compound 28, compound 27, and compound 20 of the present disclosure in MCF-7 breast cancer cells.

FIG. 11 shows the efficacy of oral treatment of compound 11 and compound 21 in inhibiting MCF-7/TamR xenograft tumor growth in nude mice.

FIG. 12 shows the efficacy of oral treatment of compound 69 in inhibiting ST1799 PDX tumor growth in nude mice.

FIG. 13 shows the single dose pharmacokinetic profile of compound 21 in Sprague Dawley rat.

FIG. 14 shows the single dose pharmacokinetic profile of compound 89 in Sprague Dawley rat.

### DETAILED DESCRIPTION OF THE DISCLOSURE

The present disclosure relates to compounds that bind competitively and/or non-competitively to the estrogen receptor alpha (ERα), and the E3 ubiquitin ligase, cereblon (CRBN) to effect ubiquitination and subsequent degradation of the ERα protein, thereby blocking the estrogen signaling pathways and inhibiting the growth of estrogen receptor (ER) dependent cells. The disclosure also relates to pharmaceutical compositions comprising these ER degrading compounds, and methods for using the same for treatment of diseases and conditions mediated by the estrogen receptor, including breast cancer.

Based on the ideas of the invention described above, the disclosed bifunctional compounds can be applied for targeted degradation of ER, and be used to treat or prevent ER+ breast cancer. The following detailed description is furnished to assist those skilled in the art in joining the present disclosure.

Before the subject disclosure is further described, it is to be understood that the disclosure is not limited to the particular embodiments of the disclosure described below, as variations of the particular embodiments may be made by those of ordinary skill in the art and still maintain the spirit and scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

In this specification and the claims reported herein, the phrase “and/or,” as used is construed to mean “either or both” of the elements, i.e., either the elements can be conjunctively present in some cases or the elements can be disjunctively present in other cases.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “cancer” refers to any of various types of malignant neoplasms, most of which invade surrounding tissues, may metastasize to several sites and are likely to recur after attempted removal and to cause death of the patient unless adequately treated. As used herein, neoplasia comprises cancer. Representative cancers include, for example, squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias, including non-acute and acute leukemias, such as acute myelogenous leukemia, acute lymphocytic leukemia, acute promyelocytic leukemia (APL), acute T-cell lymphoblastic leukemia, T-lineage acute lymphoblastic leukemia (T-ALL), adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas, among others, which may be treated by one or more compounds of the present invention.

A disease or disorder is “alleviated” if the severity of a sign or symptom of the disease or disorder, the frequency with which such a sign or symptom is experienced by a patient, or both, is reduced.

As used herein, the term “minimize” or “reduce”, or derivatives thereof, include a complete or partial degradation of a target protein (ER) and/or inhibition of a specified biological effect and/or reduction of ER expression at the transcript or protein level. (which is apparent from the context in which the terms “minimize” or “reduce” are used).

The term “inhibit,” as used herein, means to suppress or block an activity or function by at least about ten percent relative to a control value. Preferably, the activity is suppressed or blocked by 50% compared to a control value, more preferably by 75%, and even more preferably by 95% or more.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell from a patient (e.g., for diagnosis or ex vivo applications), who has a disease or disorder contemplated herein, a sign or symptom of a disease or disorder contemplated herein or the potential to develop a disease or disorder contemplated herein, with the purpose to cure, heal, alleviate, relieve, alter, remedy, ameliorate, improve or affect a disease or disorder contemplated herein, the signs or symptoms of a disease or disorder contemplated herein or the potential to develop a disease or disorder contemplated herein. Such treatments may be specifically tailored or modified, based on knowledge obtained from the field of pharmacogenomics. To “treat” a disease as the term is used herein, means to reduce the frequency or severity of at least one sign or symptom of a disease or disorder experienced by a subject.

“Parenteral” administration of a composition includes, e.g., subcutaneous (s.c.), intravenous (i.v.), intramuscular (i.m.), or intrasternal injection, or infusion techniques.

The compounds according to the disclosure are isolated and purified in a manner known per se, e.g. by distilling off the solvent in vacuo and recrystallizing the residue obtained from a suitable solvent or subjecting it to one of the customary purification methods, such as chromatography on a suitable support material. Furthermore, reverse phase preparative HPLC of compounds of the present disclosure which possess a sufficiently basic or acidic functionality, may result in the formation of a salt, such as, in the case of a compound of the present disclosure which is sufficiently basic, a trifluoroacetate or formate salt for example, or, in the case of a compound of the present disclosure which is sufficiently acidic, an ammonium salt for example. Salts of this type can either be transformed into its free base or free acid form, respectively, by various methods known to the person skilled in the art, or be used as salts in subsequent biological assays. Additionally, the drying process during the isolation of compounds of the present disclosure may not fully remove traces of cosolvents, especially such as formic acid or trifluoroacetic acid, to give solvates or inclusion complexes. The person skilled in the art will recognize which solvates or inclusion complexes are acceptable to be used in subsequent biological assays. It is to be understood that the specific form (e.g., salt, free base, solvate, inclusion complex) of a compound of the present disclosure as isolated as described herein is not necessarily the only form in which said compound can be applied to a biological assay in order to quantify the specific biological activity.

One aspect of the disclosure is salts of the compounds according to the disclosure including all inorganic and organic salts, especially all pharmaceutically acceptable inorganic and organic salts, particularly all pharmaceutically acceptable inorganic and organic salts customarily used in pharmacy.

Examples of salts include, but are not limited to, lithium, sodium, potassium, calcium, aluminum, magnesium, titanium, meglumine, ammonium, salts optionally derived from NH₃ or organic amines having from 1 to 16 C-atoms such as, e.g., ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, ethylendiamine, N-methylpiperindine, arginine, lysine, and guanidinium salts.

The salts of the disclosed compounds include pharmaceutically acceptable water-insoluble and, particularly, water-soluble salts.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, “pharmaceutically acceptable salts” refer to derivatives of the compounds disclosed herein wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt may be 1:1, or any ratio other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

Salts of the compounds of Formula (I) according to the disclosure can be obtained by dissolving the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or base, or to which the desired acid or base is then added. The acid or base can be employed in salt preparation, depending on whether a mono- or polybasic acid or base is concerned and depending on which salt is desired, in an equimolar quantitative ratio or one differing therefrom. The salts are obtained by filtering, reprecipitating, precipitating with a non-solvent for the salt or by evaporating the solvent. Salts obtained can be converted into the free compounds which, in turn, can be converted into salts. In this manner, pharmaceutically unacceptable salts, which can be obtained, for example, as process products in the manufacturing on an industrial scale, can be converted into pharmaceutically acceptable salts by processes known to the person skilled in the art.

According to the person skilled in the art the compounds of Formula (I) according to this disclosure as well as their salts may contain, e.g., when isolated in crystalline form, varying amounts of solvents. Included within the scope of the disclosure are therefore all solvates and in particular all hydrates of the compounds of Formula (I) according to this disclosure as well as all solvates and in particular all hydrates of the salts of the compounds of Formula (I) according to this disclosure.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

The compounds according to the disclosure and their salts can exist in the form of tautomers which are included in the embodiments of the disclosure.

The term “tautomer” refers to one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH conditions. The concept of tautomers that are interconvertible by tautomerizations is called tautomerism.

Where the present specification depicts a compound prone to tautomerization, but only depicts one of the tautomers, it is understood that all tautomers are included as part of the meaning of the chemical depicted. It is to be understood that the compounds disclosed herein may be depicted as different tautomers. It should also be understood that when compounds have tautomeric forms, all tautomeric forms are intended to be included, and the naming of the compounds does not exclude any tautomer form.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine.

The compounds of the disclosure may, depending on their structure, exist in different stereoisomeric forms. These forms include configurational isomers or optically conformational isomers (enantiomers and/or diastereoisomers including those of atropisomers). The present disclosure therefore includes enantiomers, diastereoisomers as well as mixtures thereof. From those mixtures of enantiomers and/or disastereoisomers pure stereoisomeric forms can be isolated with methods known in the art, preferably methods of chromatography, especially high performance liquid chromatography (HPLC) using achiral or chiral phase. The disclosure further includes all mixtures of the stereoisomers mentioned above independent of the ratio, including the racemates.

The compounds of the disclosure may, depending on their structure, exist in various stable isotopic forms. These forms include those in which one or more hydrogen atoms have been replaced with deuterium atoms, those in which one or more nitrogen atoms have been replaced with ¹⁵N atoms, or those in which one or more atoms of carbon, fluorine, chlorine, bromine, sulfur, or oxygen have been replaced by the stable isotope of the respective, original atoms.

Some of the compounds and salts according to the disclosure may exist in different crystalline forms (polymorphs) which are within the scope of the disclosure.

It is a further object of the disclosure to provide ER degrading compounds, methods of synthesizing the ER degrading bifunctional compounds, methods of manufacturing the ER degrading compounds, and methods of using the ER degrading compounds.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration. The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration.

Non-limiting examples of agents suitable for formulation with the, e.g., compounds provided by the instant invention include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26): biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

A “therapeutic” treatment is a treatment administered to a subject who exhibits signs or symptoms of pathology disease or disorder, for the purpose of diminishing or eliminating those signs or symptoms.

As used herein, the terms “effective amount,” “pharmaceutically effective amount” and “therapeutically effective amount” refer to a sufficient amount of an agent to provide the desired biological or physiologic result. That result may be reduction and/or alleviation of a sign, a symptom, or a cause of a disease or disorder, or any other desired alteration of a biological system. An appropriate effective amount in any individual case may be determined by one of ordinary skill in the art using routine experimentation.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “halo” or “halogen” alone or as part of another substituent means, unless otherwise stated, a fluorine, chlorine, bromine, or iodine atom.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

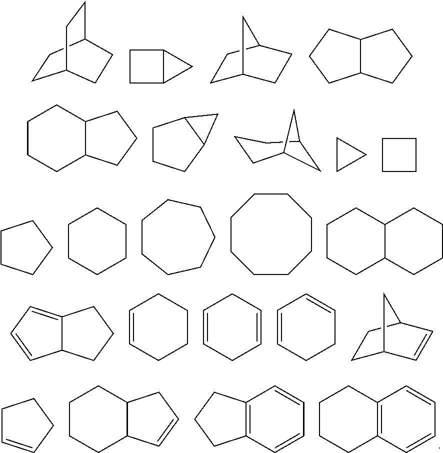

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃. As used herein, the terms “heteroalkyl” refers to “alkoxy,” “alkylamino” and “alkylthio” that are used in their conventional sense, and refer to alkyl groups linked to molecules via an oxygen atom, an amino group, a sulfur atom, respectively.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

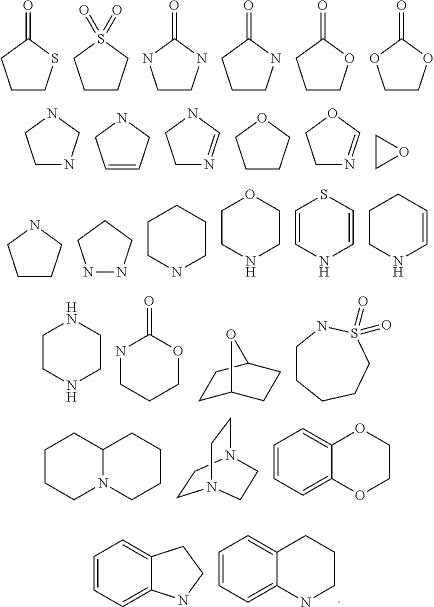

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “heteroaryl” or “heteroaromatic” refers to a heterocycle having aromatic character. A polycyclic heteroaryl may include one or more rings that are partially saturated. Examples include the following moieties:

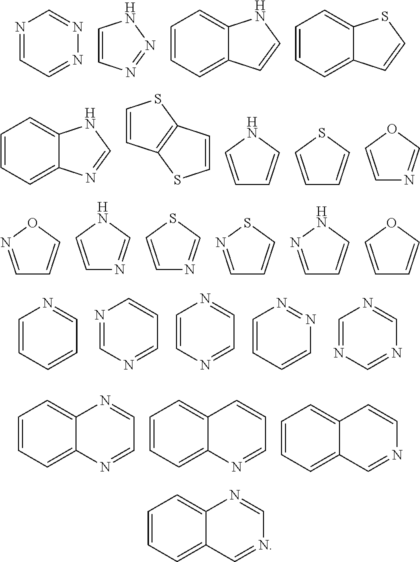

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

As used herein, the term “potency” refers to the dose needed to produce half the maximal response (ED₅₀).

As used herein, the term “efficacy” refers to the maximal effect (Eₘₐₓ) achieved within an assay.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

In some embodiments, provided herein are compounds of Formula I, or a tautomer, stereoisomer, mixture of stereoisomers, pharmaceutically acceptable salt, or solvate thereof:

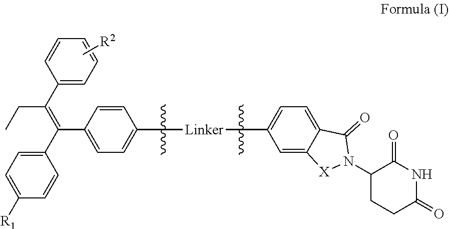

In some embodiments, R¹ and R² are each independently hydrogen, deuterium, halogen, hydroxyl, alkyl, alkoxy,

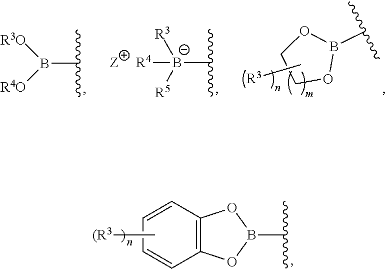

or any combination thereof.

In some embodiments, R¹ is

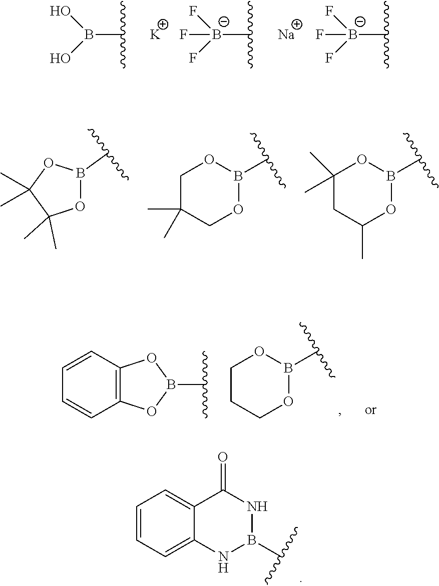

In some embodiments, the R¹ substituent point of attachment is on the substituent boron atom of R¹.

In some embodiments, R² is hydrogen, F, Cl, Br, or I.

In some embodiments, X is O, S, C(R³)(R⁴), or C═O. In some embodiments, X is CH₂ or C═O.

In some embodiments, each occurrence of R³, R⁴, and R⁵ is independently hydrogen, deuterium, halogen, hydroxyl, alkyl, cycloalkyl, heterocycloalkyl, alkenyl, cycloalkenyl, alkynyl, cycloalkynyl, aryl, aryl alkyl, heteroaryl, heteroaryl alkyl, or any combination thereof.

In some embodiments, each occurrence of Z is independently Li, Na, or K.

In some embodiments, the linker is an optionally substituted linking moiety. In some embodiments, the linker comprises a branched or unbranched, cyclized or uncyclized, saturated or unsaturated chain of 5 to 22 carbon atoms in length, or any combination thereof. In some embodiments, the linker is an optionally substituted linking moiety. In some embodiments, the linker comprises a branched or unbranched, cyclized or uncyclized, saturated or unsaturated chain of 5 to 16 carbon atoms in length, or any combination thereof.

In some embodiments, the linking moiety comprises 1 to 6 of the carbon atoms that are optionally replaced with a heteroatom. In some embodiments, the heteroatom is independently O, N, and S.

In some embodiments, the linking moiety comprises a branched or linear C₅-C₂₂ alkyl, branched or linear amino-C₅-C₂₂ alkyl, branched or linear C₅-C₂₂ alkoxy, branched or linear thio-C₅-C₂₂ alkyl, C₅-C₂₂ cycloalkyl, amino-C₅-C₂₂ cycloalkyl, hydroxy-C₅-C₂₂ cycloalky, thio-C₅-C₂₂ cycloalkyl, —Y¹—(CH₂)ₐ—(NH)_{b}—W—(CH₂)_{c}—Y²—, or any combination thereof. In some embodiments, the linking moiety comprises a branched or linear C₅-C₁₆ alkyl, branched or linear amino-C₅-C₁₆ alkyl, branched or linear C₅-C₁₆ alkoxy, branched or linear thio-C₅-C₁₆ alkyl, C₅-C₁₆ cycloalkyl, amino-C₅-C₁₆ cycloalkyl, hydroxy-C₅-C₁₆ cycloalky, thio-C₅-C₁₆ cycloalkyl, —Y¹—(CH₂)ₐ—(NH)_{b}—W—(CH₂)_{c}—Y²—, or any combination thereof.

In some embodiments, the linker is —Y¹—(CH₂)ₐ—(NH)_{b}—W—(CH₂)_{c}—Y²—.

In some embodiments, each occurrence of Y¹ and Y² is independently O, —NH—, 5 to 9 membered heterocycloalkyl having one or two heteroatoms selected from N, O, and S, or any combination thereof, or absent.

In some embodiments, Y¹ is absent or O.

In some embodiments, Y² is —NH— or 5 to 9 membered heterocycloalkyl having one or two heteroatoms selected from N, O, and S. For example, in some embodiments, said 5 to 9 membered heterocycloalkyl is piperidine or piperazine.

In some embodiments, each occurrence of a, b, and c is independently an integer of 0 to 7.

In some embodiments, each occurrence of a and c is independently an integer of 0 to 7.

In some embodiments, each occurrence of b is independently an integer of 0 or 1.

In some embodiments, each occurrence of W is independently 5 to 9 membered heterocycloalkyl having one or two heteroatoms selected from N, O, and S.

For example, in some embodiments, the linker is selected from:

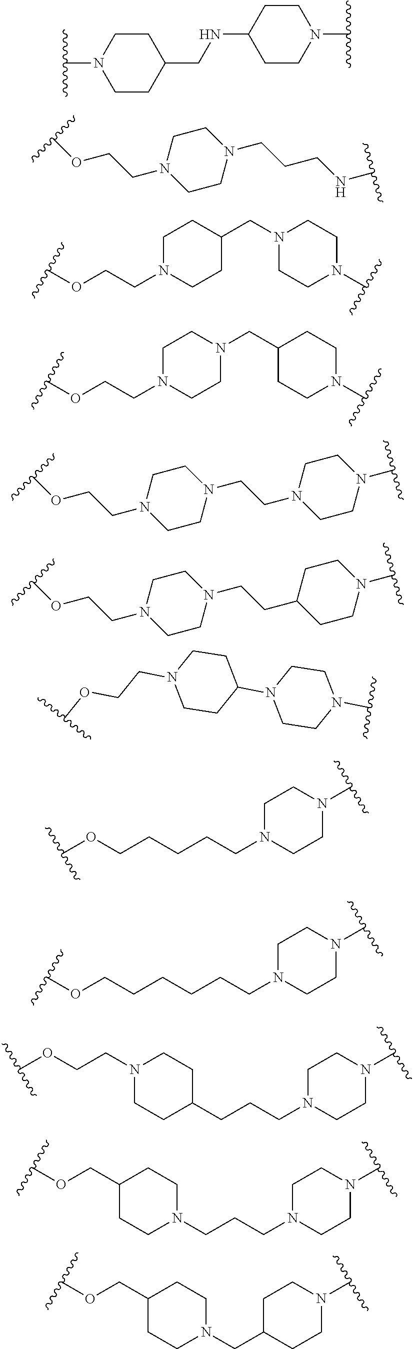

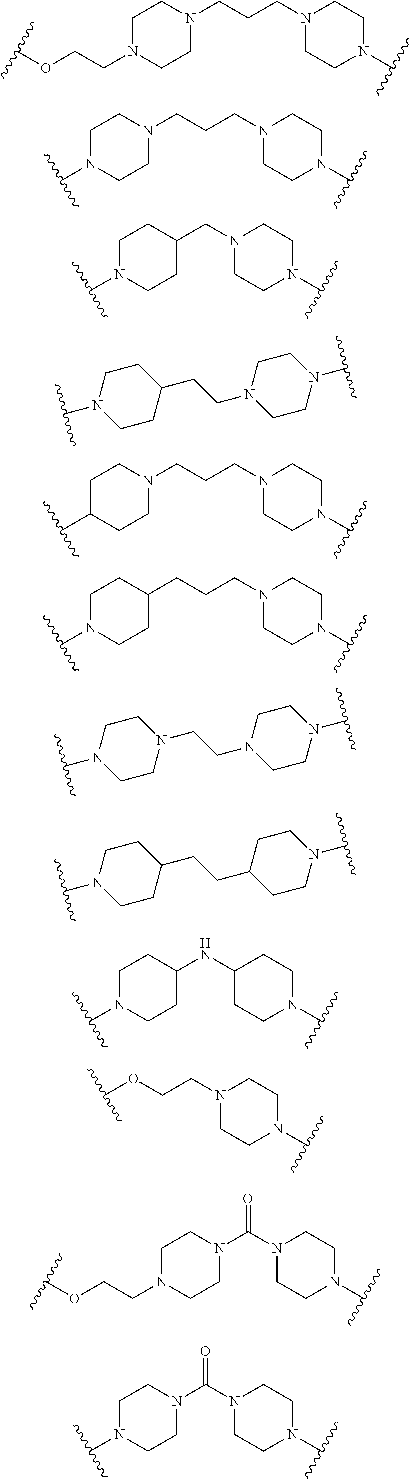

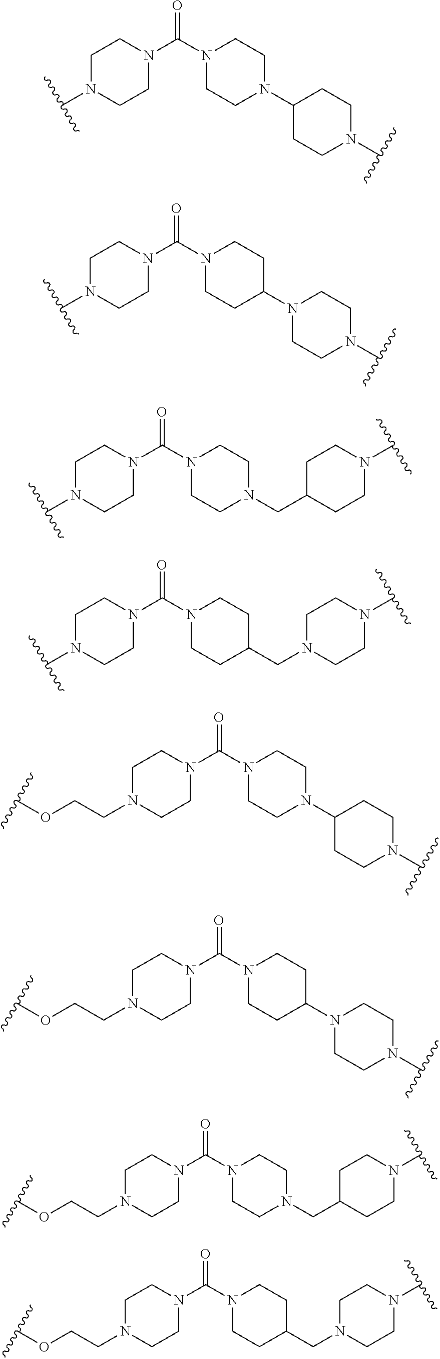

In some embodiments, the compound having the structure of Formula (I) is selected from:
(Z)-(4-(1-(4-(4-((4-(2-(2,6-dioxopiperidin-3-yl)-1-oxoisoindolin-5-yl)piperazin-1-yl) methyl)piperidin-1-yl)phenyl)-2-phenylbut-1-en-1-yl)phenyl)boronic acid;
(E)-(4-(1-(4-(4-((4-(2-(2,6-dioxopiperidin-3-yl)-1-oxoisoindolin-5-yl)piperazin-1-yl) methyl)piperidin-1-yl)phenyl)-2-phenylbut-1-en-1-yl)phenyl)boronic acid;
(Z)-(4-(1-(4-(4-((4-(2-(2,6-dioxopiperidin-3-yl)-3-oxoisoindolin-5-yl)piperazin-1-yl) methyl)piperidin-1-yl)phenyl)-2-phenylbut-1-en-1-yl)phenyl)boronic acid;
(E)-(4-(1-(4-(4-((4-(2-(2,6-dioxopiperidin-3-yl)-3-oxoisoindolin-5-yl)piperazin-1-yl) methyl)piperidin-1-yl)phenyl)-2-phenylbut-1-en-1-yl)phenyl)boronic acid;
(Z)-(4-(1-(4-(4-((4-(2-(2,6-dioxopiperidin-3-yl)-1,3-dioxoisoindolin-5-yl)piperazin-1-yl) methyl)piperidin-1-yl)phenyl)-2-phenylbut-1-en-1-yl)phenyl)boronic acid; (E)-(4-(1-(4-(4-((4-(2-(2,6-dioxopiperidin-3-yl)-1,3-dioxoisoindolin-5-yl)piperazin-1-yl)methyl)piperidin-1-yl)phenyl)-2-phenylbut-1-en-1-yl)phenyl)boronic acid;
(Z)-(4-(1-(4-(4-(2-(4-(2-(2,6-dioxopiperidin-3-yl)-1-oxoisoindolin-5-yl)piperazin-1-yl) ethyl)piperidin-1-yl)phenyl)-2-phenylbut-1-en-1-yl)phenyl)boronic acid;
(E)-(4-(1-(4-(4-(2-(4-(2-(2,6-dioxopiperidin-3-yl)-1-oxoisoindolin-5-yl)piperazin-1-yl) ethyl)piperidin-1-yl)phenyl)-2-phenylbut-1-en-1-yl)phenyl)boronic acid;
(Z)-3-(1-oxo-5-(4-(2-(1-(4-(2-phenyl-1-(4-(4,4,5,5-tetramethyl-1,3,2-dioxaborolan-2-yl)phenyl)but-1-en-1-yl)phenyl)piperidin-4-yl)ethyl)piperazin-1-yl)isoindolin-2-yl)piperidine-2,6-dione;
(E)-3-(1-oxo-5-(4-(2-(1-(4-(2-phenyl-1-(4-(4,4,5,5-tetramethyl-1,3,2-dioxaborolan-2-yl) phenyl)but-1-en-1-yl)phenyl)piperidin-4-yl)ethyl)piperazin-1-yl)isoindolin-2-yl)piperidine-2,6-dione;
(Z)-(4-(1-(4-(4-(((1-(2-(2,6-dioxopiperidin-3-yl)-1-oxoisoindolin-5-yl)piperidin-4-yl) amino)methyl)piperidin-1-yl)phenyl)-2-phenylbut-1-en-1-yl)phenyl)boronic acid;
(E)-(4-(1-(4-(4-(((1-(2-(2,6-dioxopiperidin-3-yl)-1-oxoisoindolin-5-yl)piperidin-4-yl) amino)methyl)piperidin-1-yl)phenyl)-2-phenylbut-1-en-1-yl)phenyl)boronic acid;
(Z)-(4-(1-(4-(4-(((1-(2-(2,6-dioxopiperidin-3-yl)-3-oxoisoindolin-5-yl)piperidin-4-yl) amino)methyl)piperidin-1-yl)phenyl)-2-phenylbut-1-en-1-yl)phenyl)boronic acid;
(E)-(4-(1-(4-(4-(((1-(2-(2,6-dioxopiperidin-3-yl)-3-oxoisoindolin-5-yl)piperidin-4-yl) amino)methyl)piperidin-1-yl)phenyl)-2-phenylbut-1-en-1-yl)phenyl)boronic acid;
(Z)-(4-(1-(4-(2-(4-(3-((2-(2,6-dioxopiperidin-3-yl)-1,3-dioxoisoindolin-5-yl)amino)propyl)piperazin-1-yl)ethoxy)phenyl)-2-phenylbut-1-en-1-yl)phenyl)boronic acid;
(E)-(4-(1-(4-(2-(4-((2-(2,6-dioxopiperidin-3-yl)-1,3-dioxoisoindolin-5-yl)amino)propyl)piperazin-1-yl)ethoxy)phenyl)-2-phenylbut-1-en-1-yl)phenyl)boronic acid;
(Z)-(4-(1-(4-(2-(4-((1-(2-(2,6-dioxopiperidin-3-yl)-1,3-dioxoisoindolin-5-yl)piperidin-4-yl) methyl)piperazin-1-yl)ethoxy)phenyl)-2-phenylbut-1-en-1-yl)phenyl)boronic acid;
(E)-(4-(1-(4-(2-(4-((1-(2-(2,6-dioxopiperidin-3-yl)-1,3-dioxoisoindolin-5-yl)piperidin-4-yl) methyl)piperazin-1-yl)ethoxy)phenyl)-2-phenylbut-1-en-1-yl)phenyl)boronic acid;
(Z)-(4-(1-(4-(2-(4-((4-(2-(2,6-dioxopiperidin-3-yl)-1,3-dioxoisoindolin-5-yl)piperazin-1-yl) methyl)piperidin-1-yl)ethoxy)phenyl)-2-phenylbut-1-en-1-yl)phenyl)boronic acid;
(E)-(4-(1-(4-(2-(4-((4-(2-(2,6-dioxopiperidin-3-yl)-1,3-dioxoisoindolin-5-yl)piperazin-1-yl) methyl)piperidin-1-yl)ethoxy)phenyl)-2-phenylbut-1-en-1-yl)phenyl)boronic acid;
(Z)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid;
(E)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid;
(Z)-(4-(1-(4-(2-(4-(2-(4-(2-(2,6-dioxopiperidin-3-yl)-1,3-dioxoisoindolin-5-yl)piperazin-1-yl) ethyl)piperazin-1-yl)ethoxy)phenyl)-2-phenylbut-1-en-1-yl)phenyl)boronic acid;
(E)-(4-(1-(4-(2-(4-(2-(4-(2-(2,6-dioxopiperidin-3-yl)-1,3-dioxoisoindolin-5-yl)piperazin-1-yl) ethyl)piperazin-1-yl)ethoxy)phenyl)-2-phenylbut-1-en-1-yl)phenyl)boronic acid;
(Z)-(4-(1-(4-(2-(4-(2-(1-(2-(2,6-dioxopiperidin-3-yl)-1,3-dioxoisoindolin-5-yl)piperidin-4-yl) ethyl)piperazin-1-yl)ethoxy)phenyl)-2-phenylbut-1-en-1-yl)phenyl)boronic acid;
(E)-(4-(1-(4-(2-(4-(2-(1-(2-(2,6-dioxopiperidin-3-yl)-1,3-dioxoisoindolin-5-yl)piperidin-4-yl) ethyl)piperazin-1-yl)ethoxy)phenyl)-2-phenylbut-1-en-1-yl)phenyl)boronic acid;
(Z)-(4-(1-(4-(2-(4-(2-(4-(2-(2,6-dioxopiperidin-3-yl)-1,3-dioxoisoindolin-5-yl)piperazin-1-yl) ethyl)piperidin-1-yl)ethoxy)phenyl)-2-phenylbut-1-en-1-yl)phenyl)boronic acid;
(E)-(4-(1-(4-(2-(4-(2-(4-(2-(2,6-dioxopiperidin-3-yl)-1,3-dioxoisoindolin-5-yl)piperazin-1-yl) ethyl)piperidin-1-yl)ethoxy)phenyl)-2-phenylbut-1-en-1-yl)phenyl)boronic acid;
(Z)-(4-(1-(4-(2-(4-(2-(4-(2-(2,6-dioxopiperidin-3-yl)-1-oxoisoindolin-5-yl)piperazin-1-yl) ethyl)piperidin-1-yl)ethoxy)phenyl)-2-phenylbut-1-en-1-yl)phenyl)boronic acid;
(E)-(4-(1-(4-(2-(4-(2-(4-(2-(2,6-dioxopiperidin-3-yl)-1-oxoisoindolin-5-yl)piperazin-1-yl) ethyl)piperidin-1-yl)ethoxy)phenyl)-2-phenylbut-1-en-1-yl)phenyl) boronic acid;
(Z)-(4-(1-(4-(2-(4-(4-(2-(2,6-dioxopiperidin-3-yl)-1,3-dioxoisoindolin-5-yl)piperazin-1-yl)piperidin-1-yl)ethoxy)phenyl)-2-phenylbut-1-en-1-yl)phenyl)boronic acid;
(E)-(4-(1-(4-(2-(4-(4-(2-(2,6-dioxopiperidin-3-yl)-1,3-dioxoisoindolin-5-yl)piperazin-1-yl)piperidin-1-yl)ethoxy)phenyl)-2-phenylbut-1-en-1-yl)phenyl)boronic acid;
(Z)-(4-(1-(4-(2-(1′-(2-(2,6-dioxopiperidin-3-yl)-1,3-dioxoisoindolin-5-yl)-[4,4′-bipiperidin]-1-yl) ethoxy)phenyl)-2-phenylbut-1-en-1-yl)phenyl)boronic acid;
(E)-(4-(1-(4-(2-(1′-(2-(2,6-dioxopiperidin-3-yl)-1,3-dioxoisoindolin-5-yl)-[4,4′-bipiperidin]-1-yl) ethoxy)phenyl)-2-phenylbut-1-en-1-yl)phenyl)boronic acid;
(Z)-(4-(1-(4-((5-(4-(2-(2,6-dioxopiperidin-3-yl)-1-oxoisoindolin-5-yl)piperazin-1-yl) pentyl)oxy)phenyl)-2-phenylbut-1-en-1-yl)phenyl)boronic acid;
(E)-(4-(1-(4-((5-(4-(2-(2,6-dioxopiperidin-3-yl)-1-oxoisoindolin-5-yl)piperazin-1-yl) pentyl)oxy)phenyl)-2-phenylbut-1-en-1-yl)phenyl)boronic acid;
(Z)-(4-(1-(4-(2-(4-(3-(4-(2-(2,6-dioxopiperidin-3-yl)-1,3-dioxoisoindolin-5-yl)piperazin-1-yl) propyl)piperazin-1-yl)ethoxy)phenyl)-2-phenylbut-1-en-1-yl)phenyl)boronic acid;
(E)-(4-(1-(4-(2-(4-(3-(4-(2-(2,6-dioxopiperidin-3-yl)-1,3-dioxoisoindolin-5-yl)piperazin-1-yl) propyl)piperazin-1-yl)ethoxy)phenyl)-2-phenylbut-1-en-1-yl)phenyl)boronic acid;
(Z)-(4-(1-(4-(4-(((1-(2-(2,6-dioxopiperidin-3-yl)-1,3-dioxoisoindolin-5-yl)piperidin-4-yl) amino)methyl)piperidin-1-yl)phenyl)-2-phenylbut-1-en-1-yl)phenyl)boronic acid;
(E)-(4-(1-(4-(4-(((1-(2-(2,6-dioxopiperidin-3-yl)-1,3-dioxoisoindolin-5-yl)piperidin-4-yl) amino)methyl)piperidin-1-yl)phenyl)-2-phenylbut-1-en-1-yl)phenyl)boronic acid;
(Z)-(4-(1-(4-(2-(4-(2-(2,6-dioxopiperidin-3-yl)-1,3-dioxoisoindolin-5-yl)piperazin-1-yl) ethoxy)phenyl)-2-phenylbut-1-en-1-yl)phenyl)boronic acid;
(E)-(4-(1-(4-(2-(4-(2-(2,6-dioxopiperidin-3-yl)-1,3-dioxoisoindolin-5-yl)piperazin-1-yl) ethoxy)phenyl)-2-phenylbut-1-en-1-yl)phenyl)boronic acid;
(Z)-(4-(1-(4-(4-(3-(4-(2-(2,6-dioxopiperidin-3-yl)-1,3-dioxoisoindolin-5-yl)piperazin-1-yl) propyl)piperazin-1-yl)phenyl)-2-phenylbut-1-en-1-yl)phenyl)boronic acid;
(E)-(4-(1-(4-(4-(3-(4-(2-(2,6-dioxopiperidin-3-yl)-1,3-dioxoisoindolin-5-yl)piperazin-1-yl) propyl)piperazin-1-yl)phenyl)-2-phenylbut-1-en-1-yl)phenyl)boronic acid;
(Z)-(4-(1-(4-(4-(3-(4-(2-(2,6-dioxopiperidin-3-yl)-1-oxoisoindolin-5-yl)piperazin-1-yl) propyl)piperazin-1-yl)phenyl)-2-phenylbut-1-en-1-yl)phenyl)boronic acid;
(E)-(4-(1-(4-(4-(3-(4-(2-(2,6-dioxopiperidin-3-yl)-1-oxoisoindolin-5-yl)piperazin-1-yl) propyl)piperazin-1-yl)phenyl)-2-phenylbut-1-en-1-yl)phenyl)boronic acid;
(Z)-(4-(1-(4-(1-(3-(4-(2-(2,6-dioxopiperidin-3-yl)-1,3-dioxoisoindolin-5-yl)piperazin-1-yl) propyl)piperidin-4-yl)phenyl)-2-phenylbut-1-en-1-yl)phenyl)boronic acid;
(E)-(4-(1-(4-(1-(3-(4-(2-(2,6-dioxopiperidin-3-yl)-1,3-dioxoisoindolin-5-yl)piperazin-1-yl) propyl)piperidin-4-yl)phenyl)-2-phenylbut-1-en-1-yl)phenyl)boronic acid;
(Z)-(4-(1-(4-(1-(3-(4-(2-(2,6-dioxopiperidin-3-yl)-1-oxoisoindolin-5-yl)piperazin-1-yl) propyl)piperidin-4-yl)phenyl)-2-phenylbut-1-en-1-yl)phenyl)boronic acid;
(E)-(4-(1-(4-(1-(3-(4-(2-(2,6-dioxopiperidin-3-yl)-1-oxoisoindolin-5-yl)piperazin-1-yl) propyl)piperidin-4-yl)phenyl)-2-phenylbut-1-en-1-yl)phenyl)boronic acid;
(Z)-(4-(1-(4-(4-(3-(4-(2-(2,6-dioxopiperidin-3-yl)-1,3-dioxoisoindolin-5-yl)piperazin-1-yl) propyl)piperidin-1-yl)phenyl)-2-phenylbut-1-en-1-yl)phenyl)boronic acid;
(E)-(4-(1-(4-(4-(3-(4-(2-(2,6-dioxopiperidin-3-yl)-1,3-dioxoisoindolin-5-yl)piperazin-1-yl) propyl)piperidin-1-yl)phenyl)-2-phenylbut-1-en-1-yl)phenyl)boronic acid;
(Z)-(4-(1-(4-(4-(3-(4-(2-(2,6-dioxopiperidin-3-yl)-1-oxoisoindolin-5-yl)piperazin-1-yl) propyl)piperidin-1-yl)phenyl)-2-phenylbut-1-en-1-yl)phenyl)boronic acid;
(E)-(4-(1-(4-(4-(3-(4-(2-(2,6-dioxopiperidin-3-yl)-1-oxoisoindolin-5-yl)piperazin-1-yl) propyl)piperidin-1-yl)phenyl)-2-phenylbut-1-en-1-yl)phenyl)boronic acid;
(Z)-(4-(1-(4-(4-(3-(1-(2-(2,6-dioxopiperidin-3-yl)-1,3-dioxoisoindolin-5-yl)piperidin-4-yl) propyl)piperazin-1-yl)phenyl)-2-phenylbut-1-en-1-yl)phenyl)boronic acid;
(E)-(4-(1-(4-(4-(3-(1-(2-(2,6-dioxopiperidin-3-yl)-1,3-dioxoisoindolin-5-yl)piperidin-4-yl) propyl)piperazin-1-yl)phenyl)-2-phenlbut-1-en-1-yl)phenyl)boronic acid;
(Z)-(4-(1-(4-(4-(3-(1-(2-(2,6-dioxopiperidin-3-yl)-1-oxoisoindolin-5-yl)piperidin-4-yl) propyl)piperazin-1-yl)phenyl)-2-phenylbut-1-en-1-yl)phenyl)boronic acid;
(E)-(4-(1-(4-(4-(3-(1-(2-(2,6-dioxopiperidin-3-yl)-1-oxoisoindolin-5-yl)piperidin-4-yl) propyl)piperazin-1-yl)phenyl)-2-phenylbut-1-en-1-yl)phenyl)boronic acid;
(Z)-(4-(1-(4-(4-(2-(4-(2-(2,6-dioxopiperidin-3-yl)-1,3-dioxoisoindolin-5-yl)piperazin-1-yl) ethyl)piperazin-1-yl)phenyl)-2-phenylbut-1-en-1-yl)phenyl)boronic acid;
(E)-(4-(1-(4-(4-(2-(4-(2-(2,6-dioxopiperidin-3-yl)-1,3-dioxoisoindolin-5-yl)piperazin-1-yl) ethyl)piperazin-1-yl)phenyl)-2-phenylbut-1-en-1-yl)phenyl)boronic acid;
(Z)-(4-(1-(4-(4-(2-(4-(2-(2,6-dioxopiperidin-3-yl)-1-oxoisoindolin-5-yl)piperazin-1-yl) ethyl)piperazin-1-yl)phenyl)-2-phenylbut-1-en-1-yl)phenyl)boronic acid;
(E)-(4-(1-(4-(4-(2-(4-(2-(2,6-dioxopiperidin-3-yl)-1-oxoisoindolin-5-yl)piperazin-1-yl) ethyl)piperazin-1-yl)phenyl)-2-phenylbut-1-en-1-yl)phenyl)boronic acid;
(Z)-(4-(1-(4-(4-(2-(4-(2-(2,6-dioxopiperidin-3-yl)-1,3-dioxoisoindolin-5-yl)piperazin-1-yl) ethyl)piperidin-1-yl)phenyl)-2-phenylbut-1-en-1-yl)phenyl)boronic acid;
(E)-(4-(1-(4-(4-(2-(4-(2-(2,6-dioxopiperidin-3-yl)-1,3-dioxoisoindolin-5-yl)piperazin-1-yl) ethyl)piperidin-1-yl)phenyl)-2-phenylbut-1-en-1-yl)phenyl)boronic acid;
(Z)-(4-(1-(4-(4-(2-(1-(2-(2,6-dioxopiperidin-3-yl)-1,3-dioxoisoindolin-5-yl)piperidin-4-yl) ethyl)piperazin-1-yl)phenyl)-2-phenylbut-1-en-1-yl)phenyl)boronic acid;
(E)-(4-(1-(4-(4-(2-(1-(2-(2,6-dioxopiperidin-3-yl)-1,3-dioxoisoindolin-5-yl)piperidin-4-yl) ethyl)piperazin-1-yl)phenyl)-2-phenylbut-1-en-1-yl)phenyl)boronic acid;
(Z)-(4-(1-(4-(4-(2-(1-(2-(2,6-dioxopiperidin-3-yl)-1-oxoisoindolin-5-yl)piperidin-4-yl) ethyl)piperazin-1-yl)phenyl)-2-phenylbut-1-en-1-yl)phenyl)boronic acid;
(E)-(4-(1-(4-(4-(2-(1-(2-(2,6-dioxopiperidin-3-yl)-1-oxoisoindolin-5-yl)piperidin-4-yl) ethyl)piperazin-1-yl)phenyl)-2-phenylbut-1-en-1-yl)phenyl)boronic acid;
(S,Z)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid;
(R,Z)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid;
(S,E)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid;
(R,E)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid;
(S,Z)-(4-(1-(4-(2-(4-((4-(2-(2,6-dioxopiperidin-3-yl)-1-oxoisoindolin-5-yl)piperazin-1-yl) methyl)piperidin-1-yl)ethoxy)phenyl)-2-phenylbut-1-en-1-yl)phenyl)boronic acid;
(R,Z)-(4-(1-(4-(2-(4-((4-(2-(2,6-dioxopiperidin-3-yl)-1-oxoisoindolin-5-yl)piperazin-1-yl) methyl)piperidin-1-yl)ethoxy)phenyl)-2-phenylbut-1-en-1-yl)phenyl)boronic acid;
(S,E)-(4-(1-(4-(2-(4-((4-(2-(2,6-dioxopiperidin-3-yl)-1-oxoisoindolin-5-yl)piperazin-1-yl) methyl)piperidin-1-yl)ethoxy)phenyl)-2-phenylbut-1-en-1-yl)phenyl)boronic acid;
(R,E)-(4-(1-(4-(2-(4-((4-(2-(2,6-dioxopiperidin-3-yl)-1-oxoisoindolin-5-yl)piperazin-1-yl) methyl)piperidin-1-yl)ethoxy)phenyl)-2-phenylbut-1-en-1-yl)phenyl)boronic acid;
(Z)-3-(5-(4-(4-(2-(4-(1-(4-hydroxyphenyl)-2-phenylbut-1-en-1-yl)phenoxy)ethyl)piperazine-1-carbonyl) piperazin-1-yl)-1-oxoisoindolin-2-yl)piperidine-2,6-dione;
(S,Z)-3-(5-(4-(4-(2-(4-(1-(4-hydroxyphenyl)-2-phenylbut-1-en-1-yl)phenoxy)ethyl)piperazine-1-carbonyl) piperazin-1-yl)-1-oxoisoindolin-2-yl)piperidine-2,6-dione;
(R,Z)-3-(5-(4-(4-(2-(4-(1-(4-hydroxyphenyl)-2-phenylbut-1-en-1-yl)phenoxy)ethyl)piperazine-1-carbonyl) piperazin-1-yl)-1-oxoisoindolin-2-yl)piperidine-2,6-dione;
(E)-3-(5-(4-(4-(2-(4-(1-(4-hydroxyphenyl)-2-phenylbut-1-en-1-yl)phenoxy)ethyl)piperazine-1-carbonyl) piperazin-1-yl)-1-oxoisoindolin-2-yl)piperidine-2,6-dione;
(S,E)-3-(5-(4-(4-(2-(4-(1-(4-hydroxyphenyl)-2-phenylbut-1-en-1-yl)phenoxy)ethyl)piperazine-1-carbonyl) piperazin-1-yl)-1-oxoisoindolin-2-yl)piperidine-2,6-dione;
(R,E)-3-(5-(4-(4-(2-(4-(1-(4-hydroxyphenyl)-2-phenylbut-1-en-1-yl)phenoxy)ethyl)piperazine-1-carbonyl) piperazin-1-yl)-1-oxoisoindolin-2-yl)piperidine-2,6-dione;
(Z)-(4-(1-(4-(2-(4-(4-(2-(2,6-dioxopiperidin-3-yl)-1-oxoisoindolin-5-yl)piperazine-1-carbonyl) piperazin-1-yl)ethoxy)phenyl)-2-phenylbut-1-en-1-yl)phenyl)boronic acid;
(S,Z)-(4-(1-(4-(2-(4-(4-(2-(2,6-dioxopiperidin-3-yl)-1-oxoisoindolin-5-yl)piperazine-1-carbonyl) piperazin-1-yl)ethoxy)phenyl)-2-phenylbut-1-en-1-yl)phenyl)boronic acid;
(R,Z)-(4-(1-(4-(2-(4-(4-(2-(2,6-dioxopiperidin-3-yl)-1-oxoisoindolin-5-yl)piperazine-1-carbonyl) piperazin-1-yl)ethoxy)phenyl)-2-phenylbut-1-en-1-yl)phenyl)boronic acid;
(E)-(4-(1-(4-(2-(4-(4-(2-(2,6-dioxopiperidin-3-yl)-1-oxoisoindolin-5-yl)piperazine-1-carbonyl) piperazin-1-yl)ethoxy)phenyl)-2-phenylbut-1-en-1-yl)phenyl)boronic acid;
(S,E)-(4-(1-(4-(2-(4-(4-(2-(2,6-dioxopiperidin-3-yl)-1-oxoisoindolin-5-yl)piperazine-1-carbonyl) piperazin-1-yl)ethoxy)phenyl)-2-phenylbut-1-en-1-yl)phenyl)boronic acid;
(R,E)-(4-(1-(4-(2-(4-(4-(2-(2,6-dioxopiperidin-3-yl)-1-oxoisoindolin-5-yl)piperazine-1-carbonyl) piperazin-1-yl)ethoxy)phenyl)-2-phenylbut-1-en-1-yl)phenyl)boronic acid;
(Z)-3-(5-(4-((1-(4-(2-(4-(1-(4-hydroxyphenyl)-2-phenylbut-1-en-1-yl)phenoxy)ethyl)piperazine-1-carbonyl) piperidin-4-yl)methyl)piperazin-1-yl)-1-oxoisoindolin-2-yl)piperidine-2,6-dione;
(E)-3-(5-(4-((1-(4-(2-(4-(1-(4-hydroxyphenyl)-2-phenylbut-1-en-1-yl)phenoxy)ethyl)piperazine-1-carbonyl) piperidin-4-yl)methyl)piperazin-1-yl)-1-oxoisoindolin-2-yl)piperidine-2,6-dione;
(Z)-(4-(1-(4-(2-(4-(4-((4-(2-(2,6-dioxopiperidin-3-yl)-1-oxoisoindolin-5-yl)piperazin-1-yl) methyl)piperidine-1-carbonyl)piperazin-1-yl)ethoxy)phenyl)-2-phenylbut-1-en-1-yl) phenyl)boronic acid;
(E)-(4-(1-(4-(2-(4-(4-((4-(2-(2,6-dioxopiperidin-3-yl)-1-oxoisoindolin-5-yl)piperazin-1-yl) methyl)piperidine-1-carbonyl)piperazin-1-yl)ethoxy)phenyl)-2-phenylbut-1-en-1-yl) phenyl)boronic acid;
(Z)-3-(5-(4-(1-(4-(2-(4-(1-(4-hydroxyphenyl)-2-phenylbut-1-en-1-yl)phenoxy)ethyl)piperazine-1-carbonyl) piperidin-4-yl)piperazin-1-yl)-1-oxoisoindolin-2-yl)piperidine-2,6-dione;
(E)-3-(5-(4-(1-(4-(2-(4-(1-(4-hydroxyphenyl)-2-phenylbut-1-en-1-yl)phenoxy)ethyl)piperazine-1-carbonyl) piperidin-4-yl)piperazin-1-yl)-1-oxoisoindolin-2-yl)piperidine-2,6-dione;
(Z)-(4-(1-(4-(2-(4-(4-(4-(2-(2,6-dioxopiperidin-3-yl)-1-oxoisoindolin-5-yl)piperazin-1-yl)piperidine-1-carbonyl)piperazin-1-yl)ethoxy)phenyl)-2-phenylbut-1-en-1-yl)phenyl)boronic acid;
(E)-(4-(1-(4-(2-(4-(4-(4-(2-(2,6-dioxopiperidin-3-yl)-1-oxoisoindolin-5-yl)piperazin-1-yl)piperidine-1-carbonyl)piperazin-1-yl)ethoxy)phenyl)-2-phenylbut-1-en-1-yl)phenyl)boronic acid;
(E)-3-(5-(4-(4-(4-(1-(4-hydroxyphenyl)-2-phenylbut-1-en-1-yl)phenyl)piperazine-1-carbonyl) piperazin-1-yl)-1-oxoisoindolin-2-yl)piperidine-2,6-dione;
(S,E)-3-(5-(4-(4-(4-(1-(4-hydroxyphenyl)-2-phenylbut-1-en-1-yl)phenyl)piperazine-1-carbonyl) piperazin-1-yl)-1-oxoisoindolin-2-yl)piperidine-2,6-dione;
(R,E)-3-(5-(4-(4-(4-(1-(4-hydroxyphenyl)-2-phenylbut-1-en-1-yl)phenyl)piperazine-1-carbonyl) piperazin-1-yl)-1-oxoisoindolin-2-yl)piperidine-2,6-dione;
(Z)-3-(5-(4-(4-(4-(1-(4-hydroxyphenyl)-2-phenylbut-1-en-1-yl)phenyl)piperazine-1-carbonyl) piperazin-1-yl)-1-oxoisoindolin-2-yl)piperidine-2,6-dione;
(S,Z)-3-(5-(4-(4-(4-(1-(4-hydroxyphenyl)-2-phenylbut-1-en-1-yl)phenyl)piperazine-1-carbonyl) piperazin-1-yl)-1-oxoisoindolin-2-yl)piperidine-2,6-dione;
(R,Z)-3-(5-(4-(4-(4-(1-(4-hydroxyphenyl)-2-phenylbut-1-en-1-yl)phenyl)piperazine-1-carbonyl) piperazin-1-yl)-1-oxoisoindolin-2-yl)piperidine-2,6-dione;
(Z)-(4-(1-(4-(4-(4-(2-(2,6-dioxopiperidin-3-yl)-1-oxoisoindolin-5-yl)piperazine-1-carbonyl) piperazin-1-yl)phenyl)-2-phenylbut-1-en-1-yl)phenyl)boronic acid;
(S,Z)-(4-(1-(4-(4-(4-(2-(2,6-dioxopiperidin-3-yl)-1-oxoisoindolin-5-yl)piperazine-1-carbonyl) piperazin-1-yl)phenyl)-2-phenylbut-1-en-1-yl)phenyl)boronic acid;
(R,Z)-(4-(1-(4-(4-(4-(2-(2,6-dioxopiperidin-3-yl)-1-oxoisoindolin-5-yl)piperazine-1-carbonyl) piperazin-1-yl)phenyl)-2-phenylbut-1-en-1-yl)phenyl)boronic acid;
(E)-(4-(1-(4-(4-(4-(2-(2,6-dioxopiperidin-3-yl)-1-oxoisoindolin-5-yl)piperazine-1-carbonyl) piperazin-1-yl)phenyl)-2-phenylbut-1-en-1-yl)phenyl)boronic acid;
(S,E)-(4-(1-(4-(4-(4-(2-(2,6-dioxopiperidin-3-yl)-1-oxoisoindolin-5-yl)piperazine-1-carbonyl) piperazin-1-yl)phenyl)-2-phenylbut-1-en-1-yl)phenyl)boronic acid;
(R,E)-(4-(1-(4-(4-(4-(2-(2,6-dioxopiperidin-3-yl)-1-oxoisoindolin-5-yl)piperazine-1-carbonyl) piperazin-1-yl)phenyl)-2-phenylbut-1-en-1-yl)phenyl)boronic acid;
(E)-3-(5-(4-(1-(4-(4-(1-(4-hydroxyphenyl)-2-phenylbut-1-en-1-yl)phenyl)piperazine-1-carbonyl) piperidin-4-yl)piperazin-1-yl)-1-oxoisoindolin-2-yl)piperidine-2,6-dione;
(Z)-(4-(1-(4-(4-(4-(4-(2-(2,6-dioxopiperidin-3-yl)-1-oxoisoindolin-5-yl)piperazin-1-yl)piperidine-1-carbonyl) piperazin-1-yl)phenyl)-2-phenylbut-1-en-1-yl)phenyl)boronic acid;
(Z)-3-(5-(4-(1-(4-(4-(1-(4-hydroxyphenyl)-2-phenylbut-1-en-1-yl)phenyl)piperazine-1-carbonyl) piperidin-4-yl)piperazin-1-yl)-1-oxoisoindolin-2-yl)piperidine-2,6-dione;
(E)-(4-(1-(4-(4-(4-(4-(2-(2,6-dioxopiperidin-3-yl)-1-oxoisoindolin-5-yl)piperazin-1-yl)piperidine-1-carbonyl) piperazin-1-yl)phenyl)-2-phenylbut-1-en-1-yl)phenyl)boronic acid;
(E)-3-(5-(4-((1-(4-(4-(1-(4-hydroxyphenyl)-2-phenylbut-1-en-1-yl)phenyl)piperazine-1-carbonyl) piperidin-4-yl)methyl)piperazin-1-yl)-1-oxoisoindolin-2-yl)piperidine-2,6-dione;
(Z)-(4-(1-(4-(4-(4-((4-(2-(2,6-dioxopiperidin-3-yl)-1-oxoisoindolin-5-yl)piperazin-1-yl) methyl)piperidine-1-carbonyl)piperazin-1-yl)phenyl)-2-phenylbut-1-en-1-yl)phenyl)boronic acid;
(Z)-3-(5-(4-((1-(4-(4-(1-(4-hydroxyphenyl)-2-phenylbut-1-en-1-yl)phenyl)piperazine-1-carbonyl) piperidin-4-yl)methyl)piperazin-1-yl)-1-oxoisoindolin-2-yl)piperidine-2,6-dione;
(E)-(4-(1-(4-(4-(4-((4-(2-(2,6-dioxopiperidin-3-yl)-1-oxoisoindolin-5-yl)piperazin-1-yl) methyl)piperidine-1-carbonyl)piperazin-1-yl)phenyl)-2-phenylbut-1-en-1-yl)phenyl)boronic acid; or any combination thereof.

In some embodiments, the compound of Formula (I) may encompass both the E and Z isomers. In some embodiments, the compound of Formula (I) may be a mixture of trans- and - cis olefin.

In some embodiments, provided herein is a compound, or pharmaceutically acceptable salt thereof, chosen from the compounds listed in Table 1:

TABLE 1
Exemplary Compounds of the Disclosure
ER antagonism (T47D ERE luciferase): A - IC₅₀ < 10 nM; B - IC₅₀ = 10-50 nM; C - IC₅₀ > 50 nM
MCF-7 proliferation with 0.1 nM 17-beta estradiol: A - IC50 < 10 nM; B - IC₅₀ = 10-50 nM; C - IC₅₀ > 50 nM
ER degradation (DC₅₀): A < 10 nM; B: 10-50 nM; C: >50 nM
Maximal ER degradation (Dₘₐₓ): A -> 90%; B > 75%; C > 50%; D <50%

| | | | | IC₅₀ | IC₅₀ | | |
|---|---|---|---|---|---|---|---|
| | | | | (ER | (anti- | | |
| | | | | antag- | prolifer- | | |
| # | Structure | IUPAC Name | M + H | onistm) | ation) | DC₅₀ | Dₘₐₓ |
| 1 | 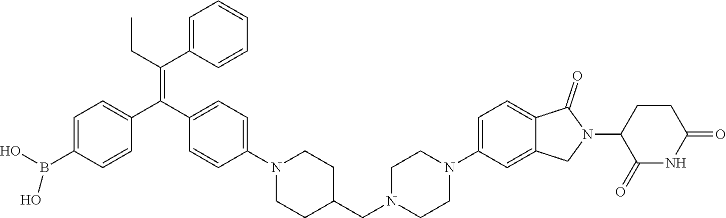 [SMILES: CC/C(=C(\c1ccc(B(O)O)cc1)c1ccc(N2CCC(CN3CCN(c4ccc5c(c4)CN(C4CCC(=O)NC4=O)C5=O)CC3)CC2)cc1)c1ccccc1] | (Z)-(4-(1-(4-(4-((4-(2-(2,6- dioxopiperidin-3-yl)-1-oxoisoindolin-5- yl)piperazin-1-yl)methyl)piperidin-1- yl)phenyl-2-phenylbut-1-en-1- yl)phenyl)boronic acid | 752.4 | B | B | A | A |
| 2 | 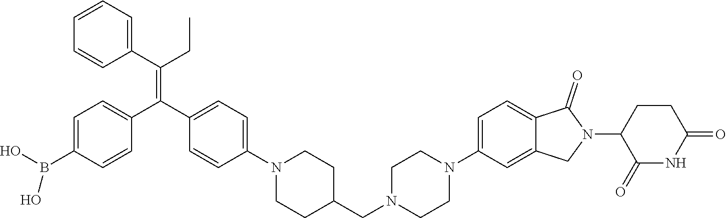 [SMILES: CC/C(=C(/c1ccc(B(O)O)cc1)c1ccc(N2CCC(CN3CCN(c4ccc5c(c4)CN(C4CCC(=O)NC4=O)C5=O)CC3)CC2)cc1)c1ccccc1] | (E)-(4-(1-(4-(4-((4-(2-(2,6- dioxopiperidin-3-yl)-1-oxoisoindolin-5- yl)piperazin-1-yl)methyl)piperidin-1- yl)phenyl)-2-phenylbut-1-en-1- yl)phenyl)boronic acid | 752.4 | B | B | A | A |
| 3 | 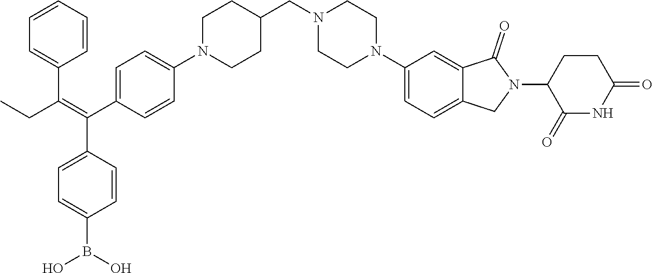 [SMILES: CC/C(=C(\c1ccc(B(O)O)cc1)c1ccc(N2CCC(CN3CCN(c4ccc5c(c4)C(=O)N(C4CCC(=O)NC4=O)C5)CC3)CC2)cc1)c1ccccc1] | (Z)-(4-(1-(4-(4-((4-(2-(2,6- dioxopiperidin-3-yl)-3-oxoisoindolin-5- yl)piperazin-1-yl)methyl)piperidin-1- yl)phenyl)-2-phenylbut-1-en-1- yl)phenyl)boronic acid | 752.3 | A | A | C | B |
| 4 | 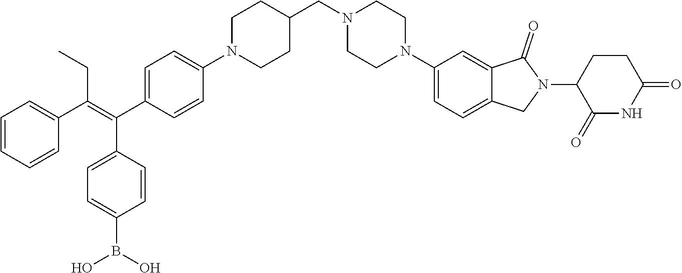 [SMILES: CC/C(=C(/c1ccc(B(O)O)cc1)c1ccc(N2CCC(CN3CCN(c4ccc5c(c4)C(=O)N(C4CCC(=O)NC4=O)C5)CC3)CC2)cc1)c1ccccc1] | (E)-(4-(1-(4-(4-((4-(2-(2,6- dioxopiperidin-3-yl)-3-oxoisoindolin-5- yl)piperazin-1-yl)methyl)piperidin-1- yl)phenyl)-2-phenylbut-1-en-1- yl)phenyl)boronic acid | 752.3 | A | A | C | B |
| 5 | 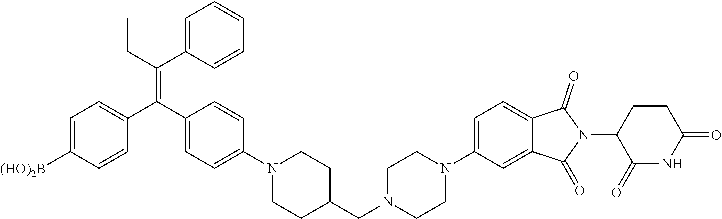 [SMILES: CC/C(=C(\c1ccc(B(O)O)cc1)c1ccc(N2CCC(CN3CCN(c4ccc5c(c4)C(=O)N(C4CCC(=O)NC4=O)C5=O)CC3)CC2)cc1)c1ccccc1] | (Z)-(4-(1-(4-(4-((4-(2-(2,6- dioxopiperidin-3-yl)-1,3- dioxoisoindolin-5-yl)piperazin-1- yl)methyl)piperidin-1-yl)phenyl)-2- phenylbut-1-en-1-yl)phenyl)boronic acid | 766.4 | B | C | B | A |
| 6 | 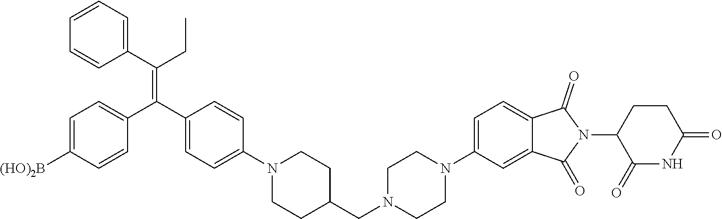 [SMILES: CC/C(=C(/c1ccc(B(O)O)cc1)c1ccc(N2CCC(CN3CCN(c4ccc5c(c4)C(=O)N(C4CCC(=O)NC4=O)C5=O)CC3)CC2)cc1)c1ccccc1] | (E)-(4-(1-(4-(4-((4-(2-(2,6- dioxopiperidin-3-yl)-1,3- dioxoisoindolin-5-yl)piperazin-1- yl)methyl)piperidin-1-yl)phenyl)-2- phenylbut-1-en-1-yl)phenyl)boronic acid | 766.4 | B | C | B | A |
| 7 | 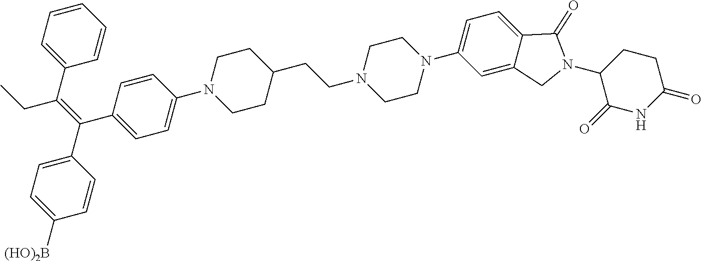 [SMILES: CC/C(=C(\c1ccc(B(O)O)cc1)c1ccc(N2CCC(CCN3CCN(c4ccc5c(c4)CN(C4CCC(=O)NC4=O)C5=O)CC3)CC2)cc1)c1ccccc1] | (Z)-(4-(1-(4-(4-(2-(4-(2-(2,6- dioxopiperidin-3-yl)-1-oxoisoindolin- 5-yl)piperazin-1-yl)ethyl)piperidin-1- yl)phenyl)-2-phenylbut-1-en-1- yl)phenyl)boronic acid | 766.4 | A | B | B | A |
| 8 | 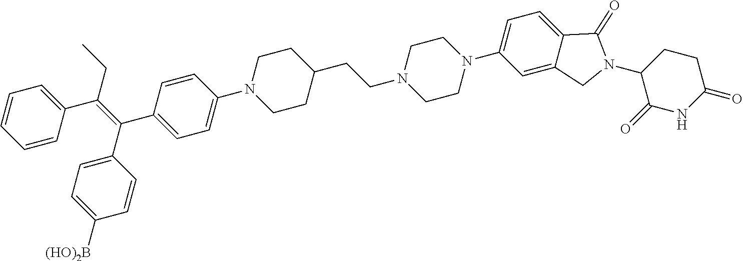 [SMILES: CC/C(=C(/c1ccc(B(O)O)cc1)c1ccc(N2CCC(CCN3CCN(c4ccc5c(c4)CN(C4CCC(=O)NC4=O)C5=O)CC3)CC2)cc1)c1ccccc1] | (E)-(4-(1-(4-(4-(2-(4-(2-(2,6- dioxopiperidin-3-yl)-1-oxoisoindolin- 5-yl)piperazin-1-yl)ethyl)piperidin-1- yl)phenyl)-2-phenylbut-1-en-1- yl)phenyl)boronic acid | 766.4 | A | B | B | A |
| 9 | 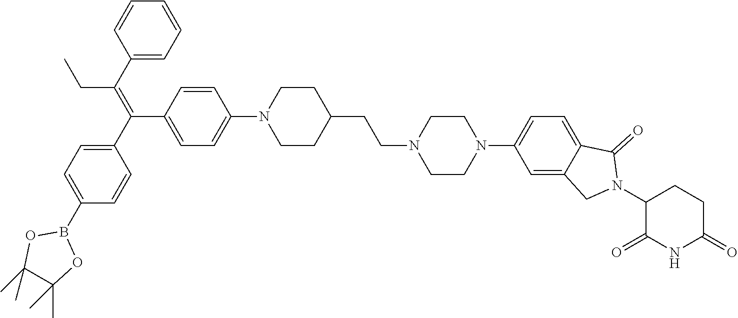 [SMILES: CC/C(=C(\c1ccc(B2OC(C)(C)C(C)(C)O2)cc1)c1ccc(N2CCC(CCN3CCN(c4ccc5c(c4)CN(C4CCC(=O)NC4=O)C5=O)CC3)CC2)cc1)c1ccccc1] | (Z)-3-(1-oxo-5-(4-(2-(1-(4-(2-phenyl-1- (4-(4,4,5,5-tetramethyl-1,3,2- dioxaborolan-2-yl)phenyl)but-1-en-1- yl)phenyl)piperidin-4-yl)ethyl)piperazin- 1-yl)isoindolin-2-yl)piperidine-2,6-dione | 848.5 | A | A | B | A |
| 10 | 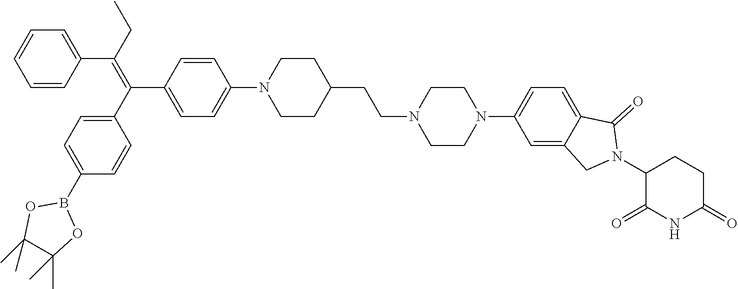 [SMILES: CC/C(=C(/c1ccc(B2OC(C)(C)C(C)(C)O2)cc1)c1ccc(N2CCC(CCN3CCN(c4ccc5c(c4)CN(C4CCC(=O)NC4=O)C5=O)CC3)CC2)cc1)c1ccccc1] | (E)-3-(1-oxo-5-(4-(2-(1-(4-(2-phenyl-1- (4-(4,4,5,5-tetramethyl-1,3,2- dioxaborolan-2-yl)phenyl)but-1-en-1- yl)phenyl)piperidin-4-yl)ethyl)piperazin- 1-yl)isoindolin-2-yl)piperidine-2,6-dione | 848.5 | A | A | B | A |
| 11 | 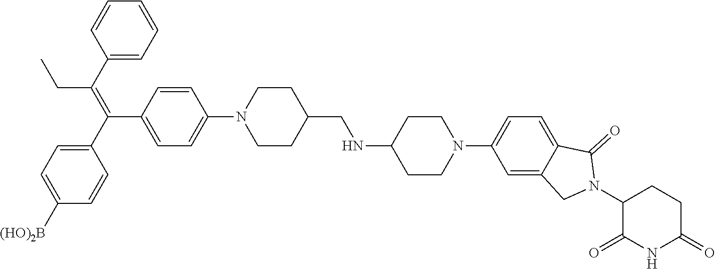 [SMILES: CC/C(=C(\c1ccc(B(O)O)cc1)c1ccc(N2CCC(CNC3CCN(c4ccc5c(c4)CN(C4CCC(=O)NC4=O)C5=O)CC3)CC2)cc1)c1ccccc1] | (Z)-(4-(1-(4-(4-((1-(2-2,6- dioxopiperidin-3-yl)-1-oxoisoindolin- 5-yl)piperidin-4-yl)amino)methyl) piperidin-1-yl)phenyl)-2-phenylbut-1- en-1-yl)phenyl)boronic acid | 766.4 | A | A | A | A |
| 12 | 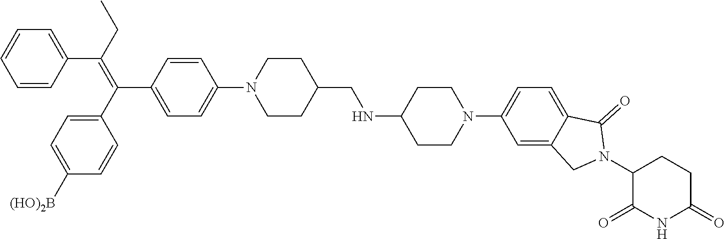 [SMILES: CC/C(=C(/c1ccc(B(O)O)cc1)c1ccc(N2CCC(CNC3CCN(c4ccc5c(c4)CN(C4CCC(=O)NC4=O)C5=O)CC3)CC2)cc1)c1ccccc1] | (E)-(4-(1-(4-(4-(((1-(2-(2,6- dioxopiperidin-3-yl)-1-oxoisoindolin-5- yl)piperidin-4- yl)amino)methyl)piperidin-1-yl(phenyl)- 2-phenylbut-1-en-1-yl)phenyl)boronic acid | 766.4 | A | A | A | A |
| 13 | 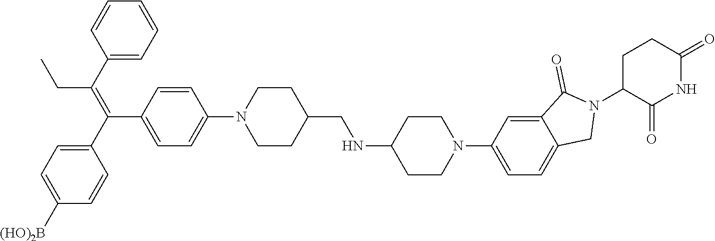 [SMILES: CC/C(=C(\c1ccc(B(O)O)cc1)c1ccc(N2CCC(CNC3CCN(c4ccc5c(c4)C(=O)N(C4CCC(=O)NC4=O)C5)CC3)CC2)cc1)c1ccccc1] | (Z)-(4-(1-(4-(4-(((1-(2-(2,6- dioxopiperidin-3-yl)-3-oxoisoindolin-5- yl)piperidin-4- yl)amino)methyl)piperidin-1-yl)phenyl)- 2-phenylbut-1-en-1-yl)phenyl)boronic acid | 766.4 | A | A | A | A |
| 14 | 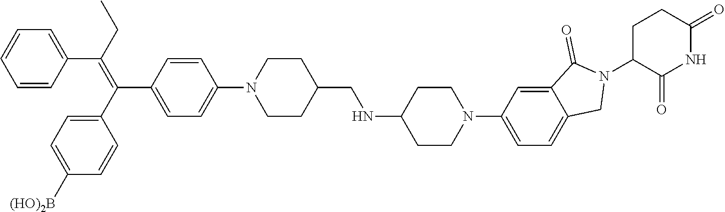 [SMILES: CC/C(=C(/c1ccc(B(O)O)cc1)c1ccc(N2CCC(CNC3CCN(c4ccc5c(c4)C(=O)N(C4CCC(=O)NC4=O)C5)CC3)CC2)cc1)c1ccccc1] | (E)-(4-(1-(4-(4-(((1-(2-(2,6- dioxopiperidin-3-yl)-3-oxoisoindolin-5- yl)piperidin-4- yl)amino)methyl)piperidin-1-yl)phenyl)- 2-phenylbut-1-en-1-yl)phenyl)boronic acid | 766.4 | A | A | A | A |
| 15 | 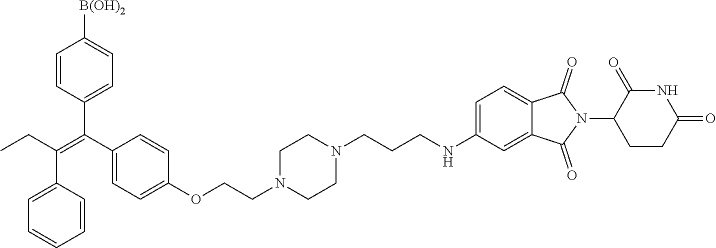 [SMILES: CC/C(=C(/c1ccc(OCCN2CCN(CCCNc3ccc4c(c3)C(=O)N(C3CCC(=O)NC3=O)C4=O)CC2)cc1)c1ccc(B(O)O)cc1)c1ccccc1] | (Z)-(4-(1-(4-(2-(4-(3-((2-(2,6- dioxopiperidin-3-yl)-1,3- dioxoisoindolin-5- yl)amino)propyl)piperazin-1- yl)ethoxy)phenyl)-2-phenylbut-1- en-1-yl)phenyl)boronic acid | 770.3 | B | B | B | B |
| 16 | 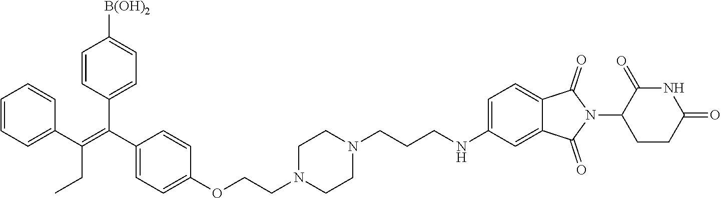 [SMILES: CC/C(=C(\c1ccc(OCCN2CCN(CCCNc3ccc4c(c3)C(=O)N(C3CCC(=O)NC3=O)C4=O)CC2)cc1)c1ccc(B(O)O)cc1)c1ccccc1] | (E)-(4-(1-(4-(2-(4-(3-((2-(2,6- dioxopiperidin-3-yl)-1,3- dioxoisoindolin-5-yl)amino)propyl) piperazin-1-yl)ethoxy)phenyl)-2- phenylbut-1-en-1-yl)phenyl)boronic acid | 770.3 | B | B | B | B |
| 17 | 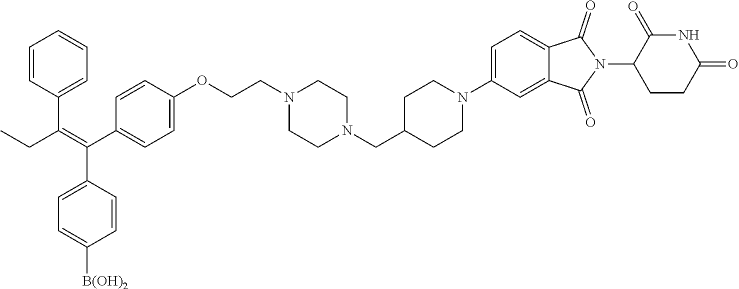 [SMILES: CC/C(=C(/c1ccc(OCCN2CCN(CC3CCN(c4ccc5c(c4)C(=O)N(C4CCC(=O)NC4=O)C5=O)CC3)CC2)cc1)c1ccc(B(O)O)cc1)c1ccccc1] | (Z)-(4-(1-(4-(2-(4-((1-(2-(2,6- dioxopiperidin-3-yl)-1,3- dioxoisoindolin-5-yl)piperidin-4- yl)methyl)piperazin-1- yl)ethoxy)phenyl)-2-phenylbut-1- en-1-yl)phenyl)boronic acid | 810.4 | B | B | A | A |
| 18 | 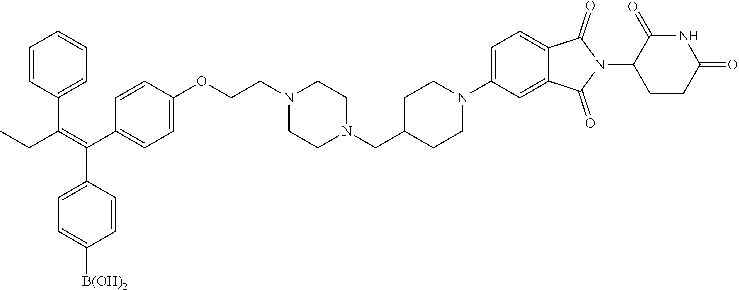 [SMILES: CC/C(=C(/c1ccc(OCCN2CCN(CC3CCN(c4ccc5c(c4)C(=O)N(C4CCC(=O)NC4=O)C5=O)CC3)CC2)cc1)c1ccc(B(O)O)cc1)c1ccccc1] | (E)-(4-(1-(4-(2-(4-((1-(2-(2,6- dioxopiperidin-3-yl)-1,3- dioxoisoindolin-5-yl)piperidin-4- yl)methyl)piperazin-1- yl)ethoxy)phenyl)-2-phenylbut- 1-en-1-yl)phenyl)boronic acid | 810.4 | B | B | A | A |
| 19 | 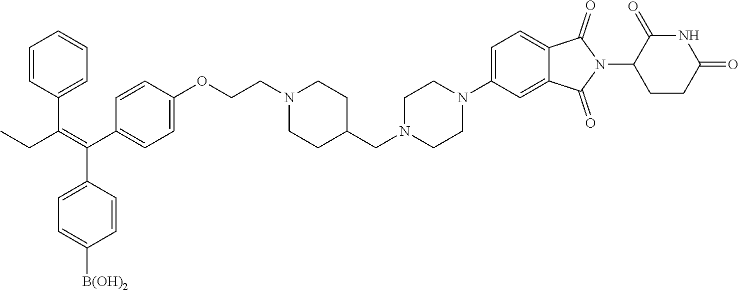 [SMILES: CC/C(=C(/c1ccc(OCCN2CCC(CN3CCN(c4ccc5c(c4)C(=O)N(C4CCC(=O)NC4=O)C5=O)CC3)CC2)cc1)c1ccc(B(O)O)cc1)c1ccccc1] | (Z)-(4-(1-(4-(2-(4-((4-(2-(2,6- dioxopiperidin-3-yl)-1,3- dioxoisoindolin-5-yl)piperazin-1- yl)methyl)piperidin-1- yl)ethoxy)phenyl)-2-phenylbut-1-en-1- yl)phenyl)boronic acid | 810.4 | C | C | B | B |
| 20 | 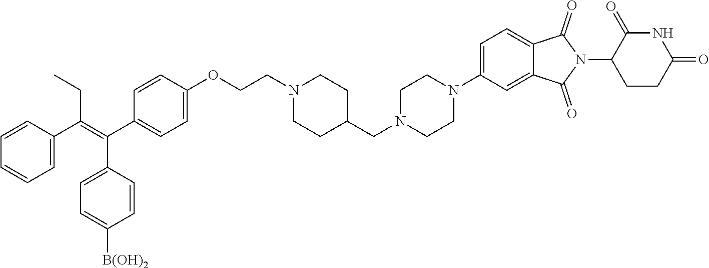 [SMILES: CC/C(=C(\c1ccc(OCCN2CCC(CN3CCN(c4ccc5c(c4)C(=O)N(C4CCC(=O)NC4=O)C5=O)CC3)CC2)cc1)c1ccc(B(O)O)cc1)c1ccccc1] | (E)-(4-(1-(4-(2-(4-((4-(2-(2,6- dioxopiperidin-3-yl)-1,3- dioxoisoindolin-5-yl)piperazin-1- yl)methyl)piperidin-1- yl)ethoxy)phenyl)-2-phenylbut- 1-en-1-yl)phenyl)boronic acid | 810.4 | C | C | B | B |
| 21 | 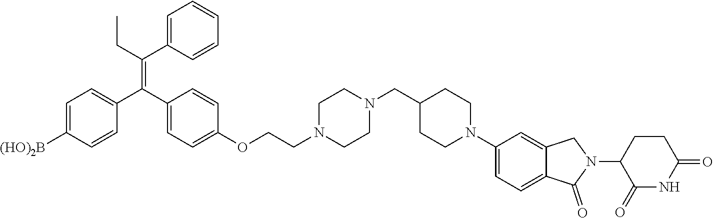 [SMILES: CC/C(=C(/c1ccc(OCCN2CCN(CC3CCN(c4ccc5c(c4)CN(C4CCC(=O)NC4=O)C5=O)CC3)CC2)cc1)c1ccc(B(O)O)cc1)c1ccccc1] | (Z)-(4-(1-(4-(2-(4-((1-(2-(2,6- dioxopiperidin-3-yl)-1-oxoisoindolin-5- yl)piperidin-4-yl)methyl)piperazin-1- yl)ethoxy)phenyl)-2-phenylbut-1-en-1- yl)phenyl)boronic acid | 796.4 | A | A | A | A |
| 22 | 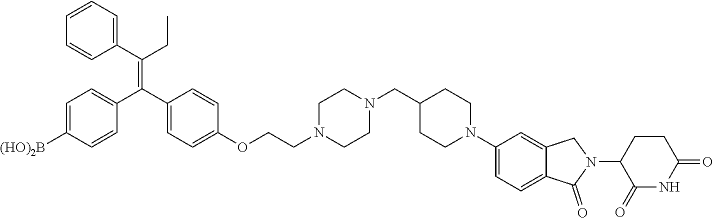 [SMILES: CC/C(=C(\c1ccc(OCCN2CCN(CC3CCN(c4ccc5c(c4)CN(C4CCC(=O)NC4=O)C5=O)CC3)CC2)cc1)c1ccc(B(O)O)cc1)c1ccccc1] | (E)-(4-(1-(4-(2-(4-((1-(2-(2,6- dioxopiperidin-3-yl)-1-oxoisoindolin-5- yl)piperidin-4-yl)methyl)piperazin-1- yl)ethoxy)phenyl)-2-phenylbut-1-en-1- yl)phenyl)boronic acid | 796.4 | A | A | A | A |
| 23 | 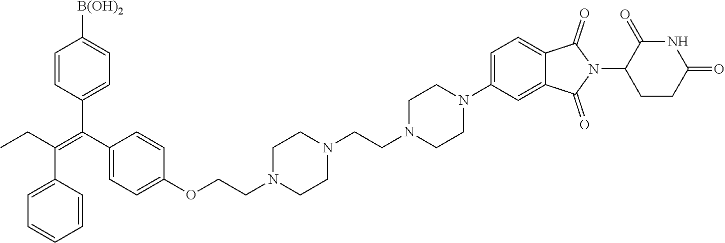 [SMILES: CC/C(=C(/c1ccc(OCCN2CCN(CCN3CCN(c4ccc5c(c4)C(=O)N(C4CCC(=O)NC4=O)C5=O)CC3)CC2)cc1)c1ccc(B(O)O)cc1)c1ccccc1] | (Z)-(4-(1-(4-(2-(4-(2-(4-(2-(2,6- dioxopiperidin-3-yl)-1,3- dioxoisoindolin-5-yl)piperazin-1- yl)ethyl)piperazin-1-yl)ethoxy)phenyl)- 2-phenylbut-1-en-1-yl)phenyl)boronic acid | 825.3 | B | B | A | A |
| 24 | 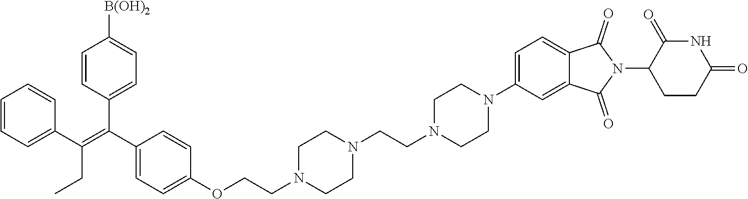 [SMILES: CC/C(=C(\c1ccc(OCCN2CCN(CCN3CCN(c4ccc5c(c4)C(=O)N(C4CCC(=O)NC4=O)C5=O)CC3)CC2)cc1)c1ccc(B(O)O)cc1)c1ccccc1] | (E)-(4-(1-(4-(2-(4-(2-(4-(2-(2,6- dioxopiperidin-3-yl)-1,3- dioxoisoindolin-5-yl)piperazin-1- yl)ethyl)piperazin-1-ylethoxy)phenyl)- 2-phenylbut-1-en-1-yl)phenyl)boronic acid | 825.3 | B | B | A | A |
| 25 | 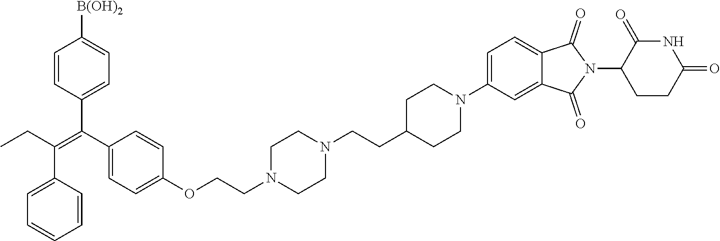 [SMILES: CC/C(=C(/c1ccc(OCCN2CCN(CCC3CCN(c4ccc5c(c4)C(=O)N(C4CCC(=O)NC4=O)C5=O)CC3)CC2)cc1)c1ccc(B(O)O)cc1)c1ccccc1] | (E)-(4-(1-(4-(2-(4-(2-(4-(2-(2,6- dioxopiperidin-3-yl)-1,3- dioxoisoindolin-5-yl)piperidin-4- yl)ethyl)piperazin-1-yl)ethoxy)phenyl)- 2-phenylbut-1-en-1-yl)phenyl)boronic acid | 824.4 | B | B | A | A |
| 26 | 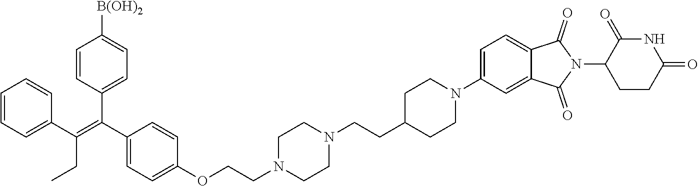 [SMILES: CC/C(=C(\c1ccc(OCCN2CCN(CCC3CCN(c4ccc5c(c4)C(=O)N(C4CCC(=O)NC4=O)C5=O)CC3)CC2)cc1)c1ccc(B(O)O)cc1)c1ccccc1] | (E)-(4-(1-(4-(2-(4-(2-(1-(2-(2,6- dioxopiperidin-3-yl)-1,3- dioxoisoindolin-5-yl)piperidin-4- yl)ethyl)piperazin-1-yl)ethoxy)phenyl)- 2-phenylbut-1-en-1-yl)phenyl)boronic acid | 824.4 | B | B | A | A |
| 27 | 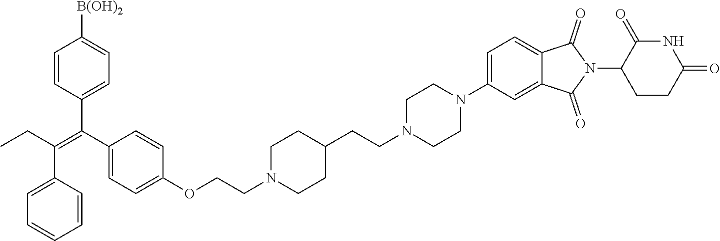 [SMILES: CC/C(=C(/c1ccc(OCCN2CCC(CCN3CCN(c4ccc5c(c4)C(=O)N(C4CCC(=O)NC4=O)C5=O)CC3)CC2)cc1)c1ccc(B(O)O)cc1)c1ccccc1] | (Z)-(4-(1-(4-(2-(4-(2-(4-(2-(2,6- dioxopiperidin-3-yl)-1,3- dioxoisoindolin-5-yl)piperazin-1- yl)ethyl)piperidin-1-yl)ethoxy)phenyl)- 2-phenylbut-1-en-1-yl)phenyl)boronic acid | 824.4 | C | B | A | A |
| 28 | 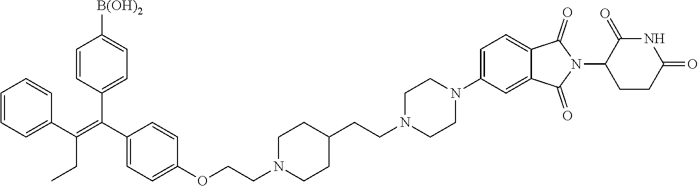 [SMILES: CC/C(=C(\c1ccc(OCCN2CCC(CCN3CCN(c4ccc5c(c4)C(=O)N(C4CCC(=O)NC4=O)C5=O)CC3)CC2)cc1)c1ccc(B(O)O)cc1)c1ccccc1] | (E)-(4-(1-(4-(2-(4-(2-(4-(2-(2,6- dioxopiperidin-3-yl)-1,3- dioxoisoindolin-5-yl)piperazin-1- yl)ethyl)piperidin-1-yl)ethoxy)phenyl)- 2-phenylbut-1-en-1-yl)phenyl)boronic acid | 824.4 | C | B | A | A |
| 29 | 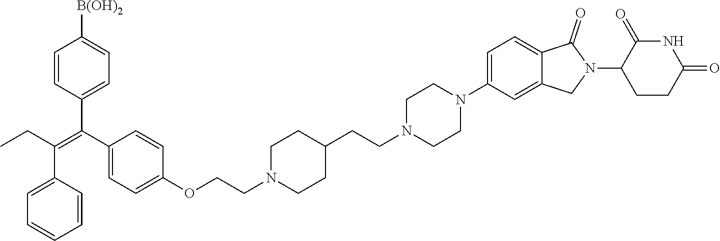 [SMILES: CC/C(=C(/c1ccc(OCCN2CCC(CCN3CCN(c4ccc5c(c4)CN(C4CCC(=O)NC4=O)C5=O)CC3)CC2)cc1)c1ccc(B(O)O)cc1)c1ccccc1] | (Z)-(4-(1-(4-(2-(4-(2-(4-(2-(2,6- dioxopiperidin-3-yl)-1-oxoisoindolin-5- yl)piperazin-1-yl)ethyl)piperidin-1- yl)ethoxy)phenyl)-2-phenylbut-1-en-1- yl)phenyl)boronic acid | 810.5 | B | B | B | A |
| 30 | 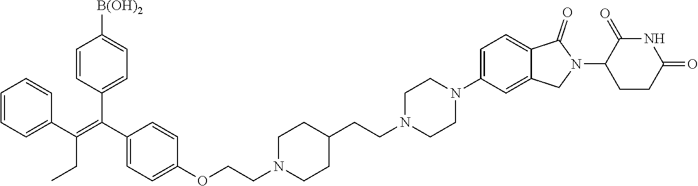 [SMILES: CC/C(=C(\c1ccc(OCCN2CCC(CCN3CCN(c4ccc5c(c4)CN(C4CCC(=O)NC4=O)C5=O)CC3)CC2)cc1)c1ccc(B(O)O)cc1)c1ccccc1] | (E)-(4-(1-(4-(2-(4-(2-(4-(2-(2,6- dioxopiperidin-3-yl)-1- oxoisoindolin-5-yl)piperazin-1- yl)ethyl)piperidin-1- yl)ethoxy)phenyl)-2-phenylbut-1-en- l-yl)phenyl)boronic acid | 810.5 | B | B | A | A |
| 31 | 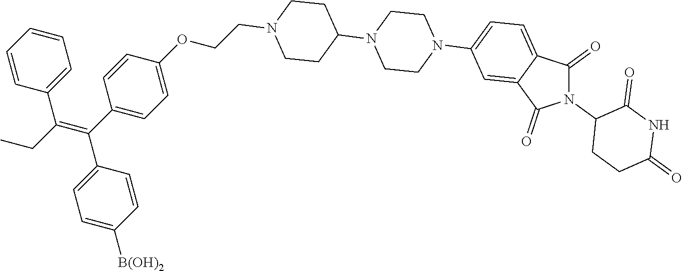 [SMILES: CC/C(=C(/c1ccc(OCCN2CCC(N3CCN(c4ccc5c(c4)C(=O)N(C4CCC(=O)NC4=O)C5=O)CC3)CC2)cc1)c1ccc(B(O)O)cc1)c1ccccc1] | (Z)-(4-(1-(4-(2-(4-(4-(2-(2,6- dioxopiperidin-3-yl)-1,3- dioxoisoindolin-5-yl)piperazin-1- yl)piperidin-1-yl)ethoxy)phenyl)-2- phenylbut-1-en-1-yl)phenyl)boronic acid | 796.3 | C | C | B | A |
| 32 | 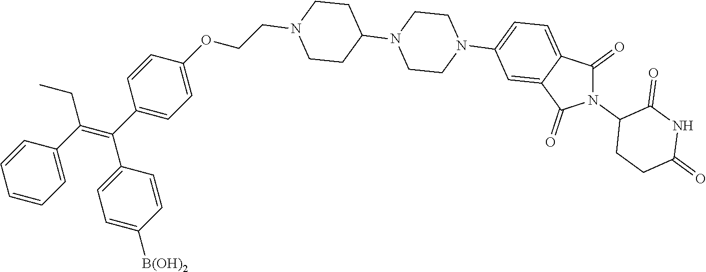 [SMILES: CC/C(=C(\c1ccc(OCCN2CCC(N3CCN(c4ccc5c(c4)C(=O)N(C4CCC(=O)NC4=O)C5=O)CC3)CC2)cc1)c1ccc(B(O)O)cc1)c1ccccc1] | (E)-(4-(1-(4-(2-(4-(4-(2-(2,6- dioxopiperidin-3-yl)-1,3- dioxoisoindolin-5-yl)piperazin-1- yl)piperidin-1-yl)ethoxy)phenyl)-2- phenylbut-1-en-1-yl)phenyl)boronic acid | 796.3 | C | C | B | A |
| 33 | 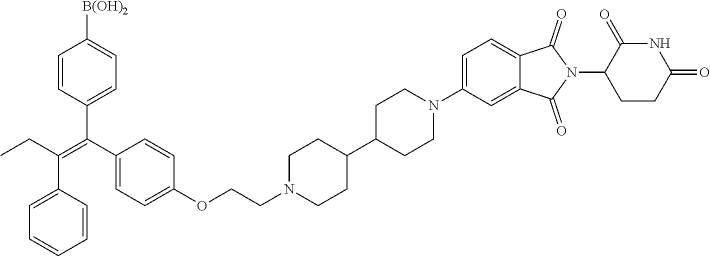 [SMILES: CC/C(=C(/c1ccc(OCCN2CCC(C3CCN(c4ccc5c(c4)C(=O)N(C4CCC(=O)NC4=O)C5=O)CC3)CC2)cc1)c1ccc(B(O)O)cc1)c1ccccc1] | (Z)-(4-(1-(4-(2-(1′-(2-(2,6- dioxopiperidin-3-yl)-1,3- dioxoisoindolin-5-yl)-[4,4′-bipiperidin]- 1-yl)ethoxy)phenyl)-2-phenylbut-1-en- 1-yl)phenyl)boronic acid | 795.4 | B | B | A | A |
| 34 | 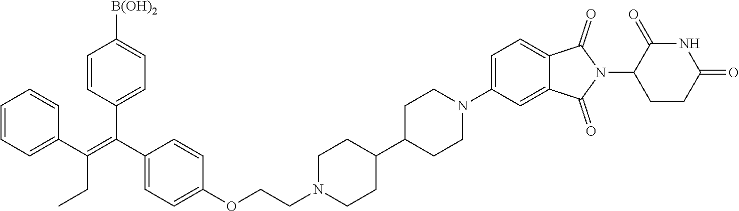 [SMILES: CC/C(=C(\c1ccc(OCCN2CCC(C3CCN(c4ccc5c(c4)C(=O)N(C4CCC(=O)NC4=O)C5=O)CC3)CC2)cc1)c1ccc(B(O)O)cc1)c1ccccc1] | (E)-(4-(1-(4-(2-(1′-(2-(2,6- dioxopiperidin-3-yl)-1,3- dioxoisoindolin-5-yl)-[4,4′-bipiperidin]- 1-yl)ethoxy)phenyl)-2-phenylbut-1-en- 1-yl)phenyl)boronic acid | 795.4 | B | B | A | A |
| 35 | 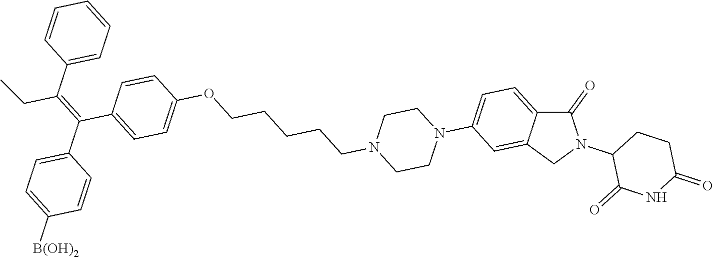 [SMILES: CC/C(=C(/c1ccc(OCCCCCN2CCN(c3ccc4c(c3)CN(C3CCC(=O)NC3=O)C4=O)CC2)cc1)c1ccc(B(O)O)cc1)c1ccccc1] | (Z)-(4-(1-(4-((5-(4-(2-(2,6- dioxopiperidin-3-yl)-1-oxoisoindolin-5- yl)piperazin-1-yl)pentyl)oxy)phenyl)-2- phenylbut-1-en-1-yl)phenyl)boronic acid | 741.3 | B | B | B | A |
| 36 | 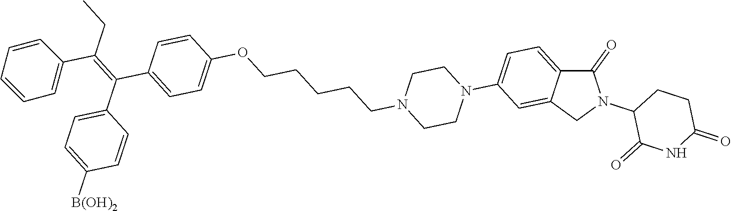 [SMILES: CC/C(=C(\c1ccc(OCCCCCN2CCN(c3ccc4c(c3)CN(C3CCC(=O)NC3=O)C4=O)CC2)cc1)c1ccc(B(O)O)cc1)c1ccccc1] | (E)-(4-(1-(4-((5-(4-(2-(2,6- dioxopiperidin-3-yl-1-oxoisoindolin-5- yl)piperazin-1-yl)pentyl)oxy)phenyl)-2- phenylbut-1-en-1-yl)phenyl)boronic acid | 741.3 | B | B | B | A |
| 37 | 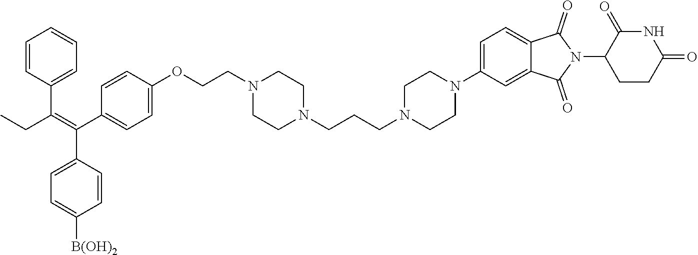 [SMILES: CC/C(=C(/c1ccc(OCCN2CCN(CCCN3CCN(c4ccc5c(c4)C(=O)N(C4CCC(=O)NC4=O)C5=O)CC3)CC2)cc1)c1ccc(B(O)O)cc1)c1ccccc1] | (Z)-(4-(1-(4-(2-(4-(3-(4-(2-(2,6- dioxopiperidin-3-yl)-1,3- dioxoisoindolin-5-yl)piperazin-1- yl)propyl)piperazin-1- yl)ethoxy)phenyl)-2-phenylbut-1-en-1- yl)phenyl)boronic acid | 839.4 | B | B | A | A |
| 38 | 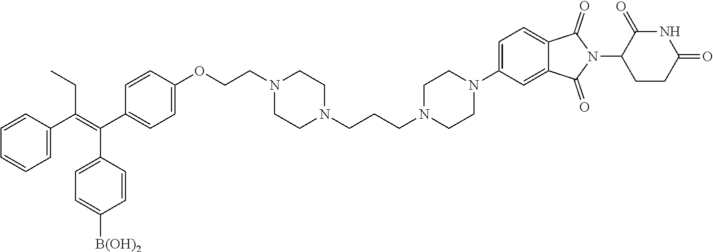 [SMILES: CC/C(=C(\c1ccc(OCCN2CCN(CCCN3CCN(c4ccc5c(c4)C(=O)N(C4CCC(=O)NC4=O)C5=O)CC3)CC2)cc1)c1ccc(B(O)O)cc1)c1ccccc1] | (E)-(4-(1-(4-(2-(4-(3-(4-(2-(2,6- dioxopiperidin-3-yl)-1,3- dioxoisoindolin-5-yl)piperazin-1- yl)propyl)piperazin-1- yl)ethoxy)phenyl)-2-phenylbut- 1-en-1-yl)phenyl)boronic acid | 839.4 | B | B | A | A |
| 39 | 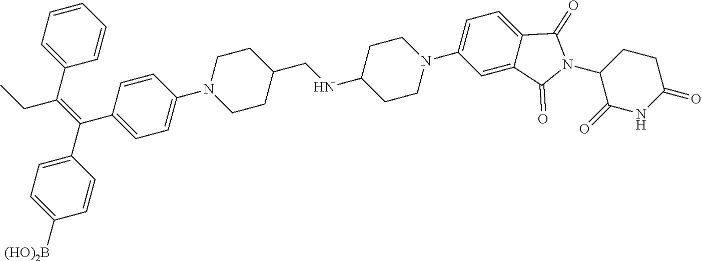 [SMILES: CC/C(=C(\c1ccc(B(O)O)cc1)c1ccc(N2CCC(CNC3CCN(c4ccc5c(c4)C(=O)N(C4CCC(=O)NC4=O)C5=O)CC3)CC2)cc1)c1ccccc1] | (Z)-(4-(1-(4-(4-(((1-(2-(2,6- dioxopiperidin-3-yl)-1,3- dioxoisoindolin-5-yl)piperidin-4- yl)amino)methyl)piperidin-1-yl)phenyl)- 2-phenylbut-1-en-1-yl)phenyl)boronic acid | 780.5 | B | B | A | A |
| 40 | 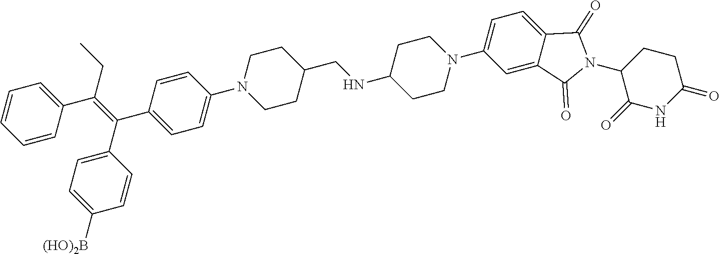 [SMILES: CC/C(=C(/c1ccc(B(O)O)cc1)c1ccc(N2CCC(CNC3CCN(c4ccc5c(c4)C(=O)N(C4CCC(=O)NC4=O)C5=O)CC3)CC2)cc1)c1ccccc1] | (E)-(4-(1-(4-(4-(((1-(2-(2,6- dioxopiperidin-3-yl)-1,3- dioxoisoindolin-5-yl)piperidin-4- yl)amino)methyl)piperidin-1-yl)phenyl)- 2-phenylbut-1-en-1-yl)phenyl)boronic acid | 780.5 | B | B | A | A |
| 41 | 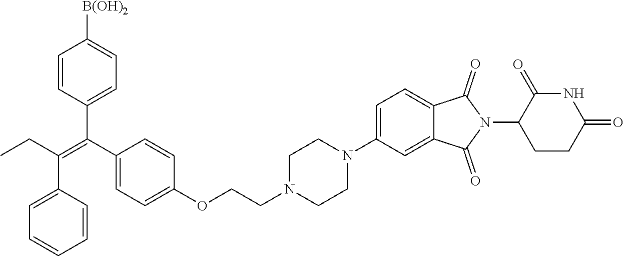 [SMILES: CC/C(=C(/c1ccc(OCCN2CCN(c3ccc4c(c3)C(=O)N(C3CCC(=O)NC3=O)C4=O)CC2)cc1)c1ccc(B(O)O)cc1)c1ccccc1] | (Z)-(4-(1-(4-(2-(4-(2-(2,6- dioxopiperidin-3-yl)-1,3- dioxoisoindolin-5-yl)piperazin-1- yl)ethoxy)phenyl)-2-phenylbut-1- en-1-yl)phenyl)boronic acid | 713.3 | B | B | B | C |
| 42 | 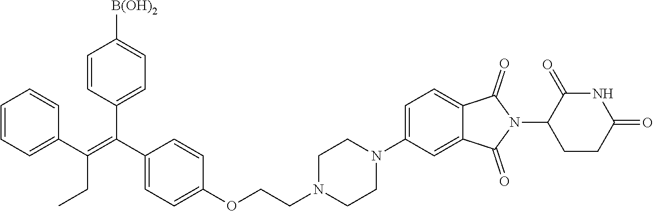 [SMILES: CC/C(=C(\c1ccc(OCCN2CCN(c3ccc4c(c3)C(=O)N(C3CCC(=O)NC3=O)C4=O)CC2)cc1)c1ccc(B(O)O)cc1)c1ccccc1] | (E)-(4-(1-(4-(2-(4-(2-(2,6- dioxopiperidin-3-yl)-1,3- dioxoisoindolin-5-yl)piperazin-1 yl)ethoxy)phenyl)-2-phenylbut-1-en-1- yl)phenyl)boronic acid | 713.3 | B | B | B | C |
| 43 | 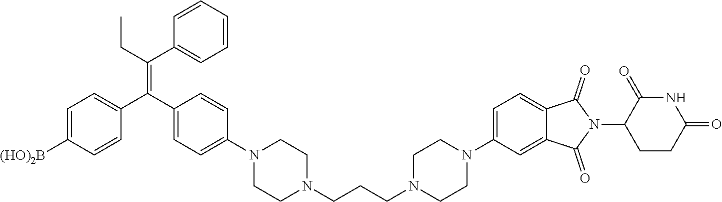 [SMILES: CC/C(=C(\c1ccc(B(O)O)cc1)c1ccc(N2CCN(CCCN3CCN(c4ccc5c(c4)C(=O)N(C4CCC(=O)NC4=O)C5=O)CC3)CC2)cc1)c1ccccc1] | (Z)-(4-(1-(4-(4-(3-(4-(2-(2,6- dioxopiperidin-3-yl)-1,3- dioxoisoindolin-5-yl)piperazin-1- yl)propyl)piperazin-1-yl)phenyl)-2- phenylbut-1-en-1-yl)phenyl)boronic acid | 795.4 | B | A | A | A |
| 44 | 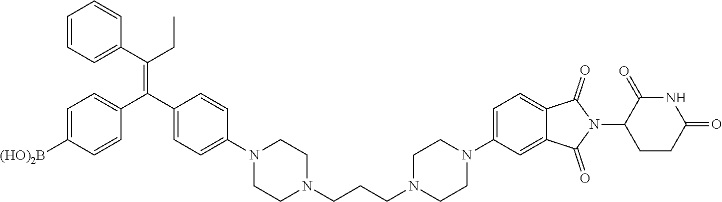 [SMILES: CC/C(=C(/c1ccc(B(O)O)cc1)c1ccc(N2CCN(CCCN3CCN(c4ccc5c(c4)C(=O)N(C4CCC(=O)NC4=O)C5=O)CC3)CC2)cc1)c1ccccc1] | (E)-(4-(1-(4-(4-(3-(4-(2-(2,6- dioxopiperidin-3-yl)-1,3- dioxoisoindolin-5-yl)piperazin-1- yl)propyl)piperazin-1-yl)phenyl)-2- phenylbut-1-en-1-yl)phenyl)boronic acid | 795.3 | A | B | A | A |
| 45 | 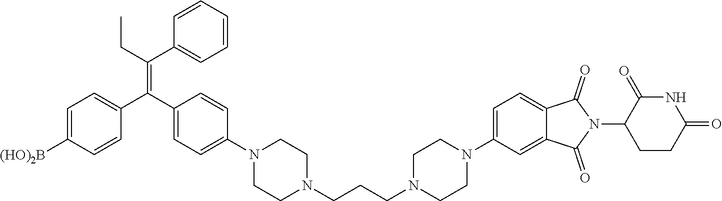 [SMILES: CC/C(=C(\c1ccc(B(O)O)cc1)c1ccc(N2CCN(CCCN3CCN(c4ccc5c(c4)C(=O)N(C4CCC(=O)NC4=O)C5=O)CC3)CC2)cc1)c1ccccc1] | (Z)-(4-(1-(4-(4-(3-(4-(2-(2,6- dioxopiperidin-3-yl)-1-oxoisoindolin- 5-yl)piperazin-1-yl)propyl)piperazin-1- yl)phenyl)-2-phenylbut-1-en-1- yl)phenyl)boronic acid | 781.3 | A | B | A | A |
| 46 | 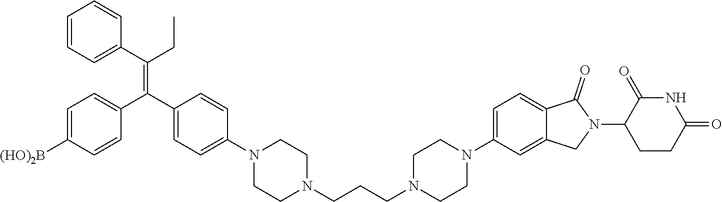 [SMILES: CC/C(=C(/c1ccc(B(O)O)cc1)c1ccc(N2CCN(CCCN3CCN(c4ccc5c(c4)CN(C4CCC(=O)NC4=O)C5=O)CC3)CC2)cc1)c1ccccc1] | (E)-(4-(1-(4-(4-(3-(4-(2-(2,6- dioxopiperidin-3-yl)-1-oxoisoindolin- 5-yl)piperazin-1-yl)propyl)piperazin-1- yl)phenyl)-2-phenylbut-1-en-1- yl)phenyl)boronic acid | 781.4 | A | B | A | A |
| 47 | 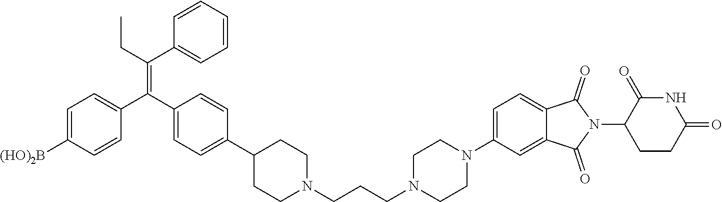 [SMILES: CC/C(=C(\c1ccc(B(O)O)cc1)c1ccc(C2CCN(CCCN3CCN(c4ccc5c(c4)C(=O)N(C4CCC(=O)NC4=O)C5=O)CC3)CC2)cc1)c1ccccc1] | (Z)-(4-(1-(4-(1-(3-(4-(2-(2,6- dioxopiperidin-3-yl)-1,3- dioxoisoindolin-5-yl)piperazin-1- yl)propyl)piperidin-4-yl)phenyl)-2- phenylbut-1-en-3-yl)phenyl)boronic acid | 794.5 | B | B | A | A |
| 48 | 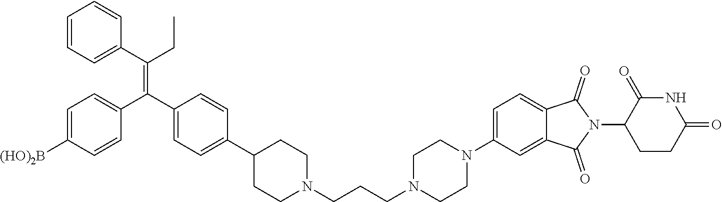 [SMILES: CC/C(=C(/c1ccc(B(O)O)cc1)c1ccc(C2CCN(CCCN3CCN(c4ccc5c(c4)C(=O)N(C4CCC(=O)NC4=O)C5=O)CC3)CC2)cc1)c1ccccc1] | (E)-(4-(1-(4-(1-(3-(4-(2-(2,6- dioxopiperidin-3-yl)-1,3- dioxoisoindolin-5-yl)piperazin-1- yl)propyl)piperidin-4-yl)phenyl)-2- phenylbut-1-en-1-yl)phenyl)boronic acid | 794.5 | B | B | A | A |
| 49 | 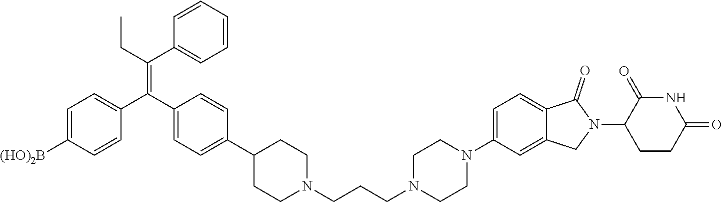 [SMILES: CC/C(=C(\c1ccc(B(O)O)cc1)c1ccc(C2CCN(CCCN3CCN(c4ccc5c(c4)CN(C4CCC(=O)NC4=O)C5=O)CC3)CC2)cc1)c1ccccc1] | (Z)-(4-(1-(4-(1-(3-(4-(2-(2,6- dioxopiperidin-3-yl)-1-oxoisoindolin-5- yl)piperazin-1-yl)propyl)piperidin-4- yl)phenyl)-2-phenylbut-1-en-1- yl)phenyl)boronic acid | 780.4 | A | B | A | A |
| 50 | 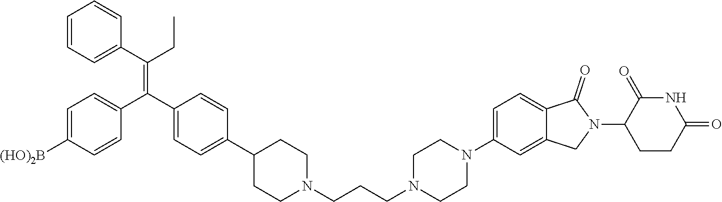 [SMILES: CC/C(=C(/c1ccc(B(O)O)cc1)c1ccc(C2CCN(CCCN3CCN(c4ccc5c(c4)CN(C4CCC(=O)NC4=O)C5=O)CC3)CC2)cc1)c1ccccc1] | (E)-(4-(1-(4-(1-(3-(4-(2-(2,6- dioxopiperidin-3-yl)-1-oxoisoindolin-5- yl)piperazin-1-yl)propyl)piperidin-4- yl)phenyl)-2-phenylbut-1-en-1- yl)phenyl)boronic acid | 780.4 | A | B | A | A |
| 51 | 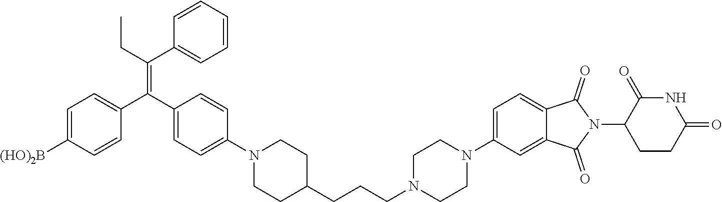 [SMILES: CC/C(=C(\c1ccc(B(O)O)cc1)c1ccc(N2CCC(CCCN3CCN(c4ccc5c(c4)C(=O)N(C4CCC(=O)NC4=O)C5=O)CC3)CC2)cc1)c1ccccc1] | (Z)-(4-(1-(4-(4-(3-(4-(2-(2,6- dioxopiperidin-3-yl)-1,3- dioxoisoindolin-5-yl)piperazin-1- yl)propyl)piperidin-1-yl)phenyl)-2- phenylbut-1-en-1-yl)phenyl)boronic acid | 794.5 | B | B | A | A |
| 52 | 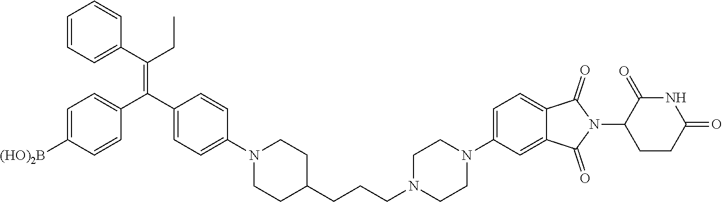 [SMILES: CC/C(=C(/c1ccc(B(O)O)cc1)c1ccc(N2CCC(CCCN3CCN(c4ccc5c(c4)C(=O)N(C4CCC(=O)NC4=O)C5=O)CC3)CC2)cc1)c1ccccc1] | (E)-(4-(1-(4-(4-(3-(4-(2-(2,6- dioxopiperidin-3-yl)-1,3- dioxoisoindolin-5-yl)piperazin-1- yl)propyl)piperidin-1-yl)phenyl)-2- phenylbut-1-en-1-yl)phenyl)boronic acid | 794.5 | B | B | A | A |
| 53 | 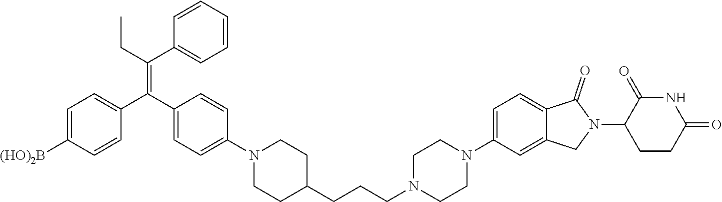 [SMILES: CC/C(=C(\c1ccc(B(O)O)cc1)c1ccc(N2CCC(CCCN3CCN(c4ccc5c(c4)CN(C4CCC(=O)NC4=O)C5=O)CC3)CC2)cc1)c1ccccc1] | (Z)-(4-(1-(4-(4-(3-(4-(2-(2,6- dioxopiperidin-3-yl)-1-oxoisoindolin-5- yl)piperazin-1-yl)propyl)piperidin-1- yl)phenyl)-2-phenylbut-1-en-1- yl)phenyl)boronic acid | 780.3 | A | B | A | A |
| 54 | 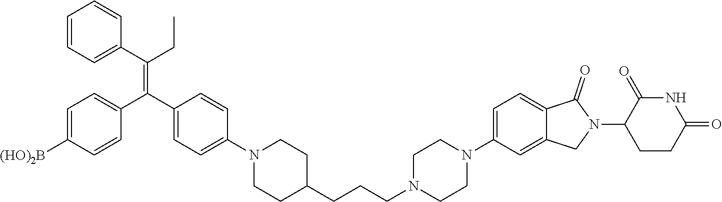 [SMILES: CC/C(=C(/c1ccc(B(O)O)cc1)c1ccc(N2CCC(CCCN3CCN(c4ccc5c(c4)CN(C4CCC(=O)NC4=O)C5=O)CC3)CC2)cc1)c1ccccc1] | (E)-(4-(1-(4-(4-(3-(4-(2-(2,6- dioxopiperidin-3-yl)-1-oxoisoindolin-5- yl)piperazin-1-yl)propyl)piperidin-1- yl)phenyl-2-phenylbut-1- en-1-yl)phenyl)boronic acid | 780.3 | A | B | A | A |
| 55 | 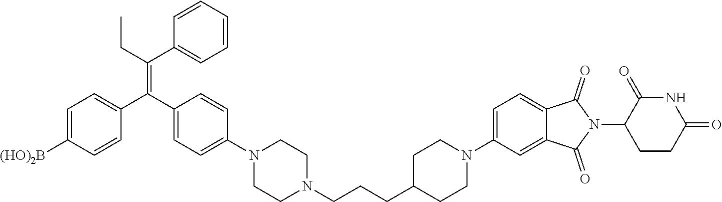 [SMILES: CC/C(=C(\c1ccc(B(O)O)cc1)c1ccc(N2CCN(CCCC3CCN(c4ccc5c(c4)C(=O)N(C4CCC(=O)NC4=O)C5=O)CC3)CC2)cc1)c1ccccc1] | (Z)-(4-(1-(4-(4-(3-(1-(2-(2,6- dioxopiperidin-3-yl)-1,3- dioxoisoindolin-5-yl)piperidin-4- yl)propyl)piperazin-1-yl)phenyl)-2- phenylbut-1-en-1-yl)phenyl)boronic acid | 794.4 | B | B | A | B |
| 56 | 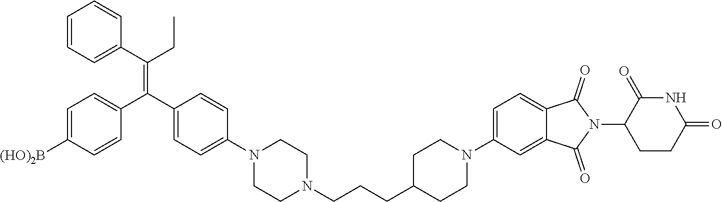 [SMILES: CC/C(=C(/c1ccc(B(O)O)cc1)c1ccc(N2CCN(CCCC3CCN(c4ccc5c(c4)C(=O)N(C4CCC(=O)NC4=O)C5=O)CC3)CC2)cc1)c1ccccc1] | (E)-(4-(1-(4-(4-(3-(1-(2-(2,6- dioxopiperidin-3-yl)-1,3- dioxoisoindolin-5-yl)piperidin-4- yl)propyl)piperazin-1-yl)phenyl)-2- phenlbut-1-en-1-yl)phenyl)boronic acid | 794.4 | B | B | A | B |
| 57 | 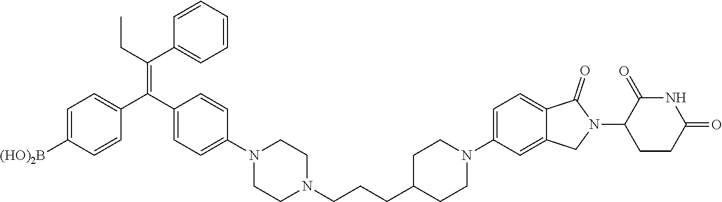 [SMILES: CC/C(=C(\c1ccc(B(O)O)cc1)c1ccc(N2CCN(CCCC3CCN(c4ccc5c(c4)CN(C4CCC(=O)NC4=O)C5=O)CC3)CC2)cc1)c1ccccc1] | (Z)-(4-(1-(4-(4-(3-(1-(2-(2,6- dioxopiperidin-3-yl)-1-oxoisoindolin-5- yl)piperidin-4-yl)propyl)piperazin-1- yl)phenyl)-2-phenylbut-1-en-1- yl)phenyl)boronic acid | 780.5 | B | B | A | A |
| 58 | 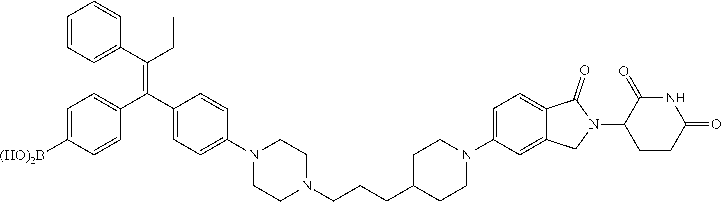 [SMILES: CC/C(=C(/c1ccc(B(O)O)cc1)c1ccc(N2CCN(CCCC3CCN(c4ccc5c(c4)CN(C4CCC(=O)NC4=O)C5=O)CC3)CC2)cc1)c1ccccc1] | (E)-(4-(1-(4-(4-(3-(1-(2-(2,6- dioxopiperidin-3-yl)-1-oxoisoindolin-5- yl)piperidin-4-yl)propylpiperazin-1- yl)phenyl)-2-phenylbut-1-en-1- yl)phenyl)boronic acid | 780.5 | B | B | A | A |
| 59 | 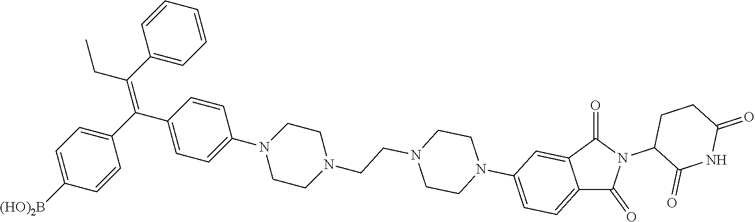 [SMILES: CC/C(=C(\c1ccc(B(O)O)cc1)c1ccc(N2CCN(CCN3CCN(c4ccc5c(c4)C(=O)N(C4CCC(=O)NC4=O)C5=O)CC3)CC2)cc1)c1ccccc1] | (Z)-(4-(1-(4-(4-(2-(4-(2-(2,6- dioxopiperidin-3-yl)-1,3- dioxoisoindolin-5-yl)piperazin-1- yl)ethyl)piperazin-1-yl)phenyl)-2- phenylbut-1-en-1-yl)phenyl)boronic acid | 781.3 | B | B | B | A |
| 60 | 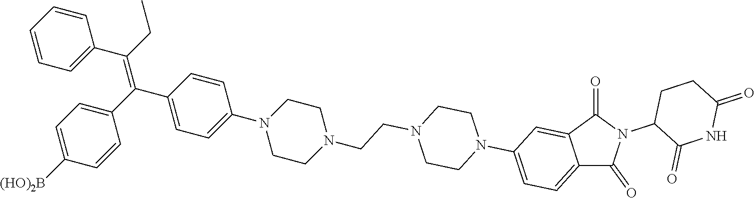 [SMILES: CC/C(=C(/c1ccc(B(O)O)cc1)c1ccc(N2CCN(CCN3CCN(c4ccc5c(c4)C(=O)N(C4CCC(=O)NC4=O)C5=O)CC3)CC2)cc1)c1ccccc1] | (E)-(4-(1-(4-(4-(2-(4-(2-(2,6- dioxopiperidin-3-yl)-1,3- dioxoisoindolin-5-yl)piperazin-1- yl)ethyl)piperazin-1-yl)phenyl)-2- phenylbut-1-en-1-yl)phenyl)boronic acid | 781.3 | B | B | B | A |
| 61 | 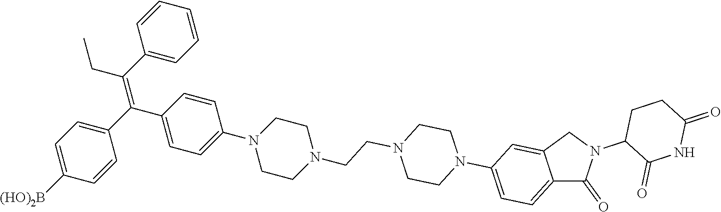 [SMILES: CC/C(=C(\c1ccc(B(O)O)cc1)c1ccc(N2CCN(CCN3CCN(c4ccc5c(c4)CN(C4CCC(=O)NC4=O)C5=O)CC3)CC2)cc1)c1ccccc1] | (Z)-(4-(1-(4-(4-(2-(4-(2-(2,6- dioxopiperidin-3-yl)-1-oxoisoindolin- 5-yl)piperazin-1-yl)ethyl)piperazin-1- yl)phenyl)-2-phenylbut-1-en-1- yl)phenyl)boronic acid | 767.4 | B | B | A | A |
| 62 | 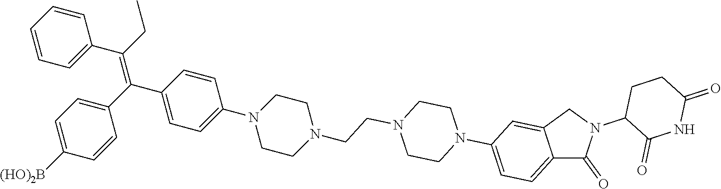 [SMILES: CC/C(=C(/c1ccc(B(O)O)cc1)c1ccc(N2CCN(CCN3CCN(c4ccc5c(c4)CN(C4CCC(=O)NC4=O)C5=O)CC3)CC2)cc1)c1ccccc1] | (E)-(4-(1-(4-(4-(2-(4-(2-(2,6- dioxopiperidin-3-yl)-1-oxoisoindolin-5- yl)piperazin-1-yl)ethyl)piperazin-1- yl)phenyl)-2-phenylbut-1-en-1- yl)phenyl)boronic acid | 767.4 | B | B | A | A |
| 63 | 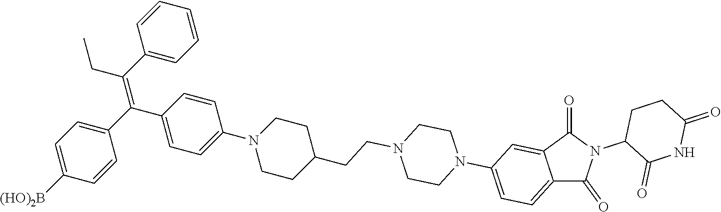 [SMILES: CC/C(=C(\c1ccc(B(O)O)cc1)c1ccc(N2CCC(CCN3CCN(c4ccc5c(c4)C(=O)N(C4CCC(=O)NC4=O)C5=O)CC3)CC2)cc1)c1ccccc1] | (Z)-(4-(1-(4-(4-(2-(4-(2-(2,6- dioxopiperidin-3-yl)-1,3- dioxoisoindolin-5-yl)piperazin-1- yl)ethyl)piperidin-1-yl)phenyl)-2- phenylbut-1-en-1-yl)phenyl)boronic acid | 780.4 | B | B | B | A |
| 64 | 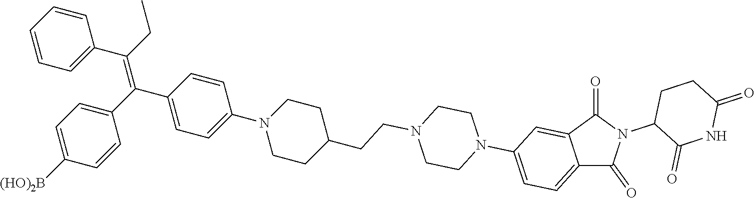 [SMILES: CC/C(=C(/c1ccc(B(O)O)cc1)c1ccc(N2CCC(CCN3CCN(c4ccc5c(c4)C(=O)N(C4CCC(=O)NC4=O)C5=O)CC3)CC2)cc1)c1ccccc1] | (E)-(4-(1-(4-(4-(2-(1-(2-(2,6- dioxopiperidin-3-yl)-1,3- dioxoisoindolin-5-yl)piperazin-1- yl)ethyl)piperidin-1-yl)pheny])-2- phenylbut-1-en-1-yl)phenyl)boronic acid | 780.4 | B | B | B | A |
| 65 | 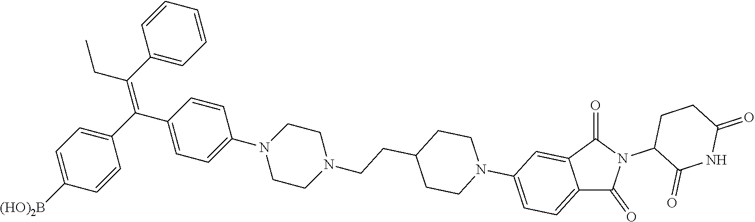 [SMILES: CC/C(=C(\c1ccc(B(O)O)cc1)c1ccc(N2CCN(CCC3CCN(c4ccc5c(c4)C(=O)N(C4CCC(=O)NC4=O)C5=O)CC3)CC2)cc1)c1ccccc1] | (Z)-(4-(1-(4-(4-(2-(1-(2-(2,6- dioxopiperidin-3-yl)-1,3- dioxoisoindolin-5-yl)piperidin-4- yl)ethyl)piperazin-1-yl)phenyl)-2- phenylbut-1-en-1-yl)phenyl)boronic acid | 780.4 | A | B | A | A |
| 66 | 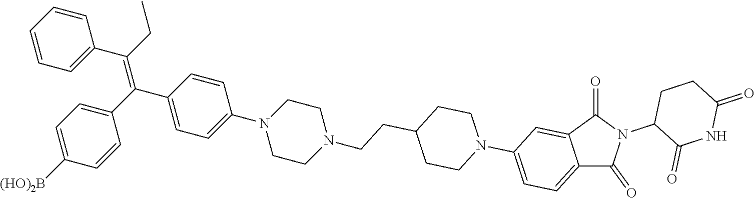 [SMILES: CC/C(=C(/c1ccc(B(O)O)cc1)c1ccc(N2CCN(CCC3CCN(c4ccc5c(c4)C(=O)N(C4CCC(=O)NC4=O)C5=O)CC3)CC2)cc1)c1ccccc1] | (E)-(4-(1-(4-(4-(2-(1-(2-(2,6- dioxopiperidin-3-yl)-1,3- dioxoisoindolin-5-yl)piperidin-4- yl)ethyl)piperazin-1-yl)phenyl)-2- phenylbut-1-en-1-yl)phenyl)boronic acid | 780.4 | B | B | A | A |
| 67 | 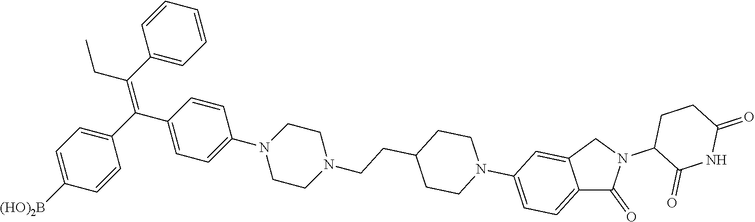 [SMILES: CC/C(=C(\c1ccc(B(O)O)cc1)c1ccc(N2CCN(CCC3CCN(c4ccc5c(c4)CN(C4CCC(=O)NC4=O)C5=O)CC3)CC2)cc1)c1ccccc1] | (Z)-(4-(1-(4-(4-(2-(1-(2-(2,6- dioxopiperidin-3-yl)-1-oxoisoindolin-5- yl)piperidin-4-yl)ethyl)piperazin-1- yl)phenyl)-2-phenylbut-1-en-1- yl)phenyl)boronic acid | 766.3 | A | A | A | A |
| 68 | 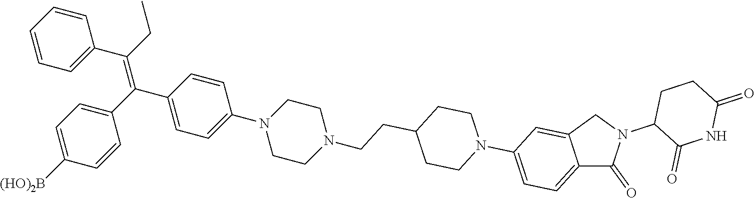 [SMILES: CC/C(=C(/c1ccc(B(O)O)cc1)c1ccc(N2CCN(CCC3CCN(c4ccc5c(c4)CN(C4CCC(=O)NC4=O)C5=O)CC3)CC2)cc1)c1ccccc1] | (E)-(4-(1-(4-(4-(2-(1-(2-(2,6- dioxopiperidin-3-yl)-1-oxoisoindolin-5- yl)piperidin-4-yl)ethyl)piperazin-1- yl)phenyl)-2-phenylbut-1-en-1- yl)phenyl)boronic acid | 766.3 | A | A | A | A |
| 69 | 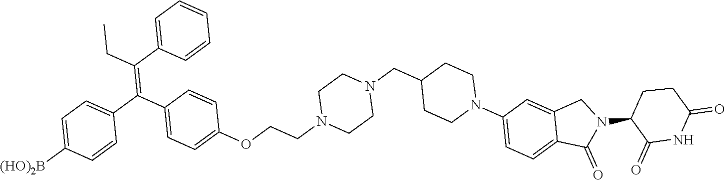 [SMILES: CC/C(=C(/c1ccc(OCCN2CCN(CC3CCN(c4ccc5c(c4)CN([C@H]4CCC(=O)NC4=O)C5=O)CC3)CC2)cc1)c1ccc(B(O)O)cc1)c1ccccc1] | (S,Z)-(4-(1-(4-(2-(4-((1-(2-2,6- dioxopiperidin-3-yl)-1-oxoisoindolin-5- yl)piperidin-4-yl)methyl)piperazin-1- yl)ethoxy)phenyl)-2-phenylbut-1-en-1- yl)phenyl)boronic acid | 796.42 | A | A | A | A |
| 70 | 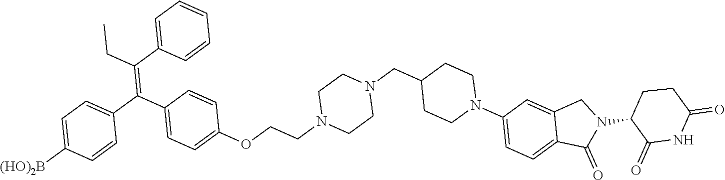 [SMILES: CC/C(=C(/c1ccc(OCCN2CCN(CC3CCN(c4ccc5c(c4)CN([C@@H]4CCC(=O)NC4=O)C5=O)CC3)CC2)cc1)c1ccc(B(O)O)cc1)c1ccccc1] | (R,Z)-(4-(1-(4-(2-(4-((1-(2-(2,6- dioxopiperidin-3-yl)-1-oxoisoindolin-5- yl)piperidin-4-yl)methypiperazin-1- yl)ethoxy)phenyl)-2-phenylbut-1-en-1- yl)phenyl)boronic acid | 796.42 | A | A | A | A |
| 71 | 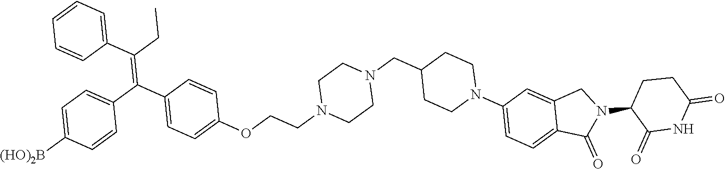 [SMILES: CC/C(=C(\c1ccc(OCCN2CCN(CC3CCN(c4ccc5c(c4)CN([C@H]4CCC(=O)NC4=O)C5=O)CC3)CC2)cc1)c1ccc(B(O)O)cc1)c1ccccc1] | (S,E)-(4-(1-(4-(2-(4-((1-(2-(2,6- dioxopiperidin-3-yl-1-oxoisoindolin-5- yl)piperidin-4-yl)methyl)piperazin-1- yl)ethoxy)phenyl)-2-phenylbut-1-en-1- yl)phenyl)boronic acid | 796.42 | A | A | A | A |
| 72 | 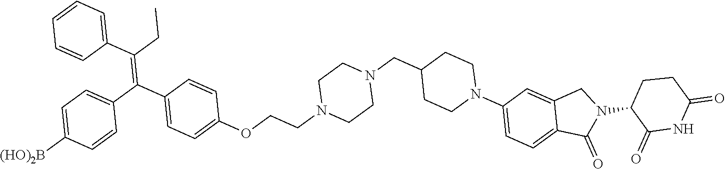 [SMILES: CC/C(=C(\c1ccc(OCCN2CCN(CC3CCN(c4ccc5c(c4)CN([C@@H]4CCC(=O)NC4=O)C5=O)CC3)CC2)cc1)c1ccc(B(O)O)cc1)c1ccccc1] | (R,E)-(4-1-(4-(2-(4-((1-(2-(2,6- dioxopiperidin-3-yl)-1-oxoisoindolin-5- yl)piperidin-4-yl)methyl)piperazin-1- yl)ethoxy)phenyl)-2-phenylbut-1-en-1- yl)phenyl)boronic acid | 796.42 | A | A | A | A |
| 73 | 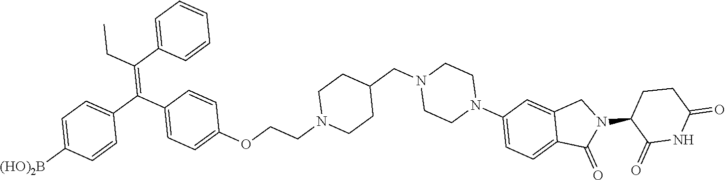 [SMILES: CC/C(=C(/c1ccc(OCCN2CCC(CN3CCN(c4ccc5c(c4)CN([C@H]4CCC(=O)NC4=O)C5=O)CC3)CC2)cc1)c1ccc(B(O)O)cc1)c1ccccc1] | (S,Z)-(4-(1-(4-(2-(4-((4-(2-(2,6- dioxopiperidin-3-yl)-1-oxoisoindolin-5- yl)piperazin-1-yl)methyl)piperidin-1- yl)ethoxy)phenyl)-2-phenylbut-1-en-1- yl)phenyl)boronic acid | 796.42 | A | A | A | A |
| 74 | 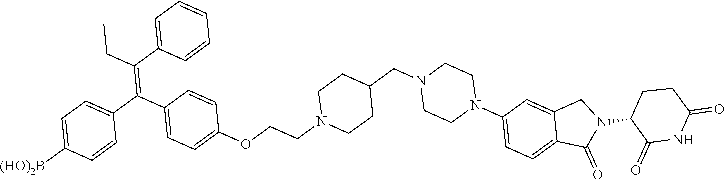 [SMILES: CC/C(=C(/c1ccc(OCCN2CCC(CN3CCN(c4ccc5c(c4)CN([C@@H]4CCC(=O)NC4=O)C5=O)CC3)CC2)cc1)c1ccc(B(O)O)cc1)c1ccccc1] | (R,Z)-(4-(1-(4-(2-(4-((4-(2-(2,6- dioxopiperidin-3-yl)-1-oxoisoindolin-5- yl)piperazin-1-yl)methyl)piperidin-1- yl)ethoxy)phenyl)-2-phenylbut-1-en-1- yl)phenyl)boronic acid | 796.42 | A | A | A | A |
| 75 | 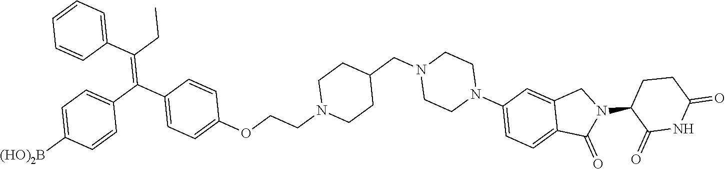 [SMILES: CC/C(=C(\c1ccc(OCCN2CCC(CN3CCN(c4ccc5c(c4)CN([C@H]4CCC(=O)NC4=O)C5=O)CC3)CC2)cc1)c1ccc(B(O)O)cc1)c1ccccc1] | (S,E)-(4-(1-(4-(2-(4-((4-(2-(2,6- dioxopiperidin-3-yl)-1-oxoisoindolin-5- yl)piperazin-1-yl)methyl)piperidin-1- yl)ethoxy)phenyl)-2-phenylbut-1-en-1- yl)phenyl)boronic acid | 796.42 | A | A | A | A |
| 76 | 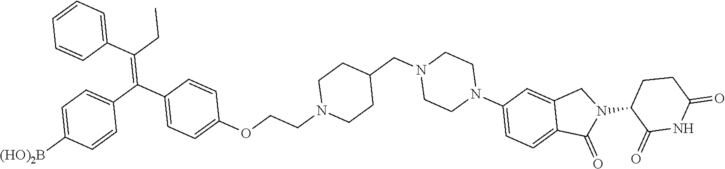 [SMILES: CC/C(=C(\c1ccc(OCCN2CCC(CN3CCN(c4ccc5c(c4)CN([C@@H]4CCC(=O)NC4=O)C5=O)CC3)CC2)cc1)c1ccc(B(O)O)cc1)c1ccccc1] | (R,E)-(4-(1-(4-(2-(4-(4-(2-(2,6- dioxopiperidin-3-yl)-1-oxoisoindolin-5- yl)piperazin-1-yl)methyl)piperidin-1- yl)ethoxy)phenyl)-2-phenylbut-1-en-1- yl)phenyl)boronic acid | 796.42 | A | A | A | A |
| 77 | 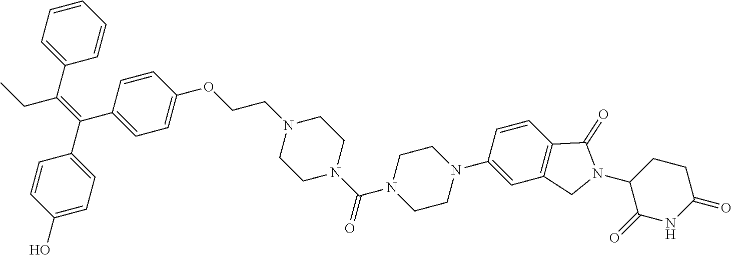 [SMILES: CC/C(=C(\c1ccc(O)cc1)c1ccc(OCCN2CCN(C(=O)N3CCN(c4ccc5c(c4)CN(C4CCC(=O)NC4=O)C5=O)CC3)CC2)cc1)c1ccccc1] | (Z)-3-(5-(4-(4-(2-(4-(1-(4- hydroxyphenyl)-2-phenylbut-1-en-1- yl)phenoxy)ethyl)piperazine-1- carbonyl)piperazin-1-yl-1- oxoisoindolin-2-yl)piperidine-2,6-dione | 783.38 | A | A | A | A |
| 78 | 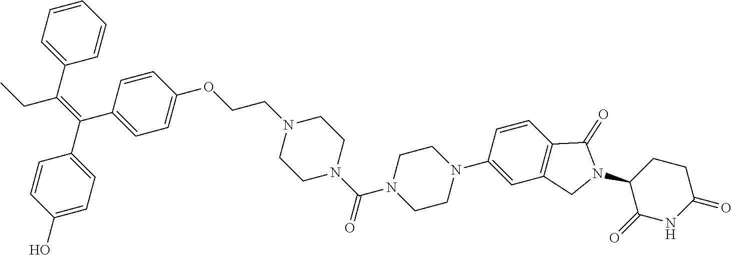 [SMILES: CC/C(=C(\c1ccc(O)cc1)c1ccc(OCCN2CCN(C(=O)N3CCN(c4ccc5c(c4)CN([C@H]4CCC(=O)NC4=O)C5=O)CC3)CC2)cc1)c1ccccc1] | (S,Z)-3-(5-(4-(4-(2-(4-(1-(4- hydroxyphenyl)-2-phenylbut-1-en-1- yl)phenoxy)ethyl)piperazine-1- carbonyl)piperazin-1-yl)-1- oxoisoindolin-2-yl)piperidine-2,6-dione | 783.38 | A | A | A | A |
| 79 | 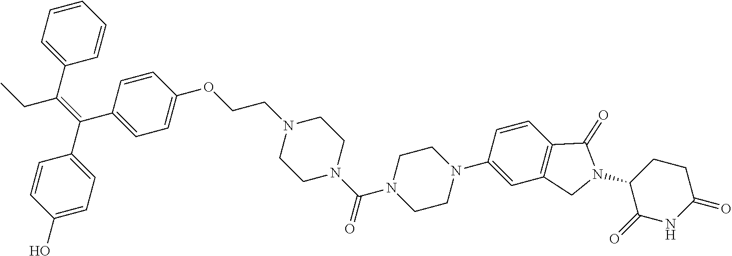 [SMILES: CC/C(=C(\c1ccc(O)cc1)c1ccc(OCCN2CCN(C(=O)N3CCN(c4ccc5c(c4)CN([C@@H]4CCC(=O)NC4=O)C5=O)CC3)CC2)cc1)c1ccccc1] | (R,Z)-3-(5-(4-(4-(2-(4-(1-(4- hydroxyphenyl)-2-phenylbut-1-en-1- yl)phenoxy)ethyl)piperazine-1- carbonyl)piperazin-1-yl)-1- oxoisoindolin-2-yl)piperidine-2,6-dione | 783.38 | A | A | A | A |
| 80 | 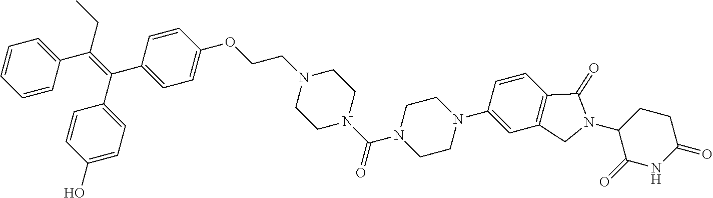 [SMILES: CC/C(=C(/c1ccc(O)cc1)c1ccc(OCCN2CCN(C(=O)N3CCN(c4ccc5c(c4)CN(C4CCC(=O)NC4=O)C5=O)CC3)CC2)cc1)c1ccccc1] | (E)-3-(5-(4-(4-(2-(4-(1-(4- hydroxyphenyl)-2-phenylbut-1-en-1- yl)phenoxy)ethyl)piperazine-1- carbonyl)piperazin-1-yl)-1- oxoisoindolin-2-yl)piperidine-2,6-dione | 783.38 | A | A | A | A |
| 81 | 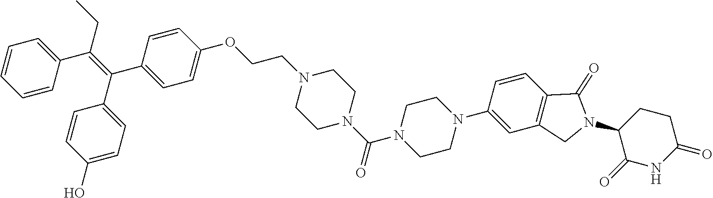 [SMILES: CC/C(=C(/c1ccc(O)cc1)c1ccc(OCCN2CCN(C(=O)N3CCN(c4ccc5c(c4)CN([C@H]4CCC(=O)NC4=O)C5=O)CC3)CC2)cc1)c1ccccc1] | (S,E)-3-(5-(4-(4-(2-(4-(1-(4- hydroxyphenyl)-2-phenylbut-1-en-1- yl)phenoxy)ethyl)piperazine-1- carbonyl)piperazin-1-yl)-1- oxoisoindolin-2-yl)piperidine-2,6-dione | 783.38 | A | A | A | A |
| 82 | 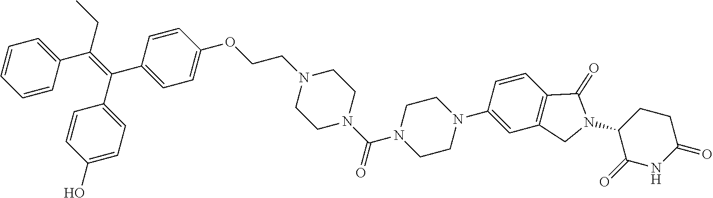 [SMILES: CC/C(=C(/c1ccc(O)cc1)c1ccc(OCCN2CCN(C(=O)N3CCN(c4ccc5c(c4)CN([C@@H]4CCC(=O)NC4=O)C5=O)CC3)CC2)cc1)c1ccccc1] | (R,E)-3-(5-(4-(4-(2-(4-(1-(4- hydroxyphenyl)-2-phenylbut-1-en-1- yl)phenoxy)ethyl)piperazine-1- carbonyl)piperazin-1-yl-1- oxoisoindolin-2-yl)piperidine-2,6-dione | 783.38 | A | A | A | A |
| 83 | 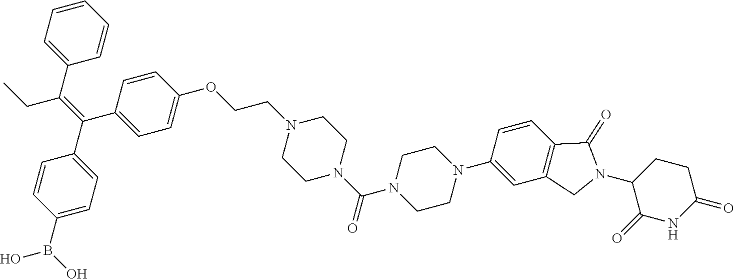 [SMILES: CC/C(=C(/c1ccc(OCCN2CCN(C(=O)N3CCN(c4ccc5c(c4)CN(C4CCC(=O)NC4=O)C5=O)CC3)CC2)cc1)c1ccc(B(O)O)cc1)c1ccccc1] | (Z)-(4-(1-(4-(2-(4-(4-(2-(2,6- dioxopiperidin-3-yl)-1-oxoisoindolin-5- yl)piperazine-1-carbonyl)piperazin-1- yl)ethoxy)phenyl)-2-phenylbut-1-en-1- yl)phenyl)boronic acid | 811.39 | A | A | A | A |
| 84 | 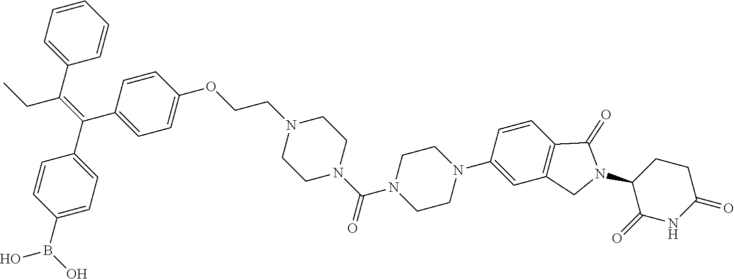 [SMILES: CC/C(=C(/c1ccc(OCCN2CCN(C(=O)N3CCN(c4ccc5c(c4)CN([C@H]4CCC(=O)NC4=O)C5=O)CC3)CC2)cc1)c1ccc(B(O)O)cc1)c1ccccc1] | (S,Z)-(4-(1-(4-(2-(4-(4-(2-(2,6- dioxopiperidin-3-yl)-1-oxoisoindolin-5- yl)piperazine-1-carbonyl)piperazin-1- yl)ethoxy)phenyl)-2-phenylbut-1-en-1- ylphenyl)boronic acid | 811.39 | A | A | A | A |
| 85 | 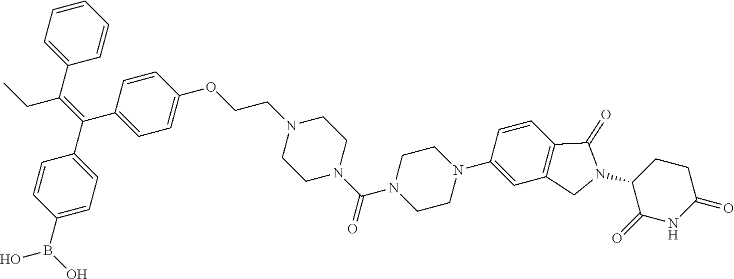 [SMILES: CC/C(=C(/c1ccc(OCCN2CCN(C(=O)N3CCN(c4ccc5c(c4)CN([C@@H]4CCC(=O)NC4=O)C5=O)CC3)CC2)cc1)c1ccc(B(O)O)cc1)c1ccccc1] | (R,Z)-(4-(1-(4-(2-(4-(4-(2-(2,6- dioxopiperidin-3-yl)-1-oxoisoindolin-5- yl)piperazine-1-carbonyl)piperazin-1- yl)ethoxy)phenyl)-2-phenylbut-1-en-1- yl)phenyl)boronic acid | 811.39 | A | A | A | A |
| 86 | 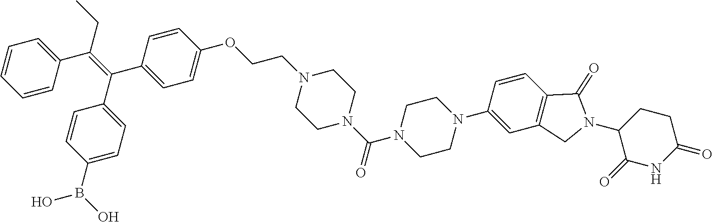 [SMILES: CC/C(=C(\c1ccc(OCCN2CCN(C(=O)N3CCN(c4ccc5c(c4)CN(C4CCC(=O)NC4=O)C5=O)CC3)CC2)cc1)c1ccc(B(O)O)cc1)c1ccccc1] | (E)-(4-(1-(4-(2-(4-(4-(2-(2,6- dioxopiperidin-3-yl)-1-oxoisoindolin-5- yl)piperazine-1-carbonyl)piperazin-1- yl)ethoxy)phenyl)-2-phenylbut-1-en-1- yl)phenyl)boronic acid | 811.39 | A | A | A | A |
| 87 | 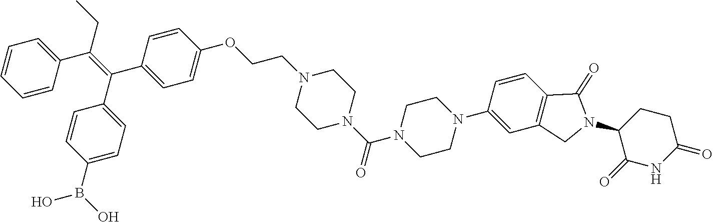 [SMILES: CC/C(=C(\c1ccc(OCCN2CCN(C(=O)N3CCN(c4ccc5c(c4)CN([C@H]4CCC(=O)NC4=O)C5=O)CC3)CC2)cc1)c1ccc(B(O)O)cc1)c1ccccc1] | (S,E)-(4-(1-(4-(2-(4-(4-(2-(2,6- dioxopiperidin-3-yl)-1-oxoisoindolin-5- yl)piperazine-1-carbonyl)piperazin-1- yl)ethoxy)phenyl)-2-phenylbut-1-en-1- yl)phenyl)boronic acid | 811.39 | A | A | A | A |
| 88 | 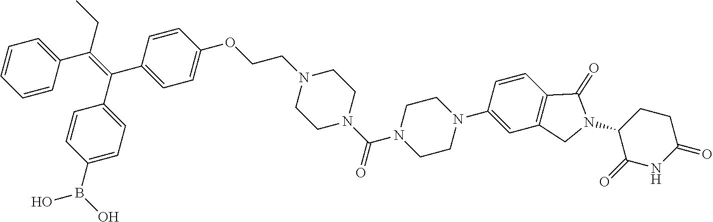 [SMILES: CC/C(=C(\c1ccc(OCCN2CCN(C(=O)N3CCN(c4ccc5c(c4)CN([C@@H]4CCC(=O)NC4=O)C5=O)CC3)CC2)cc1)c1ccc(B(O)O)cc1)c1ccccc1] | (R,E)-(4-(1-(4-(2-(4-(4-(2-(2,6- dioxopiperidin-3-yl)-1-oxoisoindolin-5- yl)piperazine-1-carbonyl)piperazin-1- yl)ethoxy)phenyl)-2-phenylbut-1-en-1- ylphenyl)boronic acid | 811.39 | A | A | A | A |
| 89 | 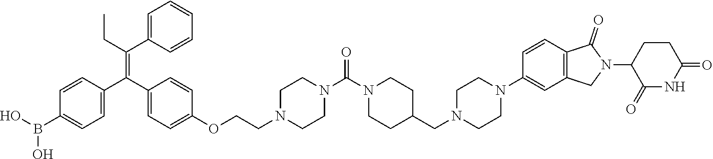 [SMILES: CC/C(=C(/c1ccc(OCCN2CCN(C(=O)N3CCC(CN4CCN(c5ccc6c(c5)CN(C5CCC(=O)NC5=O)C6=O)CC4)CC3)CC2)cc1)c1ccc(B(O)O)cc1)c1ccccc1] | (Z)-3-(5-(4-((1-(4-(2-(4-(1-(4- hydroxyphenyl)-2-phenylbut-1-en-1- yl)phenoxy)ethyl)piperazine-1- carbonyl)piperidin-4- yl)methylpiperazin-1-yl)-1- oxoisoindolin-2-yl)piperidine-2,6-dione | 880.47 | A | A | A | A |
| 90 | 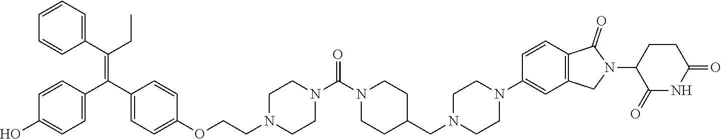 [SMILES: CC/C(=C(/c1ccc(O)cc1)c1ccc(OCCN2CCN(C(=O)N3CCC(CN4CCN(c5ccc6c(c5)CN(C5CCC(=O)NC5=O)C6=O)CC4)CC3)CC2)cc1)c1ccccc1] | (E)-3-(5-(4-((1-(4-(2-(4-(1-(4- hydroxyphenyl)-2-phenylbut-1-en-1- yl)phenoxy)ethyl)piperazine-1- carbonyl)piperidin-4- yl)methyl)piperazin-1-yl)-1- oxoisoindolin-2-yl)piperidine-2,6-dione | 880.47 | A | A | A | A |
| 91 | 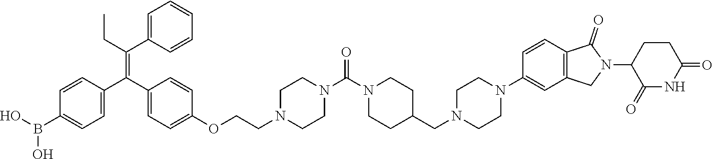 [SMILES: CC/C(=C(/c1ccc(OCCN2CCN(C(=O)N3CCC(CN4CCN(c5ccc6c(c5)CN(C5CCC(=O)NC5=O)C6=O)CC4)CC3)CC2)cc1)c1ccc(B(O)O)cc1)c1ccccc1] | (Z)-(4-(1-(4-(2-(4-(4-((4-(2-(2,6- dioxopiperidin-3-yl)-1-oxoisoindolin-5- yl)piperazin-1-yl)methyl)piperidine-1- carbonyl)piperazin-1-yl)ethoxy)phenyl)- 2-phenylbut-1-en-1-yl)phenyl)boronic acid | 908.48 | A | A | A | A |
| 92 | 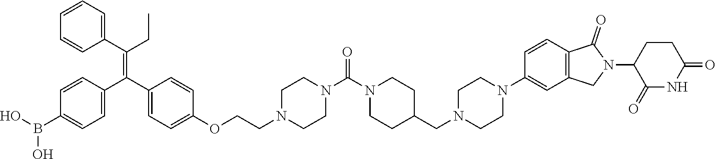 [SMILES: CC/C(=C(\c1ccc(OCCN2CCN(C(=O)N3CCC(CN4CCN(c5ccc6c(c5)CN(C5CCC(=O)NC5=O)C6=O)CC4)CC3)CC2)cc1)c1ccc(B(O)O)cc1)c1ccccc1] | (E)-(4-(1-(4-(2-(4-(4-((4-(2-(2,6- dioxopiperidin-3-yl)-1-oxoisoindolin-5- yl)piperazin-1-yl)methyl)piperidine-1- carbonyl)piperazin-1-yl)ethoxy)phenyl)- 2-phenylbut-1-en-1-yl)phenyl)boronic acid | 908.48 | A | A | A | A |
| 93 | 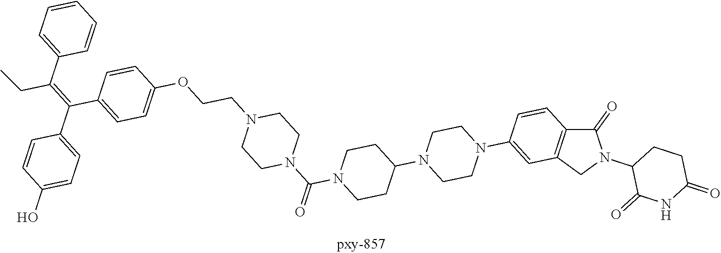 [SMILES: CC/C(=C(\c1ccc(O)cc1)c1ccc(OCCN2CCN(C(=O)N3CCC(N4CCN(c5ccc6c(c5)CN(C5CCC(=O)NC5=O)C6=O)CC4)CC3)CC2)cc1)c1ccccc1] | (Z)-3-(5-(4-(1-(4-(2-(4-(1-(4- hydroxyphenyl)-2-phenylbut-1-en-1- yl)phenoxy)ethyl)piperazine-1- carbonyl)piperidin-4-yl)piperazin-1-yl)- 1-oxoisoindolin-2-yl)piperidine-2,6- dione | 866.45 | A | A | B | A |
| 94 | 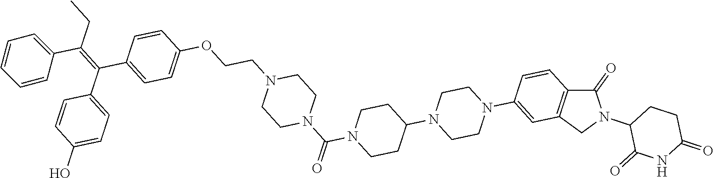 [SMILES: CC/C(=C(/c1ccc(O)cc1)c1ccc(OCCN2CCN(C(=O)N3CCC(N4CCN(c5ccc6c(c5)CN(C5CCC(=O)NC5=O)C6=O)CC4)CC3)CC2)cc1)c1ccccc1] | (E)-3-(5-(4-(1-(4-(2-(4-(1-(4- hydroxyphenyl)-2-phenylbut-1-en-1- yl)phenoxy)ethyl)piperazine-1- carbonyl)piperidin-4-yl)piperazin-1-yl)- 1-oxoisoindolin-2-yl)piperidine-2,6- dione | 866.45 | A | A | B | A |
| 95 | 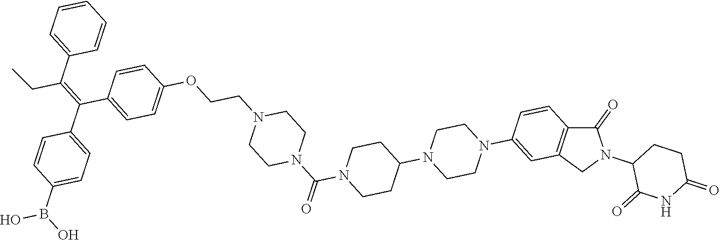 [SMILES: CC/C(=C(/c1ccc(OCCN2CCN(C(=O)N3CCC(N4CCN(c5ccc6c(c5)CN(C5CCC(=O)NC5=O)C6=O)CC4)CC3)CC2)cc1)c1ccc(B(O)O)cc1)c1ccccc1] | (Z)-(4-(1-(4-(2-(4-(4-(4-(2-(2,6- dioxopiperidin-3-yl-1-oxoisoindolin-5- yl)piperazin-1-yl)piperidine-1- carbonyl)piperazin-1-yl)ethoxy)phenyl)- 2-phenylbut-1-en-1-yl)phenyl)boronic acid | 894.46 | A | A | B | A |
| 96 | 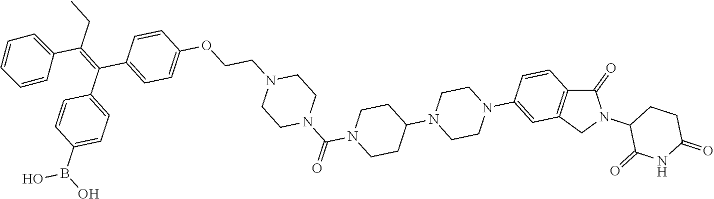 [SMILES: CC/C(=C(\c1ccc(OCCN2CCN(C(=O)N3CCC(N4CCN(c5ccc6c(c5)CN(C5CCC(=O)NC5=O)C6=O)CC4)CC3)CC2)cc1)c1ccc(B(O)O)cc1)c1ccccc1] | (E)-(4-(1-(4-(2-(4-(4-(4-(2-(2,6- dioxopiperidin-3-yl)-1-oxoisoindolin-5- yl)piperazin-1-yl)piperidine-1-carbonyl) piperazin-1-yl)ethoxyl)phenyl)- 2-phenylbut-1-en-1-yl)phenyl)boronic acid | 894.46 | A | A | B | A |
| 97 | 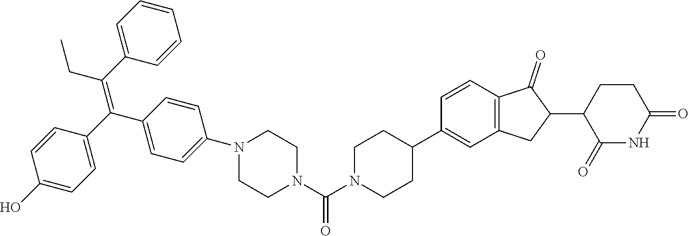 [SMILES: CC/C(=C(\c1ccc(O)cc1)c1ccc(N2CCN(C(=O)N3CCC(c4ccc5c(c4)CC(C4CCC(=O)NC4=O)C5=O)CC3)CC2)cc1)c1ccccc1] | (E)-3-(5-(4-(4-(4-(1-(4-hydroxyphenyl)- 2-phenylbut-1-en-1- yl)phenyl)piperazine-1- carbonyl)piperazin-1-yl)-1- oxoisoindolin-2-yl)piperidine-2,6-dione | 739.35 | A | B | B | A |
| 98 | 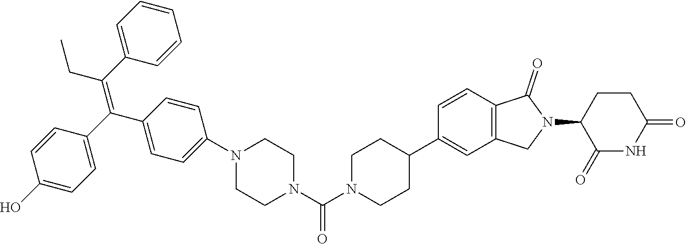 [SMILES: CC/C(=C(\c1ccc(O)cc1)c1ccc(N2CCN(C(=O)N3CCC(c4ccc5c(c4)CN([C@H]4CCC(=O)NC4=O)C5=O)CC3)CC2)cc1)c1ccccc1] | (S,E)-3-(5-(4-(4-(4-(1-(4- hydroxyphenyl)-2-phenylbut-1-en-1- yl)phenyl)piperazine-1- carbonyl)piperazin-1-yl-1- oxoisoindolin-2-yl)piperidine-2,6-dione | 739.35 | A | B | B | A |
| 99 | 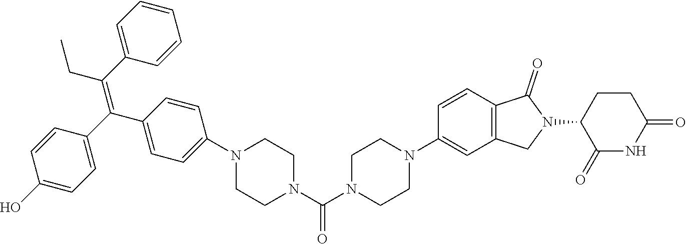 [SMILES: CC/C(=C(\c1ccc(O)cc1)c1ccc(N2CCN(C(=O)N3CCN(c4ccc5c(c4)CN([C@@H]4CCC(=O)NC4=O)C5=O)CC3)CC2)cc1)c1ccccc1] | (R,E)-3-(5-(4-(4-(4-(1-(4- hydroxyphenyl)-2-phenylbut-1-en-1- yl)phenyl)piperazine-1- carbonyl)piperazin-1-yl)-1- oxoisoindolin-2-yl)piperidine-2,6-dione | 739.35 | A | B | B | A |
| 100 | 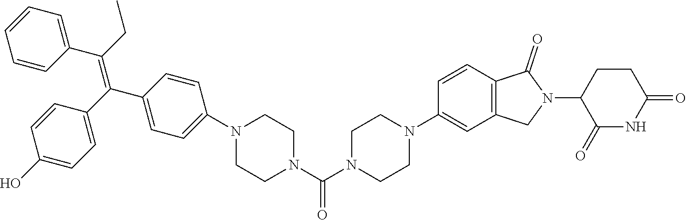 [SMILES: CC/C(=C(/c1ccc(O)cc1)c1ccc(N2CCN(C(=O)N3CCN(c4ccc5c(c4)CN(C4CCC(=O)NC4=O)C5=O)CC3)CC2)cc1)c1ccccc1] | (Z)-3-(5-(4-(4-(4-(1-(4- hydroxyphenyl)-2-phenylbut-1-en-1- yl)phenyl)piperazine-1- carbonyl)piperazin-1-yl-1- oxoisoindolin-2-yl)piperidine-2,6-dione | 739.35 | A | B | A | A |
| 101 | 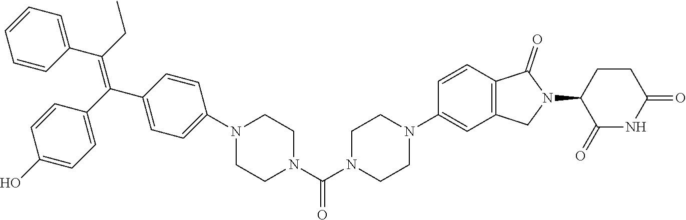 [SMILES: CC/C(=C(/c1ccc(O)cc1)c1ccc(N2CCN(C(=O)N3CCN(c4ccc5c(c4)CN([C@H]4CCC(=O)NC4=O)C5=O)CC3)CC2)cc1)c1ccccc1] | (S,Z)-3-(5-(4-(4-(4-(1-(4- hydroxyphenyl)-2-phenylbut-1-en-1- yl)phenyl)piperazine-1- carbonyl)piperazin-1-yl)-1- oxoisoindolin-2-yl)piperidine-2,6-dione | 739.35 | A | B | A | A |
| 102 | 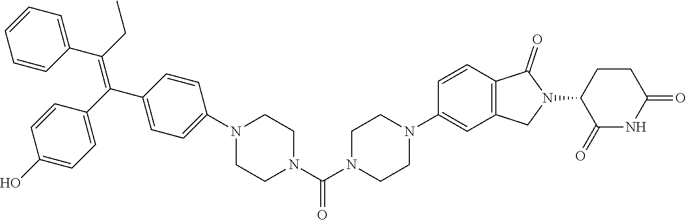 [SMILES: CC/C(=C(/c1ccc(O)cc1)c1ccc(N2CCN(C(=O)N3CCN(c4ccc5c(c4)CN([C@@H]4CCC(=O)NC4=O)C5=O)CC3)CC2)cc1)c1ccccc1] | (R,Z)-3-(5-(4-(4-(4-(1-(4- hydroxyphenyl)-2-phenylbut-1-en-1- yl)phenyl)piperazine-1- carbonyl)piperazin-1-yl)-1- oxoisoindolin-2-yl)piperidine-2,6-dione | 739.35 | A | B | A | A |
| 103 | 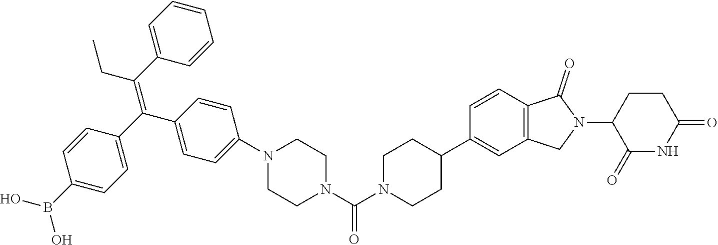 [SMILES: CC/C(=C(\c1ccc(B(O)O)cc1)c1ccc(N2CCN(C(=O)N3CCC(c4ccc5c(c4)CN(C4CCC(=O)NC4=O)C5=O)CC3)CC2)cc1)c1ccccc1] | (Z)-(4-(1-(4-(4-(4-(2-(2,6- dioxopiperidin-3-yl)-1-oxoisoindolin-5- yl)piperazine-1-carbonyl)piperazin-1- yl)phenyl)-2-phenylbut-1-en-1- yl)phenyl)boronic acid | 767.37 | A | A | A | A |
| 104 | 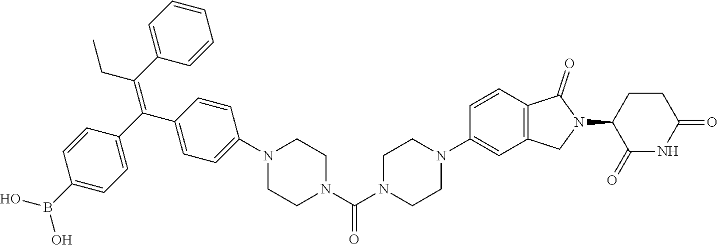 [SMILES: CC/C(=C(\c1ccc(B(O)O)cc1)c1ccc(N2CCN(C(=O)N3CCN(c4ccc5c(c4)CN([C@H]4CCC(=O)NC4=O)C5=O)CC3)CC2)cc1)c1ccccc1] | (S,Z)-(4-(1-(4-(4-(4-(2-(2,6- dioxopiperidin-3-yl)-1-oxoisoindolin-5- yl)piperazine-1-carbonyl)piperazin-1- yl)phenyl)-2-phenylbut-1-en-1- yl)phenyl)boronic acid | 767.37 | A | A | A | A |
| 105 | 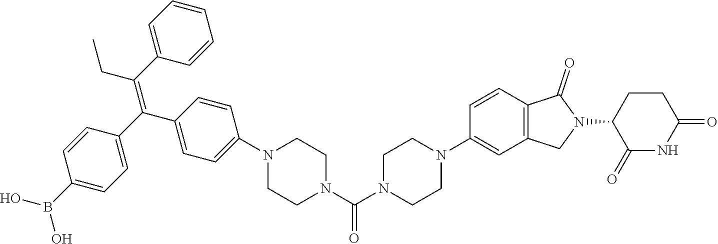 [SMILES: CC/C(=C(\c1ccc(B(O)O)cc1)c1ccc(N2CCN(C(=O)N3CCN(c4ccc5c(c4)CN([C@@H]4CCC(=O)NC4=O)C5=O)CC3)CC2)cc1)c1ccccc1] | (R,Z)-(4-(1-(4-(4-(4-(2-(2,6- dioxopiperidin-3-yl-1-oxoisoindolin-5- yl)piperazine-1-carbonyl)piperazin-1- yl)phenyl)-2-phenylbut-1-en-1- yl)phenyl)boronic acid | 767.37 | A | A | A | A |
| 106 | 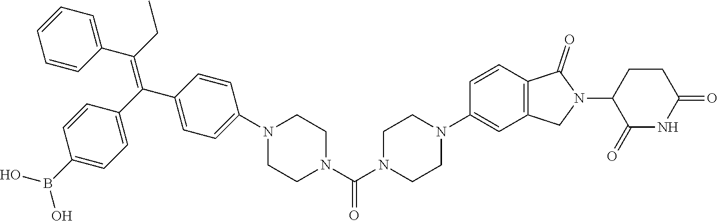 [SMILES: CC/C(=C(/c1ccc(B(O)O)cc1)c1ccc(N2CCN(C(=O)N3CCN(c4ccc5c(c4)CN(C4CCC(=O)NC4=O)C5=O)CC3)CC2)cc1)c1ccccc1] | (E)-(4-(1-(4-(4-(4-(2-(2,6- dioxopiperidin-3-yl)-1-oxoisoindolin-5- yl)piperazine-1-carbonyl)piperazin-1- yl)phenyl)-2-phenylbut-1-en-1- ylphenyl)boronic acid | 767.37 | A | A | A | A |
| 107 | 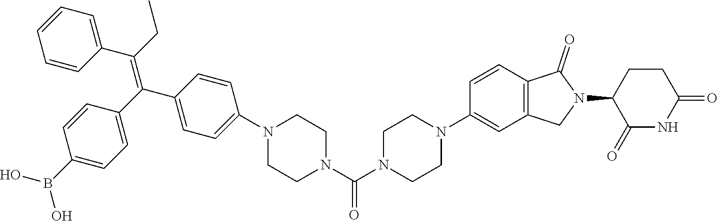 [SMILES: CC/C(=C(/c1ccc(B(O)O)cc1)c1ccc(N2CCN(C(=O)N3CCN(c4ccc5c(c4)CN([C@H]4CCC(=O)NC4=O)C5=O)CC3)CC2)cc1)c1ccccc1] | (S,E)-(4-(1-(4-(4-(4-(2-(2,6- dioxopiperidin-3-yl)-1-oxoisoindolin-5- yl)piperazine-1-carbonyl)piperazin-1- yl)phenyl)-2-phenylbut-1-en-1- yl)phenyl)boronic acid | 767.37 | A | A | A | A |
| 108 | 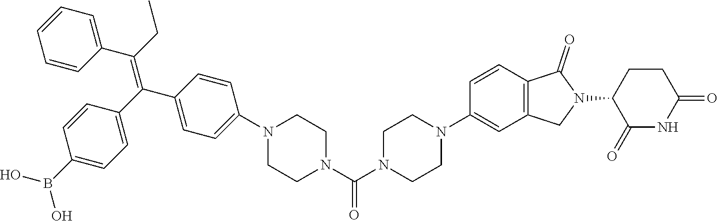 [SMILES: CC/C(=C(/c1ccc(B(O)O)cc1)c1ccc(N2CCN(C(=O)N3CCN(c4ccc5c(c4)CN([C@@H]4CCC(=O)NC4=O)C5=O)CC3)CC2)cc1)c1ccccc1] | (R,E)-(4-(1-(4-(4-(4-(2-(2,6- dioxopiperidin-3-yl)-1-oxoisoindolin-5- yl)piperazine-1-carbonyl)piperazin-1- yl)phenyl)-2-phenylbut-1-en-1- yl)phenyl)boronic acid | 767.37 | A | A | A | A |
| 109 | 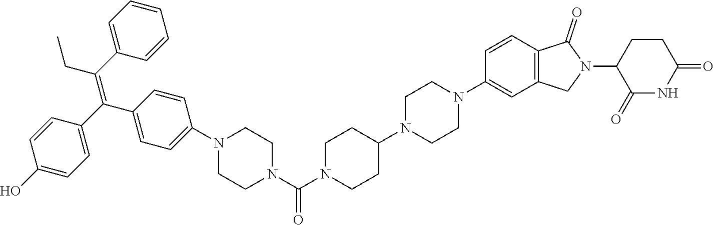 [SMILES: CC/C(=C(\c1ccc(O)cc1)c1ccc(N2CCN(C(=O)N3CCC(N4CCN(c5ccc6c(c5)CN(C5CCC(=O)NC5=O)C6=O)CC4)CC3)CC2)cc1)c1ccccc1] | (E)-3-(5-(4-(1-(4-(4-(1-(4- hydroxyphenyl)-2-phenylbut-1-en-1- yl)phenyl)piperazine-1- carbonyl)piperidin-4-yl)piperazin-1-yl)- 1-oxoisoindolin-2-yl)piperidine-2,6- dione | 822.43 | A | B | B | A |
| 110 | 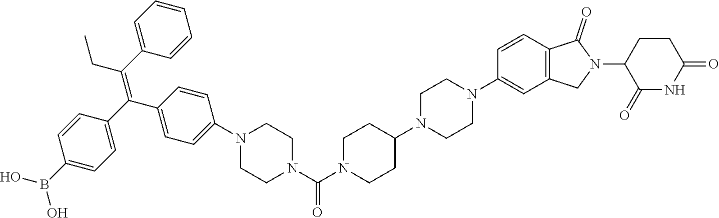 [SMILES: CC/C(=C(\c1ccc(B(O)O)cc1)c1ccc(N2CCN(C(=O)N3CCC(N4CCN(c5ccc6c(c5)CN(C5CCC(=O)NC5=O)C6=O)CC4)CC3)CC2)cc1)c1ccccc1] | (Z)-(4-(1-(4-(4-(4-(4-(2-(2,6- dioxopiperidin-3-yl)-1-oxoisoindolin-5- yl)piperazin-1-yl)piperidine-1- carbonyl)piperazin-1-yl)phenyl)-2- phenylbut-1-en-1-yl)phenyl)boronic acid | 822.43 | A | B | B | A |
| 111 | 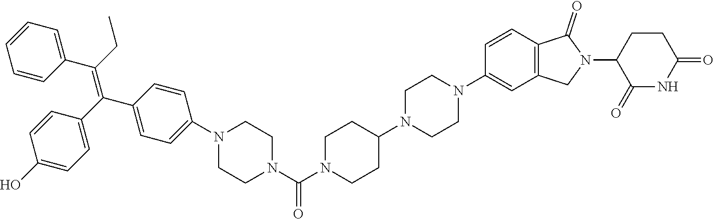 [SMILES: CC/C(=C(/c1ccc(O)cc1)c1ccc(N2CCN(C(=O)N3CCC(N4CCN(c5ccc6c(c5)CN(C5CCC(=O)NC5=O)C6=O)CC4)CC3)CC2)cc1)c1ccccc1] | (Z)-3-(5-(4-(1-(4-(4-(1-(4- hydroxyphenyl)-2-phenylbut-1-en-1- yl)phenyl)piperazine-1- carbonyl)piperidin-4-yl)piperazin-1-yl)- 1-oxoisoindolin-2-yl)piperidine-2,6- dione | 822.43 | A | B | B | A |
| 112 | 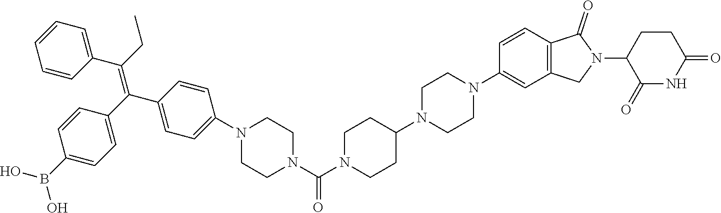 [SMILES: CC/C(=C(/c1ccc(B(O)O)cc1)c1ccc(N2CCN(C(=O)N3CCC(N4CCN(c5ccc6c(c5)CN(C5CCC(=O)NC5=O)C6=O)CC4)CC3)CC2)cc1)c1ccccc1] | (E)-(4-(1-(4-(4-(4-(4-(2-(2,6- dioxopiperidin-3-yl)-1-oxoisoindolin-5- yl)piperazin-1-yl)piperidine-1- carbonyl)piperazin-1-yl)phenyl)-2- phenylbut-1-en-1-yl)phenyl)boronic acid | 822.43 | A | B | B | A |
| 113 | 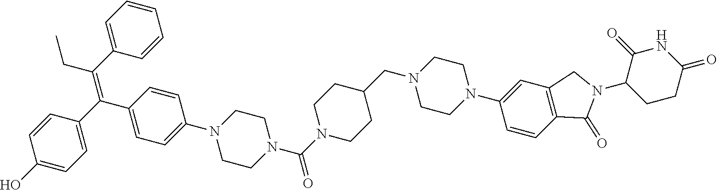 [SMILES: CC/C(=C(\c1ccc(O)cc1)c1ccc(N2CCN(C(=O)N3CCC(CN4CCN(c5ccc6c(c5)CN(C5CCC(=O)NC5=O)C6=O)CC4)CC3)CC2)cc1)c1ccccc1] | (E)-3-(5-(4-((1-(4-(4-(1-(4- hydroxyphenyl)-2-phenylbut-1-en-1- yl)phenyl)piperazine-1- carbonyl)piperidin-4- yl)methyl)piperazin-1-yl)-1- oxoisoindolin-2-yl)piperidine-2,6-dione | 835.45 | A | A | A | A |
| 114 | 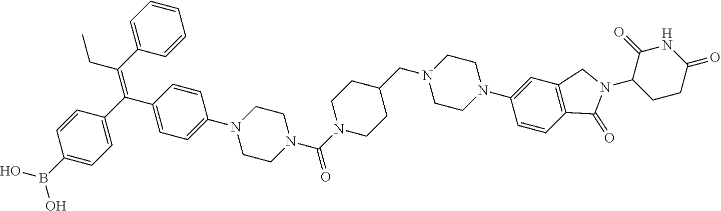 [SMILES: CC/C(=C(\c1ccc(B(O)O)cc1)c1ccc(N2CCN(C(=O)N3CCC(CN4CCN(c5ccc6c(c5)CN(C5CCC(=O)NC5=O)C6=O)CC4)CC3)CC2)cc1)c1ccccc1] | (Z)-(4-(1-(4-(4-(4-((4-(2-(2,6- dioxopiperidin-3-yl)-1-oxoisoindolin-5- yl)piperazin-1-yl)methyl)piperidine-1- carbonyl)piperazin-1-yl)phenyl)-2- phenylbut-1-en-1-yl)phenyl)boronic acid | 835.45 | A | A | A | A |
| 115 | 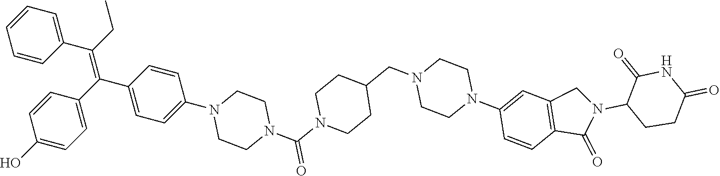 [SMILES: CC/C(=C(/c1ccc(O)cc1)c1ccc(N2CCN(C(=O)N3CCC(CN4CCN(c5ccc6c(c5)CN(C5CCC(=O)NC5=O)C6=O)CC4)CC3)CC2)cc1)c1ccccc1] | (Z)-3-(5-(4-((1-(4-(4-(1-(4- hydroxyphenyl)-2-phenylbut-1-en-1- yl)phenyl)piperazine-1- carbonyl)piperidin-4- yl)methyl)piperazin-1-yl)-1- oxoisoindolin-2-yl)piperidine-2,6-dione | 835.45 | A | A | A | A |
| 116 | 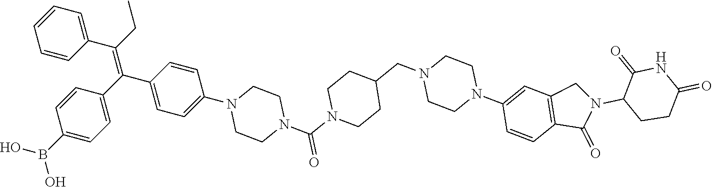 [SMILES: CC/C(=C(/c1ccc(B(O)O)cc1)c1ccc(N2CCN(C(=O)N3CCC(CN4CCN(c5ccc6c(c5)CN(C5CCC(=O)NC5=O)C6=O)CC4)CC3)CC2)cc1)c1ccccc1] | (E)-(4-(1-(4-(4-(4-((4-(2-(2,6- dioxopiperidin-3-yl)-1-oxoisoindolin-5- yl)piperazin-1-yl)methyl)piperidine-1- carbonyl)piperazin-1-yl)phenyl)-2- phenylbut-1-en-1-yl)phenyl)boronic acid | 835.45 | A | A | A | A |

### Pharmaceutical Compositions

This specification also describes, in part, a composition which comprises a compound of Formula (I), or a derivative, tautomer, stereoisomer, mixture of stereoisomers, pharmaceutically acceptable salt, or solvate thereof.

This specification also describes, in part, a pharmaceutical composition which comprises a compound of Formula (I), or a pharmaceutically acceptable salt thereof, and at least one pharmaceutically acceptable excipient.

This specification also describes, in part, a compound of Formula (I), or a pharmaceutically acceptable salt thereof, for use in reducing the level or activity of a target protein (e.g., an estrogen receptor).

This specification also describes, in part, a compound of Formula (I), or a pharmaceutically acceptable salt thereof, for use in inhibiting a target protein (e.g., an estrogen receptor).

This specification also describes, in part, a compound of Formula (I), or a pharmaceutically acceptable salt thereof, for use in therapy.

This specification also describes, in part, a compound of Formula (I), or a pharmaceutically acceptable salt thereof, for use in the treatment of cancer.

This specification also describes, in part, a method for treating cancer in a warm-blooded animal in need of such treatment, which comprises administering to the warm-blooded animal a therapeutically effective amount of a compound of Formula (I), or a pharmaceutically acceptable salt thereof.

In one embodiment, the disclosure provides for a pharmaceutical composition comprising at least one compound of Formula (I) or a pharmaceutically acceptable salt or solvate thereof. In an embodiment, the pharmaceutical compound is for use in treatment of a proliferative disease, such as a cancer, for example, a breast cancer. A further embodiment may provide a method of treating breast cancer comprising administering to a subject in need of treatment or amelioration a compound according to any one of the preceding paragraphs. The breast cancer may be an ER-positive breast cancer. The subject may express a mutant ER-α protein or any variant of ER-α, such as ERα-36. An embodiment may provide proper and effective use of a compound as in the paragraphs above for treating and/or preventing breast cancer. In some embodiments the breast cancer is an ER-positive breast cancer. In some embodiments said subject expresses a mutant ER-α protein or a variant of ERα, such as ERα-36. In some embodiments a compound as presented above is used in the preparation of a medicament for treatment of breast cancer in a patient or subject, such as a human or animal.

The pharmaceutical compositions of the present disclosure can be in any form known to those of skill in the art, and a suitable dosage form of the compound(s) can be administered by an appropriate route. For instance, in some embodiments the pharmaceutical compositions are in a form of a product for oral delivery, said product form being selected from a group consisting of a concentrate, dried powder, liquid, capsule, pellet, and pill. In other embodiments, the pharmaceutical compositions of the disclosure are in the form of a product for parenteral administration including intravenous, intradermal, intramuscular, intraarticular, intra-synovial, intrasternal, intrathecal and subcutaneous administration. The compounds described herein may be administered as a single dose or a divided dose over a period of time. The pharmaceutical compositions disclosed herein may also further comprise carriers, binders, diluents, and excipients. The described carriers, diluents and excipients may include dried corn starch or lactose, the binder may include microcrystalline cellulose, gum tragacanth or gelatin, in addition, the excipients may also include a dispersing agent, a lubricant, a glidant, a sweetening agent or a flavoring agent.

Also, in other aspects, the present disclosure relates to new ER degrading composition comprising one or more compounds selected from the group consisting of a compound of Formula (I) and pharmaceutically acceptable salts and solvates thereof. In an embodiment, said compound has a purity of about ≥75%, ≥80%, ≥85%, ≥90%, ≥95%, ≥96%, ≥97%, or ≥98%, and ≥99%. In an embodiment, a pharmaceutical composition is provided comprising the new ER degrading composition, either alone or in combination with at least one additional therapeutic agent, with a pharmaceutically acceptable carrier; and uses of the new ER degrading compositions, either alone or in combination with at least one additional therapeutic agent, in the treatment of proliferative diseases including breast cancer at any stage of the disease diagnosis. The combination with an additional therapeutic agent may take the form of combining the new ER degrading compounds with any known therapeutic agent.

The disclosed compounds can be used to slow the rate of primary tumor growth. The disclosed compounds can also be used to prevent, abate, minimize, control, and/or lessen tumor metastasis in humans and animals. The disclosed compounds when administered to a subject in need of treatment can be used to stop the spread of cancer cells. As such, the compounds disclosed herein can be administered as part of a combination therapy with one or more drugs or other pharmaceutical agents. When used as part of the combination therapy, the decrease in metastasis and reduction in primary tumor growth afforded by the disclosed compounds allows for a more effective and efficient use of any pharmaceutical or drug therapy being used to treat the patient. In addition, control of metastasis by the disclosed compound affords the subject a greater ability to concentrate the disease in one location.

The following are non-limiting examples of cancers that can be treated by the disclosed methods and compositions: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytotna/Malignant Glioma; Craniopharyngioma; Ependymoblastoma; Ependymoma; Medulloblastoma; Medulloepithelioma; Pineal Parenchymal Tumors of intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocvtoma of Bone and Osteosarcoma; Medulloblastoma; Melanoma; Melanoma, intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis; Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Celt Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor.

### Methods of Treatment

The methods for treating a clinical indication by the ER degrading compounds disclosed herein, may be effectuated by administering a therapeutically effective amount of the ER degrading compounds to a patient in need thereof, this therapeutically effective amount may comprise administration of the prodrug to the patient at about 1 mg/kg/day, 2 mg/kg/day, 3 mg/kg/day, 4 mg/kg/day, 5 mg/kg/day, 10 mg/kg/day and 20 mg/kg/day. Alternatively, amounts ranging from about 0.001 mg/kg/day to about 0.01 mg/kg/day, or about 0.01 mg/kg/day to about 0.1 mg/kg/day, or about 0.1 mg/kg/day to about 1 mg/kg/day, or about 1 mg/kg/day to 10 mg/kg/day, or about 10 mg/kg/day to about 100 mg/kg/day are also contemplated.

A further object of the disclosure is a kit, comprising a composition containing at least one ER degrading compound for treatment and prevention of cancer and cancer related morbidities. The composition of the kit may comprise at least one carrier, at least one binder, at least one diluent, at least one excipient, at least one other therapeutic agent, or mixtures thereof. The kit may be designed, developed, distributed, or sold as a unit for performing the methods of the present invention and to deliver the drugs to the targeted cells for the treatment and prevention of cancer and related diseases. The kits may also include instructions to customers for proper usage of the kit to treat patients exhibiting the symptoms of the desired disease, e.g., cancer or breast cancer.

One aspect of the present disclosure is the compounds disclosed herein as well as the intermediates as used for their synthesis, and the synthetic scheme for the preparation of the disclosed final compounds and the intermediates resulted before the final compound is generated.

While certain features of this invention shown and described below are pointed out in the annexed claims, the invention is not intended to be limited to the details specified, since a person of ordinary skill in the relevant art will understand that various omissions, modifications, substitutions, and changes in the forms and details of the invention illustrated and in its operation may be made without departing in any way from the spirit of the present invention. No feature of the invention is critical or essential unless it is specifically stated as being “critical” or “essential”.

These and other features, aspects, and advantages of embodiments of the present disclosure will become more evident with regard to the following descriptions, claims, and accompanying drawings explained below.

Another object of the disclosure is to provide a composition, for example a pharmaceutical composition, comprising at least one ER degrader compound in an amount effective for the indication of proliferative diseases such as cancer, including but not limited to endocrine related cancer. In an embodiment, the cancer is an ER-positive tumor, such as a tumor of the breast, endometrium, uterus, or ovary. In an embodiment, the tumor is an ER-positive tumor of the breast. In an embodiment, the breast tumor is determined to be ER-positive by an immunohistochemical method described by Hammond et al.[8],

In an embodiment, the object of such treatment is to degrade estrogen receptor and/or inhibit estrogen-induced proliferation of a cell. In a further embodiment, said object is to inhibit estrogen-induced proliferation of a cell by a mechanism selected from SERM, SERD, and PROTAC.

As used herein, “treating” means administering to a subject a pharmaceutical composition to ameliorate, reduce or lessen the symptoms of a disease. As used herein, “treating” or “treat” describes the management and care of a subject for the purpose of combating a disease, condition, or disorder and includes the administration of a compound disclosed herein, or a pharmaceutically acceptable salt, polymorph or solvate thereof, to alleviate the symptoms or complications of a disease, condition or disorder, or to eliminate the disease, condition or disorder. The term “treat” may also include treatment of a cell in vitro or an animal model. As used herein, “subject” or “subjects” refers to any animal, such as mammals including rodents (e.g., mice or rats), dogs, primates, lemurs or humans.

Treating cancer may result in a reduction in size of a tumor. A reduction in size of a tumor may also be referred to as “tumor regression.” Preferably, after treatment, tumor size is reduced by about 5% or greater relative to its size prior to treatment; more preferably, tumor size is reduced by about 10% or greater; more preferably, reduced by about 20% or greater; more preferably, reduced by about 30% or greater; more preferably, reduced by about 40% or greater; even more preferably, reduced by about 50% or greater; and most preferably, reduced by greater than about 75% or greater. Size of a tumor may be measured by any reproducible means of measurement. The size of a tumor may be measured as a diameter of the tumor.

Treating cancer may result in a reduction in tumor volume. Preferably, after treatment, tumor volume is reduced by about 5% or greater relative to its size prior to treatment; more preferably, tumor volume is reduced by about 10% or greater; more preferably, reduced by about 20% or greater; more preferably, reduced by about 30% or greater; more preferably, reduced by about 40% or greater; even more preferably, reduced by about 50% or greater; and most preferably, reduced by about 75% or greater. Tumor volume may be measured by any reproducible means of measurement.

Treating cancer may result in a decrease in number of tumors. Preferably, after treatment, tumor number is reduced by about 5% or greater relative to number prior to treatment; more preferably, tumor number is reduced by about 10% or greater; more preferably, reduced by about 20% or greater; more preferably, reduced by about 30% or greater; more preferably, reduced by about 40% or greater; even more preferably, reduced by about 50% or greater; and most preferably, reduced by greater than about 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment, the number of metastatic lesions is reduced by about 5% or greater relative to number prior to treatment; more preferably, the number of metastatic lesions is reduced by about 10% or greater; more preferably, reduced by about 20% or greater; more preferably, reduced by about 30% or greater; more preferably, reduced by about 40% or greater; even more preferably, reduced by about 50% or greater; and most preferably, reduced by greater than about 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer may result in an increase in average survival time of a population of treated subjects in comparison to a population receiving carrier alone. Preferably, the average survival time is increased by more than about 30 days; more preferably, by more than about 60 days; more preferably, by more than about 90 days; and most preferably, by more than about 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in an increase in average survival time of a population of treated subjects in comparison to a population of untreated subjects. Preferably, the average survival time is increased by more than about 30 days; more preferably, by more than about 60 days; more preferably, by more than about 90 days; and most preferably, by more than about 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in increase in average survival time of a population of treated subjects in comparison to a population receiving monotherapy with a drug that is not a compound disclosed herein, or a pharmaceutically acceptable salt thereof. Preferably, the average survival time is increased by more than about 30 days; more preferably, by more than about 60 days; more preferably, by more than about 90 days; and most preferably, by more than about 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer may result in a decrease in the mortality rate of a population of treated subjects in comparison to a population receiving carrier alone. Treating cancer may result in a decrease in the mortality rate of a population of treated subjects in comparison to an untreated population. Treating cancer may result in a decrease in the mortality rate of a population of treated subjects in comparison to a population receiving monotherapy with a drug that is not a compound disclosed herein, or a pharmaceutically acceptable salt, prodrug, metabolite, analog or derivative thereof. Preferably, the mortality rate is decreased by more than about 2%; more preferably, by more than about 5%; more preferably, by more than about 10%; and most preferably, by more than about 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer may result in a decrease in tumor growth rate. Preferably, after treatment, tumor growth rate is reduced by at least about 5% relative to number prior to treatment; more preferably, tumor growth rate is reduced by at least about 10%; more preferably, reduced by at least about 20%; more preferably, reduced by at least about 30%; more preferably, reduced by at least about 40%; more preferably, reduced by at least about 50%; even more preferably, reduced by at least about 50%; and most preferably, reduced by at least about 75%. Tumor growth rate may be measured by any reproducible means of measurement. Tumor growth rate may be measured according to a change in tumor diameter per unit time.

Treating cancer may result in a decrease in tumor regrowth, for example, following attempts to remove it surgically. Preferably, after treatment, tumor regrowth is less than about 5%; more preferably, tumor regrowth is less than about 10%; more preferably, less than about 20%; more preferably, less than about 30%; more preferably, less than about 40%; more preferably, less than about 50%; even more preferably, less than about 50%; and most preferably, less than about 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder may result in a reduction in the rate of cellular proliferation. Preferably, after treatment, the rate of cellular proliferation is reduced by at least about 5%; more preferably, by at least about 10%; more preferably, by at least about 20%; more preferably, by at least about 30%; more preferably, by at least about 40%; more preferably, by at least about 50%; even more preferably, by at least about 50%; and most preferably, by at least about 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder may result in a reduction in the proportion of proliferating cells. Preferably, after treatment, the proportion of proliferating cells is reduced by at least about 5%; more preferably, by at least about 10%; more preferably, by at least about 20%; more preferably, by at least about 30%; more preferably, by at least about 40%; more preferably, by at least about 50%; even more preferably, by at least about 50%; and most preferably, by at least about 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells may be equivalent to the mitotic index.

Treating or preventing a cell proliferative disorder may result in a decrease in size of an area or zone of cellular proliferation. Preferably, after treatment, size of an area or zone of cellular proliferation is reduced by at least about 5% relative to its size prior to treatment; more preferably, reduced by at least about 10%; more preferably, reduced by at least about 20%; more preferably, reduced by at least about 30%; more preferably, reduced by at least about 40%; more preferably, reduced by at least about 50%; even more preferably, reduced by at least about 50%; and most preferably, reduced by at least about 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder may result in a decrease in the number or proportion of cells having an abnormal appearance or morphology. Preferably, after treatment, the number of cells having an abnormal morphology is reduced by at least about 5% relative to its size prior to treatment; more preferably, reduced by at least about 10%; more preferably, reduced by at least about 20%; more preferably, reduced by at least about 30%; more preferably, reduced by at least about 40%; more preferably, reduced by at least about 50%; even more preferably, reduced by at least about 50%; and most preferably, reduced by at least about 75%. An abnormal cellular appearance or morphology may be measured by any reproducible means of measurement. An abnormal cellular morphology may be measured by microscopy, e.g., using an inverted tissue culture microscope. An abnormal cellular morphology may take the form of nuclear pleiomorphism.

### EXAMPLES

Hereby are provided non-limiting examples of embodiments of compounds disclosed herein. The examples and preparations provided below further illustrate and exemplify the compounds as disclosed herein and methods of preparing such compounds. It is to be understood that the scope of the present disclosure is not limited in any way by the scope of the following examples and preparations. Unless stated otherwise, starting materials were commercially available. All solvents and commercial reagents were of laboratory grade and were used as received.

### Example 1

### Bifunctional Compounds that Perform as Modulators of Estrogen Receptor

Cellular signaling of estrogens is mediated through two estrogen receptor (ER) subtypes, ERα and ERβ, and they belong to the nuclear receptor family of transcription factors. Estrogens play central roles in the development and maintenance of normal sexual and reproductive function. In addition, both ERα and ERβ were found to have distinct biological effects in the immune, skeletal, cardiovascular, and central nervous systems [1]. Estrogen receptors are mainly expressed in ovarian, uterus, liver cells, and are found overexpressed in certain tumor cells, such as breast cancer, ovarian cancer and prostate cancer. The most potent and abundant estrogen produced in human body is 17β-estradiol. Anti-estrogens, designed to block ERα by retreating estrogens from the active site, are widely and effectively used clinically for breast cancer treatment [2].

Breast cancer remains the most common cancer in women worldwide, with over 1.7 million new cases diagnosed in 2012, and it is the second most common cancer overall. This represents about 12% of all new cancer cases and 25% of all cancers in women. Nearly 80% of breast cancer cases are estrogen receptor positive (ER+) [3, 4] and for most of these patients, endocrine therapy is an appropriate option in both the adjuvant and advanced setting. This therapy is used to prevent or block the hormones from stimulating the growth of cancer cells. Current endocrine therapy for ER+ breast cancer comprises three regimen options that can be used in varied sequences for optimal outcome: SERM (e.g., tamoxifen, raloxifene, toremifene), aromatase inhibitors (AIs, including anastrozole, exemestane, letrozole), and SERD (fulvestrant) [2].

Tamoxifen is a first-line agent for pre-menopausal patients and for women requiring secondary chemoprevention after a DCIS diagnosis. In postmenopausal women AIs are generally preferred to tamoxifen because of more favorable time to progression and less severe side effects [5, 6]. Although these synthetic anti-estrogens are the mainline therapy for treating ER+ breast cancers, these drugs cause unwanted adverse effects in non-targeted tissues, and the cancers become resistant to endocrine therapy. After a prolonged treatment, most patients with advanced metastatic breast cancer eventually develop resistance to tamoxifen or AI treatment while retaining the expression of ERα in the recurrent and/or progressive disease. This clinical information provides a viable therapeutic rationale for using effective ER-targeting antagonists that are not cross-resistant to previous endocrine agents.

The conventional anti-estrogens, such as SERMs and SERDs, cannot fully yield the full pharmacological efficacy in the treatment of breast cancer. Though the SERDs can downregulate or degrade the estrogen receptor protein level and block transcriptional activity, the existing drugs are also prone to resistance. Therefore, alternative drugs and new approaches are needed for delivering the drugs to target cells and degrade specific receptors, so as to increase the efficiency of the drugs and lower the side effects. Proteolysis targeting chimeric (PROTAC) technology has emerged as powerful tool for targeted degradation of endogenous proteins [7]. PROTAC, a bifunctional compound attached by a linker molecule, which contain a target protein binding moiety and E3 ubiquitin ligase binding moiety, can simultaneously bind the target protein and E3 ligase and promote ubiquitination of the target protein and degradation of the same by proteasome. Many publications provide the usage of PROTAC molecules as modulators of targeted proteins via ubiquitination and subsequent degradation by cell proteasome. The present invention exploits this powerful tool to specifically degrade estrogen receptor by developing bifunctional compounds consisting of ER binding moiety and CRBN E3 ligase binding moiety linked by various chemical linkers for the treatment of ER+ breast cancer. SERDs reduce the ERα protein level as well as block ER transcription activity. Another method that exploits/utilizes proteasome has offered researchers a tool to manipulate levels of a specific protein and test its function or develop treatment for diseases. In order to exploit the proteasome's role as a drug and manipulate protein levels, proteolysis targeting chimeric molecules (PROTACs) have been developed. PROTACs molecules contain a ligand that recognizes the target protein linked via a linker molecule to a ligand that binds to a specific E3 ubiquitin ligase. In the present case, the bifunctional compounds (or PROTACs) can simultaneously bind ERα and a specific E3 ubiquitin ligase, which promotes ubiquitination of ER and leads to degradation of ER by the proteasome. While small molecules can easily bind enzymes or receptors in tight and well-defined pockets, protein-protein interactions are difficult to target by small molecules. E3 ubiquitin ligases confer substrates specifically for ubiquitination, making it an attractive therapeutic strategy for protein degradation by proteasome.

The present disclosure relates to bifunctional compounds that perform as modulators of estrogen receptor (target protein), which are degraded or inhibited as a result of ubiquitination and subsequent degradation of the ubiquitinated ER by the proteasome. The present disclosure provides the methods for making these compounds and their usage in treating or ameliorating disease conditions/states associated with aggregation or accumulation of estrogen receptor. The disclosure also relates to pharmaceutical compositions comprising these ER degrading bifunctional compounds, and methods for using the same for treatment of estrogen receptor mediated pathological developments, including cancers.

The compounds described here can provide effective endocrine therapy for breast cancers, especially those that are associated with overexpression or aggregation of estrogen receptor (estrogen receptor positive or “ER+” breast cancers), including its mutant form, as the first line adjuvant treatment regimen, or as the second-line therapy for treating or ameliorating patients with disease progression owing to drug resistance after prior endocrine therapy such as selective estrogen receptor modulators (SERMs), aromatase inhibitors (AIs), SERDs, or combinations of such endocrine therapies with other anticancer agents. Thus, the present disclosed compounds herein can be used for the treatment of ER+ breast cancer, including the advanced drug-resistant ER+ breast cancer.

### Example 2

### General Synthesis

The chemical entities described herein can be synthesized according to one or more illustrative schemes herein and/or techniques well known in the art. Unless specified to the contrary, the reactions described herein take place at atmospheric pressure, generally within a temperature range from about −10° C. to about 200° C. Further, except as otherwise specified, reaction times and conditions are intended to be approximate, e.g., taking place at about atmospheric pressure within a temperature range of about −10° C. to about 200° C. over a period that can be, for example, about 1 to about 24 hours; reactions left to run overnight in some embodiments can average a period of about 16 hours. Isolation and purification of the chemical entities and intermediates described herein can be implemented, if desired, by any suitable separation or purification procedure such as, for example, filtration, extraction, crystallization, column chromatography, thin-layer chromatography or thick-layer chromatography, or a combination of these procedures. See, e.g., Carey et al. Advanced Organic Chemistry, 3rd Ed., 1990 New York: Plenum Press; Mundy et al., Name Reaction and Reagents in Organic Synthesis, 2nd Ed., 2005 Hoboken, N.J.: J. Wiley & Sons. Specific illustrations of suitable separation and isolation procedures are given by reference to the examples hereinbelow. However, other equivalent separation or isolation procedures can also be used.

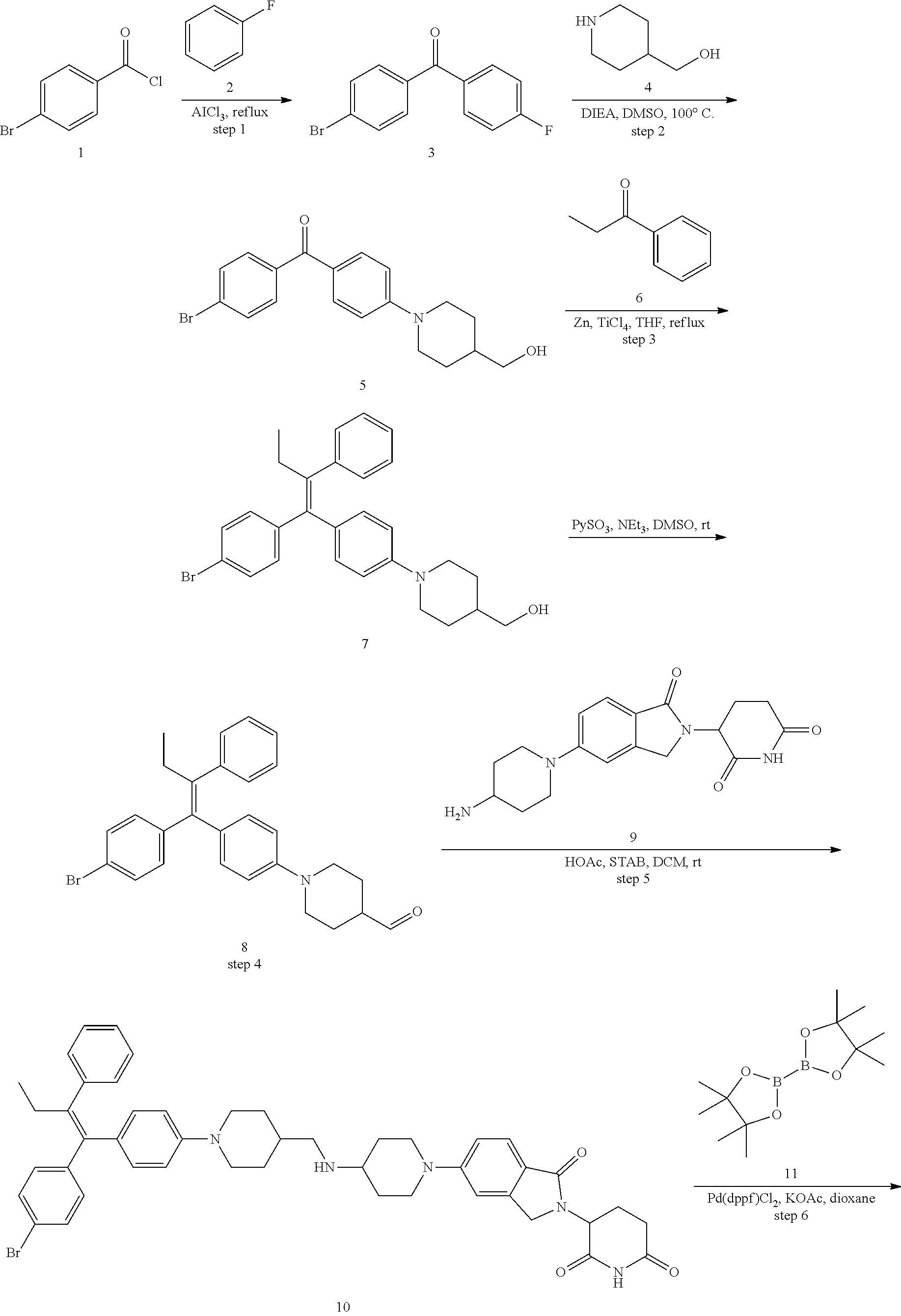

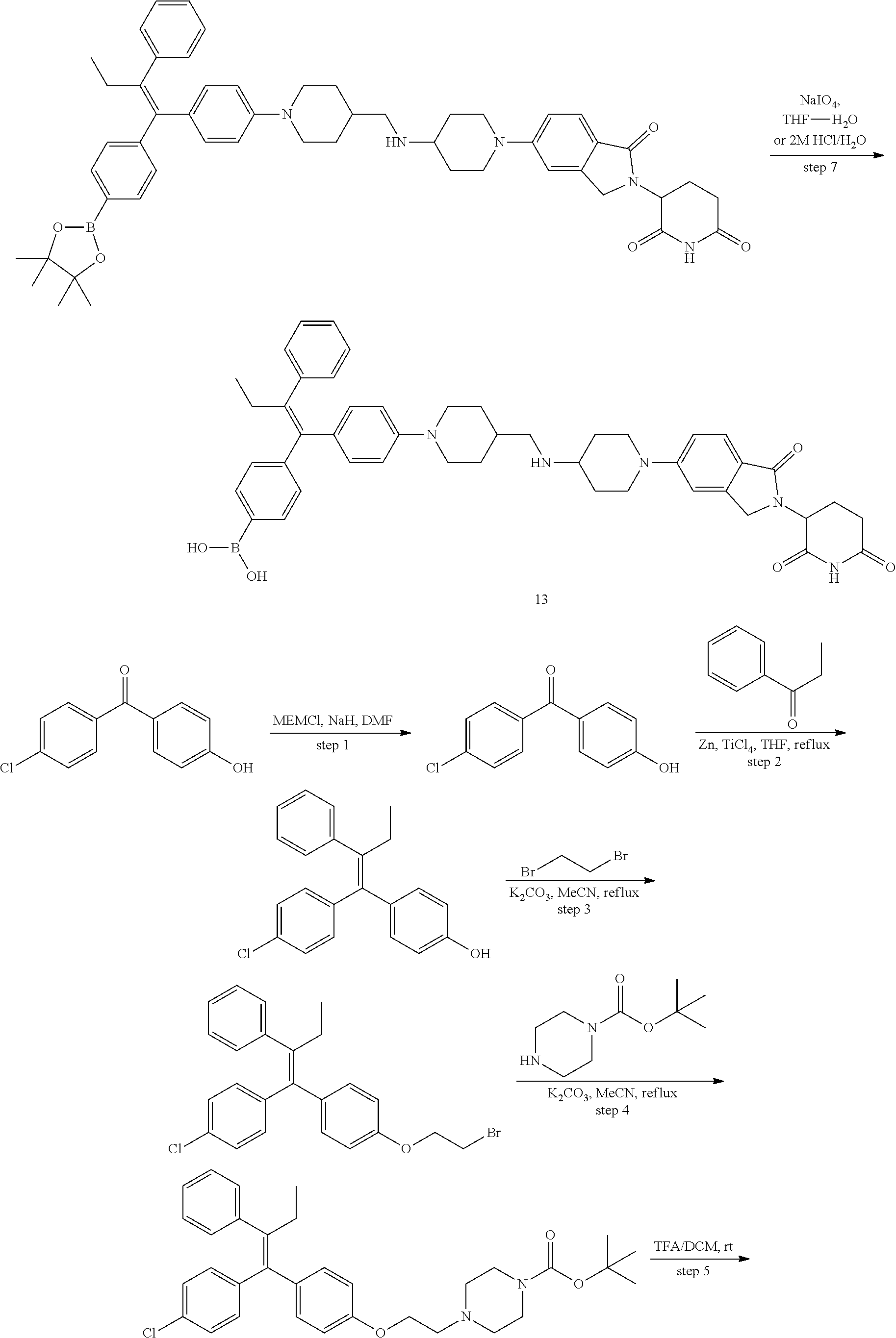

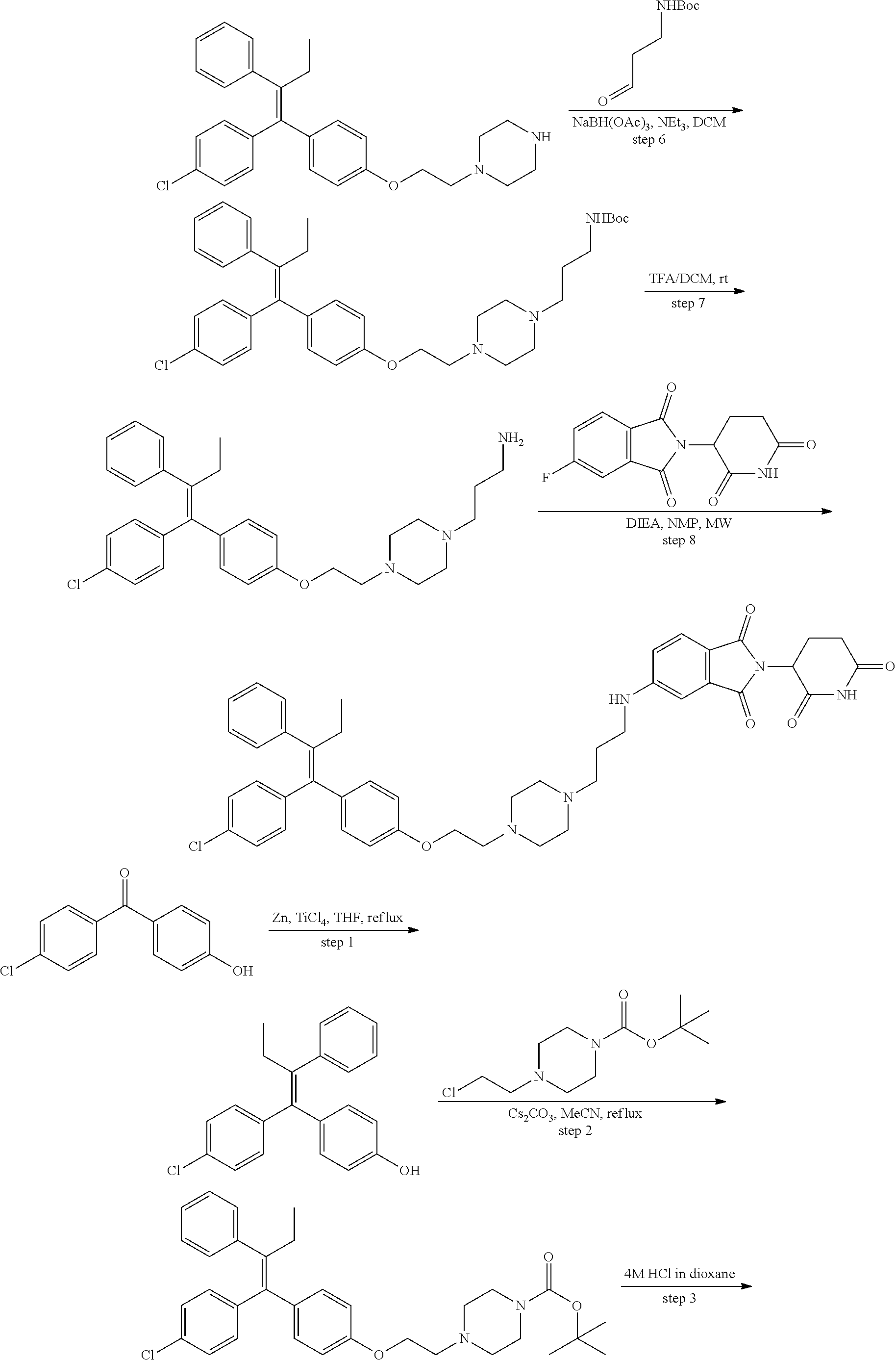

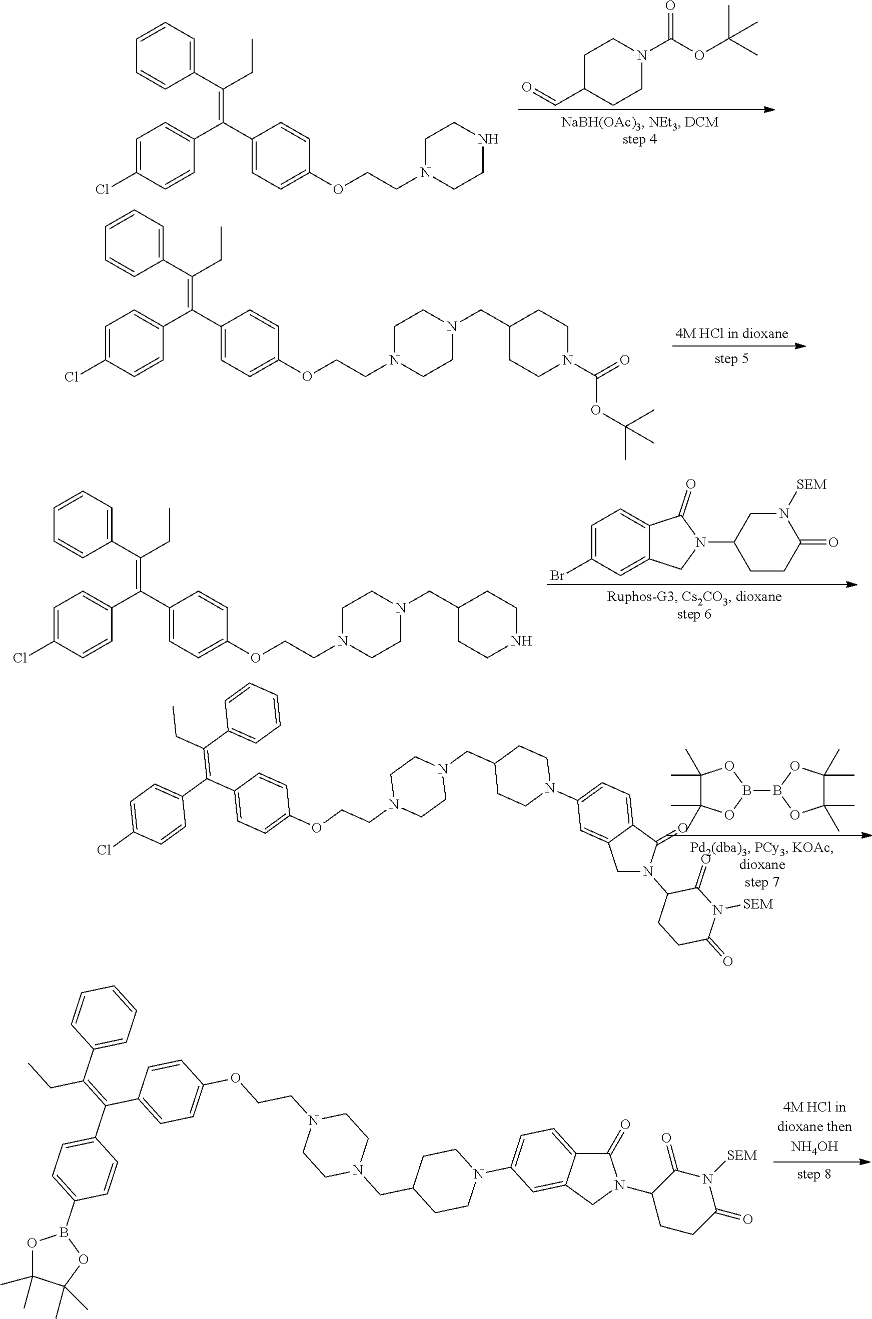

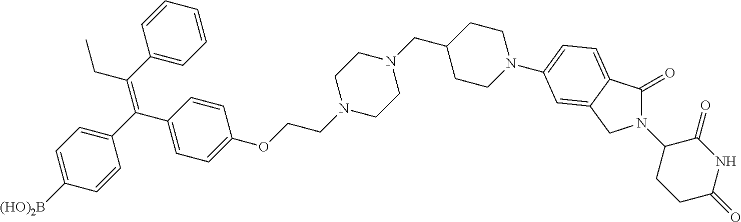 [SMILES: CC/C(=C(/c1ccc(OCCN2CCN(CC3CCN(c4ccc5c(c4)CN(C4CCC(=O)NC4=O)C5=O)CC3)CC2)cc1)c1ccc(B(O)O)cc1)c1ccccc1]

In all of the methods, it is well understood that protecting groups for sensitive or reactive groups may be employed where necessary, in accordance with general principles of chemistry. Protecting groups are manipulated according to standard methods of organic synthesis (T. W. Greene and P. G. M. Wuts (1999) Protective Groups in Organic Synthesis, 3rd Ed., John Wiley & Sons). These groups may be removed at a convenient stage of the compound synthesis using methods that are readily apparent to those skilled in the art.

In some embodiments, disclosed compounds can generally be synthesized by an appropriate combination of generally well-known synthetic methods. Techniques useful in synthesizing these chemical entities are both readily apparent and accessible to those of skill in the relevant art, based on the instant disclosure. Many of the optionally substituted starting compounds and other reactants are commercially available, or can be readily prepared by those skilled in the art using commonly employed synthetic methodology.

The discussion below is offered to illustrate certain of the diverse methods available for use in making the disclosed compounds and is not intended to limit the scope of reactions or reaction sequences that can be used in preparing the compounds provided herein. The skilled artisan will understand that standard atom valencies apply to all compounds disclosed herein in genus or named compound for unless otherwise specified.

### Example 3

### Compounds of Formula (I)—Compound 11

As an illustrative example of a compound of Formula (I), the following compound, compound 11 as denoted TABLE 1, was synthesized:

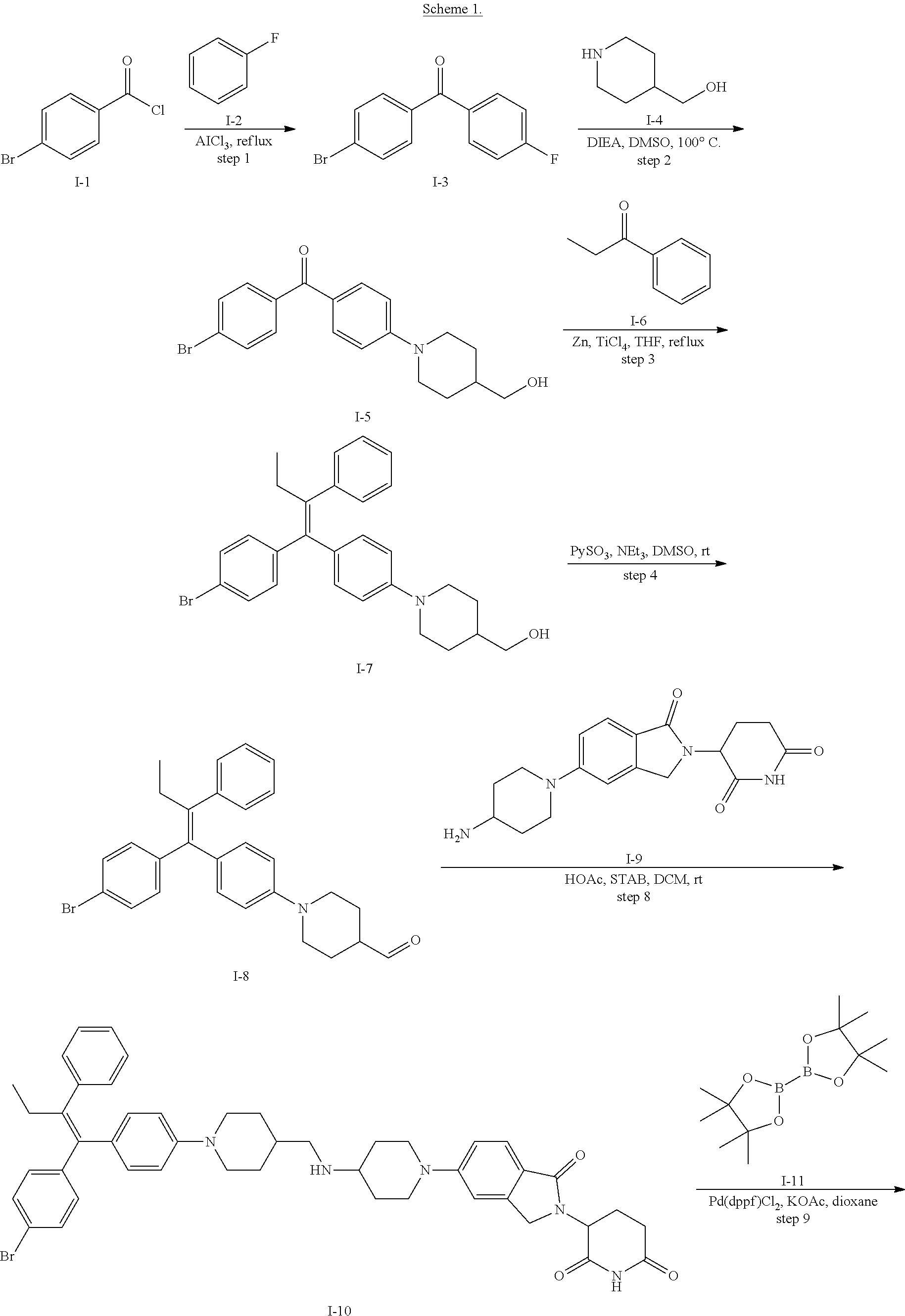

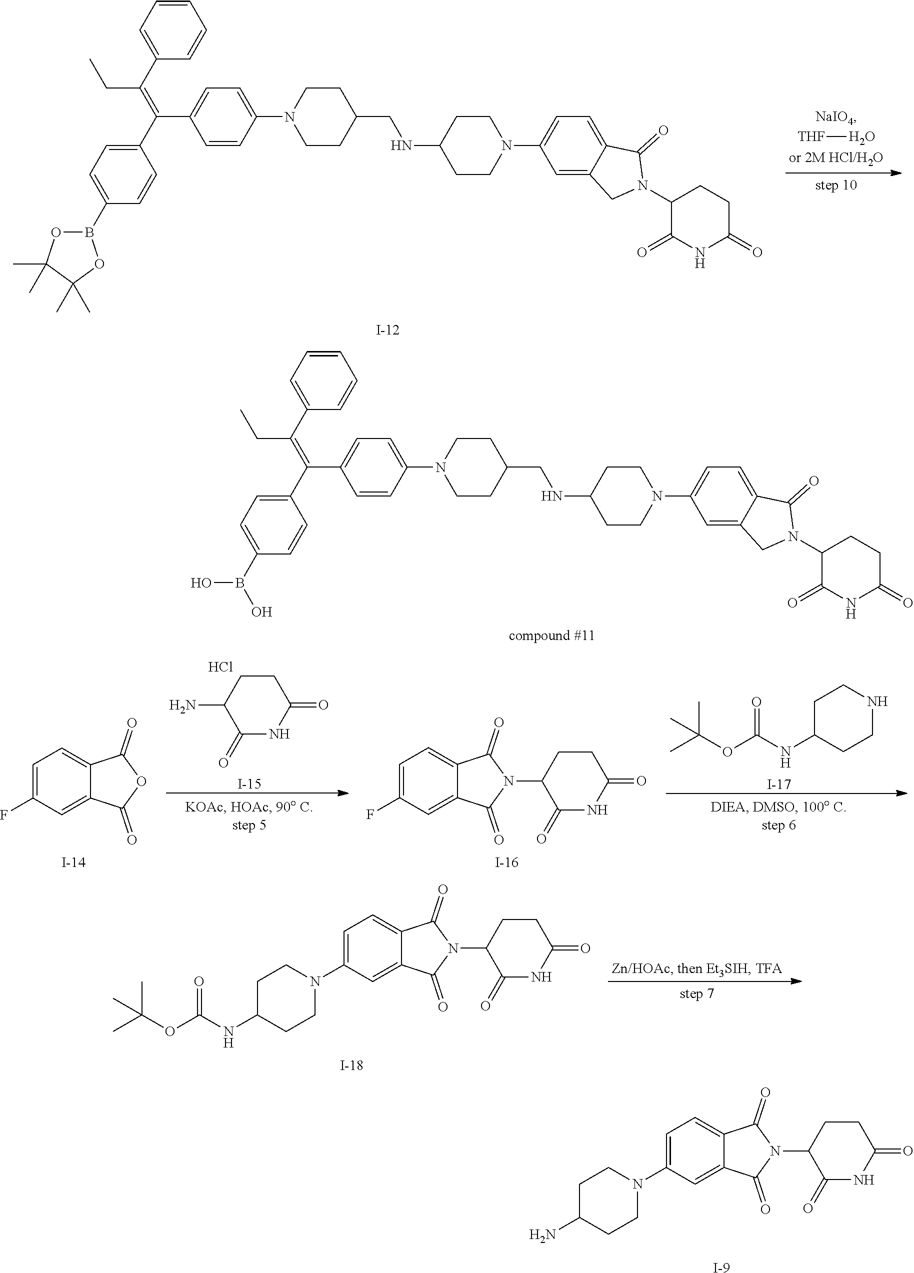

Step 1. (4-bromophenyl)(4-fluorophenyl)methanone (I-3)

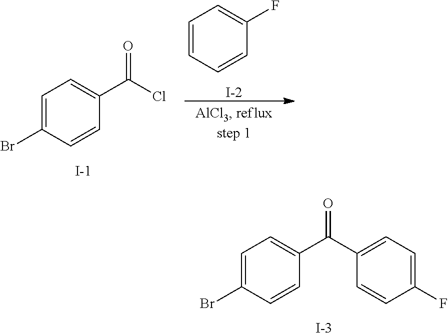

To a stirred mixture of 4-bromobenzoyl chloride (25 g, 0.114 mol) in fluorobenzene (54.7 g, 0.57 mol) was added AlCl₃ (17.6 g, 0.13 mol) portionwise at 0° C. The resulting mixture was heated at 85° C. overnight. After cooling to rt, the mixture was poured into 5% HCl aqueous solution. The product was extracted by DCM. The organic layers were combined and concentrated to provide (4-bromophenyl)(4-fluorophenyl)methanone (I-3) as light yellow solid (27.3 g). 1H NMR (CDCl3, 400 mHz) δ 7.82 (dd, 2H), 7.63-7.65 (m, 4H), 7.15-7.21 (m, 2H).

Step 2. (4-bromophenyl)(4-(4-(hydroxymethyl)piperidin-1-yl)phenyl)methanone (I-5)

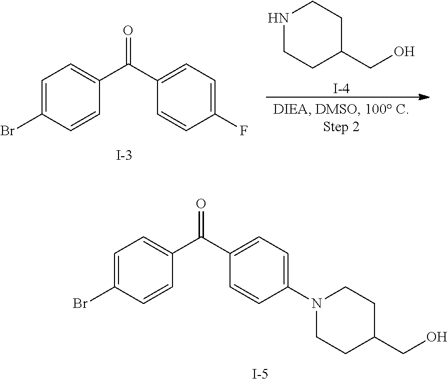

A stirred solution of obtained (4-bromophenyl)(4-fluorophenyl)methanone (I-3) (8.34 g, 30 mmol), piperidin-4-ylmethanol (5.19 g, 45 mmol) and DIEA (7.8 g, 60 mmol) in DMSO (10 mL) was heated to 100° C. overnight. After cooling to rt, the mixture was poured into ice-water and extracted with EA (3×50 mL). The combined organic layers were combined and dried over MgSO4, filtered, evaporated in vacuo. The residue was subjected onto silica gel with n-hexane:EA as eluent to provide (4-bromophenyl)(4-(4-(hydroxymethyl)piperidin-1-yl) phenyl)methanone (I-5) (7.48 g) as white solid. LCMS [M+H] 374; 1H NMR (CDCl3, 400 mHz) δ 7.22-7.53 (m, 2H), 7.60 (m, 4H), 6.85-6.89 (m, 2H), 3.94-4.15 (m, 2H), 3.54 (d, 2H), 2.95 (dt, 2H), 2.62 (s, 2H). 1.83-1.88 (m, 3H), 1.37 (dt, 2H), 1.03-1.06 (m, 4H).

Step 3. (Z/E)-(1-(4-(1-(4-bromophenyl)-2-phenylbut-1-en-1-yl)phenyl)piperidin-4-yl) methanol (I-7)

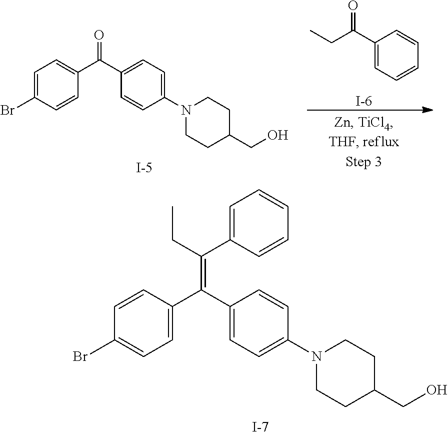

To a suspension of Zinc (17.3 g, 266 mmol) in dry THF (80 mL) was added dropwise TiCl4 (22 g, 116 mmol) under nitrogen at 0° C. When the addition was complete, the mixture was refluxed for 2 hours. The mixture was cooled to 0° C. again. A solution of (4-bromophenyl)(4-(4-(hydroxymethyl) piperidin-1-yl)phenyl)methanone (I-5) (7.48 g, 20 mmol) and propiophenone (8.0 g, 60 mmol) in THF (100 mL) was added. The mixture was brought to reflux in the dark for 2 hours. After being cooled to rt, the reaction mixture was quenched with saturated K2CO3 and extracted with EA (3×100 mL). The combined organic layers were dried over MgSO4, filtered, concentrated in vacuo. The residue was subjected onto silica gel eluting with n-hexane: EA=4:1 to afford (Z/E)-(1-(4-(1-(4-bromophenyl)-2-phenylbut-1-en-1-yl)phenyl)piperidin-4-yl) methanol (I-7) as light yellow oil (11.3 g). LCMS [M+H] 476/478. ¹H NMR (CDCl3, 300 MHz): δ 0.87 (3H, t), 1.50-1.68 (4H, m), 1.59 (2H, dd), 2.04 (1H, t), 2.30 (2H, q), 3.39-3.54 (4H, m), 3.47 (2H, dd), 3.58 (2H, d), 6.76 (2H, d), 7.04-7.20 (3H, m), 7.28-7.48 (8H, m).

Step 4. (E)-1-(4-(1-(4-bromophenyl)-2-phenylbut-1-en-1-yl)phenyl)piperidine-4-carbaldehyde (I-8)

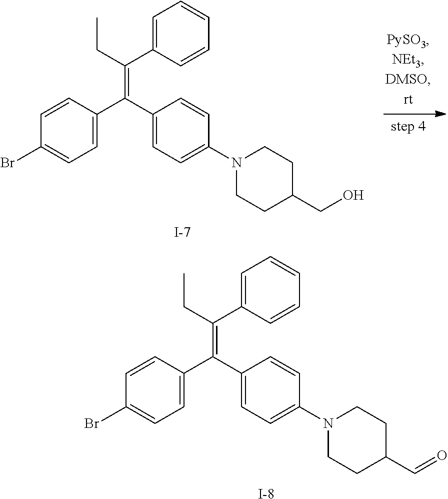

A solution of above obtained (Z/E)-(1-(4-(1-(4-bromophenyl)-2-phenylbut-1-en-1-yl) phenyl)piperidin-4-yl)methanol (I-7) (1.43 g, 3 mmol) in 2 mL DMSO was added triethylamine (0.909 g, 9 mmol) followed by SO3Py (0.954 g, 6 mmol) at rt. After 1 h, TLC indicated fully conversion of SM. DCM and aqueous NaHCO3 was added to quench the reaction. The organic layer was dried over MgSO4, filtered, evaporated to give crude product. The sample was further purified by silica gel chromatography eluting with n-hexane:EA=1:1. Finally, (Z/E)-1-(4-(1-(4-bromophenyl)-2-phenylbut-1-en-1-yl)phenyl)piperidine-4-carbaldehyde (I-8) (1.1 g) was obtained as colorless oil. LCMS [M+H] 474/476. ¹H NMR (CDCl3, 300 MHz): δ 0.90 (3H, t), 1.78-1.98 (4H, m), 2.30 (2H, q), 2.78 (1H, tq), 3.16-3.32 (4H, m), 6.76 (2H, d), 7.04-7.20 (3H, m), 7.28-7.48 (8H, m), 9.66 (1H, d).

Step 5. 2-(2,6-dioxopiperidin-3-yl)-5-fluoroisoindoline-1,3-dione (I-16)

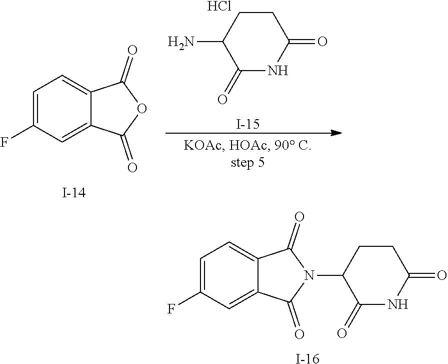

A mixture of 5-fluoroisobenzofuran-1,3-dione (10 g, 61.0 mmol), 3-aminopiperidine-2,6-dione HCl salt (10.0 g, 61.0 mmol) and KOAc (18 g, 18.3 mmol) in 120 mL HOAc was stirred at 90° C. overnight. After cooling to rt, 300 mL of water was added. The solids were collected by filtration, air dried to provide 2-(2,6-dioxopiperidin-3-yl)-5-fluoroisoindoline-1,3-dione (I-16) (15.9 g) as pale solid. LCMS [M+H] 277; 1H NMR (DMSO-d6, 400 mHz) δ 11.15 (br, 1H), 8.05(dd, 1H), 7.84 (dd, 1H), 7.72 (dt, 1H), 5.14 (m, 1H), 2.83-2.91 (m, 1H), 2.50-2.64 (m, 3H), 2.05-2.11 (m, 1H).

Step 6. tert-butyl (1-(2-(2,6-dioxopiperidin-3-yl)-1,3-dioxoisoindolin-5-yl)piperidin-4-yl) carbamate (I-18)

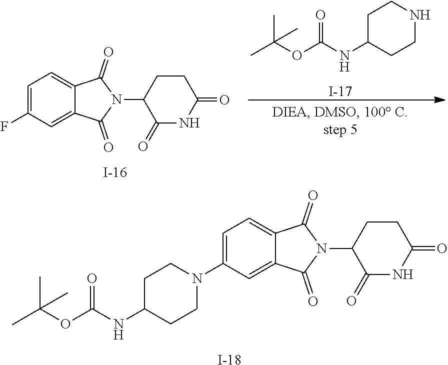

A solution of 2-(2,6-dioxopiperidin-3-yl)-5-fluoroisoindoline-1,3-dione (I-16) (5.5 g, 20 mmol), tert-butyl piperidin-4-ylcarbamate (6.0 g, 30 mmol) and DIEA (5.2 g, 40 mmol) in 10 mL of DMSO was stirred at 100° C. for 2 hours. After cooling to rt, the mixture was poured into ice-water. The solids were collected to provide tert-butyl (1-(2-(2,6-dioxopiperidin-3-yl)-1,3-dioxoisoindolin-5-yl)piperidin-4-yl)carbamate (I-18) as yellow solid (8.0 g). LCMS [M+H] 457.

Step 7. 3-(5-(4-aminopiperidin-1-yl)-1-oxoisoindolin-2-yl)piperidine-2,6-dione (I-19)

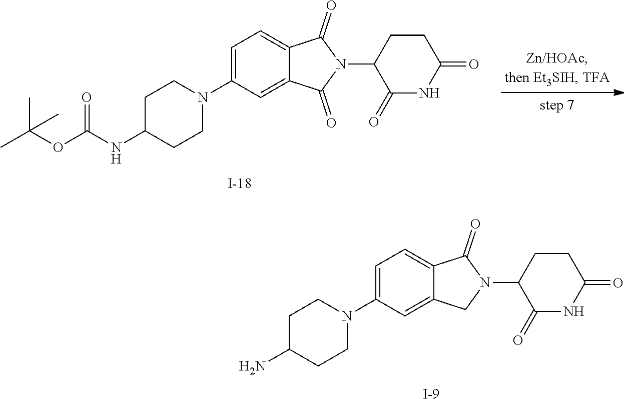

A mixture of tert-butyl (1-(2-(2,6-dioxopiperidin-3-yl)-1,3-dioxoisoindolin-5-yl)piperidin-4-yl) carbamate (I-18) (4.57 g, 10 mmol) and Zinc (3.25 g, 50 mmol) in 20 mL of HOAc was heated at 90° C. for 2 hours. The solids were filtered out. The filtrate was evaporated in vacuo to provide crude intermediate. The obtained intermediate was dissolved in 10 mL of TFA and 10 mL of DCM followed by addition of Et3SiH (5.8 g, 50 mmol) at rt. After stirring for 1 hour, the solvent was removed. The residue was subjected to prep-HPLC to provide 3-(5-(4-aminopiperidin-1-yl)-1-oxoisoindolin-2-yl)piperidine-2,6-dione (I-19) as pale solid (1.0 g). LCMS [M+H] 357.

Step 8. (Z/E)-3-(5-(4-(((1-(4-(1-(4-bromophenyl)-2-phenylbut-1-en-1-yl)phenyl)piperidin-4-yl) methyl)amino)piperidin-1-yl)-1-oxoisoindolin-2-yl)piperidine-2,6-dione (I-10)

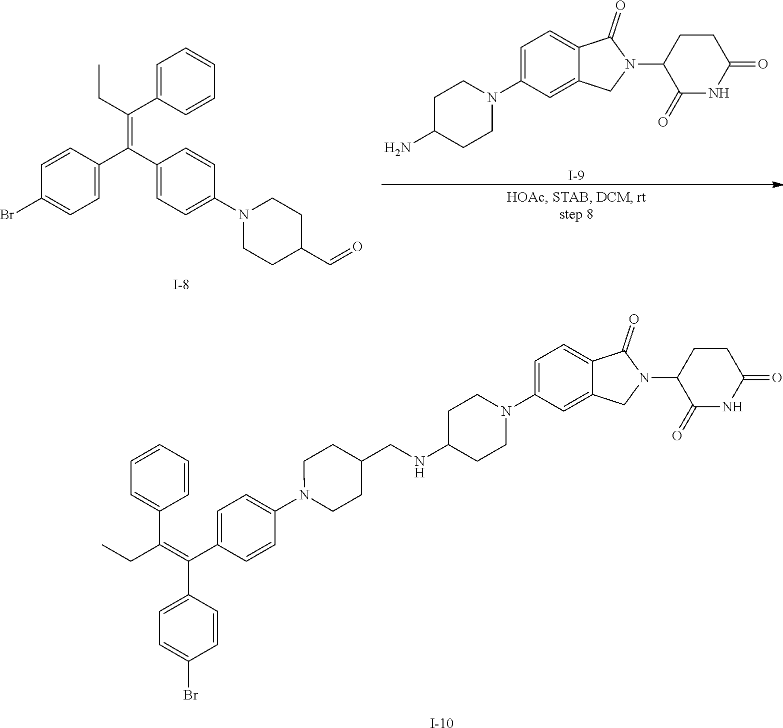

A solution of (Z/E)-1-(4-(1-(4-bromophenyl)-2-phenylbut-1-en-1-yl)phenyl)piperidine-4-carbaldehyde (I-8) (474 mg, 1 mmol), 3-(5-(4-aminopiperidin-1-yl)-1-oxoisoindolin-2-yl) piperidine-2,6-dione (I-19) (343 mg, 1 mmol) and one drop of HOAc in 5 mL of DCM was stirred overnight. STAB (424 mg, 2 mmol) was added portionwise at 0° C. The mixture was warmed to rt and stirred for further 2 hours. The solvent was removed in vacuo. The residue was subjected onto silica gel eluting with 90% EA in MeOH to provide (Z/E)-3-(5-(4-(((1-(4-(1-(4-bromophenyl)-2-phenylbut-1-en-1-yl)phenyl)piperidin-4-yl)methyl)amino)piperidin-1-yl)-1-oxoisoindolin-2-yl)piperidine-2,6-dione (I-10) (180 mg). LCMS [M+H] 800/802.

Step 9. (Z/E)-3-(1-oxo-5-(4-(((1-(4-(2-phenyl-1-(4-(4,4,5,5-tetramethyl-1,3,2-dioxaborolan-2-yl) phenyl)but-1-en-1-yl)phenyl)piperidin-4-yl)methyl)amino)piperidin-1-yl)isoindolin-2-yl) piperidine-2,6-dione (I-12)

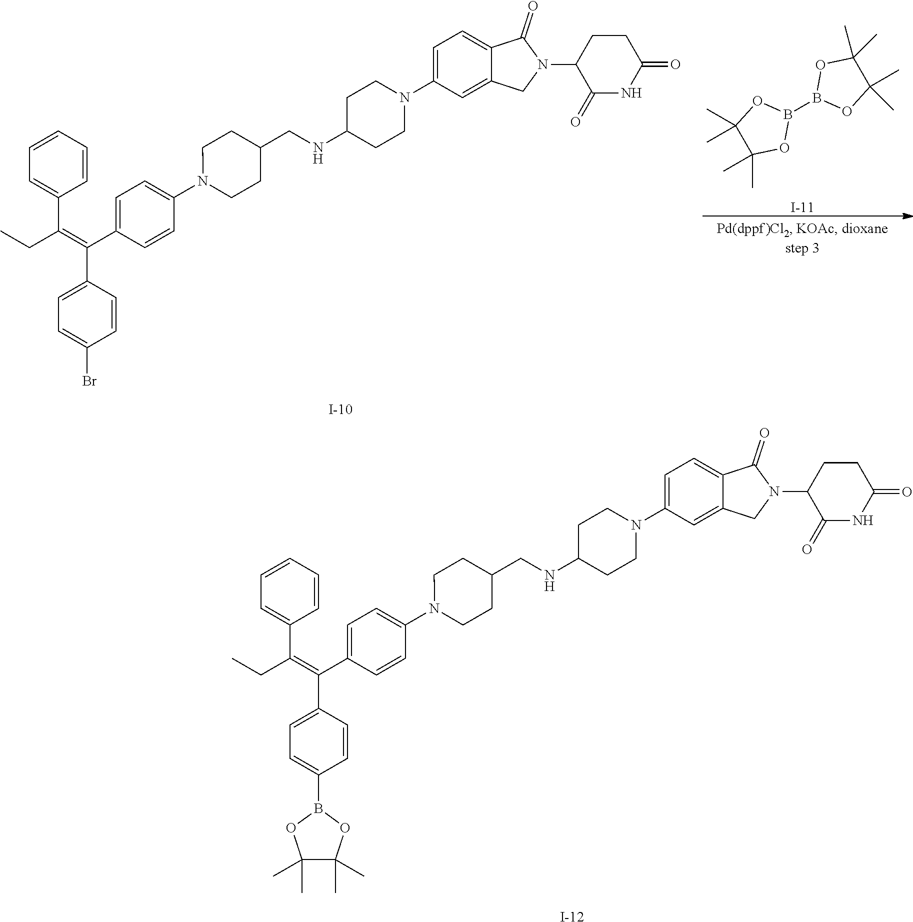

A mixture of (Z/E)-3-(5-(4-(((1-(4-(1-(4-bromophenyl)-2-phenylbut-1-en-1-yl)phenyl)piperidin-4-yl) methyl)amino)piperidin-1-yl)-1-oxoisoindolin-2-yl)piperidine-2,6-dione (I-10) (180 mg, 0.225 mmol), 4,4,4′,4′,5,5,5′,5′-octamethyl-2,2′-bi(1,3,2-dioxaborolane) (114 mg, 0.45 mmol), Pd(dppf)Cl₂ and KOAc in 1,4-dioxane was stirred at 90° C. overnight. The solvent was removed in vacuo. The residue was subjected onto silica gel eluting with 20% MeOH in EA to provide (Z/E)-3-(1-oxo-5-(4-(((1-(4-(2-phenyl-1-(4-(4,4,5,5-tetramethyl-1,3,2-dioxaborolan-2-yl) phenyl)but-1-en-1-yl)phenyl)piperidin-4-yl)methyl)amino)piperidin-1-yl)isoindolin-2-yl) piperidine-2,6-dione (I-12) as brown oil (0.11 g). LCMS [M+H] 848;

Step 10. (Z)-(4-(1-(4-(4-(((1-(2-(2,6-dioxopiperidin-3-yl)-1-oxoisoindolin-5-yl)piperidin-4-yl) amino)methyl)piperidin-1-yl)phenyl)-2-phenylbut-1-en-1-yl)phenyl)boronic acid (compound #11)

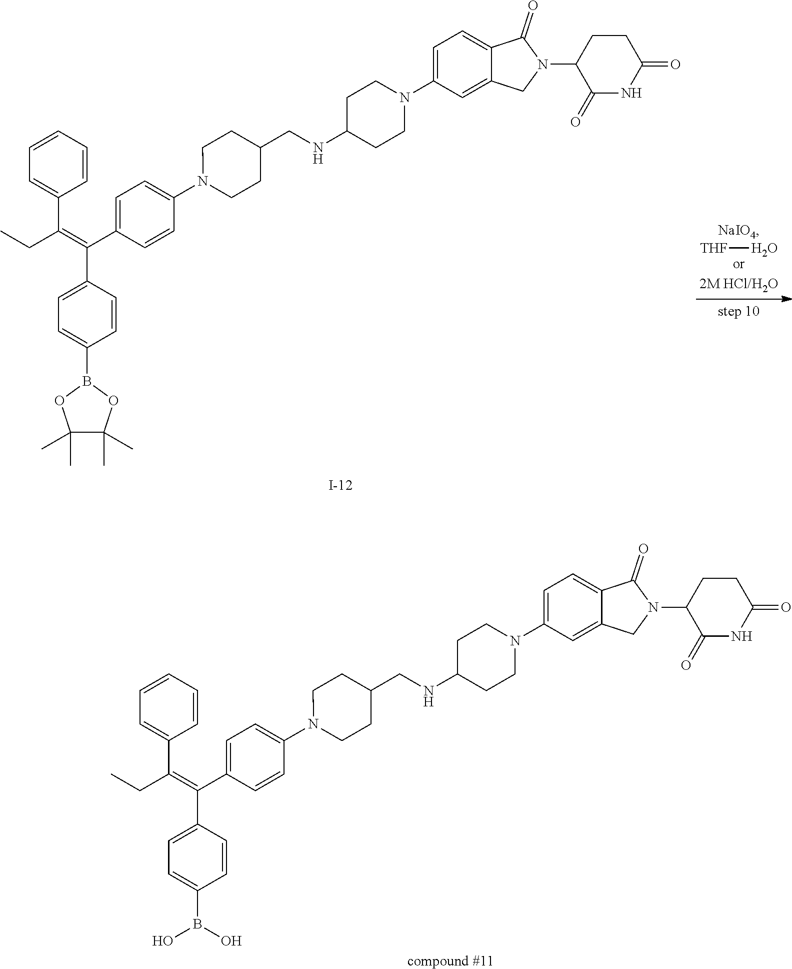

A solution of above obtained (Z/E)-3-(1-oxo-5-(4-(((1-(4-(2-phenyl-1-(4-(4,4,5,5-tetramethyl-1,3,2-dioxaborolan-2-yl)phenyl)but-1-en-1-yl)phenyl)piperidin-4-yl)methyl)amino)piperidin-1-yl) isoindolin-2-yl)piperidine-2,6-dione (I-12) was dissolved in THF and water, NaIO4 was added in one portion. After stirring for 0.5 h, the reaction mixture was purified by prep-HPLC with C18 eluting with MeCN/0.5% FA water, which provided (Z)-(4-(1-(4-(4-(((1-(2-(2,6-dioxopiperidin-3-yl)-1-oxoisoindolin-5-yl)piperidin-4-yl)amino)methyl)piperidin-1-yl)phenyl)-2-phenylbut-1-en-1-yl) phenyl)boronic acid (13 mg). LCMS [M+H] 766. 1H NMR (DMSO-d6, 300 MHz) δ 10.95 (br, 1H), 7.79 (d, 1H), 7.35-7.55 (m, 2H), 6.81-7.28 (m, 8H), 6.50-6.71 (m, 2H), 5.08 (m, 1H), 4.22 (dd, 2H), 3.34-3.45 (m, 3H), 2.88-2.98 (m, 2H), 2.33-2.58 (m, 4H), 1.01-2.02 (m, 14H), 0.85 (m, 3H). and (E)-(4-(1-(4-(4-(((1-(2-(2,6-dioxopiperidin-3-yl)-1-oxoisoindolin-5-yl)piperidin-4-yl) amino)methyl)piperidin-1-yl)phenyl)-2-phenylbut-1-en-1-yl)phenyl)boronic acid (10 mg) as white solid LCMS [M+H] 766, 1H NMR (DMSO-d6, 300 MHz) δ 10.93 (br, 1H), 7.79 (d, 1H), 7.35-7.55 (m, 2H), 6.81-7.28 (m, 8H), 6.50-6.71 (m, 2H), 5.08 (m, 1H), 4.22 (dd, 2H), 3.34-3.45 (m, 3H), 2.88-2.98 (m, 2H), 2.33-2.58 (m, 4H), 1.01-2.02 (m, 14H), 0.87 (m, 3H).

### Example 4

### Compounds of Formula (I)—Compound 21

As an illustrative example of a compound of Formula (I), the following compound, compound 21 as denoted TABLE 1, was synthesized:

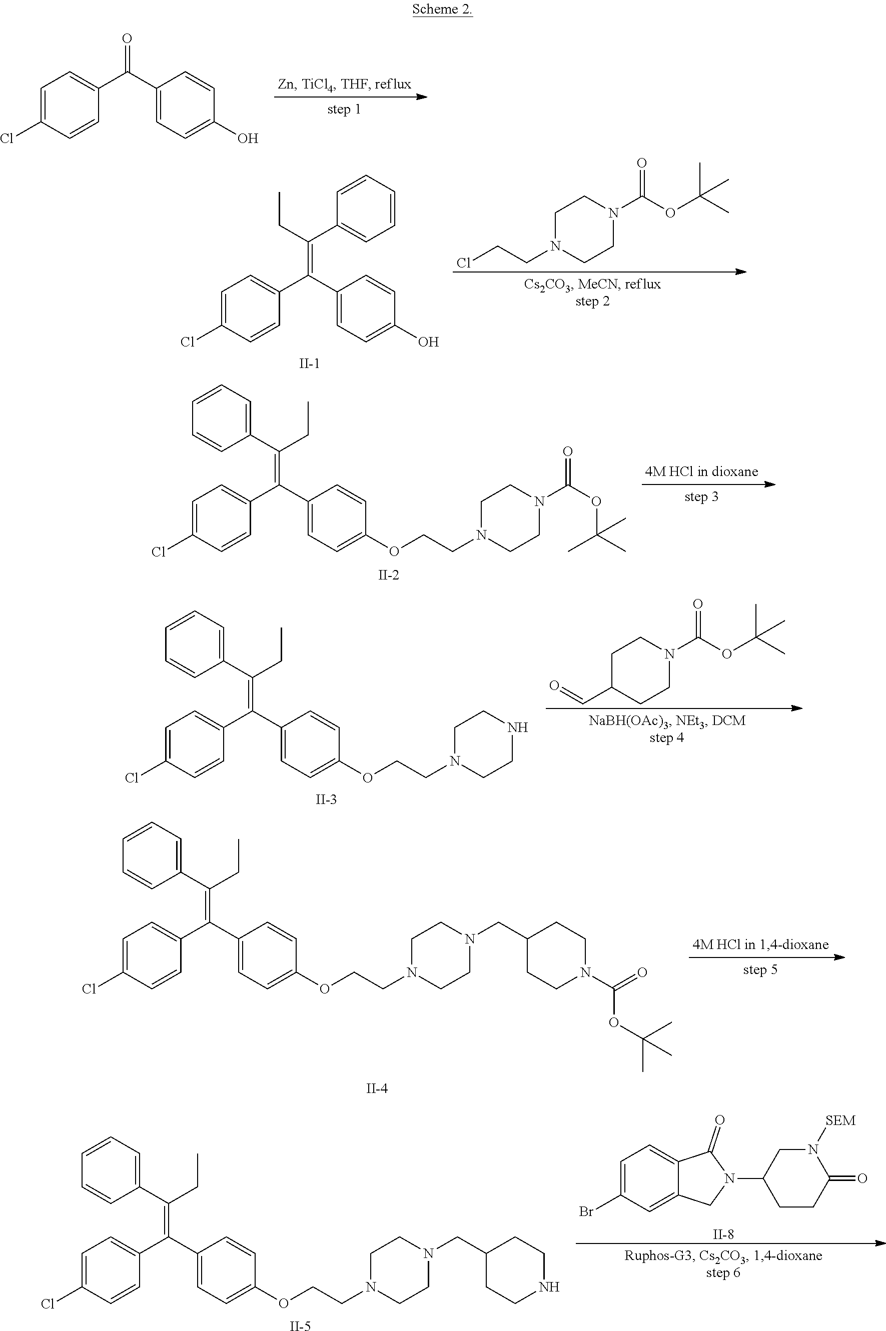

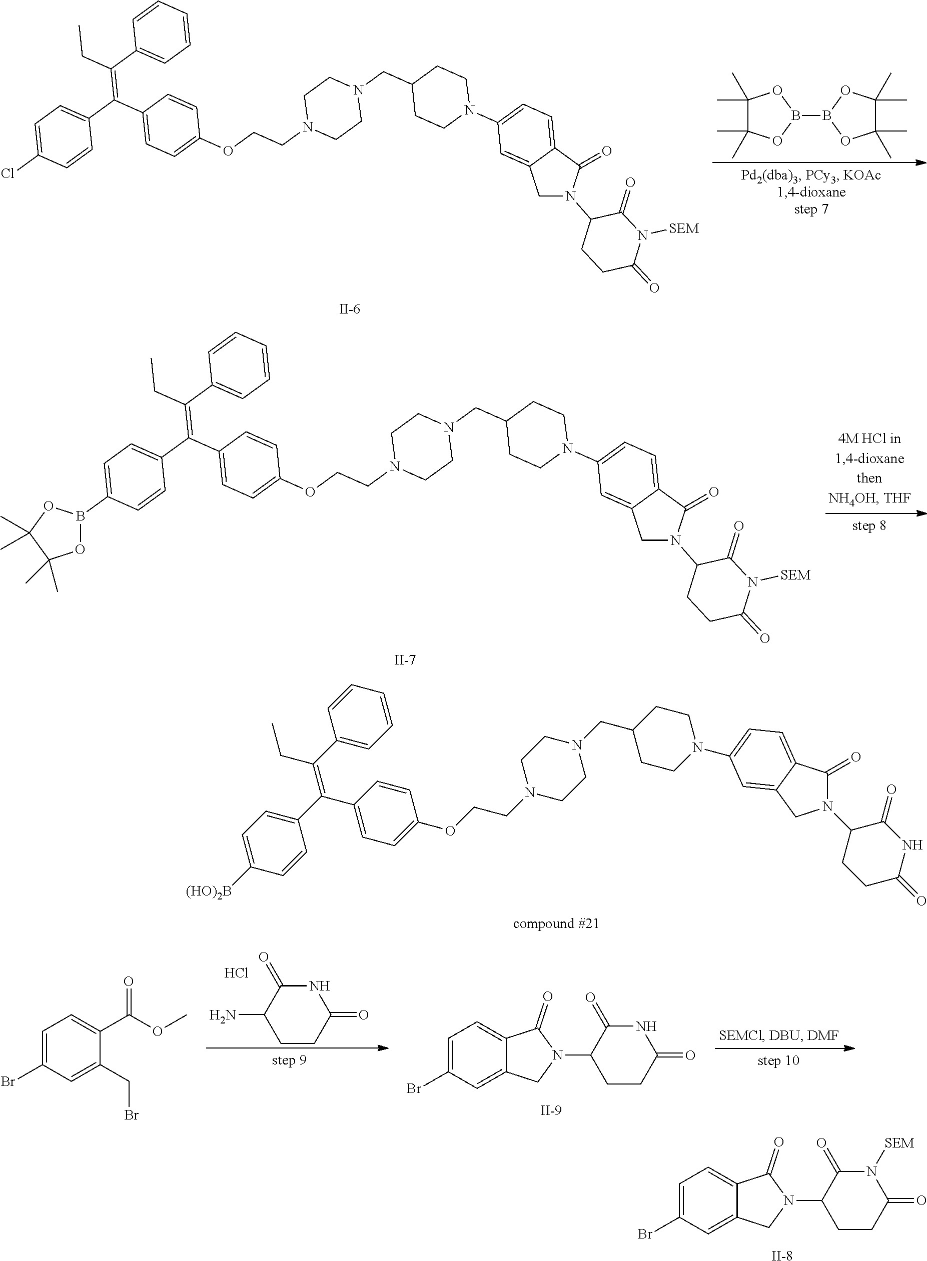

Step 1. (Z/E)-4-(1-(4-chlorophenyl)-2-phenylbut-1-en-1-yl)phenol (II-1)

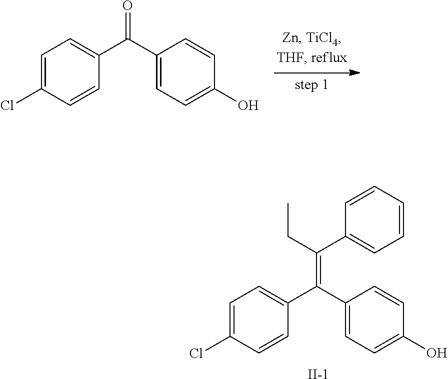

Procedure as example I step II. (4-chlorophenyl)(4-hydroxyphenyl)methanone (9.3 g, 40 mmol) gave (Z/E)-4-(1-(4-chlorophenyl)-2-phenylbut-1-en-1-yl)phenol (II-1) (7.6 g) as yellow oil. LCMS [M+H] 335. 1H NMR (CDCl3, 400 mHz) δ 7.35-7.05 (m, 9H), 7.68 (d, 2H), 6.46 (d, 2H), 4.90 (br, 1H), 2.47 (q, 2H), 0.90 (t, 3H).

Step 2. tert-butyl (Z/E)-4-(2-(4-(1-(4-chlorophenyl)-2-phenylbut-1-en-1-yl) phenoxy)ethyl)piperazine-1-carboxylate (II-2)

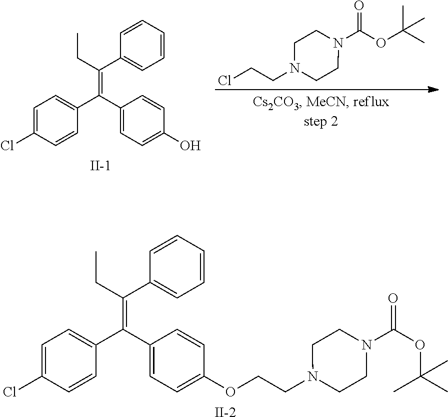

A mixture of (Z/E)-4-(1-(4-chlorophenyl)-2-phenylbut-1-en-1-yl)phenol (II-1) (6.7 g, 20 mmol), tert-butyl 4-(2-chloroethyl)piperazine-1-carboxylate (6.0 g, 24 mmol), K₂CO₃ (6.64 g, 40 mmol) and KI (0.276 g, 2 mmol) in EtOH was stirred at reflux for 10 h. After cooling to rt, the solids were filtered out. The filtrate was evaporated in vacuo. The residue was subjected onto silica gel column chromatography eluting with 20% EA in n-hexane to provide tert-butyl (Z/E)-4-(2-(4-(1-(4-chlorophenyl)-2-phenylbut-1-en-1-yl)phenoxy)ethyl)piperazine-1-carboxylate (II-2) as yellow oil (7.0 g). LCMS [M+H] 547. ¹H NMR (CDCl3, 300 MHz): δ 0.91 (3H, t, J=6.7 Hz), 1.46 (9H, s), 2.31 (2H, q), 2.63 (4H, d), 2.90 (2H, t), 3.55 (4H, m), 4.23 (2H, t), 6.52 (2H, d), 7.21 (2H, d), 7.26-7.42 (7H, m), 7.50 (2H, d).

Step 3. (Z/E)-1-(2-(4-(1-(4-chlorophenyl)-2-phenylbut-1-en-1-yl)phenoxy)ethyl)piperazine (II-3)

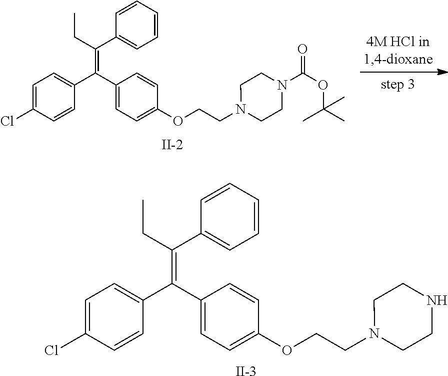

A solution of tert-butyl (Z/E)-4-(2-(4-(1-(4-chlorophenyl)-2-phenylbut-1-en-1-yl) phenoxy)ethyl)piperazine-1-carboxylate (II-2) as yellow oil (7.0 g) in 4 M HCl in 1,4-dioxane was stirred at rt for 1 h. The solvent was removed to provide (Z/E)-1-(2-(4-(1-(4-chlorophenyl)-2-phenylbut-1-en-1-yl)phenoxy)ethyl)piperazine (II-3) as white solid (8.7 g). LCMS [M+H] 447; ¹H NMR (CDCl3, 300 MHz): δ 0.90 (3H, t), 2.41 (2H, q), 2.50 (4H, m), 2.72 (4H, m), 2.88 (2H, t), 4.22 (2H, t), 6.92 (2H, d), 7.21 (2H, d), 7.26-7.42 (7H, m), 7.50 (2H, d).

Step 4. tert-butyl (Z/E)-4-((4-(2-(4-(1-(4-chlorophenyl)-2-phenylbut-1-en-1-yl) phenoxy)ethyl)piperazin-1-yl)methyl)piperidine-1-carboxylate (II-4)

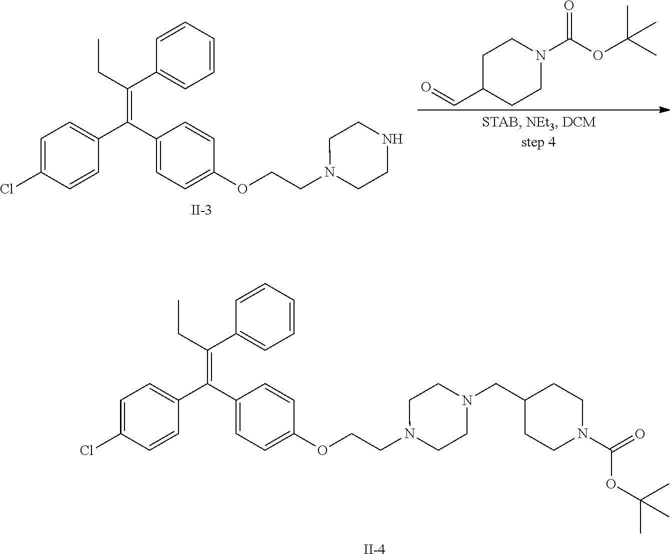

Procedure as example I, step 8. Standard reductive amination process. (Z/E)-1-(2-(4-(1-(4-chlorophenyl)-2-phenylbut-1-en-1-yl)phenoxy)ethyl)piperazine (II-3) (1.34 g, 30 mmol) provided tert-butyl (Z/E)-4-((4-(2-(4-(1-(4-chlorophenyl)-2-phenylbut-1-en-1-yl)phenoxy)ethyl)piperazin-1-yl) methyl)piperidine-1-carboxylate (II-4) (1.745 g) as yellow oil. LCMS [M+H] 644.

Step 5. (Z/E)-1-(2-(4-(1-(4-chlorophenyl)-2-phenylbut-1-en-1-yl)phenoxy)ethyl)-4-(piperidin-4-ylmethyl)piperazine (II-5)

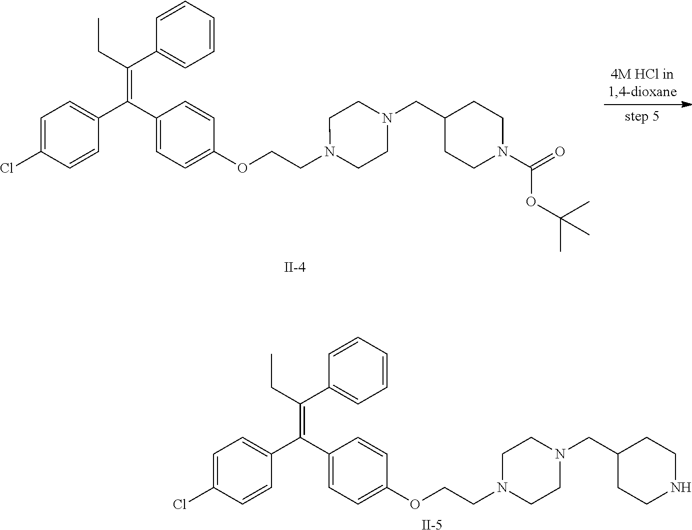

Procedure as example II, step 3. tert-butyl (Z/E)-4-((4-(2-(4-(1-(4-chlorophenyl)-2-phenylbut-1-en-1-yl)phenoxy)ethyl)piperazin-1-yl)methyl)piperidine-1-carboxylate (II-4) (1.5 g) provided (Z/E)-1-(2-(4-(1-(4-chlorophenyl)-2-phenylbut-1-en-1-yl)phenoxy)ethyl)-4-(piperidin-4-ylmethyl) piperazine (II-5) (1.94 g) as white solid. LCMS [M+H] 544. ¹H NMR (DMSO-d6, 300 MHz): δ 0.86 (3H, t), 1.62-1.93 (5H, m), 2.31 (2H, q), 2.41 (2H, d), 2.48-2.69 (10H, m), 2.85-2.93 (4H, m), 4.23 (2H, t), 6.92 (2H, d), 7.21 (2H, d), 7.26-7.42 (7H, m), 7.49 (2H, d).

Step 6. (Z/E)-3-(5-(4-((4-(2-(4-(1-(4-chlorophenyl)-2-phenylbut-1-en-1-yl) phenoxy)ethyl)piperazin-1-yl)methyl)piperidin-1-yl)-1-oxoisoindolin-2-yl)-1-((2-(trimethylsilyl) ethoxy)methyl)piperidine-2,6-dione (II-6)

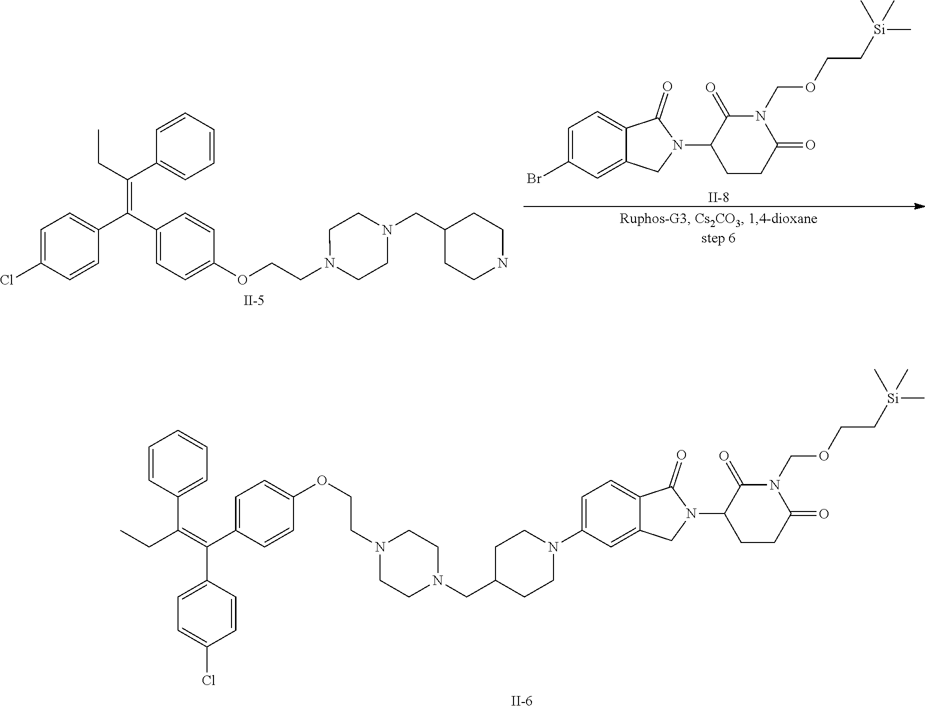

A mixture of (Z/E)-1-(2-(4-(1-(4-chlorophenyl)-2-phenylbut-1-en-1-yl)phenoxy)ethyl)-4-(piperidin-4-ylmethyl)piperazine (II-5) (0.453 g, 1.0 mmol), 3-(5-bromo-1-oxoisoindolin-2-yl)-1-((2-(trimethylsilyl) ethoxy)methyl)piperidine-2,6-dione (II-8) (0.544 g, 1.0 mmol), Ruphos-G3 (42 mg, 0.05 mmol), Ruphos (24 mg, 0.05 mmol) and Cs₂CO₃ (1.6 g, 5 mmol) in 1,4-dioxane was stirred at 100° C. under Ar. After stirring for 2 hours, the reaction mixture was cooled to rt, 100 mL DCM was added to dilute the reaction mixture. The solids were filtered out with the assistance of celite. The filtrate was evaporated in vacuo. The residue was subjected onto silica gel column chromatography with 20% MeOH in EA as eluent. (Z/E)-3-(5-(4-((4-(2-(4-(1-(4-chlorophenyl)-2-phenylbut-1-en-1-yl)phenoxy)ethyl)piperazin-1-yl)methyl)piperidin-1-yl)-1-oxoisoindolin-2-yl)-1-((2-(trimethylsilyl)ethoxy)methyl)piperidine-2,6-dione (II-6) (0.567 g) was obtained. LCMS [M+H] 916, ¹H NMR (DMSO-d6, 300 MHz): δ 0.02 (9H, s), 0.88-0.96 (2H, dt), 1.22 (3H, t), 1.59-1.70 (2H, m), 1.74-2.13 (5H, m), 2.23-2.38 (4H, m), 2.41-2.44 (2H, dd), 2.51-2.66 (8H, m), 2.85-2.94 (2H, m), 3.37-3.54 (6H, m), 4.19-4.27 (2H, m), 4.53-4.62 (3H, m), 5.44-5.45 (2H, d), 6.89-7.00 (3H, m), 7.05 (1H, d), 7.21 (2H, d), 7.26-7.42 (7H, m), 7.50 (2H, d), 7.79 (1H, d).

Step 7. (Z/E)-3-(1-oxo-5-(4-((4-(2-(4-(2-phenyl-1-(4-(4,4,5,5-tetramethyl-1,3,2-dioxaborolan-2-yl)phenyl)but-1-en-1-yl)phenoxy)ethyl)piperazin-1-yl)methyl)piperidin-1-yl)isoindolin-2-yl)-1-((2-(trimethylsilyl)ethoxy)methyl)piperidine-2,6-dione (II-7)

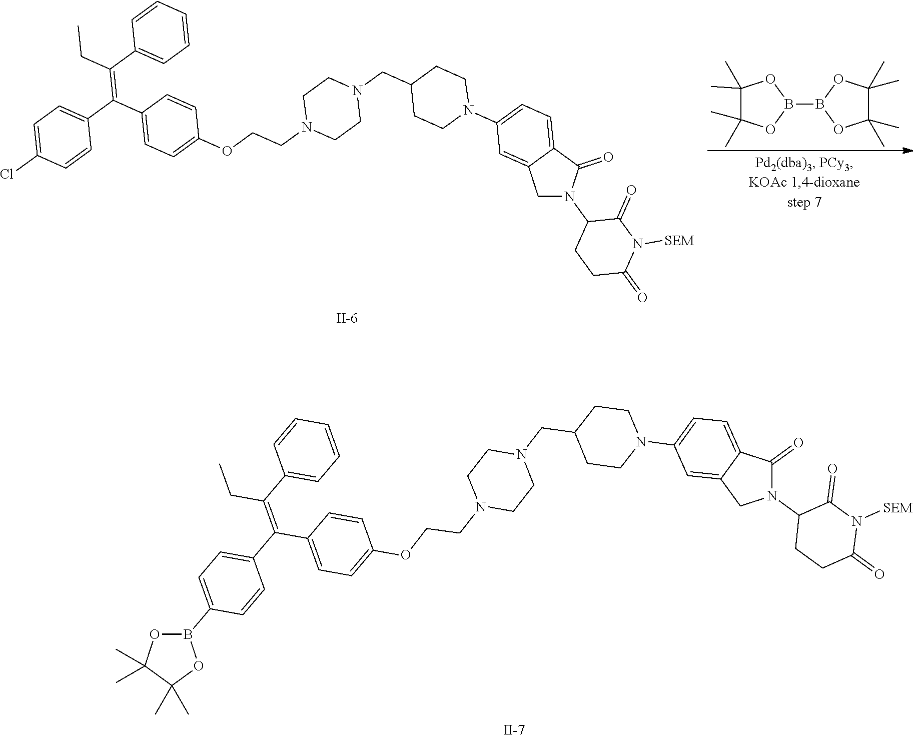

A mixture of (Z/E)-3-(5-(4-((4-(2-(4-(1-(4-chlorophenyl)-2-phenylbut-1-en-1-yl) phenoxy)ethyl)piperazin-1-yl)methyl)piperidin-1-yl)-1-oxoisoindolin-2-yl)-1-((2-(trimethylsilyl) ethoxy)methyl)piperidine-2,6-dione (II-6) (0.567 g, 0.6 mmol), 4,4,4′,4′,5,5,5′,5′-octamethyl-2,2′-bi(1,3,2-dioxaborolane) (0.305 g, 1.2 mmol), Pd₂(dba)₃ (17 mg, 0.03 mmol), PCy₃ (17 mg, 0.06 mmol) and KOAc (180 mg, 1.8 mmol) in 10 mL of 1,4-dioxane was stirred at 100° C. under Ar overnight. The solvent was removed in vacuo. The residue was subjected onto silica gel column chromatography eluting with 20% MeOH in EA to provide (Z/E)-3-(1-oxo-5-(4-((4-(2-(4-(2-phenyl-1-(4-(4,4,5,5-tetramethyl-1,3,2-dioxaborolan-2-yl)phenyl)but-1-en-1-yl) phenoxy)ethyl)piperazin-1-yl)methyl)piperidin-1-yl)isoindolin-2-yl)-1-((2-(trimethylsilyl) ethoxy)methyl)piperidine-2,6-dione (II-7) as brown solid (0.66 g). LCMS [M+H] 1009.

Step 8. (Z)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid (compound #21)

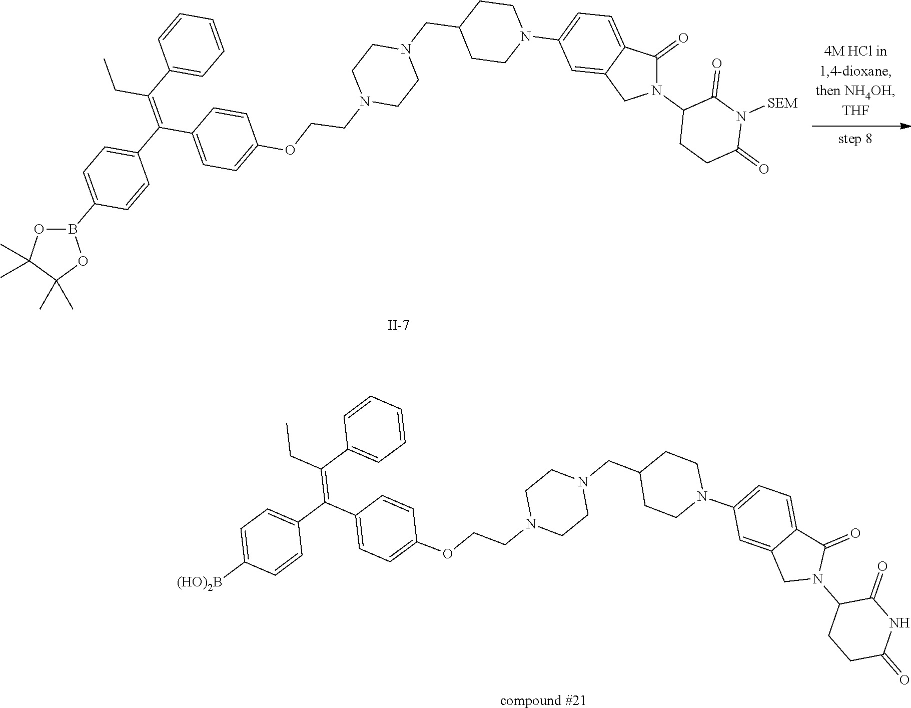

A solution of (Z/E)-3-(1-oxo-5-(4-((4-(2-(4-(2-phenyl-1-(4-(4,4,5,5-tetramethyl-1,3,2-dioxaborolan-2-yl)phenyl)but-1-en-1-yl)phenoxy)ethyl)piperazin-1-yl)methyl)piperidin-1-yl) isoindolin-2-yl)-1-((2-(trimethylsilyl)ethoxy)methyl)piperidine-2,6-dione (II-7) (0.6 g) in 4M HCl in 1,4-dioxane was stirred for 1 h. After that, the solvent was removed in vacuo. The residue was dissolved in THF and MH4OH was added and the mixture was stirred for another 1 h. The pure sample was purified by prep-HPLC with 0.5% FA-MeCN system. (Z)-(4-(1-(4-(2-(4-((1-(2-(2,6-dioxopiperidin-3-yl)-1-oxoisoindolin-5-yl)piperidin-4-yl)methyl)piperazin-1-yl)ethoxy)phenyl)-2-phenylbut-1-en-1-yl)phenyl)boronic acid (compound #21) (35 mg) as white solid. LCMS [M+H] 796; 1H NMR (DMSO-d6, 400 MHz) δ 10.94 (s, 1H), 8.11 (s, 1H), 7.66 (d, 1H), 7.32-7.45 (m, 2H), 6.47-7.25 (m, 10H), 5.05 (m, 1H), 4.22 (dd, 2H), 3.12-3.45 (m, 3H), 1.12-2.99 (m, 26H), 0.85 (t, 3H).

Step 9. 3-(5-bromo-1-oxoisoindolin-2-yl)piperidine-2,6-dione (II-9)

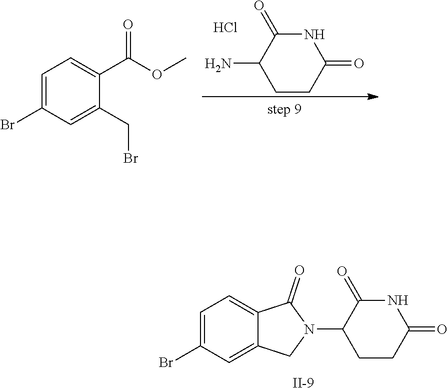

A stirred mixture of methyl 4-bromo-2-(bromomethyl)benzoate (32.9 g, 100 mmol), 3-aminopiperidine-2,6-dione HCl salt (16.5 g, 100 mmol) and DIEA (26 g, 200 mmol) in 200 mL of MeCN was stirred at 90° C. for 48 h. After cooling to rt, the solids were collected by filtration. The solids were washed with 30 mL EA, 30 mL EA-Et2O (v/v 1:1) and Et2O (30 mL). 3-(5-bromo-1-oxoisoindolin-2-yl)piperidine-2,6-dione (II-9) (15 g) was obtained as pale brown solid, LCMS [M+H] 323/325; 1H NMR (DMSO-d6, 400 MHz) δ 11.01 (s, 1H), 7.89 (s, 1H), 7.66-7.74 (m, 2H), 5.13 (m, 1H), 4.33 (dd, 2H), 2.93 (dt, 1H), 2.57-2.63 (m, 1H), 2.43 (m, 1H), 1.99-2.05 (m, 1H).

Step 10. 3-(5-bromo-1-oxoisoindolin-2-yl)-1-((2-(trimethylsilyl)ethoxy)methyl)piperidine-2,6-dione (II-8)

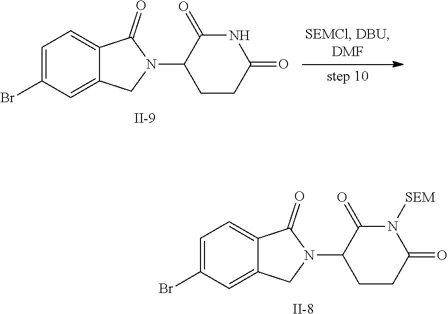

To a stirred solution of 3-(5-bromo-1-oxoisoindolin-2-yl)piperidine-2,6-dione (II-9) (3.22 g, 10 mmol) and DBU (2.3 g, 15 mmol) in 35 mL of DMF was added SEMCI (1.99 g, 12 mmol) at 0° C. The resulting mixture was allowed to warm to rt and stirred for further 2 hours. Saturated NH4Cl was added to quench the reaction. EA (3×50 mL) was used to extract the product. The combined organic layers were dried over Na2SO4, filtered, evaporated. The residue was subjected onto silica gel column chromatography with 50% EA in n-hexane. 3-(5-bromo-1-oxoisoindolin-2-yl)-1-((2-(trimethylsilyl)ethoxy)methyl)piperidine-2,6-dione (II-8) (3.47 g) was obtained. LCMS [M+H] 453/455; 1H NMR (DMSO-d6, 400 MHz) δ 7.90 (s, 1H), 7.66-7.72 (m, 3H), 5.22 (dd, 1H), 5.07 (dd, 2H), 4.38 (dd, 2H), 4.15 (d, 1H), 3.53 (dt, 2H), 3.07 (dt, 1H), 2.72-2.80 (d, 1H), 2.32-2.45 (m, 1H), 1.99-2.08 (m, 1H), 1.20-1.40 (m, 4H), 0.88 (m, 2H), 0.02 (s, 9H).

### Example 5

### Compounds of Formula (I)—Compound 41

As an illustrative example of a compound of Formula (I), the following compound, compound 41 as denoted TABLE 1, was synthesized:

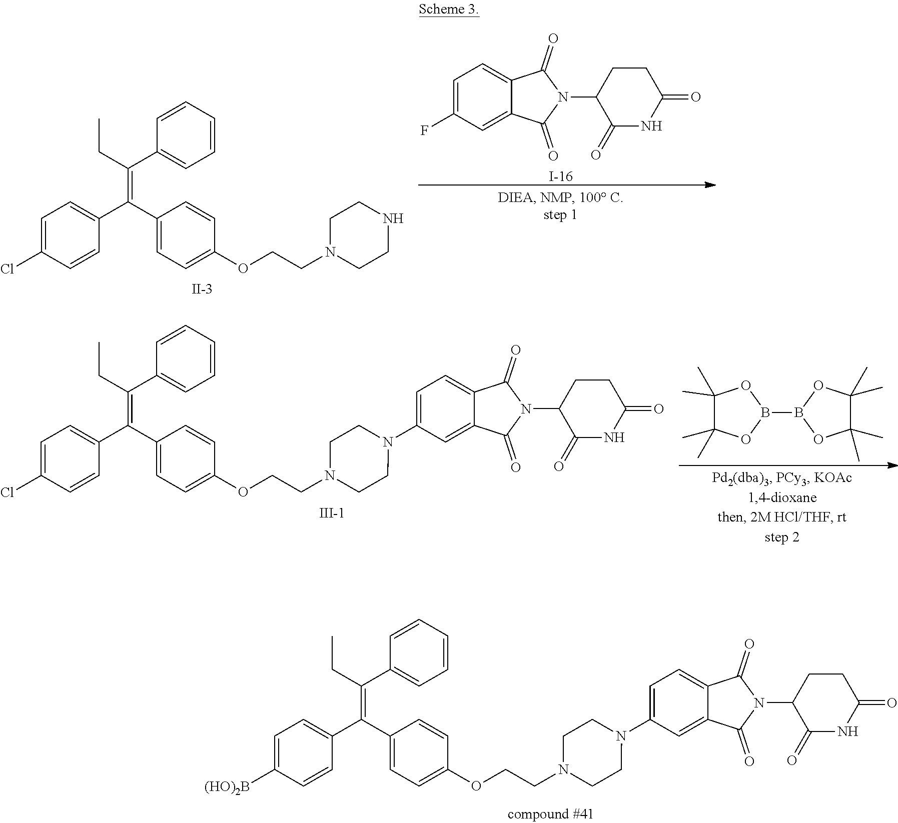

Step 1. (Z/E)-5-(4-(2-(4-(1-(4-chlorophenyl)-2-phenylbut-1-en-1-yl)phenoxy)ethyl)piperazin-1-yl)-2-(2,6-dioxopiperidin-3-yl)isoindoline-1,3-dione (III-1)

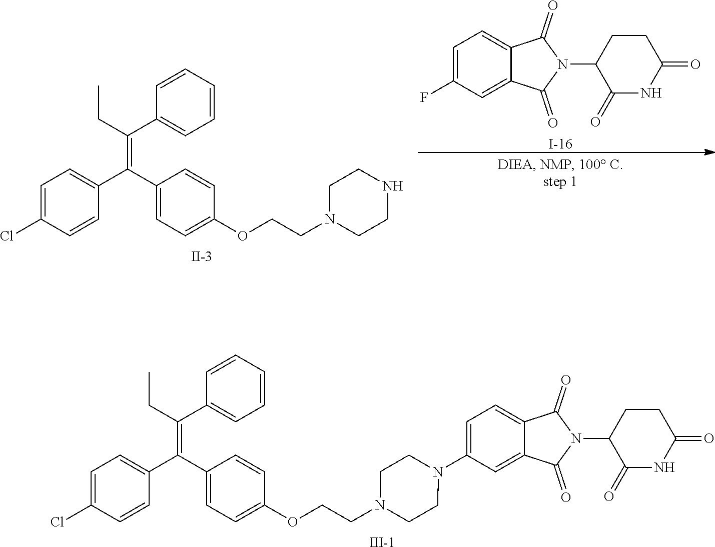

A mixture of (Z/E)-1-(2-(4-(1-(4-chlorophenyl)-2-phenylbut-1-en-1-yl)phenoxy)ethyl)piperazine (II-3) (447 mg, 1 mmol), 2-(2,6-dioxopiperidin-3-yl)-5-fluoroisoindoline-1,3-dione (I-16) (414 mg, 1.5 mmol) and DIEA (390 mg, 3 mmol) in 5 mL of NMP was heated at 100° C. for 2 hours. Most of solvent was evaporated in vacuo. The residue was subjected onto silica gel column chromatography and then prep-HPLC to provide (Z/E)-5-(4-(2-(4-(1-(4-chlorophenyl)-2-phenylbut-1-en-1-yl)phenoxy)ethyl)piperazin-1-yl)-2-(2,6-dioxopiperidin-3-yl)isoindoline-1,3-dione (III-1) (228 mg) as brown oil. LCMS [M+H] 703;

Step 2. (Z)-(4-(1-(4-(2-(4-(2-(2,6-dioxopiperidin-3-yl)-1,3-dioxoisoindolin-5-yl)piperazin-1-yl) ethoxy)phenyl)-2-phenylbut-1-en-1-yl)phenyl)boronic acid (compound #41)

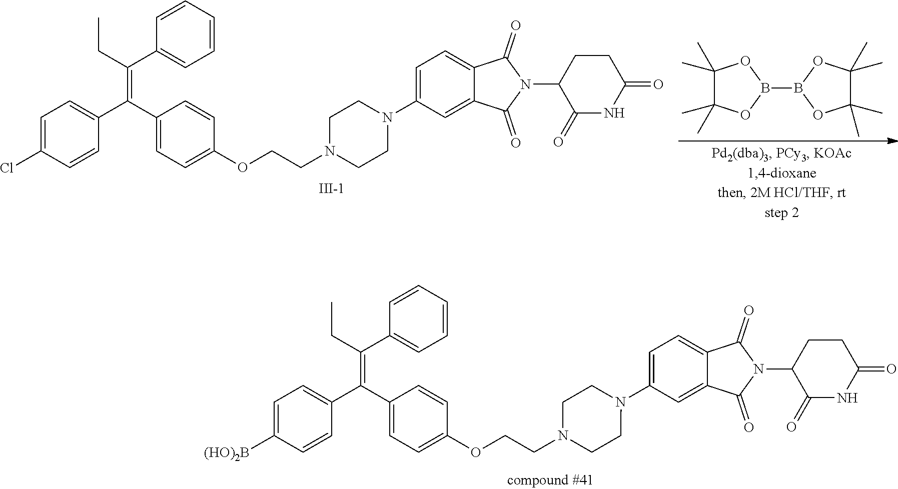

A mixture of (Z/E)-5-(4-(2-(4-(1-(4-chlorophenyl)-2-phenylbut-1-en-1-yl)phenoxy)ethyl)piperazin-1-yl)-2-(2,6-dioxopiperidin-3-yl)isoindoline-1,3-dione (III-1) (228 mg, 0.33 mmol), B₂pin₂ (168 mg, 0.66 mmol), Pd₂(dba)₃ (9.5 mg, 0.0165 mmol), PCy₃ (9.3 mg, 0.033 mmol) and KOAc (98 mg, 0.99 mmol) in 5 mL of 1,4-dioxane was heated at 90° C. under nitrogen overnight. The solvent was removed in vacuo. The residue was subjected onto silica gel column chromatography to give crude boronic ester. The obtained residue was dissolved in THF and 2 M HCl aqueous solution was added and stirred for 2 hours in room temperature. The product was purified by prep-HPLC with 0.5% FA-MeCN. (Z)-(4-(1-(4-(2-(4-(2-(2,6-dioxopiperidin-3-yl)-1,3-dioxoisoindolin-5-yl)piperazin-1-yl)ethoxy)phenyl)-2-phenylbut-1-en-1-yl)phenyl)boronic acid (compound #41) (0.030 mg) as white solid and (E)-(4-(1-(4-(2-(4-(2-(2,6-dioxopiperidin-3-yl)-1,3-dioxoisoindolin-5-yl)piperazin-1-yl)ethoxy)phenyl)-2-phenylbut-1-en-1-yl)phenyl)boronic acid (compound #42) (0.025 mg) as white solid. LCMS [M+H] 713. ¹H NMR (DMSO-d6, 300 MHz): δ 0.85 (3H, t), 1.96-2.14 (2H, m), 2.21-2.44 (4H, m), 2.64-2.94 (6H, m), 2.99-3.13 (4H, m), 3.97-4.14 (2H, dt), 5.05 (1H, dd), 6.92 (2H, d), 7.06 (1H, d), 7.26-7.40 (9H, m), 7.50 (1H, d), 7.61 (2H, d), 8.38 (1H, d), 11.92 (s, 1H).

### Example 6

### Compounds of Formula (I)—Compound 17

As an illustrative example of a compound of Formula (I), the following compound, compound 17 as denoted TABLE 1, was synthesized:

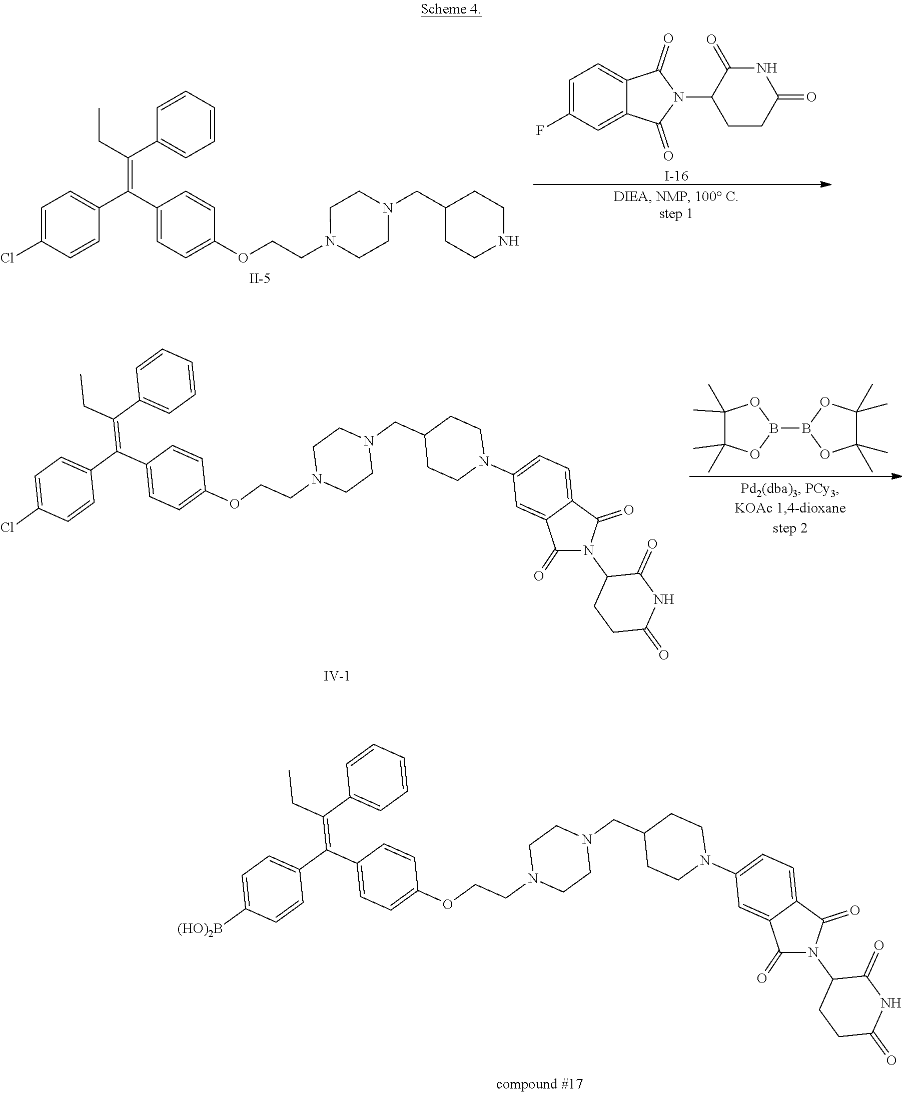

Step 1. (Z/E)-5-(4-(2-(4-(1-(4-chlorophenyl)-2-phenylbut-1-en-1-yl)phenoxy)ethyl)piperazin-1-yl)-2-(2,6-dioxopiperidin-3-yl)isoindoline-1,3-dione (IV-1)

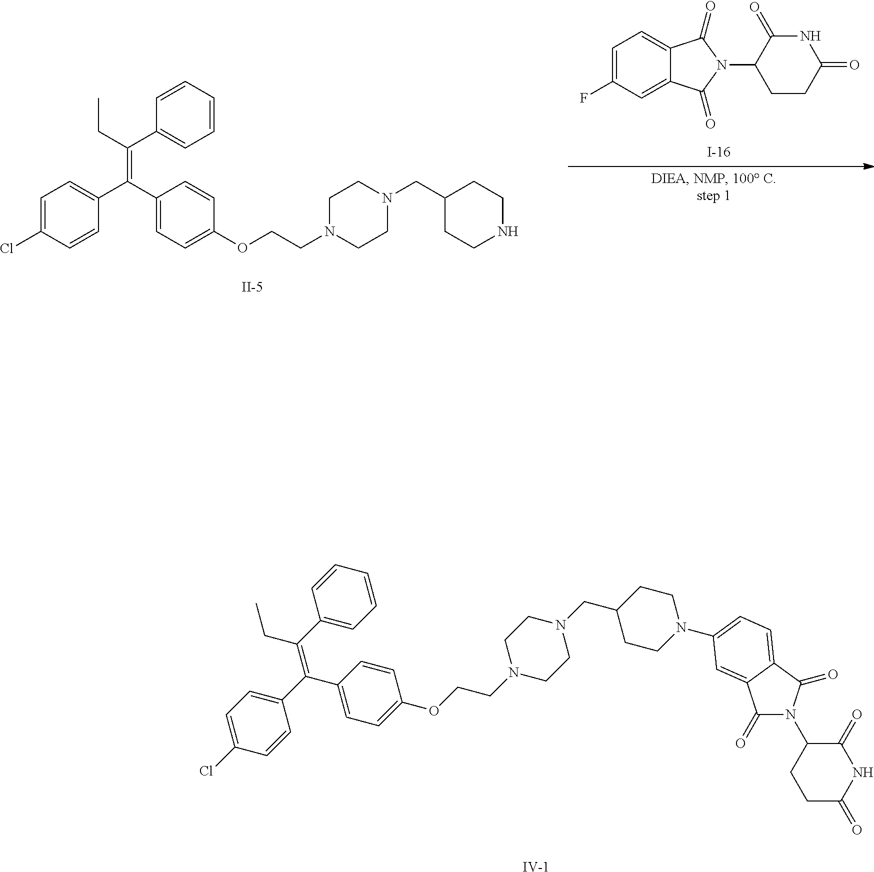

Procedure as example III, step 1. (Z/E)-1-(2-(4-(1-(4-chlorophenyl)-2-phenylbut-1-en-1-yl) phenoxy)ethyl)-4-(piperidin-4-ylmethyl)piperazine (II-5) (490 mg, 0.9 mmol) and 2-(2,6-dioxopiperidin-3-yl)-5-fluoroisoindoline-1,3-dione (I-16) (380 mg, 1.35 mmol) to give (Z/E)-5-(4-((4-(2-(4-(1-(4-chlorophenyl)-2-phenylbut-1-en-1-yl)phenoxy)ethyl)piperazin-1-yl) methyl)piperidin-1-yl)-2-(2,6-dioxopiperidin-3-yl)isoindoline-1,3-dione (IV-1) (198 mg) as tan solid. LCMS [M+H] 800.

Step 2. (Z)-(4-(1-(4-(2-(4-((1-(2-(2,6-dioxopiperidin-3-yl)-1,3-dioxoisoindolin-5-yl)piperidin-4-yl) methyl)piperazin-1-yl)ethoxy)phenyl)-2-phenylbut-1-en-1-yl)phenyl)boronic acid (compound #17) and (E)-(4-(1-(4-(2-(4-((1-(2-(2,6-dioxopiperidin-3-yl)-1,3-dioxoisoindolin-5-yl) piperidin-4-yl)methyl)piperazin-1-yl)ethoxy)phenyl)-2-phenylbut-1-en-1-yl) phenyl)boronic acid (compound #18)

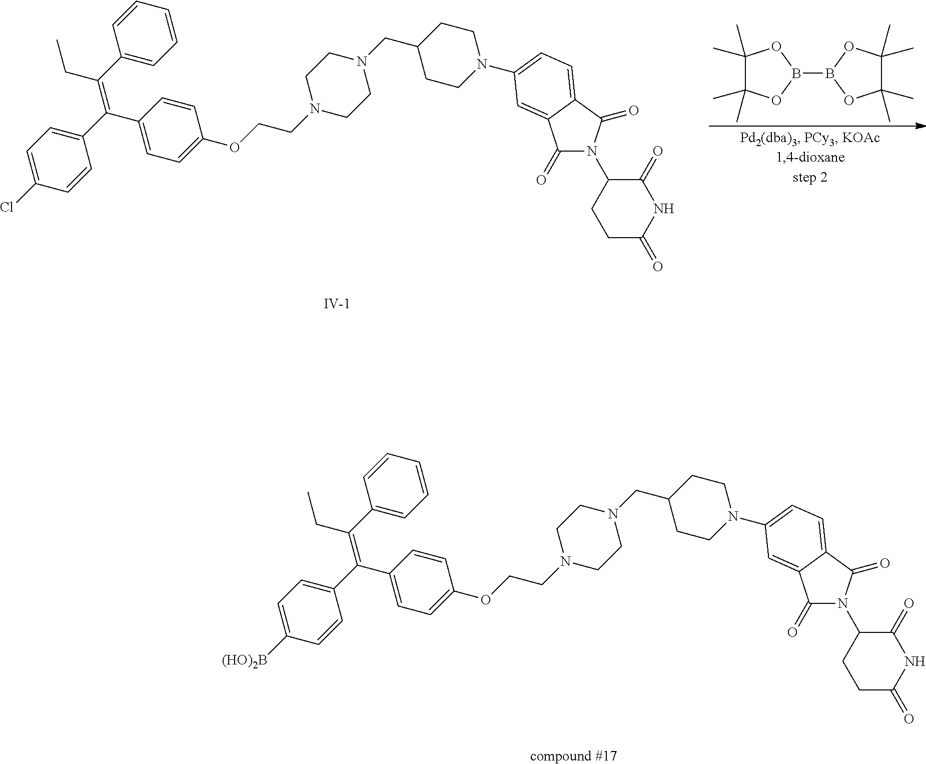

Procedure as example III, step 2. (Z/E)-5-(4-((4-(2-(4-(1-(4-chlorophenyl)-2-phenylbut-1-en-1-yl) phenoxy)ethyl)piperazin-1-yl)methyl)piperidin-1-yl)-2-(2,6-dioxopiperidin-3-yl)isoindoline-1,3-dione (IV-1) (198 mg) provide (Z)-(4-(1-(4-(2-(4-((1-(2-(2,6-dioxopiperidin-3-yl)-1,3-dioxoisoindolin-5-yl)piperidin-4-yl)methyl)piperazin-1-yl)ethoxy)phenyl)-2-phenylbut-1-en-1-yl) phenyl)boronic acid (compound #17) (20.1 mg) as white solid. LCMS [M+H] 810; 1H NMR (DMSO-d6, 400 MHz) δ 11.09 (s, 1H), 8.12 (s, 1H), 7.78 (d, 2H), 7.65 (d, 2H), 7.42 (d, 2H), 6.67-7.23 (m, 12H), 5.06 (m, 1H), 3.90-4.05 (m, 4H), 2.88-3.02 (m, 3H), 1.02-2.66 (m, 26H), 0.88 (t, 3H) and (E)-(4-(1-(4-(2-(4-((1-(2-(2,6-dioxopiperidin-3-yl)-1,3-dioxoisoindolin-5-yl)piperidin-4-yl) methyl)piperazin-1-yl)ethoxy)phenyl)-2-phenylbut-1-en-1-yl)phenyl)boronic acid (compound #18) (5 mg) as white solid. LCMS [M+H] 810. ¹H NMR (DMSO-d6, 300 MHz): δ 0.87 (3H, t), 1.59-1.70 (2H, m), 1.83-2.14 (5H, m), 2.21-2.44 (6H, m), 2.51-2.66 (8H, m), 2.85-2.94 (2H, m), 3.00-3.11 (2H, m), 3.31-3.45 (2H, m), 3.89-4.12 (2H, m), 5.05 (1H, d), 6.92 (2H, d), 7.18 (1H, d), 7.26-7.40 (9H, m), 7.50 (1H, d), 7.61 (2H, d), 8.37 (1H, d).

### Example 7

### Compounds of Formula (I)—Compound 1 and Compound 3

As an illustrative example of a compound of Formula (I), the following compound, compound 1 and compound 3 as denoted TABLE 1, was synthesized:

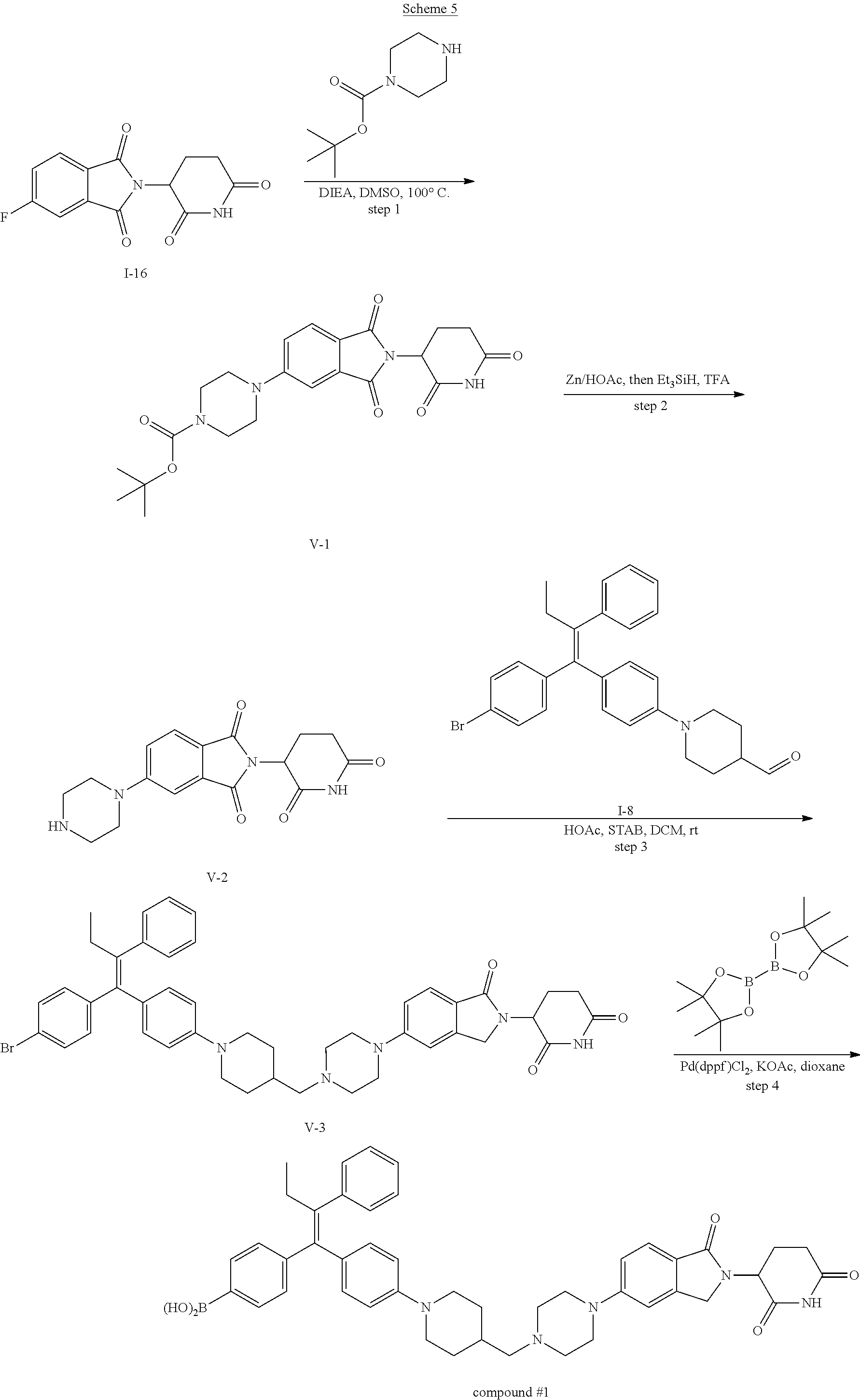

Step 1. tert-butyl 4-(2-(2,6-dioxopiperidin-3-yl)-1,3-dioxoisoindolin-5-yl)piperazine-1-carboxylate (V-1)

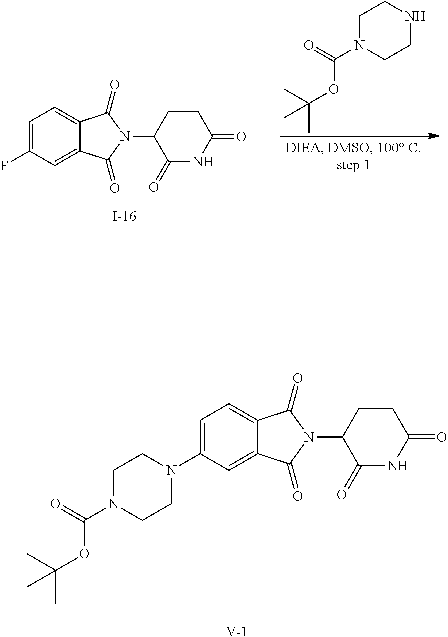

Procedure as example I, step 6. 2-(2,6-dioxopiperidin-3-yl)-5-fluoroisoindoline-1,3-dione (I-16) (5.5 g, 20 mmol) reacted with tert-butyl piperazine-1-carboxylate (5.58 g, 30 mmol) to provide tert-butyl 4-(2-(2,6-dioxopiperidin-3-yl)-1,3-dioxoisoindolin-5-yl)piperazine-1-carboxylate (V-1) (7.0 g) as yellow solid. LCMS [M+H] 443; ¹H NMR (DMSO-d6, 300 MHz): δ 1.42 (9H, s), 1.96-2.14 (2H, m), 2.52-2.98 (10H, m), 5.07 (1H, d), 7.23 (1H, d), 7.35 (1H, d), 7.70 (1H, d), 11.09 (d, 1H).

Step 2. 3-(1-oxo-5-(piperazin-1-yl)isoindolin-2-yl)piperidine-2,6-dione (V-2)

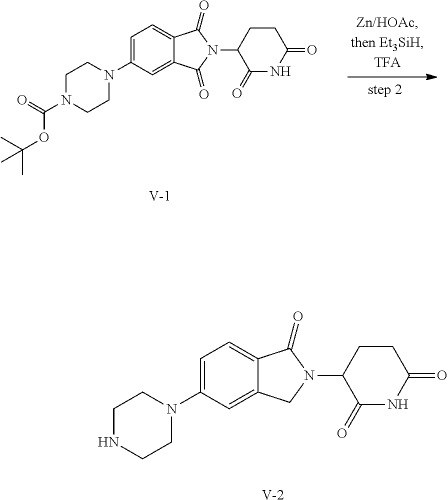

Procedure as example I, step 7, tert-butyl 4-(2-(2,6-dioxopiperidin-3-yl)-1,3-dioxoisoindolin-5-yl) piperazine-1-carboxylate (V-1) (2.0 g) provided 3-(1-oxo-5-(piperazin-1-yl)isoindolin-2-yl) piperidine-2,6-dione (V-2) (370 mg) as white solid. LCMS [M+H] 329.

Step 3. (Z/E)-3-(5-(4-((1-(4-(1-(4-bromophenyl)-2-phenylbut-1-en-1-yl)phenyl)piperidin-4-yl) methyl)piperazin-1-yl)-1-oxoisoindolin-2-yl)piperidine-2,6-dione (V-3)

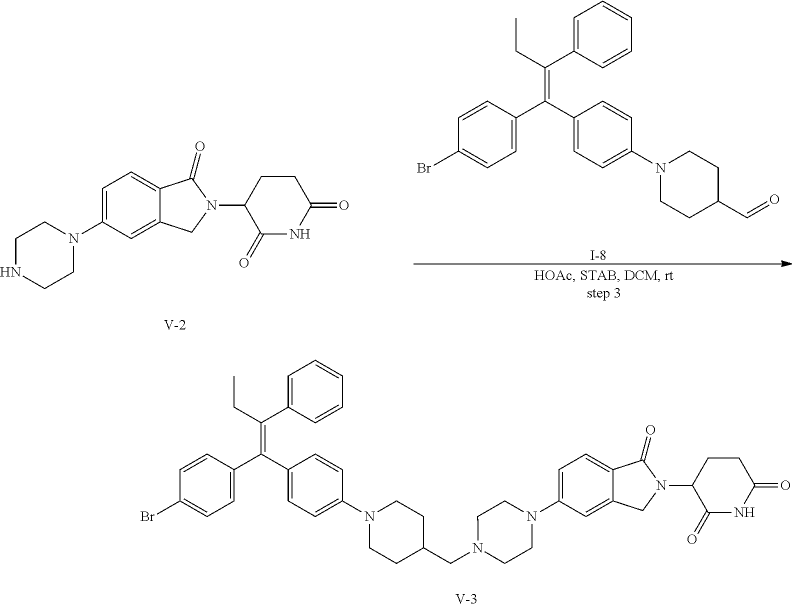

Procedure as example I, step 8. 3-(1-oxo-5-(piperazin-1-yl)isoindolin-2-yl)piperidine-2,6-dione (V-2) (510 mg) reacted with (Z/E)-1-(4-(1-(4-bromophenyl)-2-phenylbut-1-en-1-yl) phenyl)piperidine-4-carbaldehyde (I-8) (1.1 g) to provide (Z/E)-3-(5-(4-((1-(4-(1-(4-bromophenyl)-2-phenylbut-1-en-1-yl)phenyl)piperidin-4-yl)methyl)piperazin-1-yl)-1-oxoisoindolin-2-yl)piperidine-2,6-dione (V-3) (570 mg) as white foam. LCMS [M+H] 786/788.

Step 4. (Z)-(4-(1-(4-(4-((4-(2-(2,6-dioxopiperidin-3-yl)-1-oxoisoindolin-5-yl)piperazin-1-yl) methyl)piperidin-1-yl)phenyl)-2-phenylbut-1-en-1-yl)phenyl)boronic acid (compound #3) and (E)-(4-(1-(4-(4-((4-(2-(2,6-dioxopiperidin-3-yl)-1-oxoisoindolin-5-yl)piperazin-1-yl) methyl)piperidin-1-yl)phenyl)-2-phenylbut-1-en-1-yl)phenyl)boronic acid (compound #4)

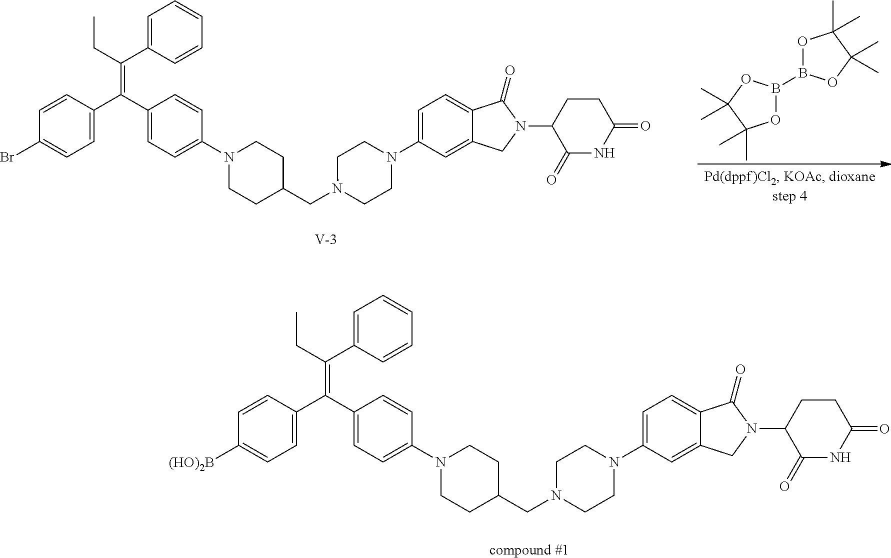

Procedure as example I, step 9 & 10. (Z/E)-3-(5-(4-((1-(4-(1-(4-bromophenyl)-2-phenylbut-1-en-1-yl)phenyl)piperidin-4-yl)methyl)piperazin-1-yl)-1-oxoisoindolin-2-yl)piperidine-2,6-dione (V-3) (570 mg) provided (Z)-(4-(1-(4-(4-((4-(2-(2,6-dioxopiperidin-3-yl)-1-oxoisoindolin-5-yl)piperazin-1-yl)methyl)piperidin-1-yl)phenyl)-2-phenylbut-1-en-1-yl)phenyl)boronic acid (compound #3) (33 mg) as white solid LCMS [M+H] 752; 1H NMR (DMSO-d6, 400 MHz) δ 11.09 (s, 1H), 8.12 (s, 1H), 7.78 (d, 2H), 7.65 (d, 2H), 7.42 (d, 2H), 6.67-7.23 (m, 12H), 5.06 (m, 1H), 3.90-4.05 (m, 4H), 2.88-3.02 (m, 3H), 1.02-2.66 (m, 26H), 0.88 (t, 3H) and (E)-(4-(1-(4-(4-((4-(2-(2,6-dioxopiperidin-3-yl)-1-oxoisoindolin-5-yl)piperazin-1-yl)methyl)piperidin-1-yl)phenyl)-2-phenylbut-1-en-1-yl)phenyl)boronic acid (compound #4) (21 mg) as white solid. LCMS [M+H] 752

### Example 8

### Compounds of Formula (I)—Compound 7

As an illustrative example of a compound of Formula (I), the following compound, compound 7 as denoted TABLE 1, was synthesized:

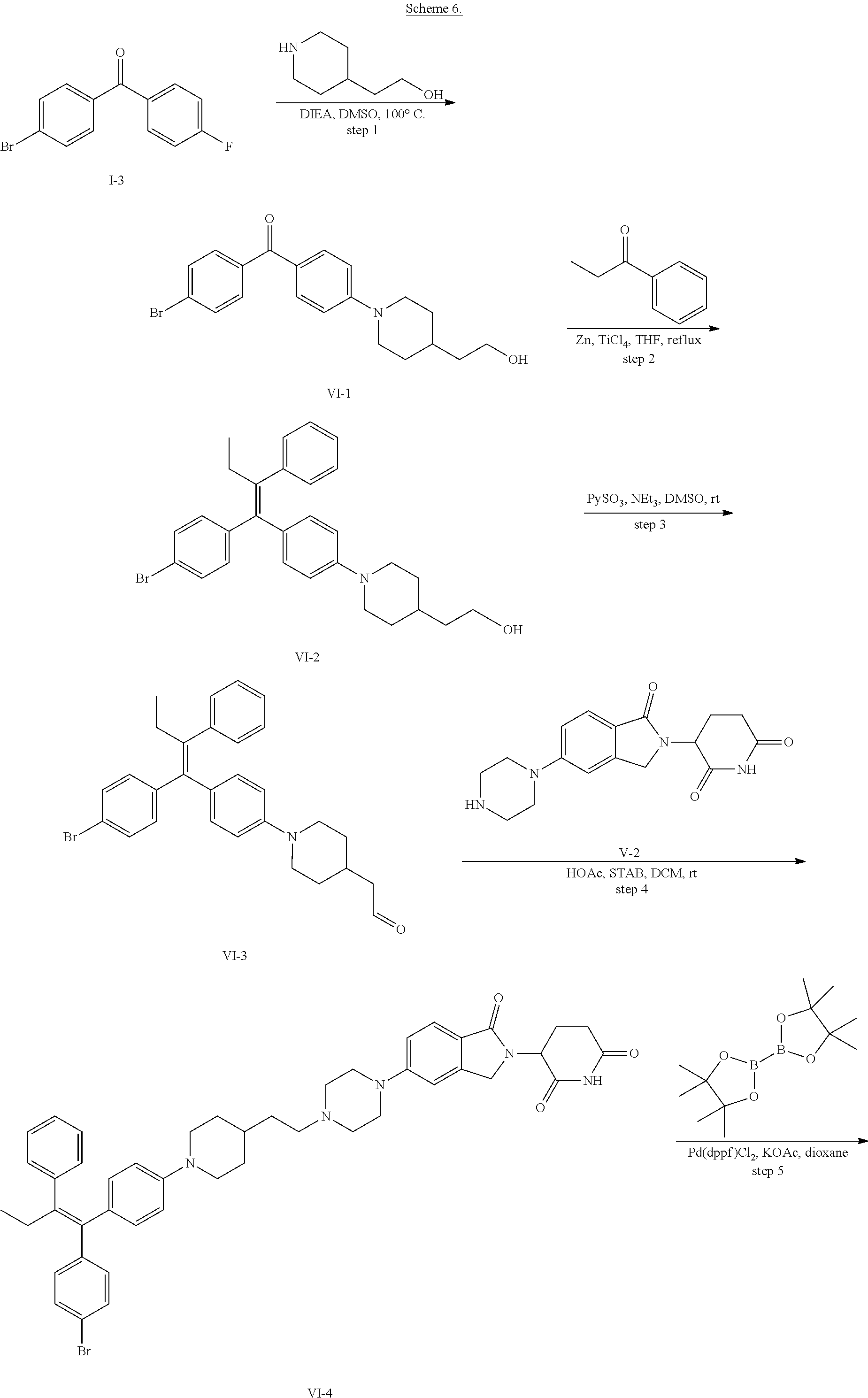

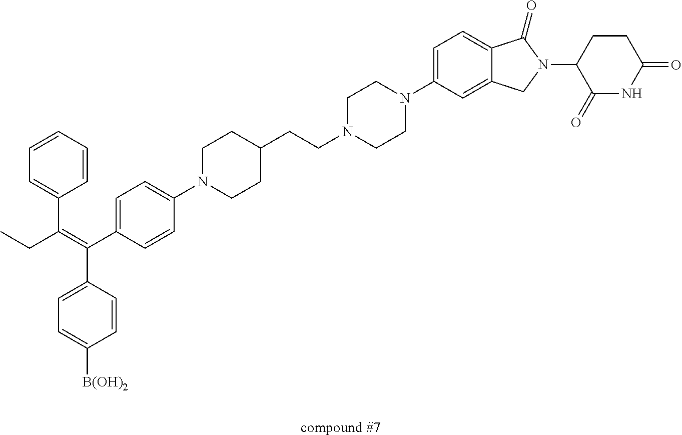

Step 1. (4-bromophenyl)(4-(4-(2-hydroxyethyl)piperidin-1-yl)phenyl)methanone (VI-1)

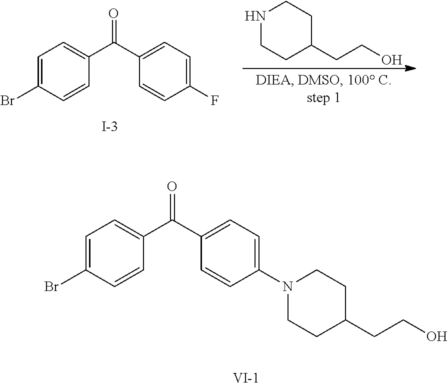

Procedure as example I, step 2. (4-bromophenyl)(4-fluorophenyl)methanone (2.78 g, 10 mmol) provided (4-bromophenyl)(4-(4-(2-hydroxyethyl)piperidin-1-yl)phenyl)methanone (VI-1) (3.8 g) as light yellow solid. LCMS [M+H] 388; 1H NMR (CDCl3, 400 MHz) δ 7.72-7.75 (m, 2H), 7.57-7.63 (m, 4H), 6.82-6.92 (m, 2H), 3.87-3.96 (m, 2H), 3.68-3.78 (m, 2H), 2.92 (dt, 2H), 1.78-1.89 (m, 2H), 1.61-1.77 (m, 1H), 1.21-1.59 (m, 4H).

Step 2. (Z/E)-2-(1-(4-(1-(4-bromophenyl)-2-phenylbut-1-en-1-yl)phenyl)piperidin-4-yl)ethan-1-ol (VI-2)

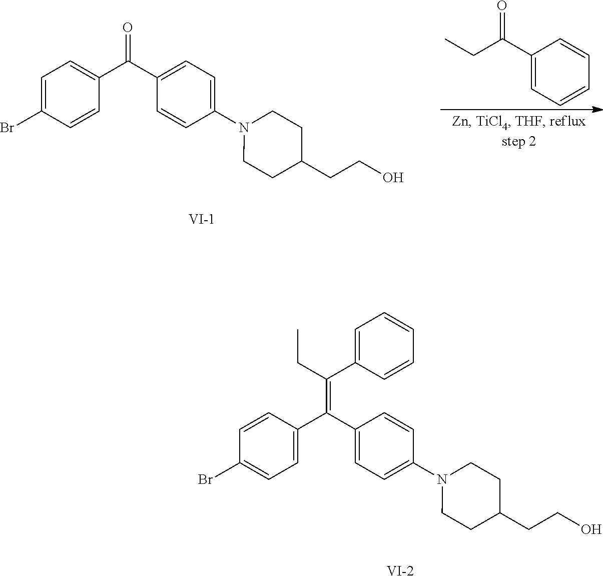

Procedure as example I, step 3. (4-bromophenyl)(4-(4-(2-hydroxyethyl)piperidin-1-yl) phenyl)methanone (VI-1) (3.8 g, 10 mmol) provided (Z/E)-2-(1-(4-(1-(4-bromophenyl)-2-phenylbut-1-en-1-yl)phenyl)piperidin-4-yl)ethan-1-ol (VI-2) (3.585 g) as light yellow oil. LCMS [M+H] 490/492; ¹H NMR (CDCl3, 300 MHz): δ 0.91 (3H, t), 1.37 (2H, t), 1.48-1.67 (5H, m), 2.30 (2H, q), 3.37 (2H, t), 3.41-3.55 (4H, m), 6.76 (2H, d), 7.04-7.20 (3H, m), 7.28-7.48 (8H, m).

Step 3. (Z/E)-2-(1-(4-(1-(4-bromophenyl)-2-phenylbut-1-en-1-yl)phenyl)piperidin-4-yl) acetaldehyde (VI-3)

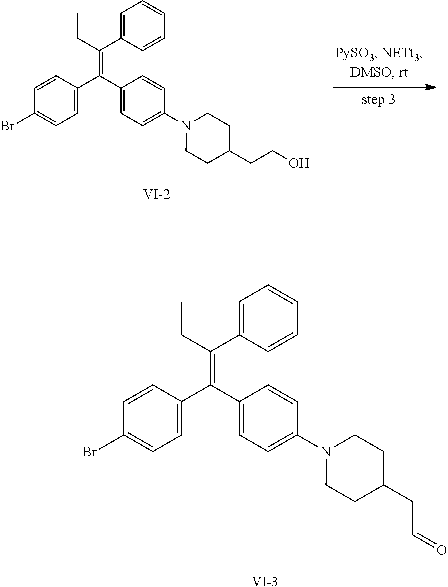 [SMILES: CC/C(=C(\c1ccc(Br)cc1)c1ccc(N2CCC(CCO)CC2)cc1)c1ccccc1 ; "VI-2" ; CC/C(=C(\c1ccc(Br)cc1)c1ccc(N2CCC(CC=O)CC2)cc1)c1ccccc1 ; "VI-3"]

Procedure as example I, step 4. (Z/E)-2-(1-(4-(1-(4-bromophenyl)-2-phenylbut-1-en-1-yl) phenyl)piperidin-4-yl)ethan-1-ol (VI-2) (3.58 g, 7.3 mmol) provided (Z/E)-2-(1-(4-(1-(4-bromophenyl)-2-phenylbut-1-en-1-yl)phenyl)piperidin-4-yl)acetaldehyde (VI-3) (2.64 g) as light yellow oil. LCMS [M+H] 488/490; ¹H NMR (DMSO-d6, 300 MHz): δ 0.90 (3H, t), 1.56-1.75 (4H, m), 2.05 (1H, t), 2.30 (2H, q), 2.52 (2H, d), 3.45-3.60 (4H, m), 6.76 (2H, d), 7.04-7.20 (3H, m), 7.28-7.48 (8H, m), 9.77 (1H, t).

Step 4. (Z/E)-3-(5-(4-(2-(1-(4-(1-(4-bromophenyl)-2-phenylbut-1-en-1-yl)phenyl)piperidin-4-yl) ethyl)piperazin-1-yl)-1-oxoisoindolin-2-yl)piperidine-2,6-dione (VI-4)

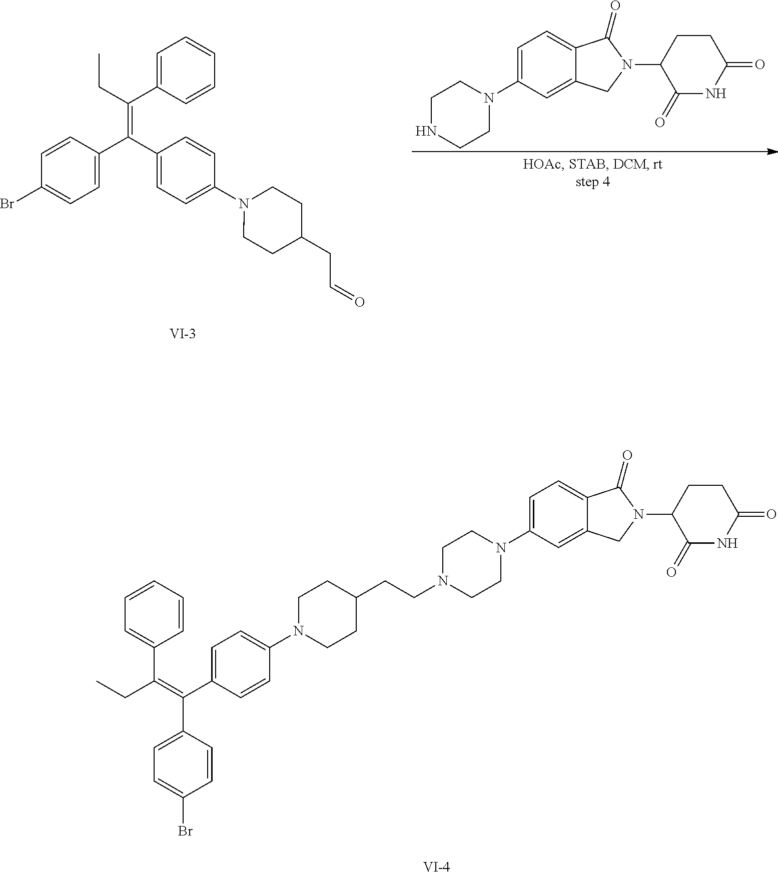 [SMILES: CC/C(=C(\c1ccc(Br)cc1)c1ccc(N2CCC(CC=O)CC2)cc1)c1ccccc1 ; O=C1CCC(N2Cc3cc(N4CCNCC4)ccc3C2=O)C(=O)N1 ; "VI-3" ; CC/C(=C(\c1ccc(Br)cc1)c1ccc(N2CCC(CCN3CCN(c4ccc5c(c4)CN(C4CCC(=O)NC4=O)C5=O)CC3)CC2)cc1)c1ccccc1 ; "VI-4"]

Procedure as example I, step 8. (Z/E)-2-(1-(4-(1-(4-bromophenyl)-2-phenylbut-1-en-1-yl) phenyl)piperidin-4-yl)acetaldehyde (VI-3) (490 mg) reacted with 3-(1-oxo-5-(piperazin-1-yl) isoindolin-2-yl)piperidine-2,6-dione (V-2) (500 mg) to provide (Z/E)-3-(5-(4-(2-(1-(4-(1-(4-bromophenyl)-2-phenylbut-1-en-1-yl)phenyl)piperidin-4-yl)ethyl)piperazin-1-yl)-1-oxoisoindolin-2-yl) piperidine-2,6-dione (VI-4) (0.737 g) as red solid. LCMS [M+H] 800/802; ¹H NMR (DMSO-d6, 300 MHz): δ 0.79-0.91 (3H, t), 1.12-1.32 (m, 2H), 1.41-1.74 (5H, m), 1.87-2.11 (2H, m), 2.31 (1H, d), 2.25-2.40 (3H, m), 2.47-2.62 (6H, m), 2.98-3.12 (4H, m), 3.38-3.56 (4H, m), 4.15-4.38 (2H, m), 5.05 (1H, d), 6.76 (2H, d), 6.98 (1H, d), 7.04-7.20 (4H, m), 7.28-7.48 (9H, m), 10.96 (1H, s).

Step 5. (Z)-(4-(1-(4-(4-(2-(4-(2-(2,6-dioxopiperidin-3-yl)-1-oxoisoindolin-5-yl)piperazin-1-yl) ethyl)piperidin-1-yl)phenyl)-2-phenylbut-1-en-1-yl)phenyl)boronic acid (compound #7) and (E)-(4-(1-(4-(4-(2-(4-(2-(2,6-dioxopiperidin-3-yl)-1-oxoisoindolin-5-yl)piperazin-1-yl) ethyl)piperidin-1-yl)phenyl)-2-phenylbut-1-en-1-yl)phenyl)boronic acid (compound #8)

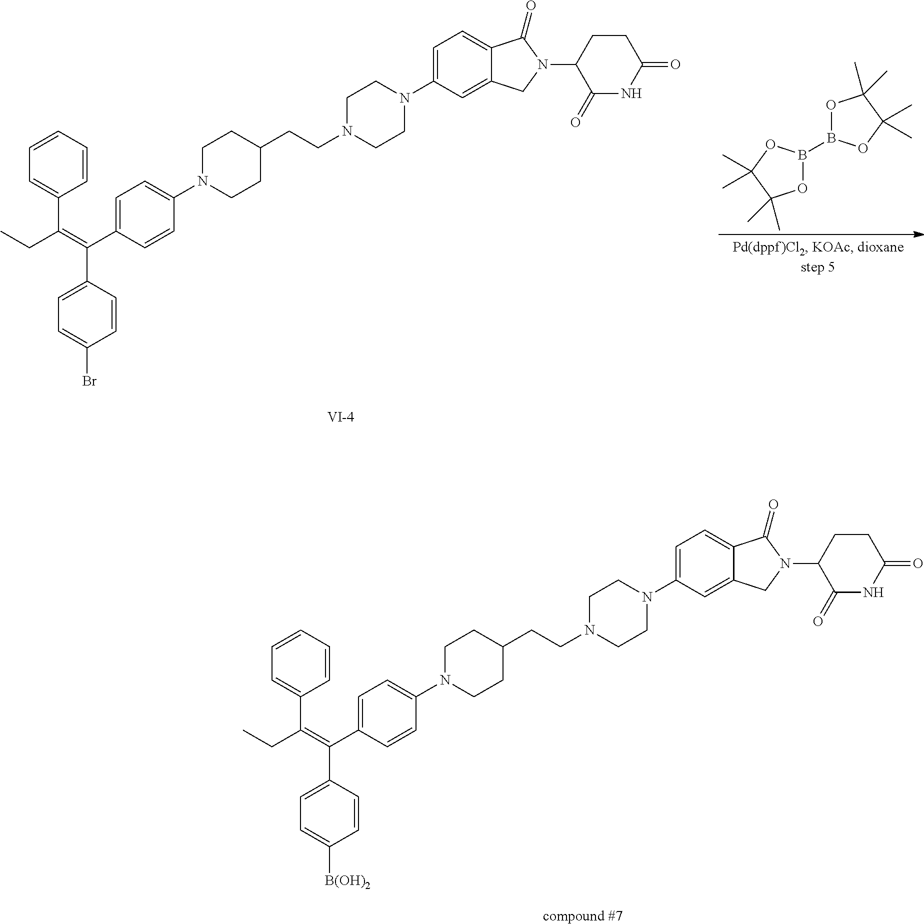

Procedure as example I, step 9 & 10. Standard Miyaura reaction conditions. (Z/E)-3-(5-(4-(2-(1-(4-(1-(4-bromophenyl)-2-phenylbut-1-en-1-yl)phenyl)piperidin-4-yl)ethyl)piperazin-1-yl)-1-oxoisoindolin-2-yl)piperidine-2,6-dione (VI-4) (0.537 g) provided (Z)-(4-(1-(4-(4-(2-(4-(2-(2,6-dioxopiperidin-3-yl)-1-oxoisoindolin-5-yl)piperazin-1-yl)ethyl)piperidin-1-yl)phenyl)-2-phenylbut-1-en-1-yl)phenyl)boronic acid (compound #7) (50 mg) LCMS [M+H] 848; as white solid 1H NMR (DMSO-d6, 300 MHz): δ 0.80-0.87 (3H, m), 1.12-1.14 (2H, m), 1.41-1.74 (5H, m), 1.87-2.11 (2H, m), 2.25-2.40 (3H, m), 2.47-2.62 (6H, m), 2.98-3.12 (4H, m), 3.38-3.56 (4H, m), 4.26 (2H, dd), 5.08 (1H, m), 6.76 (2H, d), 6.98 (1H, d), 7.06 (1H, d), 7.13-7.89 (7H, m) and (E)-(4-(1-(4-(4-(2-(4-(2-(2,6-dioxopiperidin-3-yl)-1-oxoisoindolin-5-yl)piperazin-1-yl)ethyl)piperidin-1-yl)phenyl)-2-phenylbut-1-en-1-yl)phenyl)boronic acid (compound #8) (30 mg) as white solid. LCMS [M+H] 848.

### Example 9

### Compounds of Formula (I)—Compound 15

As an illustrative example of a compound of Formula (I), the following compound, compound 15 as denoted TABLE 1, was synthesized:

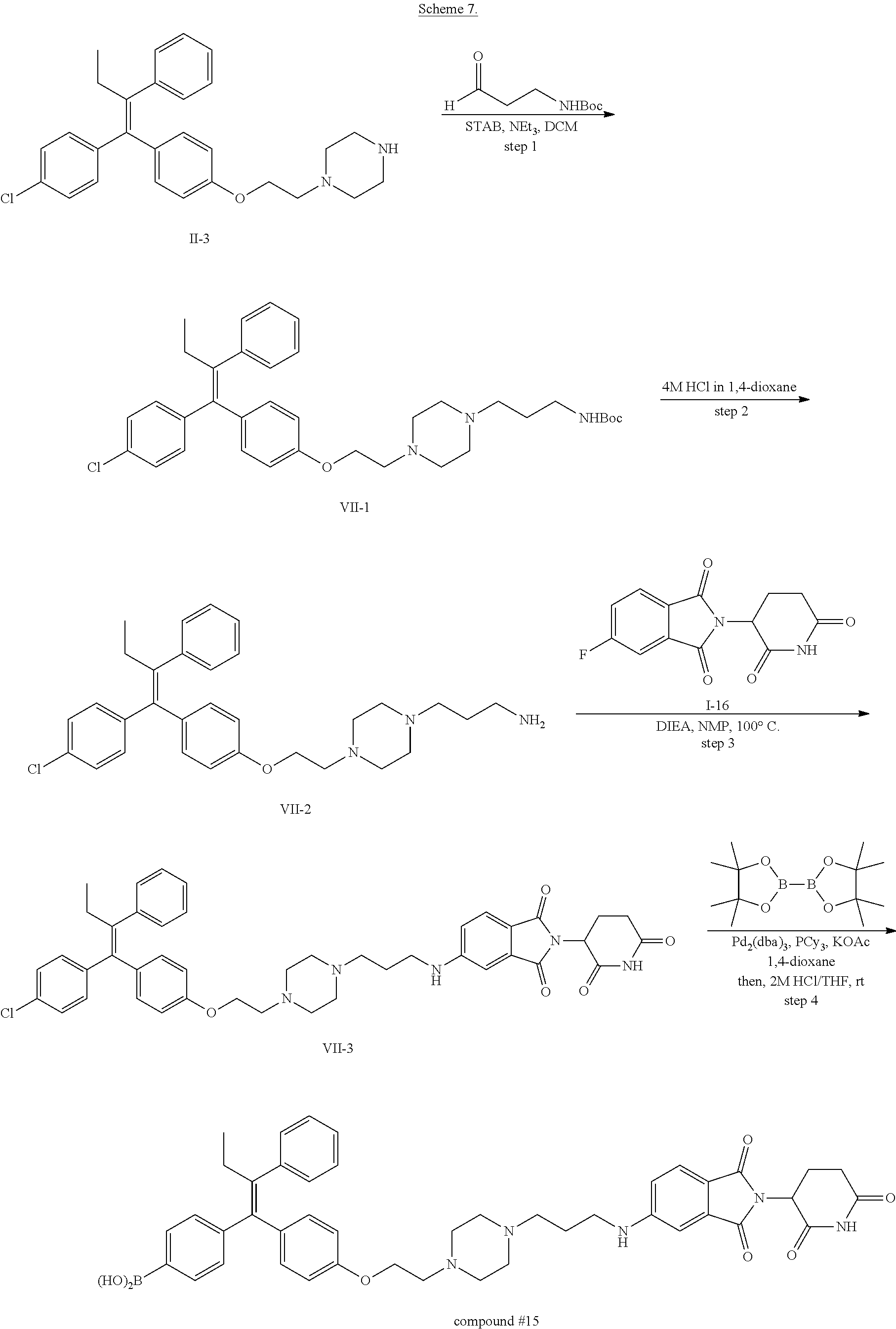

Step 1. tert-butyl (Z/E)-(3-(4-(2-(4-(1-(4-chlorophenyl)-2-phenylbut-1-en-1-yl) phenoxy)ethyl)piperazin-1-yl)propyl)carbamate (VII-1)

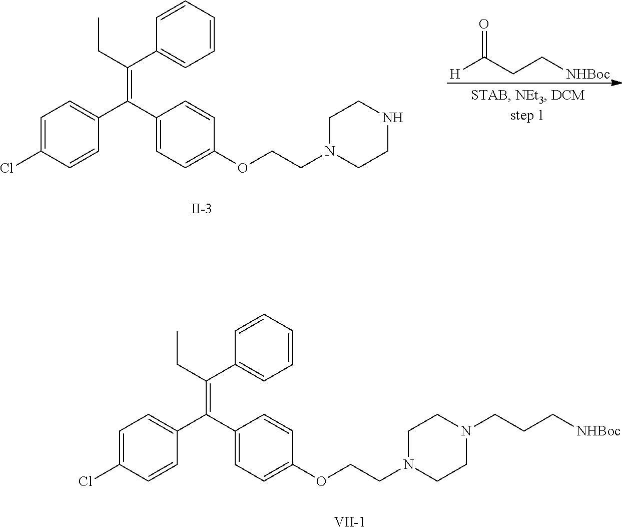

Procedure as example II, step 4. (Z/E)-1-(2-(4-(1-(4-chlorophenyl)-2-phenylbut-1-en-1-yl) phenoxy)ethyl)piperazine (II-3) (2.58 g, 5 mmol) produced tert-butyl (Z/E)-(3-(4-(2-(4-(1-(4-chlorophenyl)-2-phenylbut-1-en-1-yl)phenoxy)ethyl)piperazin-1-yl)propyl)carbamate (VII-1) (3.28 g) as colorless oil. LCMS [M+H] 604; ¹H NMR (CDCl3, 300 MHz): δ 0.91 (3H, t), 1.43 (9H, s), 1.84 (2H, t), 2.31 (2H, d), 2.46-2.63 (10H, m), 2.88 (2H, t), 3.20 (2H, t), 4.23 (2H, t), 6.92 (2H, d), 7.21 (2H, d), 7.26-7.42 (7H, m), 7.50 (2H, d).

Step 2. (Z/E)-3-(4-(2-(4-(1-(4-chlorophenyl)-2-phenylbut-1-en-1-yl)phenoxy)ethyl)piperazin-1-yl)propan-1-amine (VII-2)

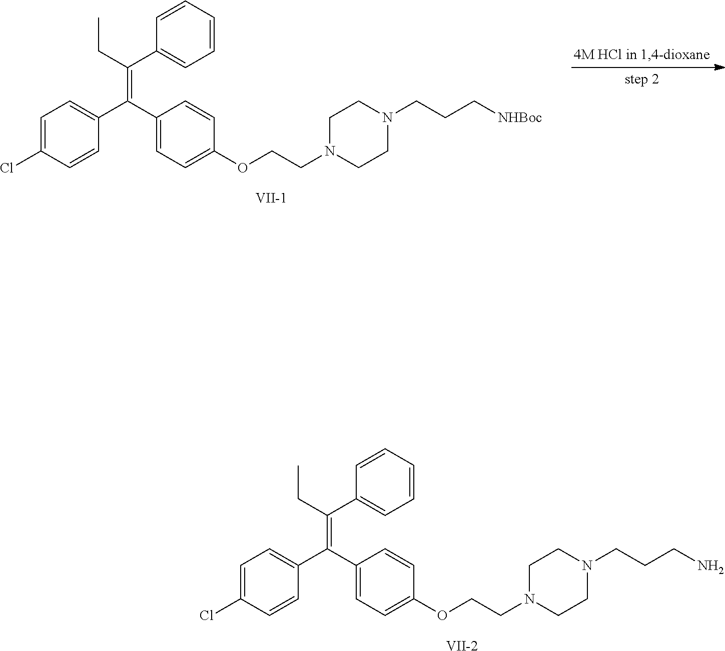

Procedure as example II, step 5. tert-butyl (Z/E)-(3-(4-(2-(4-(1-(4-chlorophenyl)-2-phenylbut-1-en-1-yl)phenoxy)ethyl)piperazin-1-yl)propyl)carbamate (VII-1) (3.28 g) produced (Z/E)-3-(4-(2-(4-(1-(4-chlorophenyl)-2-phenylbut-1-en-1-yl)phenoxy)ethyl)piperazin-1-yl)propan-1-amine (VII-2) (3.5 g) as white solid. LCMS [M+H] 504; ¹H NMR (DMSO-d6, 300 MHz): δ 0.86 (3H, t), 1.52 (2H, d), 2.31 (2H, d), 2.46-2.64 (12H, m), 2.88 (2H, d), 4.23 (2H, d), 6.92 (2H, d), 7.21 (2H, d), 7.26-7.42 (7H, m), 7.50 (2H, d).

Step 3. (Z/E)-5-((3-(4-(2-(4-(1-(4-chlorophenyl)-2-phenylbut-1-en-1-yl) phenoxy)ethyl)piperazin-1-yl)propyl)amino)-2-(2,6-dioxopiperidin-3-yl)isoindoline-1,3-dione (VII-3)

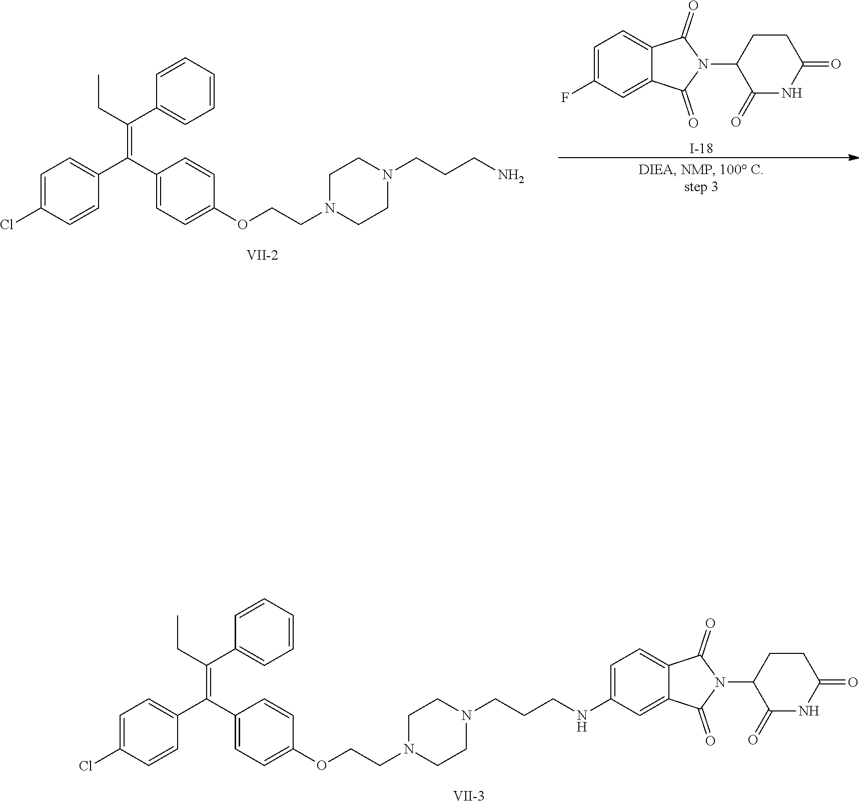

Procedure as example III, step 1. (Z/E)-3-(4-(2-(4-(1-(4-chlorophenyl)-2-phenylbut-1-en-1-yl) phenoxy)ethyl)piperazin-1-yl)propan-1-amine (VII-2) (3.5 g) produced (Z/E)-5-((3-(4-(2-(4-(1-(4-chlorophenyl)-2-phenylbut-1-en-1-yl)phenoxy)ethyl)piperazin-1-yl)propyl)amino)-2-(2,6-dioxopiperidin-3-yl)isoindoline-1,3-dione (VII-3) (238 mg) as red oil. LCMS [M+H] 760.

Step 4. (Z)-(4-(1-(4-(2-(4-(3-((2-(2,6-dioxopiperidin-3-yl)-1,3-dioxoisoindolin-5-yl) amino)propyl)piperazin-1-yl)ethoxy)phenyl)-2-phenylbut-1-en-1-yl)phenyl)boronic acid (compound #15) and (E)-(4-(1-(4-(2-(4-(3-((2-(2,6-dioxopiperidin-3-yl)-1,3-dioxoisoindolin-5-yl) amino)propyl)piperazin-1-yl)ethoxy)phenyl)-2-phenylbut-1-en-1-yl)phenyl)boronic acid (compound #16)

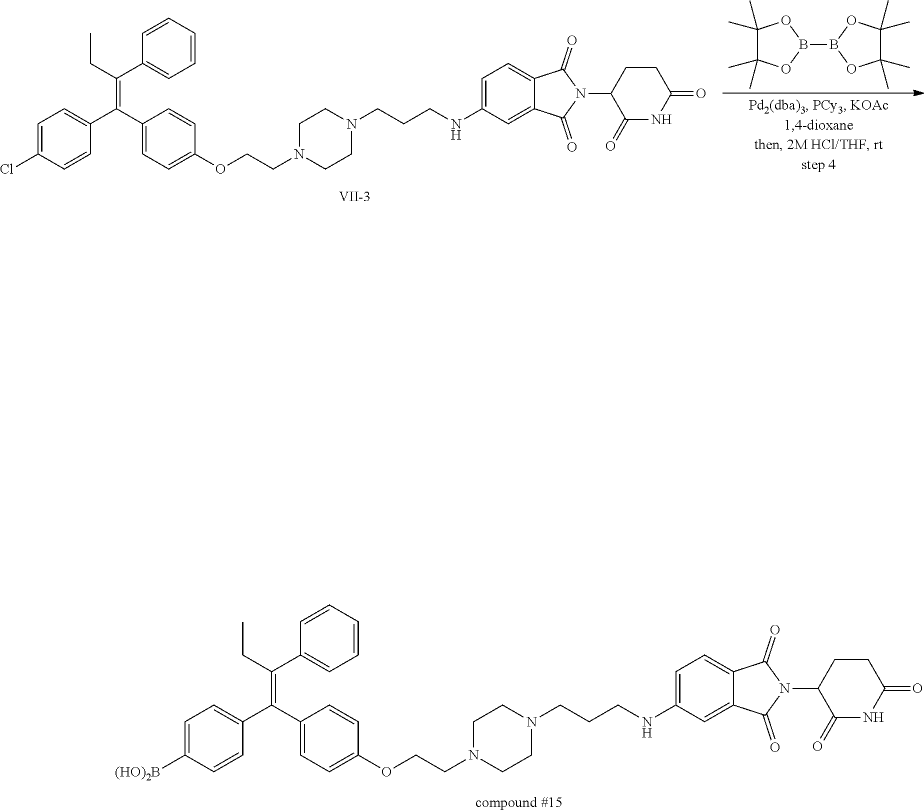

Procedure as example III, step 2. (Z/E)-5-((3-(4-(2-(4-(1-(4-chlorophenyl)-2-phenylbut-1-en-1-yl) phenoxy)ethyl)piperazin-1-yl)propyl)amino)-2-(2,6-dioxopiperidin-3-yl)isoindoline-1,3-dione (VII-3) (238 mg) produced (Z)-(4-(1-(4-(2-(4-(3-((2-(2,6-dioxopiperidin-3-yl)-1,3-dioxoisoindolin-5-yl) amino)propyl)piperazin-1-yl)ethoxy)phenyl)-2-phenylbut-1-en-1-yl)phenyl)boronic acid (6 mg) (compound #15) as white solid LCMS [M+H] 770 and (E)-(4-(1-(4-(2-(4-(3-((2-(2,6-dioxopiperidin-3-yl)-1,3-dioxoisoindolin-5-yl)amino)propyl)piperazin-1-yl)ethoxy)phenyl)-2-phenylbut-1-en-1-yl)phenyl)boronic acid (compound #16) (4 mg) as white solid, LCMS [M+H] 770. ¹H NMR (DMSO-d6, 300 MHz): δ 0.84 (3H, t), 1.74-1.84 (2H, m), 1.96-2.14 (2H, m), 2.21-2.44 (4H, m), 2.45-2.64 (10H, m), 2.85-2.94 (2H, m), 3.26-3.34 (2H, m), 4.18-4.27 (2H, m), 5.02 (1H, dd), 6.89-6.96 (3H, m), 7.26-7.40 (9H, m), 7.51 (1H, d), 7.61 (2H, d), 8.35 (1H, d), 11.03 (1H, s).

### Example 10

### Compounds of Formula (I)—Compound 19

As an illustrative example of a compound of Formula (I), the following compound, compound 19 as denoted TABLE 1, was synthesized:

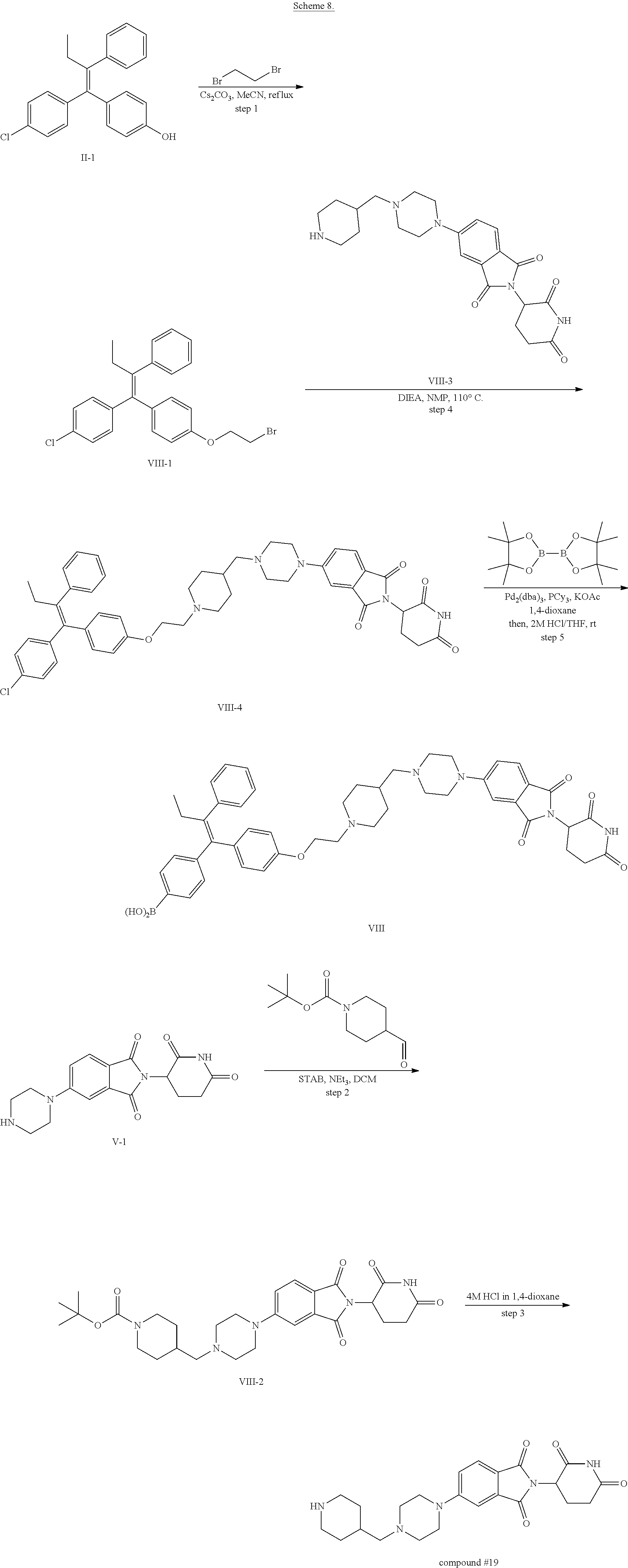

Step 1. (Z/E)-1-(2-bromoethoxy)-4-(1-(4-chlorophenyl)-2-phenylbut-1-en-1-yl)benzene (VIII-1)

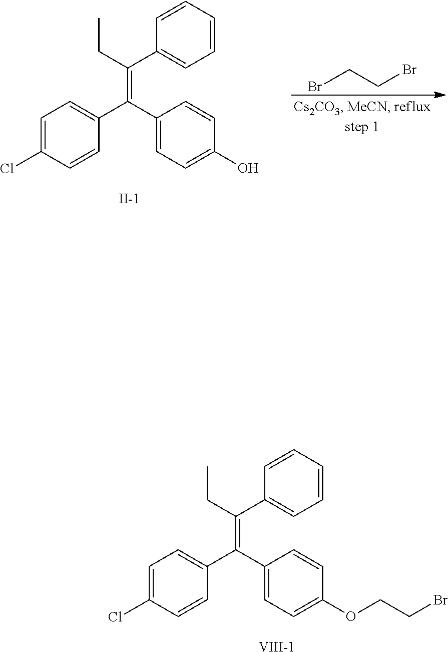

A mixture of (Z/E)-4-(1-(4-chlorophenyl)-2-phenylbut-1-en-1-yl)phenol (II-1) (2.0 g, 5 mmol), 1,2-dibromoethane (9.4 g, 50 mmol) and Cs2CO3 (6.5 g, 20 mmol) in 50 mL of MeCN was stirred at reflux overnight. The solids were filtered out and the filtrate was concentrated in vacuo. The residue was subjected onto silica gel column chromatography with 10% EA in n-hexane as eluent to provide (Z/E)-1-(2-bromoethoxy)-4-(1-(4-chlorophenyl)-2-phenylbut-1-en-1-yl)benzene (VIII-1) (4.14 g) as yellow oil. LCMS [M+H] 441/443. ¹H NMR (CDCl3, 300 MHz): δ 0.92 (3H, t), 2.15 (2H, q), 3.60 (2H, t), 4.23 (2H, t), 6.92 (2H, d), 7.21 (2H, d), 7.26-7.42 (7H, m), 7.50 (2H, d).

Step 2. tert-butyl 4-((4-(2-(2,6-dioxopiperidin-3-yl)-1,3-dioxoisoindolin-5-yl)piperazin-1-yl) methyl)piperidine-1-carboxylate (VIII-2)

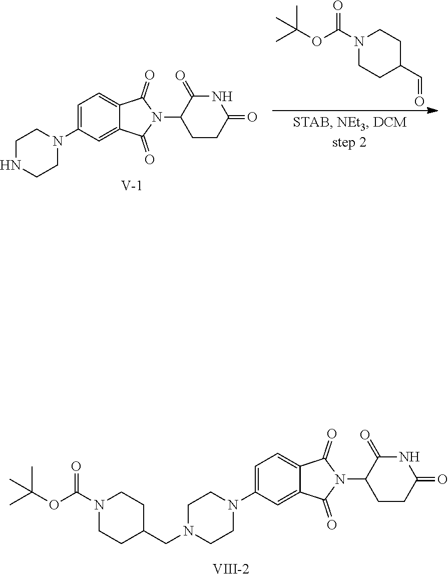

A solution of 2-(2,6-dioxopiperidin-3-yl)-5-(piperazin-1-yl)isoindoline-1,3-dione (V-1) (1.0 g, 3 mmol), tert-butyl 4-formylpiperidine-1-carboxylate (1.28 g, 6 mmol), triethylamine (910 mg, 9 mmol) in 20 mL of DCM was stirred at room temperature overnight. STAB was added portionwise at 0° C. and the resulting mixture was stirred at room temperature for 2 hours. The solvent was removed in vacuo. The residue was subjected onto silica gel column chromatography with 20% MeOH in EA as eluent to give tert-butyl 4-((4-(2-(2,6-dioxopiperidin-3-yl)-1,3-dioxoisoindolin-5-yl)piperazin-1-yl)methyl)piperidine-1-carboxylate (VIII-2) (1.1 g) as yellow solid. LCMS [M+H] 540; ¹H NMR (DMSO-d6, 300 MHz): δ 1.40 (9H, s), 1.53-1.65 (2H, m), 1.74-2.14 (5H, m), 2.27 (1H, d), 2.29-2.44 (3H, m), 2.50-2.73 (4H, m), 2.99-3.13 (4H, m), 3.89-3.96 (2H, m), 5.08 (1H, d), 7.25 (1H, d), 7.34 (1H, d), 7.67 (1H, d), 11.09 (1H, s).

Step 3. 2-(2,6-dioxopiperidin-3-yl)-5-(4-(piperidin-4-ylmethyl)piperazin-1-yl)isoindoline-1,3-dione (VIII-3)

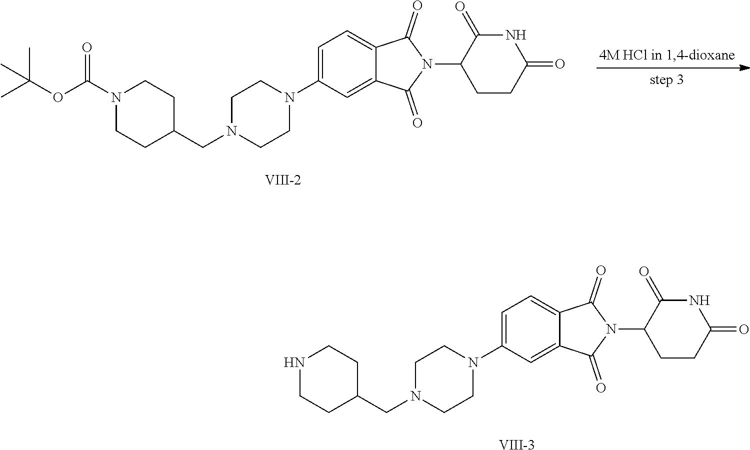

4 M HCl in 1,4-dioxane was added tert-butyl 4-((4-(2-(2,6-dioxopiperidin-3-yl)-1,3-dioxoisoindolin-5-yl)piperazin-1-yl)methyl)piperidine-1-carboxylate (VIII-2) (1.1 g) and the mixture was stirred for 1 hour. The solvent was removed in vacuo. 2-(2,6-dioxopiperidin-3-yl)-5-(4-(piperidin-4-ylmethyl)piperazin-1-yl)isoindoline-1,3-dione HCl salt (VIII-3) (1.34 g) was obtained as yellow solid and used in the next step without further purification. LCMS [M+H] 440; ¹H NMR (DMSO-d6, 300 MHz): δ 1.54-1.66 (2H, m), 1.83 (1H, d), 1.74-1.94 (2H, m), 1.96-2.14 (2H, m), 2.21-2.44 (4H, m), 2.50-2.72 (6H, m), 2.85-2.93 (2H, m), 2.99-3.13 (4H, m), 5.07 (1H, dd), 7.34 (1H, dd), 7.50 (1H, dd), 7.77 (1H, dd), 11.11 (1H, s).

Step 4. (Z/E)-5-(4-((1-(2-(4-(1-(4-chlorophenyl)-2-phenylbut-1-en-1-yl) phenoxy)ethyl)piperidin-4-yl)methyl)piperazin-1-yl)-2-(2,6-dioxopiperidin-3-yl) isoindoline-1,3-dione (VIII-4)

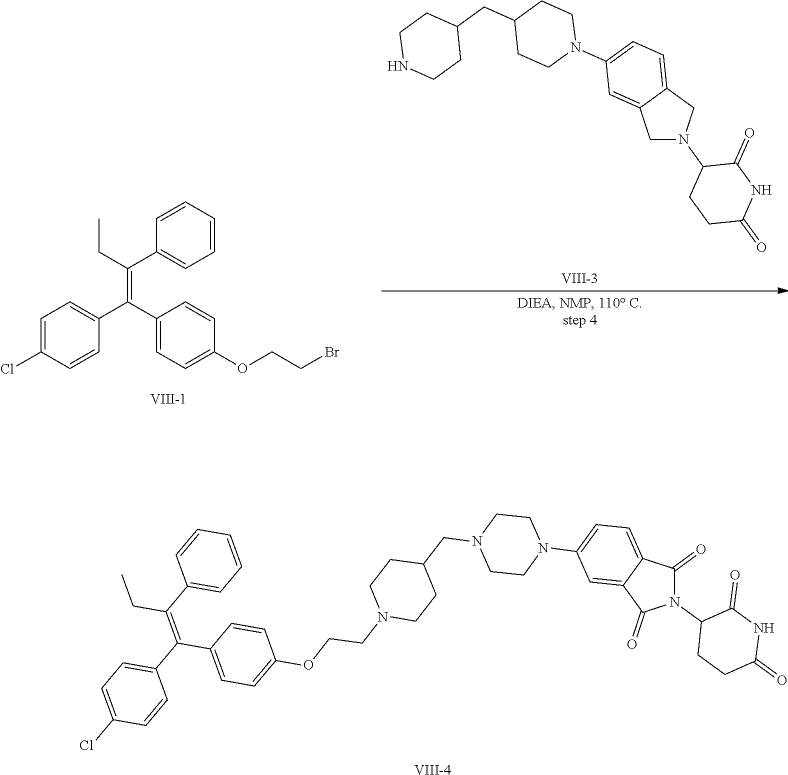

A solution of (Z/E)-1-(2-bromoethoxy)-4-(1-(4-chlorophenyl)-2-phenylbut-1-en-1-yl)benzene (VIII-1) (440 mg, 1 mmol), 2-(2,6-dioxopiperidin-3-yl)-5-(4-(piperidin-4-ylmethyl)piperazin-1-yl) isoindoline-1,3-dione HCl salt (VIII-3) (440 mg, 1 mmol) and DIEA (390 mg, 3 mmol) in 2 mL of NMP was stirred at 110° C. for 2 h. The product was purified by silica gel column chromatography with 20% MeOH in EA as eluent. The obtained crude mixture was further purified by prep-HPLC with 0.5% FA-MeCN to provide (Z/E)-5-(4-((1-(2-(4-(1-(4-chlorophenyl)-2-phenylbut-1-en-1-yl)phenoxy)ethyl)piperidin-4-yl)methyl)piperazin-1-yl)-2-(2,6-dioxopiperidin-3-yl) isoindoline-1,3-dione (VIII-4) (0.10 g) as yellow solid. LCMS [M+H] 800.

Step 5. (Z)-(4-(1-(4-(2-(4-((4-(2-(2,6-dioxopiperidin-3-yl)-1,3-dioxoisoindolin-5-yl)piperazin-1-yl)methyl)piperidin-1-yl)ethoxy)phenyl)-2-phenylbut-1-en-1-yl)phenyl)boronic acid (compound #19) and (E)-(4-(1-(4-(2-(4-((4-(2-(2,6-dioxopiperidin-3-yl)-1,3-dioxoisoindolin-5-yl) piperazin-1-yl)methyl)piperidin-1-yl)ethoxy)phenyl)-2-phenylbut-1-en-1-yl) phenyl)boronic acid (compound #20)

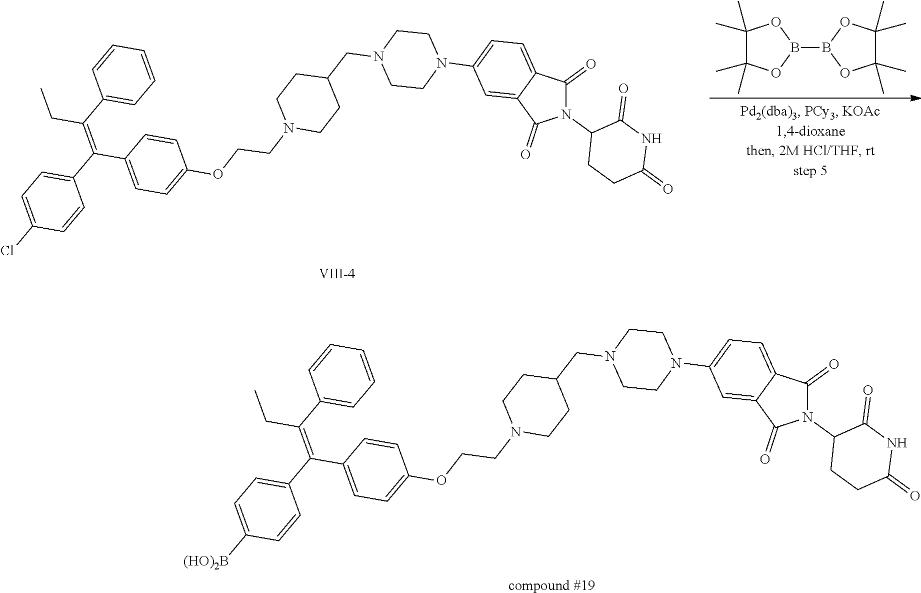

Procedure as example III, step 2. (Z/E)-5-(4-((1-(2-(4-(1-(4-chlorophenyl)-2-phenylbut-1-en-1-yl) phenoxy)ethyl)piperidin-4-yl)methyl)piperazin-1-yl)-2-(2,6-dioxopiperidin-3-yl)isoindoline-1,3-dione (VIII-4) (0.10 g) produced (Z)-(4-(1-(4-(2-(4-((4-(2-(2,6-dioxopiperidin-3-yl)-1,3-dioxoisoindolin-5-yl)piperazin-1-yl)methyl)piperidin-1-yl)ethoxy)phenyl)-2-phenylbut-1-en-1-yl) phenyl)boronic acid (compound #19) (20 mg) as white solid LCMS [M+H] 810; ¹H NMR (DMSO-d6, 300 MHz): δ 0.86 (3H, t), 1.45-1.65 (4H, m), 1.81-2.73 (17H, m), 2.88-3.13 (6H, m), 3.91-4.13 (2H, m), 5.05 (1H, d), 6.92 (2H, d), 7.06-8.22 (14H, m) and (E)-(4-(1-(4-(2-(4-((4-(2-(2,6-dioxopiperidin-3-yl)-1,3-dioxoisoindolin-5-yl)piperazin-1-yl)methyl)piperidin-1-yl)ethoxy)phenyl)-2-phenylbut-1-en-1-yl)phenyl)boronic acid (compound #20) (5 mg) as white solid. LCMS [M+H] 810.

### Example 11

### Compounds of Formula (I)—Compound 31

As an illustrative example of a compound of Formula (I), the following compound, compound 31 as denoted TABLE 1, was synthesized:

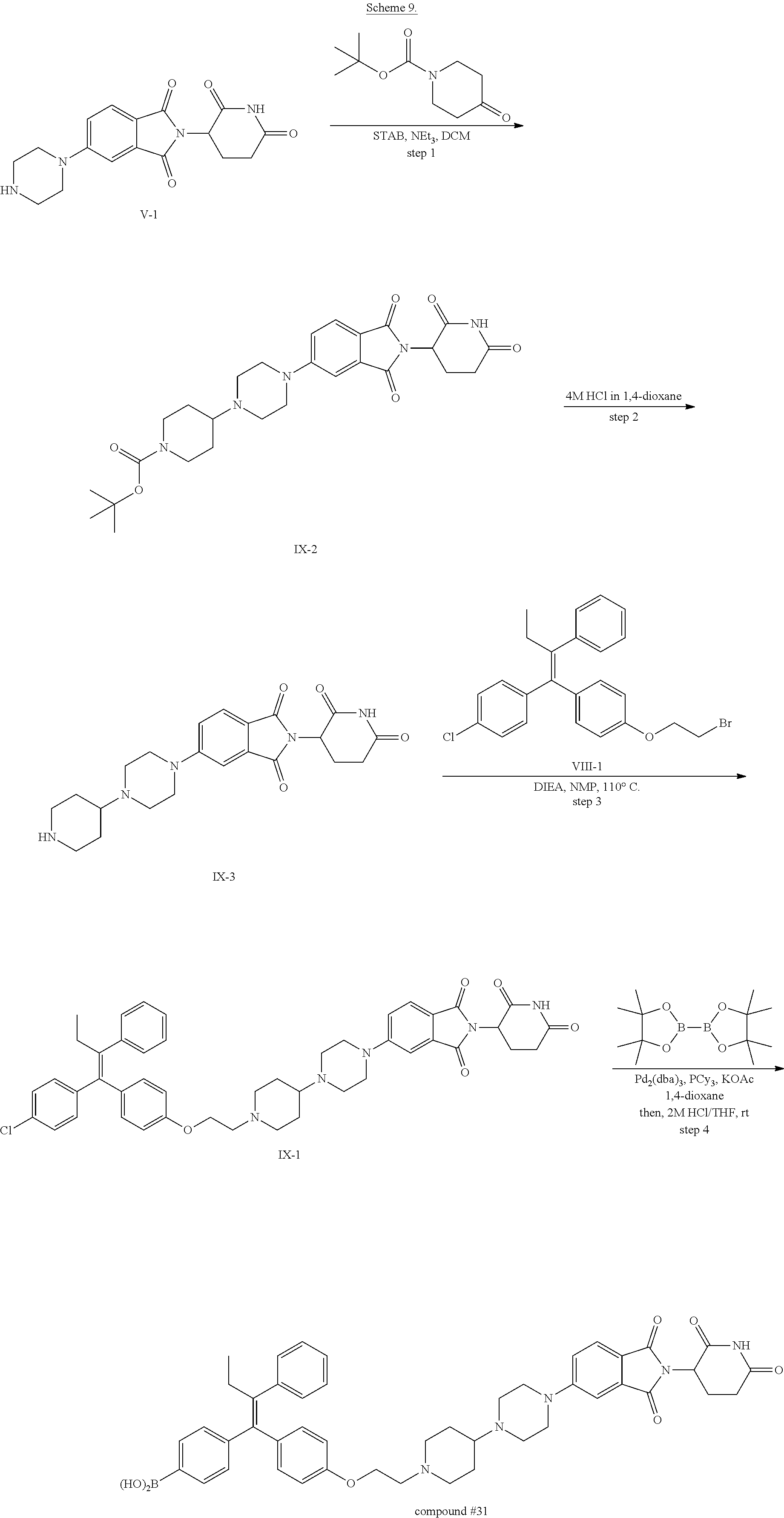

Step 1. tert-butyl 4-(4-(2-(2,6-dioxopiperidin-3-yl)-1,3-dioxoisoindolin-5-yl)piperazin-1-yl) piperidine-1-carboxylate (IX-2)

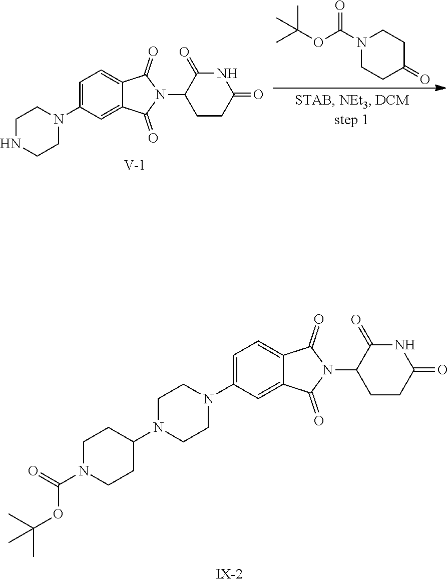

Procedure as example VIII, step 2. 2-(2,6-dioxopiperidin-3-yl)-5-(piperazin-1-yl)isoindoline-1,3-dione (V-1) (1.0 g, 3 mmol) reacted with tert-butyl 4-oxopiperidine-1-carboxylate (1.2 g, 6 mmol) to produce tert-butyl 4-(4-(2-(2,6-dioxopiperidin-3-yl)-1,3-dioxoisoindolin-5-yl)piperazin-1-yl) piperidine-1-carboxylate (IX-2) (2.1 g) as yellow oil. LCMS [M+H] 526; ¹H NMR (DMSO-d6, 300 MHz): δ 1.42 (9H, s), 1.71-1.93 (4H, 1 m), 1.96-2.14 (2H, m), 2.21-2.44 (2H, m), 2.46-2.73 (5H, m), 2.99-3.13 (4H, m), 3.27-3.45 (4H, m), 5.09 (1H, dd), 7.23 (1H, d), 7.33 (1H, d), 7.67 (1H, d).

Step 2. 2-(2,6-dioxopiperidin-3-yl)-5-(4-(piperidin-4-yl)piperazin-1-yl)isoindoline-1,3-dione (IX-3)

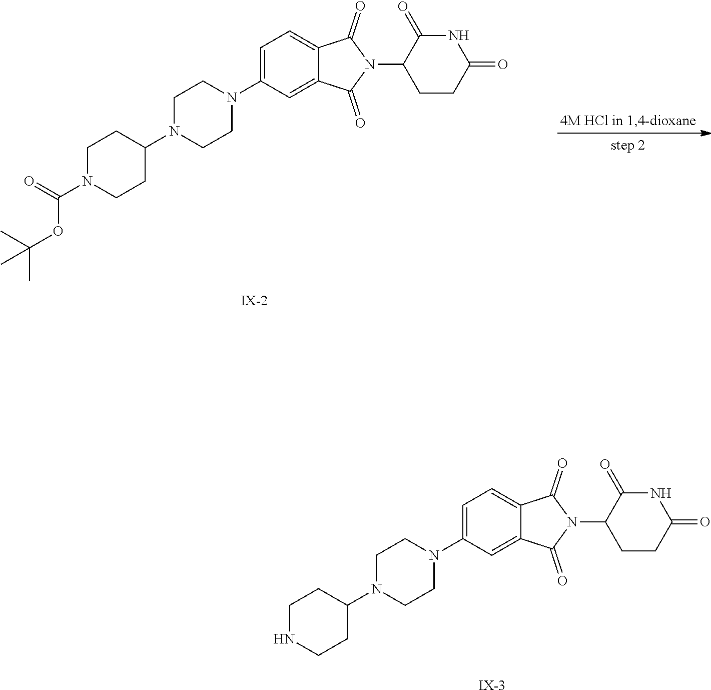

Procedure as example VIII, step 3. tert-butyl 4-(4-(2-(2,6-dioxopiperidin-3-yl)-1,3-dioxoisoindolin-5-yl)piperazin-1-yl)piperidine-1-carboxylate (IX-2) (2.1 g) produced 2-(2,6-dioxopiperidin-3-yl)-5-(4-(piperidin-4-yl)piperazin-1-yl)isoindoline-1,3-dione (IX-3) (2.2 g) as yellow solid. LCMS [M+H] 426.

Step 3. (Z/E)-5-(4-(1-(2-(4-(1-(4-chlorophenyl)-2-phenylbut-1-en-1-yl) phenoxy)ethyl)piperidin-4-yl)piperazin-1-yl)-2-(2,6-dioxopiperidin-3-yl)isoindoline-1,3-dione (IX-1)

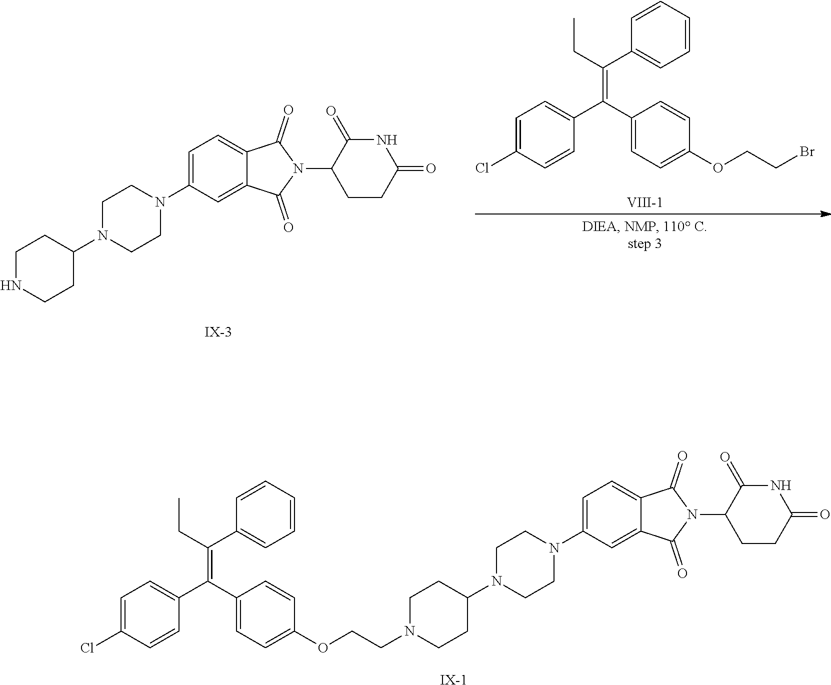

Procedure as example VIII, step 4. 2-(2,6-dioxopiperidin-3-yl)-5-(4-(piperidin-4-yl)piperazin-1-yl) isoindoline-1,3-dione (IX-3) (440 mg, 1 mmol) reacted with (Z/E)-1-(2-bromoethoxy)-4-(1-(4-chlorophenyl)-2-phenylbut-1-en-1-yl)benzene (VIII-1) (440 mg, 1 mmol) in NMP to produce (Z/E)-5-(4-(1-(2-(4-(1-(4-chlorophenyl)-2-phenylbut-1-en-1-yl)phenoxy)ethyl)piperidin-4-yl) piperazin-1-yl)-2-(2,6-dioxopiperidin-3-yl)isoindoline-1,3-dione (IX-1) (0.1 g) as yellow solid. LCMS [M+H] 786.

Step 4. (Z)-(4-(1-(4-(2-(4-(4-(2-(2,6-dioxopiperidin-3-yl)-1,3-dioxoisoindolin-5-yl)piperazin-1-yl) piperidin-1-yl)ethoxy)phenyl)-2-phenylbut-1-en-1-yl)phenyl)boronic acid (compound #31) and (E)-(4-(1-(4-(2-(4-(4-(2-(2,6-dioxopiperidin-3-yl)-1,3-dioxoisoindolin-5-yl)piperazin-1-yl)piperidin-1-yl)ethoxy)phenyl)-2-phenylbut-1-en-1-yl)phenyl)boronic acid (compound #32)

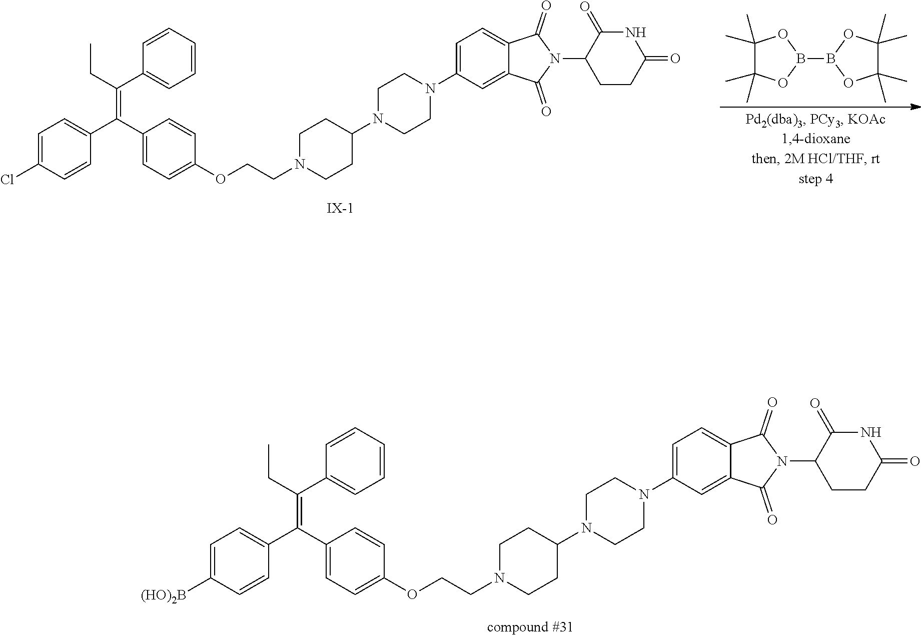

Procedure as example III, step 2. (Z/E)-5-(4-(1-(2-(4-(1-(4-chlorophenyl)-2-phenylbut-1-en-1-yl) phenoxy)ethyl)piperidin-4-yl)piperazin-1-yl)-2-(2,6-dioxopiperidin-3-yl)isoindoline-1,3-dione (IX-1) (0.1 g) produced (Z)-(4-(1-(4-(2-(4-(4-(2-(2,6-dioxopiperidin-3-yl)-1,3-dioxoisoindolin-5-yl) piperazin-1-yl)piperidin-1-yl)ethoxy)phenyl)-2-phenylbut-1-en-1-yl)phenyl)boronic acid (compound #31) (10 mg) as white solid and (E)-(4-(1-(4-(2-(4-(4-(2-(2,6-dioxopiperidin-3-yl)-1,3-dioxoisoindolin-5-yl)piperazin-1-yl)piperidin-1-yl)ethoxy)phenyl)-2-phenylbut-1-en-1-yl) phenyl)boronic acid (compound #32) (6 mg) as white solid. LCMS [M+H] 796.

### Example 12

### Compounds of Formula (I)—Compound 23

As an illustrative example of a compound of Formula (I), the following compound, compound 23 as denoted TABLE 1, was synthesized:

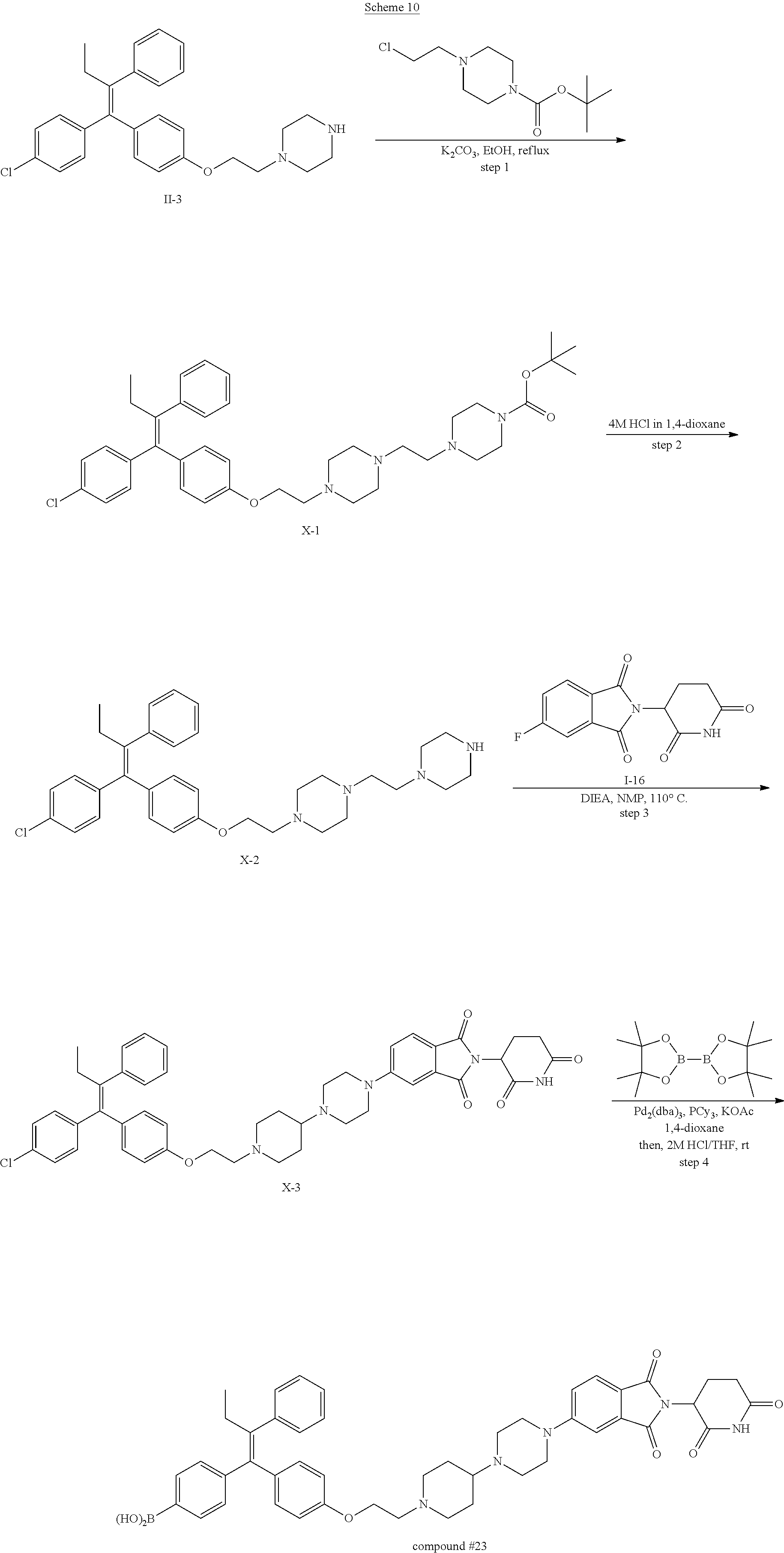

Step 1. tert-butyl (Z/E)-4-(2-(4-(2-(4-(1-(4-chlorophenyl)-2-phenylbut-1-en-1-yl) phenoxy)ethyl)piperazin-1-yl)ethyl)piperazine-1-carboxylate (X-1)

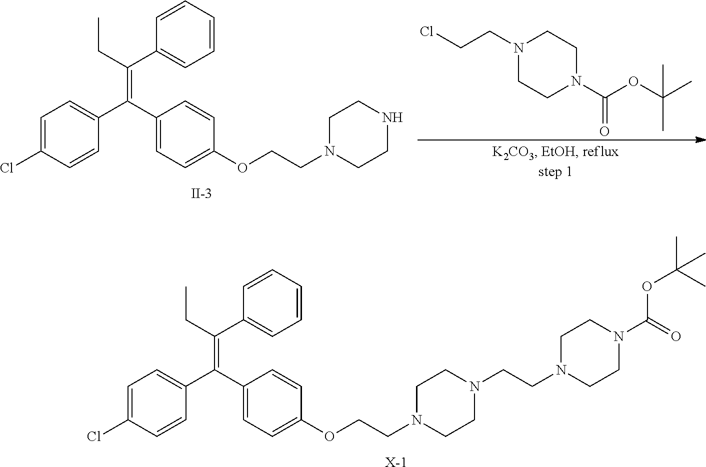

A mixture of (Z/E)-1-(2-(4-(1-(4-chlorophenyl)-2-phenylbut-1-en-1-yl)phenoxy)ethyl)piperazine (447 mg, 1 mmol), tert-butyl 4-(2-chloroethyl)piperazine-1-carboxylate (299 mg, 1.2 mmol), K₂CO₃ (276 mg, 2 mmol) in EtOH was reflux overnight. The solids were filtered out. The filtrate was evaporated in vacuo. The residue was subjected onto silica gel column chromatography with 50% EA in n-hexane to provide tert-butyl (Z/E)-4-(2-(4-(2-(4-(1-(4-chlorophenyl)-2-phenylbut-1-en-1-yl)phenoxy)ethyl)piperazin-1-yl)ethyl)piperazine-1-carboxylate (X-1) as colorless oil (1.4 g). LCMS [M+H] 659; ¹H NMR (DMSO-d6, 300 MHz): δ 0.85 (3H, t), 1.41 (9H, s), 2.31 (2H, q), 2.47-2.71 (12H, m), 2.72-2.77 (4H, m), 2.90-3.55 (6H, m), 4.13 (2H, t), 6.60 (2H, d), 6.95 (2H, d), 7.09-7.18 (7H, m), 7.45 (2H, d).

Step 2. (Z/E)-1-(2-(4-(1-(4-chlorophenyl)-2-phenylbut-1-en-1-yl)phenoxy)ethyl)-4-(2-(piperazin-1-yl)ethyl)piperazine (X-2)

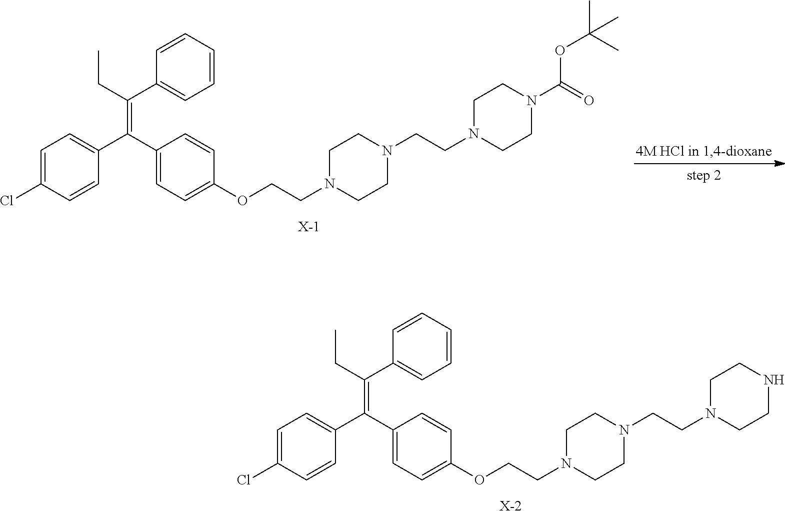

Procedure as example II, step 5. tert-butyl (Z/E)-4-(2-(4-(2-(4-(1-(4-chlorophenyl)-2-phenylbut-1-en-1-yl)phenoxy)ethyl)piperazin-1-yl)ethyl)piperazine-1-carboxylate (X-1) as colorless oil (1.4 g) produced (Z/E)-1-(2-(4-(1-(4-chlorophenyl)-2-phenylbut-1-en-1-yl)phenoxy)ethyl)-4-(2-(piperazin-1-yl)ethyl)piperazine (X-2) (2.0 g) as white solid. LCMS [M+H] 559; ¹H NMR (DMSO-d6, 300 MHz): δ 0.86 (3H, t), 2.31 (2H, q), 3.13-3.99 (12H, m), 4.28 (2H, t), 6.68 (2H, d), 6.80 (2H, d), 7.01-7.23 (7H, m), 7.43 (2H, d).

Step 3. (Z/E)-5-(4-(2-(4-(2-(4-(1-(4-chlorophenyl)-2-phenylbut-1-en-1-yl) phenoxy)ethyl)piperazin-1-yl)ethyl)piperazin-1-yl)-2-(2,6-dioxopiperidin-3-yl) isoindoline-1,3-dione (X-3)

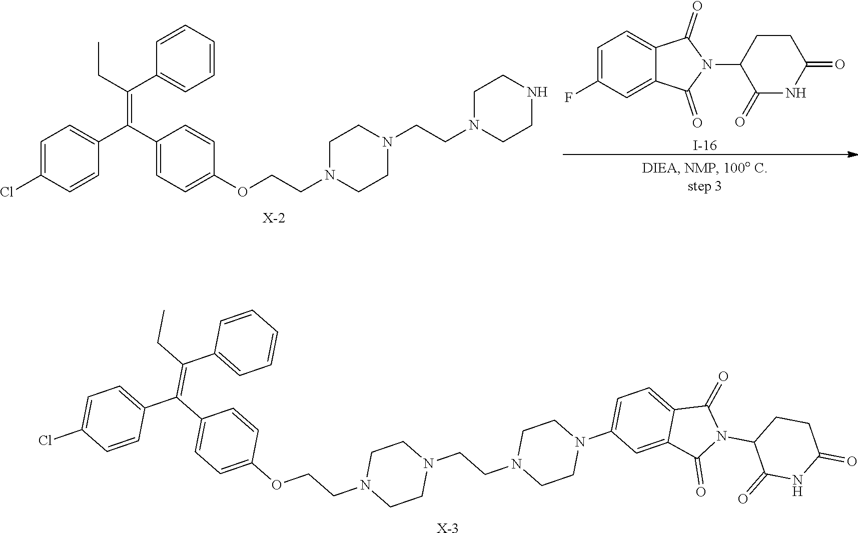

Procedure as example III, step 1. (Z/E)-1-(2-(4-(1-(4-chlorophenyl)-2-phenylbut-1-en-1-yl) phenoxy)ethyl)-4-(2-(piperazin-1-yl)ethyl)piperazine (X-2) (0.88 g) produced (Z/E)-5-(4-(2-(4-(2-(4-(1-(4-chlorophenyl)-2-phenylbut-1-en-1-yl)phenoxy)ethyl)piperazin-1-yl)ethyl)piperazin-1-yl)-2-(2,6-dioxopiperidin-3-yl)isoindoline-1,3-dione (X-3) (216 mg) as brown solid. LCMS [M+H] 815; ¹H NMR (DMSO-d6, 300 MHz): δ 0.84 (3H, t), 1.96-2.14 (2H, m), 2.21-2.44 (4H, m), 2.44-2.79 (16H, m), 2.85-2.94 (2H, m), 2.99-3.13 (4H, m), 4.18-4.27 (2H, m), 5.07 (1H, dd), 6.59 (2H, d), 6.63-7.32 (13H, m), 7.69 (2H, d), 11.10 (s, 1H).

Step 4. (Z)-(4-(1-(4-(2-(4-(2-(4-(2-(2,6-dioxopiperidin-3-yl)-1,3-dioxoisoindolin-5-yl) piperazin-1-yl)ethyl)piperazin-1-yl)ethoxy)phenyl)-2-phenylbut-1-en-1-yl)phenyl)boronic acid (compound #23) and (E)-(4-(1-(4-(2-(4-(2-(4-(2-(2,6-dioxopiperidin-3-yl)-1,3-dioxoisoindolin-5-yl)piperazin-1-yl)ethyl)piperazin-1-yl)ethoxy)phenyl)-2-phenylbut-1-en-1-yl)phenyl)boronic acid (compound #24)

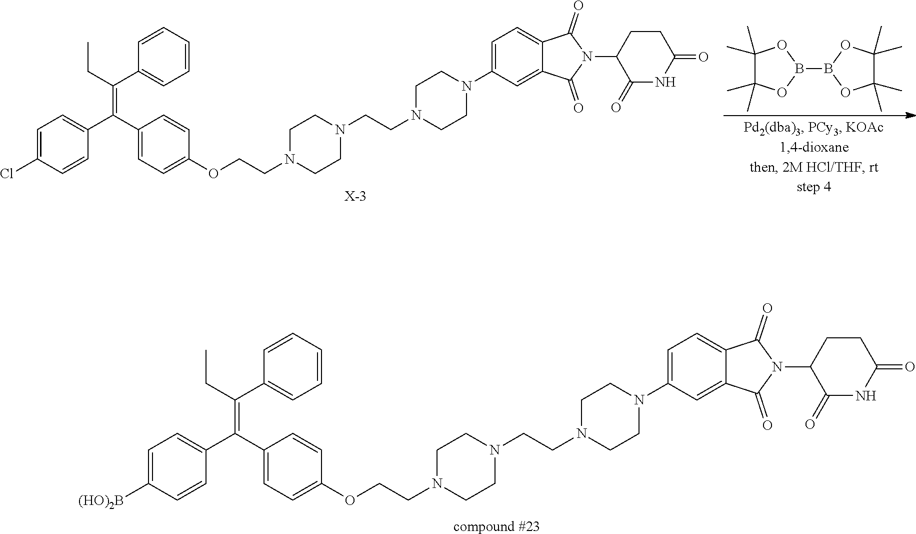

Procedure as example III, step 2. (Z/E)-5-(4-(2-(4-(2-(4-(1-(4-chlorophenyl)-2-phenylbut-1-en-1-yl) phenoxy)ethyl)piperazin-1-yl)ethyl)piperazin-1-yl)-2-(2,6-dioxopiperidin-3-yl)isoindoline-1,3-dione (X-3) (216 mg) produced (Z)-(4-(1-(4-(2-(4-(2-(4-(2-(2,6-dioxopiperidin-3-yl)-1,3-dioxoisoindolin-5-yl)piperazin-1-yl)ethyl)piperazin-1-yl)ethoxy)phenyl)-2-phenylbut-1-en-1-yl) phenyl)boronic acid (compound #23) (15 mg) as white solid and (E)-(4-(1-(4-(2-(4-(2-(4-(2-(2,6-dioxopiperidin-3-yl)-1,3-dioxoisoindolin-5-yl)piperazin-1-yl)ethyl)piperazin-1-yl) ethoxy)phenyl)-2-phenylbut-1-en-1-yl)phenyl)boronic acid (compound #24) (4 mg) as white solid. LCMS [M+H] 825; ¹H NMR (DMSO-d6, 300 MHz): δ 0.84 (3H, t), 1.96-2.14 (2H, m), 2.21-2.44 (4H, m), 2.44-2.79 (16H, m), 2.85-2.94 (2H, m), 2.99-3.13 (4H, m), 4.00 (2H, dd), 5.06 (1H, dd), 6.36 (2H, d), 6.53-7.42 (10H, m), 7.66 (2H, d), 7.92 (2H, d), 11.09 (1H, s).

### Example 13

### Compounds of Formula (I)—Compound 27

As an illustrative example of a compound of Formula (I), the following compound, compound 27 as denoted TABLE 1, was synthesized:

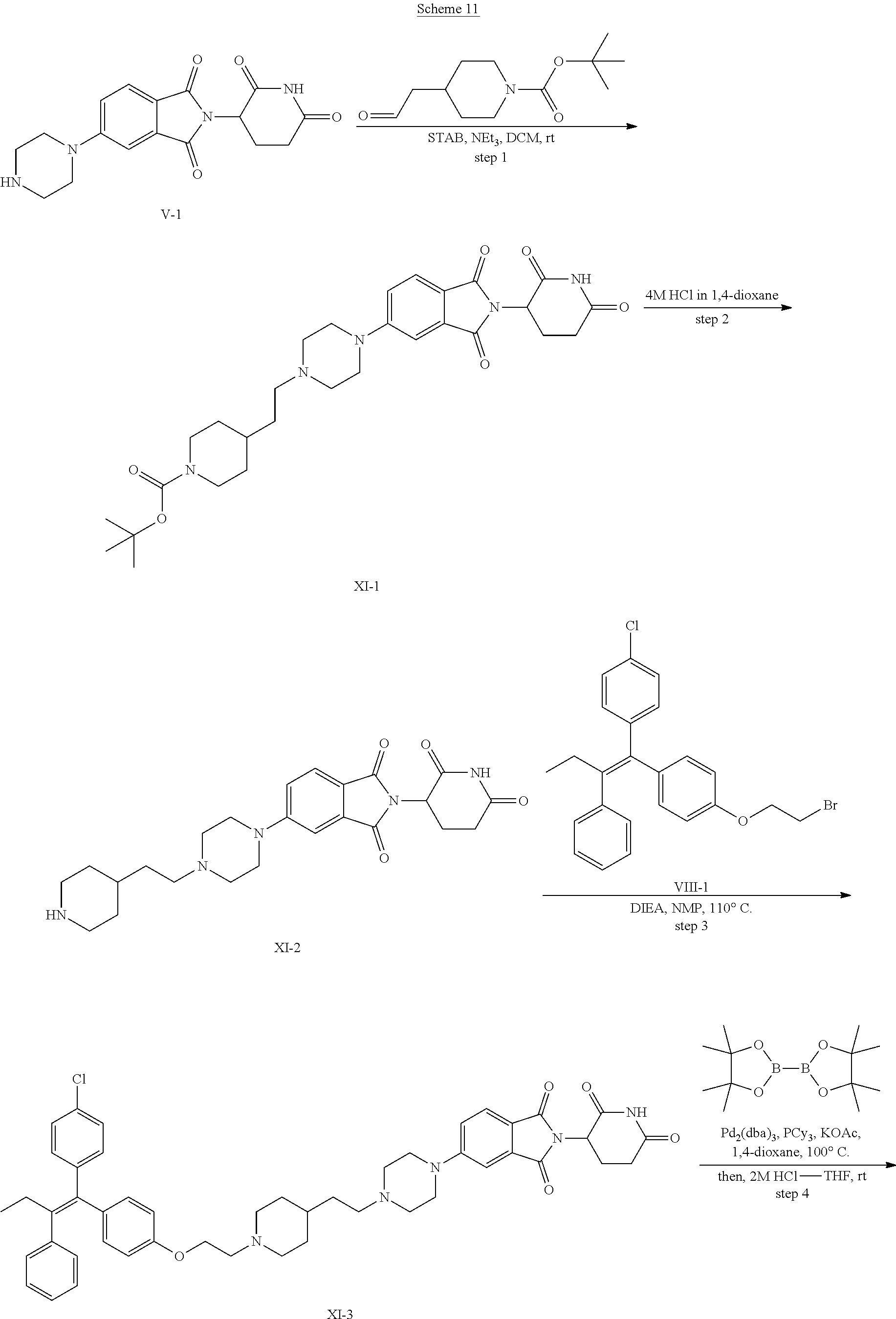

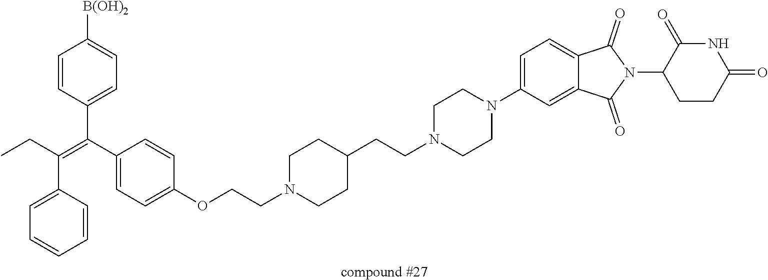

Step 1. tert-butyl 4-(2-(4-(2-(2,6-dioxopiperidin-3-yl)-1,3-dioxoisoindolin-5-yl)piperazin-1-yl) ethyl)piperidine-1-carboxylate (XI-1)

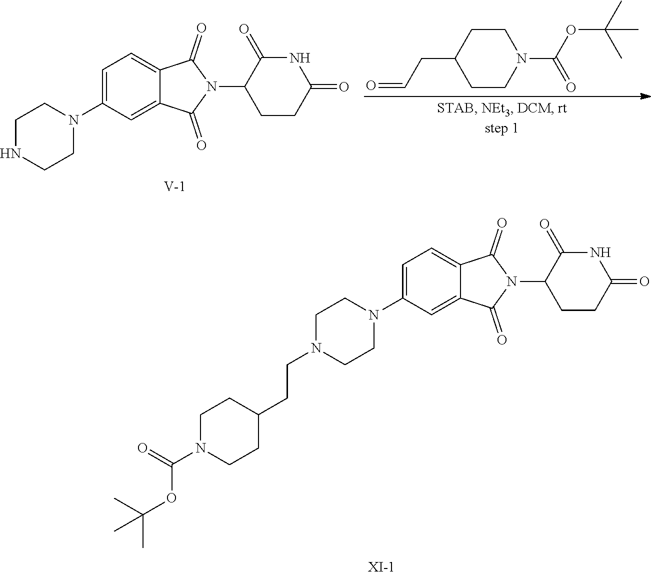

Procedure as example VIII, step 2. 2-(2,6-dioxopiperidin-3-yl)-5-(piperazin-1-yl)isoindoline-1,3-dione (V-1) (828 mg, 2 mmol) reacted with tert-butyl 4-(2-oxoethyl)piperidine-1-carboxylate (682 mg, 3 mmol) to provide tert-butyl 4-(2-(4-(2-(2,6-dioxopiperidin-3-yl)-1,3-dioxoisoindolin-5-yl) piperazin-1-yl)ethyl)piperidine-1-carboxylate (XI-1) (1.52 g) as yellow oil. LCMS [M+H] 554; ¹H NMR (DMSO-d6, 300 MHz): δ 1.26-1.32 (2H, m), 1.42-1.71 (14H, m), 1.96-2.14 (2H, m), 2.21-2.44 (2H, m), 2.50-2.65 (6H, m), 2.98-3.15 (4H, m), 3.87-3.96 (2H, m), 5.08 (1H, t), 7.24 (1H, dd), 7.33 (1H, dd), 7.67 (1H, dd), 11.10 (1H, s).

Step 2. 2-(2,6-dioxopiperidin-3-yl)-5-(4-(2-(piperidin-4-yl)ethyl)piperazin-1-yl)isoindoline-1,3-dione (XI-2)

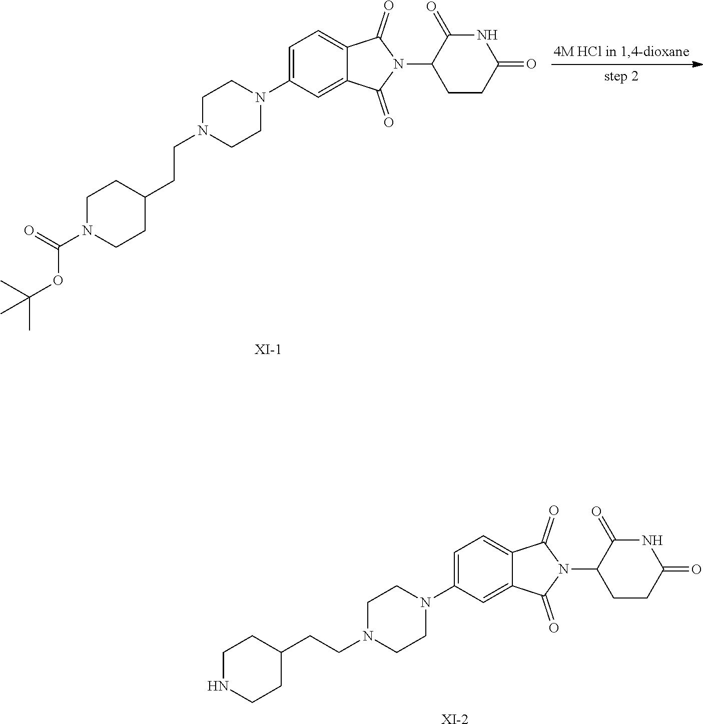

Procedure as example VIII, step 3. tert-butyl 4-(2-(4-(2-(2,6-dioxopiperidin-3-yl)-1,3-dioxoisoindolin-5-yl)piperazin-1-yl)ethyl)piperidine-1-carboxylate (XI-1) (1.52 g) provided 2-(2,6-dioxopiperidin-3-yl)-5-(4-(2-(piperidin-4-yl)ethyl)piperazin-1-yl)isoindoline-1,3-dione (XI-2) (1.25 g) as yellow solid. LCMS [M+H] 454.

Step 3. (Z/E)-5-(4-(2-(1-(2-(4-(1-(4-chlorophenyl)-2-phenylbut-1-en-1-yl) phenoxy)ethyl)piperidin-4-yl)ethyl)piperazin-1-yl)-2-(2,6-dioxopiperidin-3-yl)isoindoline-1,3-dione (XI-3)

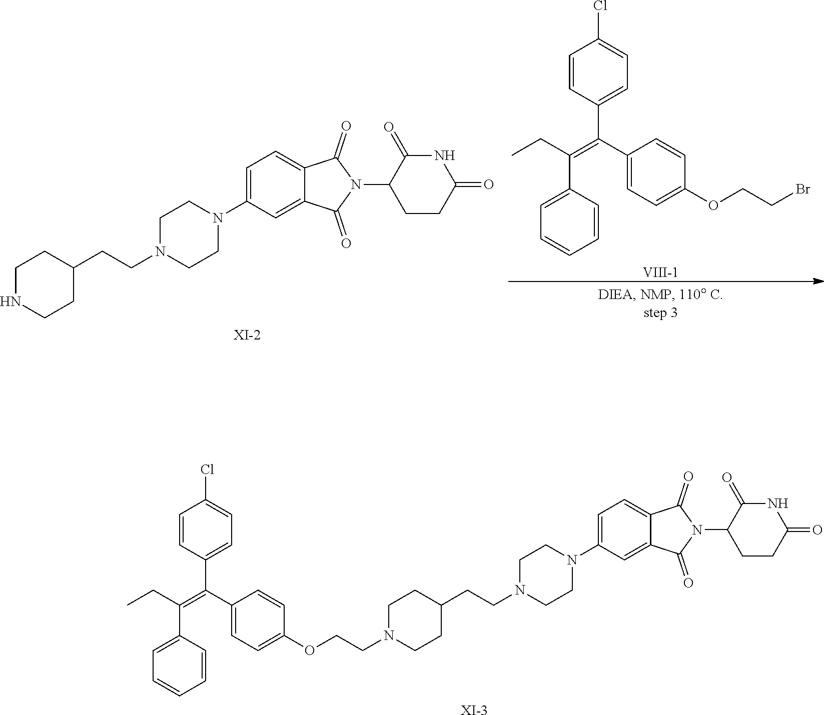

Procedure as example VIII, step 4. 2-(2,6-dioxopiperidin-3-yl)-5-(4-(2-(piperidin-4-yl) ethyl)piperazin-1-yl)isoindoline-1,3-dione (XI-2) (451 mg, 1 mmol) reacted with (Z/E)-1-(2-bromoethoxy)-4-(1-(4-chlorophenyl)-2-phenylbut-1-en-1-yl)benzene (VIII-1) (440 mg, 1 mmol) in the above condition to provide (Z/E)-5-(4-(2-(1-(2-(4-(1-(4-chlorophenyl)-2-phenylbut-1-en-1-yl) phenoxy)ethyl)piperidin-4-yl)ethyl)piperazin-1-yl)-2-(2,6-dioxopiperidin-3-yl)isoindoline-1,3-dione (XI-3) (151 mg) as brown solid. LCMS [M+H] 814.

Step 4. (Z)-(4-(1-(4-(2-(4-(2-(4-(2-(2,6-dioxopiperidin-3-yl)-1,3-dioxoisoindolin-5-yl) piperazin-1-yl)ethyl)piperidin-1-yl)ethoxy)phenyl)-2-phenylbut-1-en-1-yl)phenyl)boronic acid (compound #27) and (E)-(4-(1-(4-(2-(4-(2-(4-(2-(2,6-dioxopiperidin-3-yl)-1,3-dioxoisoindolin-5-yl)piperazin-1-yl)ethyl)piperidin-1-yl)ethoxy)phenyl)-2-phenylbut-1-en-1-yl) phenyl)boronic acid (compound #28)

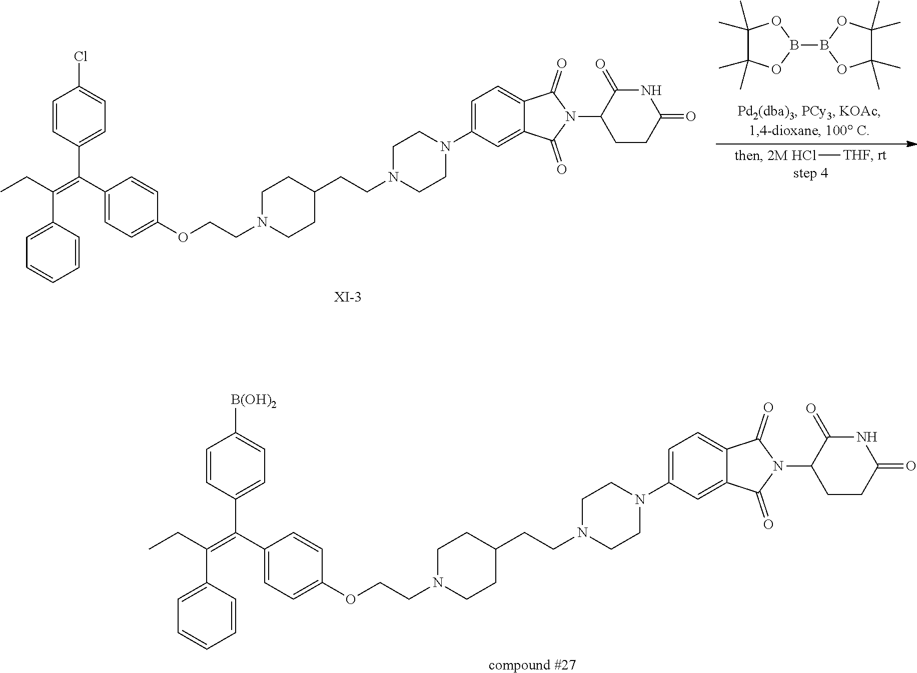

Procedure as example III, step 2. (Z/E)-5-(4-(2-(1-(2-(4-(1-(4-chlorophenyl)-2-phenylbut-1-en-1-yl) phenoxy)ethyl)piperidin-4-yl)ethyl)piperazin-1-yl)-2-(2,6-dioxopiperidin-3-yl)isoindoline-1,3-dione (XI-3) (151 mg) produced (Z)-(4-(1-(4-(2-(4-(2-(4-(2-(2,6-dioxopiperidin-3-yl)-1,3-dioxoisoindolin-5-yl)piperazin-1-yl)ethyl)piperidin-1-yl)ethoxy)phenyl)-2-phenylbut-1-en-1-yl) phenyl)boronic acid (compound #27) (20 mg) as white solid; LCMS [M+H] 824; ¹H NMR (DMSO-d6, 300 MHz): δ 0.84 (3H, t), 1.39-1.65 (5H, m), 1.96-2.14 (2H, m), 2.21-2.65 (14H, m), 2.87-2.96 (2H, m), 2.98-3.15 (4H, m), 4.02-4.23 (2H, dt), 5.05 (1H, dd), 6.57 (2H, dd), 6.71-7.40 (10H, m), 7.73 (1H, m), 7.85 (1H, d), 8.04 (1H, d), 11.09 (1H, s) and (E)-(4-(1-(4-(2-(4-(2-(4-(2-(2,6-dioxopiperidin-3-yl)-1,3-dioxoisoindolin-5-yl)piperazin-1-yl)ethyl)piperidin-1-yl)ethoxy)phenyl)-2-phenylbut-1-en-1-yl) phenyl)boronic acid (compound #28) (6 mg) as white solid. LCMS [M+H] 824.

### Example 14

### Compounds of Formula (I)—Compound 77

As an illustrative example of a compound of Formula (I), the following compound, compound 77 as denoted TABLE 1, was synthesized:

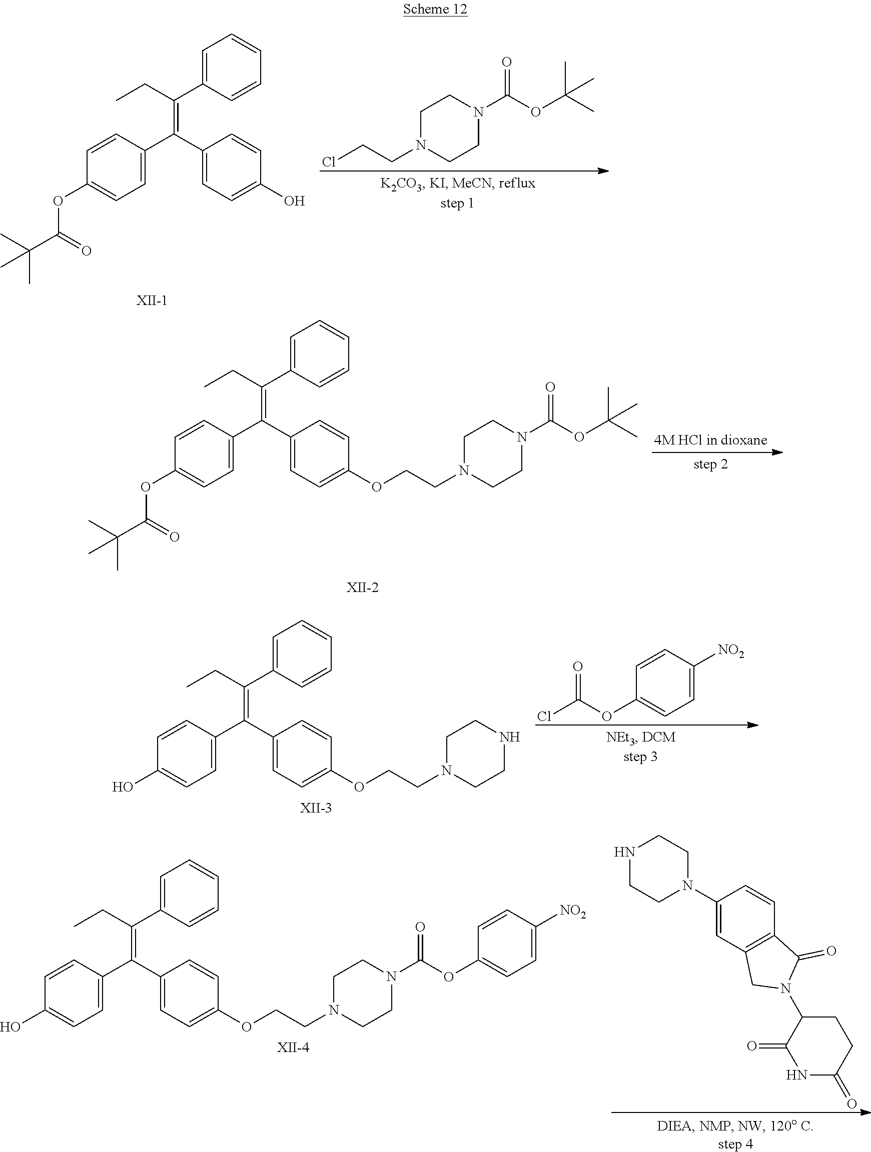

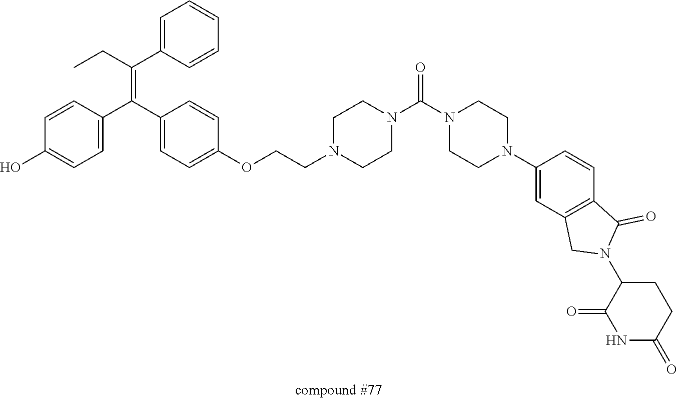

Step 1. tert-butyl (E)-4-(2-(4-(2-phenyl-1-(4-(pivaloyloxy)phenyl)but-1-en-1-yl) phenoxy)ethyl)piperazine-1-carboxylate (XII-2)

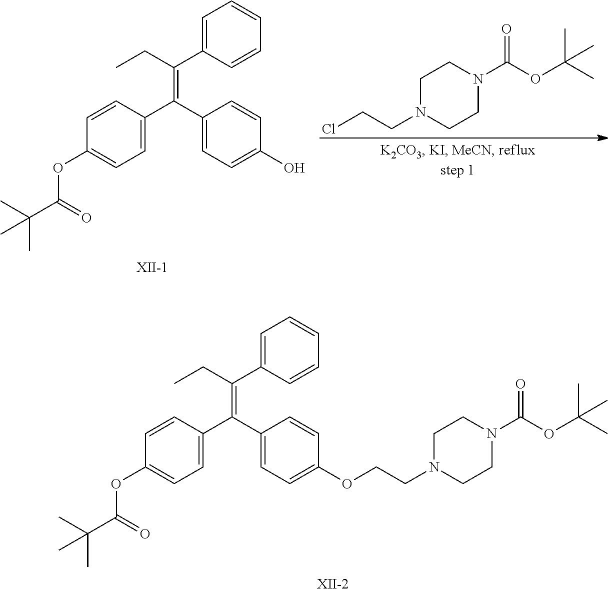

A mixture of (E)-4-(1-(4-hydroxyphenyl)-2-phenylbut-1-en-1-yl)phenyl pivalate (2.0 g, 5 mmol) (known compound, follow the synthetic route in the Bioorganic & Medicinal Chemistry Letters Volume 28, Issue 8, 1 May 2018, Pages 1352-1356), tert-butyl 4-(2-chloroethyl)piperazine-1-carboxylate (1.5 g, 6 mmol), K2CO3 (1.4 g, 10 mmol) and catalytic KI in 50 mL of MeCN was refluxed for 2 hours. After cooling to rt, the solids were filtered out, the filtrate was evaporated to dryness. The residue was subjected onto silica gel column chromatography with PE:EA as eluent to provide 1.6 g of tert-butyl (E)-4-(2-(4-(2-phenyl-1-(4-(pivaloyloxy)phenyl)but-1-en-1-yl) phenoxy)ethyl)piperazine-1-carboxylate (XII-2) as colorless oil. LCMS [M+H] 613. ¹H NMR (DMSO-d6, 300 MHz): δ 0.90 (3H, t), 1.36 (6H, s), 1.45 (12H, s), 2.22 (2H, q), 2.63 (4H, t), 2.90 (2H, t), 3.55 (4H, d), 4.23 (2H, t), 6.92 (2H, d), 7.15-7.45 (11H, m).

Step 2. (Z)-4-(2-phenyl-1-(4-(2-(piperazin-1-yl)ethoxy)phenyl)but-1-en-1-yl)phenol (XII-3)

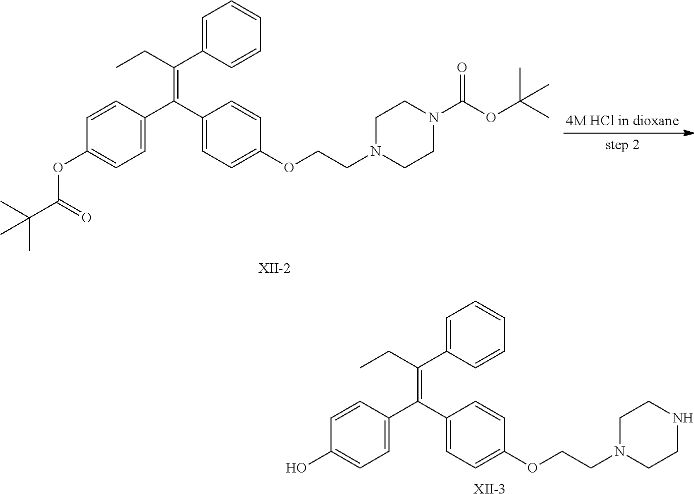

Procedure as example VIII, step 3. tert-butyl (E)-4-(2-(4-(2-phenyl-1-(4-(pivaloyloxy)phenyl)but-1-en-1-yl)phenoxy)ethyl)piperazine-1-carboxylate (1.6 g) provided (Z)-4-(2-phenyl-1-(4-(2-(piperazin-1-yl)ethoxy)phenyl)but-1-en-1-yl)phenol (XII-3) (1.7 g) as white solid as n HCl salt. LCMS [M+H] 513. 1H NMR (DMSO-d6, 300 MHz): δ 0.86 (3H, t), 1.28 (9H, s), 2.50 (2H, q), 2.63 (4H, t), 2.90 (2H, t), 3.55 (4H, d), 4.23 (2H, t), 6.92 (2H, d), 7.15-7.45 (11H, m), 9.51 (2.6, br).

Step 3. 4-nitrophenyl (Z)-4-(2-(4-(1-(4-hydroxyphenyl)-2-phenylbut-1-en-1-yl) phenoxy)ethyl)piperazine-1-carboxylate (XI-4)

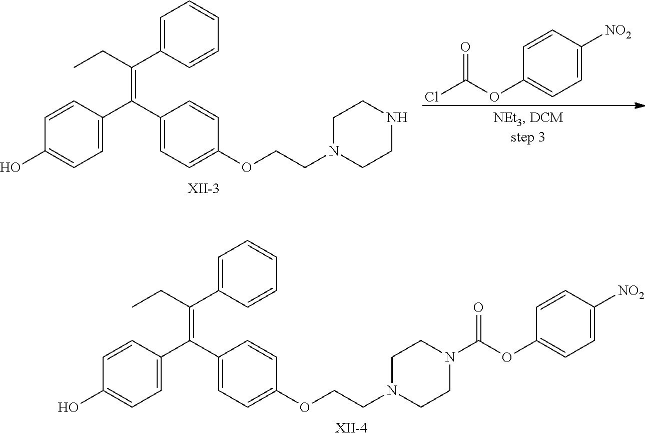

A mixture of (Z)-4-(2-phenyl-1-(4-(2-(piperazin-1-yl)ethoxy)phenyl)but-1-en-1-yl)phenol (XII-3) (1.0 g, 2.3 mmol), NEt3 (0.7 g, 6.9 mmol) in 20 mL of DCM was added 4-nitrophenyl carbonochloridate (0.47 g, 2.3 mmol) at 0° C. After stirring for 30 min, the solvent was removed in vacuo. The residue was subjected onto silica gel column chromatography to provide 0.4 g of 4-nitrophenyl (Z)-4-(2-(4-(1-(4-hydroxyphenyl)-2-phenylbut-1-en-1-yl)phenoxy)ethyl)piperazine-1-carboxylate (XII-4) as light yellow solid. LCMS [M+H] 594.

Step 4. (Z)-3-(5-(4-(4-(2-(4-(1-(4-hydroxyphenyl)-2-phenylbut-1-en-1-yl) phenoxy)ethyl)piperazine-1-carbonyl)piperazin-1-yl)-1-oxoisoindolin-2-yl)piperidine-2,6-dione (compound #77)

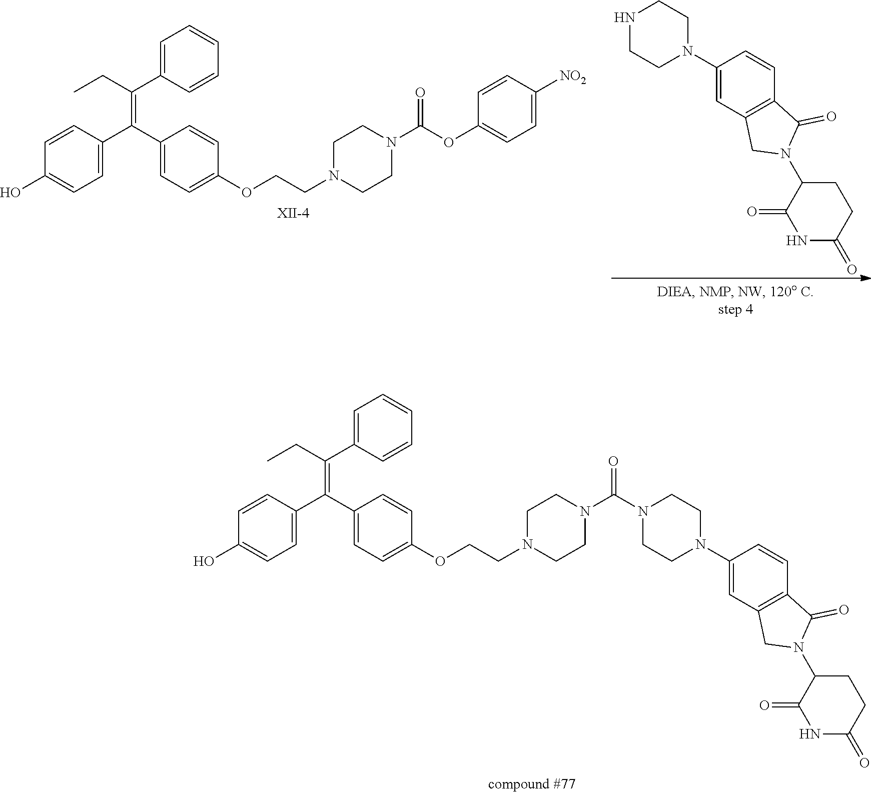

A mixture of 4-nitrophenyl (Z)-4-(2-(4-(1-(4-hydroxyphenyl)-2-phenylbut-1-en-1-yl) phenoxy)ethyl)piperazine-1-carboxylate (XII-4) (100 mg), 3-(1-oxo-5-(piperazin-1-yl)isoindolin-2-yl)piperidine-2,6-dione and DIEA in 3 mL of NMP was heated in a microwave reactor for 30 min. After cooling to rt, the mixture was subjected onto silica gel column chromatography using EA/MeOH as eluent to provide crude product. The product was further purified by prep-HPLC with 0.5% FA/MeCN, C18, to provide 33 mg of (Z)-3-(5-(4-(4-(2-(4-(1-(4-hydroxyphenyl)-2-phenylbut-1-en-1-yl)phenoxy)ethyl)piperazine-1-carbonyl)piperazin-1-yl)-1-oxoisoindolin-2-yl) piperidine-2,6-dione (compound #77) as white solid. LCMS [M+H] 783. ¹H NMR: δ 0.90 (3H, t), 1.15-1.27 (9H, m), 1.86-2.11 (2H, t), 2.23-2.41 (4H, m), 2.53-2.76 (2H, m), 2.84-2.96 (2H, m), 3.00-3.68 (14H, m), 4.17-4.29 (2H, m), 4.46-4.64 (3H, m), 6.86-7.11 (4H, m), 7.14-7.48 (12H, m), 7.79 (1H, d).

### Example 15

### Compounds of Formula (I)—Compound 89 and Compound 93

As an illustrative example of a compound of Formula (I), the following compound, compound 89 and compound 93 as denoted TABLE 1, was synthesized:

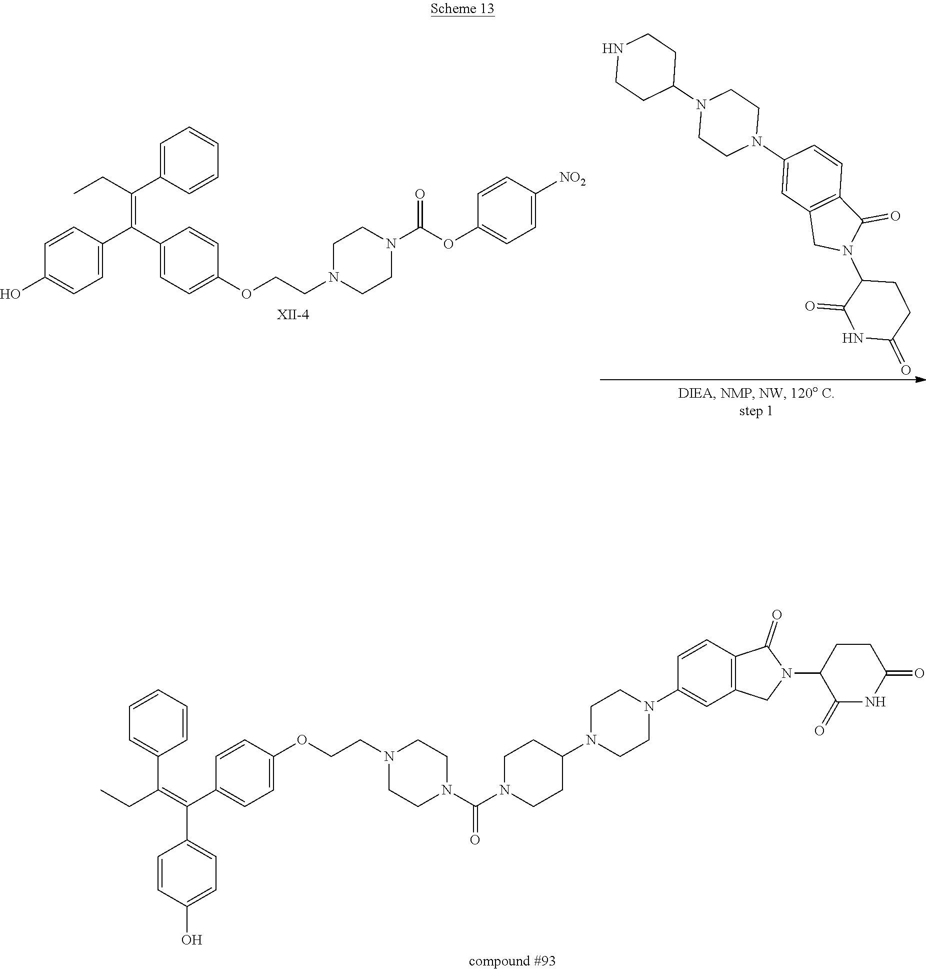

Step 1. (Z)-3-(5-(4-(1-(4-(2-(4-(1-(4-hydroxyphenyl)-2-phenylbut-1-en-1-yl) phenoxy)ethyl)piperazine-1-carbonyl)piperidin-4-yl)piperazin-1-yl)-1-oxoisoindolin-2-yl) piperidine-2,6-dione (compound #93)

Procedure as example XII, step 4. 4-nitrophenyl (Z)-4-(2-(4-(1-(4-hydroxyphenyl)-2-phenylbut-1-en-1-yl)phenoxy)ethyl)piperazine-1-carboxylate (100 mg) reacted with 3-(1-oxo-5-(4-(piperidin-4-yl) piperazin-1-yl)isoindolin-2-yl)piperidine-2,6-dione to provide 45 mg of (Z)-3-(5-(4-(1-(4-(2-(4-(1-(4-hydroxyphenyl)-2-phenylbut-1-en-1-yl)phenoxy)ethyl)piperazine-1-carbonyl)piperidin-4-yl) piperazin-1-yl)-1-oxoisoindolin-2-yl)piperidine-2,6-dione (compound # 93) as white solid. LCMS [M+H] 866.

In similar method, (Z)-3-(5-(4-((1-(4-(2-(4-(1-(4-hydroxyphenyl)-2-phenylbut-1-en-1-yl) phenoxy)ethyl)piperazine-1-carbonyl)piperidin-4-yl)methyl)piperazin-1-yl)-1-oxoisoindolin-2-yl) piperidine-2,6-dione (compound #89) was also obtained as white solid. LCMS [M+H] 880.

### Example 16

### Degradation of ER in MCF-7 Breast Cancer Cells by Exemplary Compounds of the Present Disclosure

MCF-7 cells were plated in 24-well plates at a density of 10⁵ cells/well. Media containing various drug concentrations were added on the day following plating (day 0) and allowed to incubate for 24 hours for Western blot. Media with the tested compound was changed every other day. Cells were lysed, snapped frozen in liquid nitrogen, and stored at −80° C. until assay for ERα. Media were removed and dishes were washed with 1× DPBS. Lysates were made by adding 150 μL of complete lysis solution and scraping cells into a 1.5 mL microcentrifuge tube. Lysates were placed on a rotisserie at 4° C. for 30 min and then spun at 4° C. at 12000 rcf for 10 min. Supernatants were assayed for protein content, snap-frozen, and stored a −80° C. if not run immediately. Then 50 μg of protein was subjected to Western blot protocol. Membranes were blocked and then incubated with 1:200 dilution of ERα antibody at 4° C. overnight followed by 1:10000 dilution of secondary antibody for 1 h at room temperature. They were then imaged on a LICOR infrared scanner.

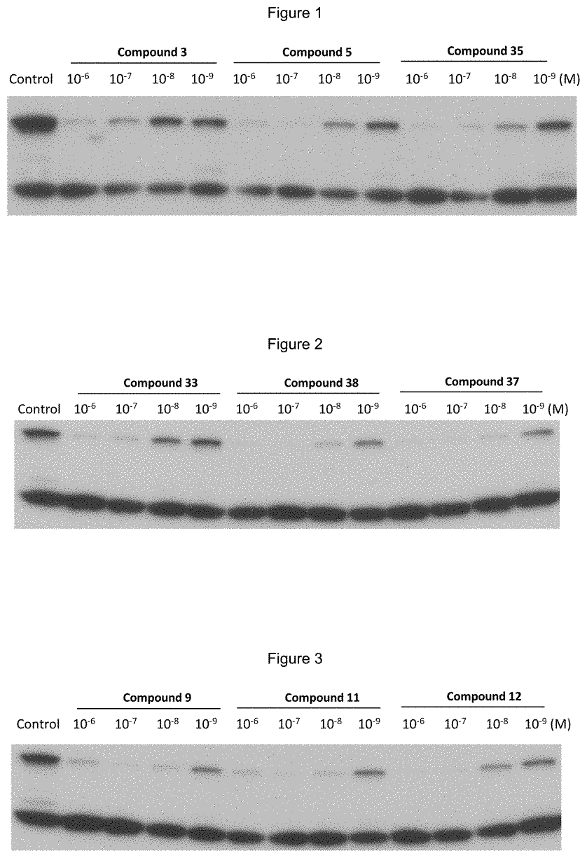

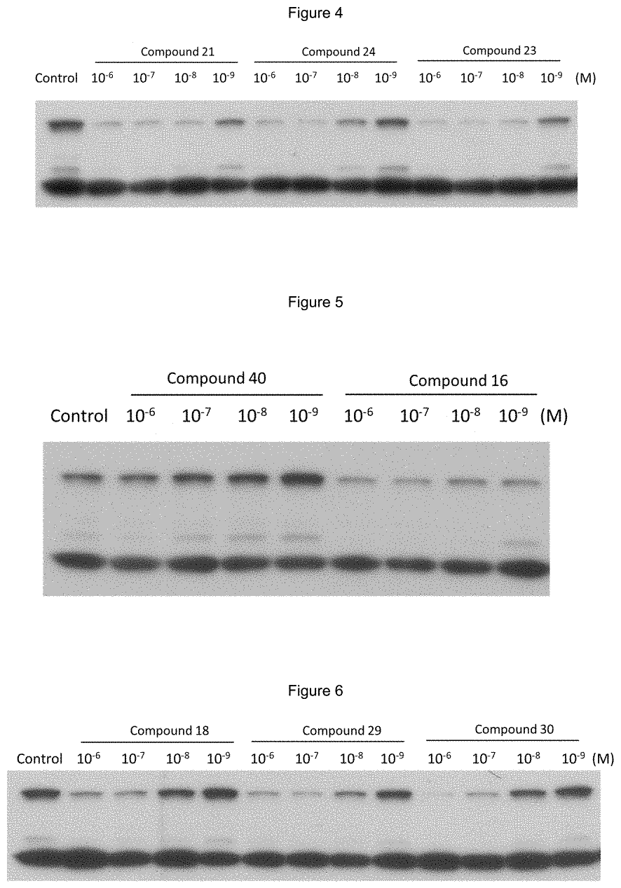

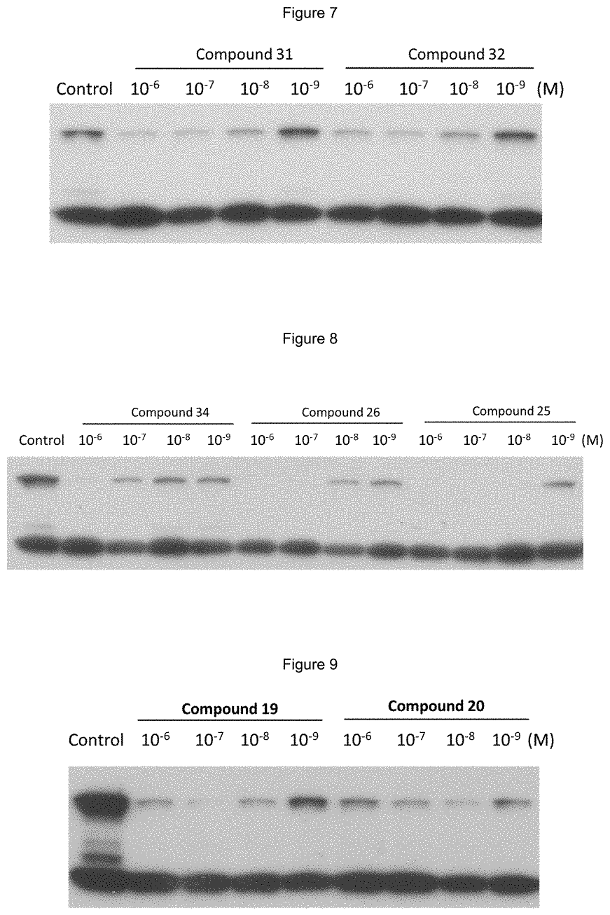

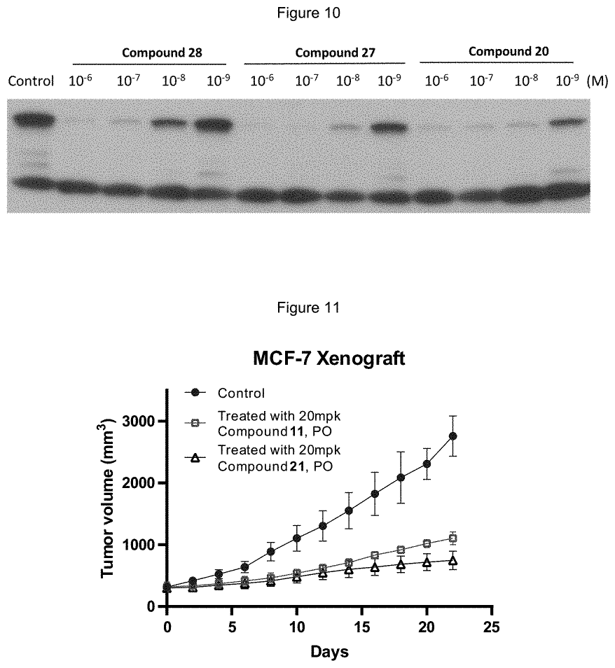

FIGS. 1 to 10 show the ER degradation efficacy of compounds 3, 5, 9, 11, 12, 16, 18, 19, 20, 21, 23, 24, 25, 26, 27, 28, 29, 30, 31, 32, 33, 34, 37, 38, and 40 in MCF-7 cells at various concentrations. Degradation of ER was expressed as an DC50 value and was determined for exemplary compounds in Table 1 by calculation of the concentration of compound that was required to give a 50% reduction of ER expression level. Maximal degradation of ER was expressed as a Dmax value by measuring the highest percentage of ER reduction achieved by exemplary compound in the treatment concentration range. DC50 value and Dmax value for each compound are listed in Table 1.

### Example 17

### The Antiestrogenic Activity of the Exemplary Compounds was Assessed in T47d-kb-Luc Stably Transfected Human Breast Cancer Cell

The T47d-kb-Luc cells are stably transfected with an artificial gene from the firefly that is only induced in the cells if estrogens bind and activate the ER to induce the gene product (Luciferase) that is then measured with a quantitative enzyme assay that produces light. Antagonist activities were measured by the compound's ability to inhibit the activity of estradiol, the natural estrogen. Data were then normalized relative to the activity of the estradiol control and determinations were performed for five concentrations of the samples in quadruplicate in at least three separate experiments.

### Example 18

### The Anti-Proliferative Activities of the Exemplary Compounds were Assessed by a Cell Viability Assay in MCF-7 Breast Cancer Cells

MCF-7 cells were plated in six-well plates at a density of 50,000 each well in 5% FBS DMEM medium. The cells were then treated with exemplary compounds or fulvestrant separately at 6 different doses ranging from 10⁻¹⁰ M to 10⁻⁵ M for 5 days, while equal volumes of DMSO were used as vehicle controls. Viable cell numbers were counted with a Z Series Coulter Counter instrument (Beckman-Coulter) following manufacturer's instructions. The ratio of drug treated viable cell numbers to vehicle treated viable cell numbers was defined as survival ratio where the control has the survival ratio of 100%. IC₅₀ values were obtained from dose-response curves for exemplary compounds that are listed in Table 1.

### Example 19

### In Vivo Efficacy of Exemplary Compounds 11 and 21 in MCF-7 Xenograft Tumors

Four to six weeks old female ovariectomized Nu/Nu mice were purchased from Jackson Laboratories (Bar Harbor, Me.). MCF-7 cells were cultured and harvested in the exponential growth phase using a PBS/EDTA solution. The animals were injected bilaterally in the mammary fat pad (MFP) with 5×10⁶ viable cells suspended in 50 μL sterile PBS mixed with 100 μL Matrigel (reduced factor; BD Biosciences, Bed-ford, Mass.). 17□-Estradiol pellets (0.72 mg, 60 day release; Innovative Research of America, Sarasota, Fla.) were implanted subcutaneously in the lateral area of the neck using a precision trochar (10 gages) at the time of cell injection. After tumor formation to palpable size, animals with tumors of similar sizes (300±100 mm³) were randomized to control or treatment groups, and treated with vehicle, compound 11 at 20 mg/kg, or compound 21 at 20 mg/kg by oral gavage. Tumor sizes were monitored and recorded every other day for three weeks of treatment duration.

FIG. 11 shows the in vivo efficacy of exemplary compound 11 and 21 in inhibiting MCF-7 xenograft tumors after 21 daily doses.

### Example 20

### In Vivo Efficacy of Exemplary Compound 69 in ST1799 Patient Derived Breast Tumor Xenocraft

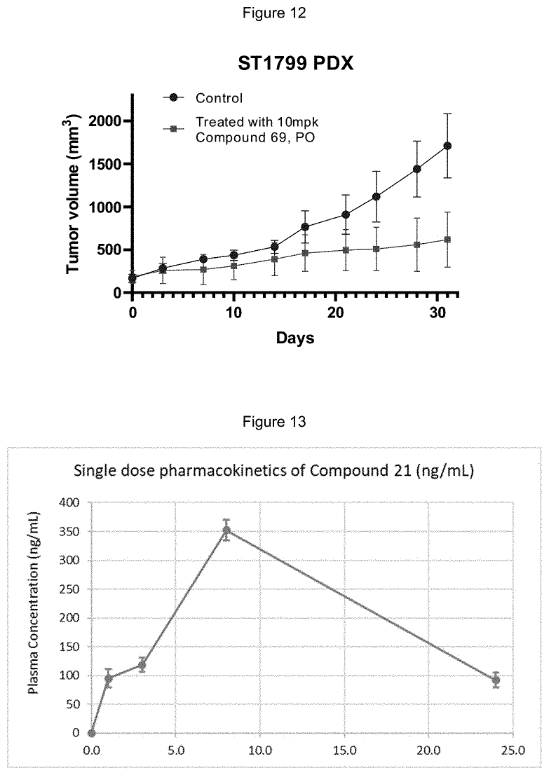

Patient-Derived Xenograft tumor fragments were harvested from host animals and implanted into 6-12 week old athymic nude female mice (Charles River NU(NCr)-Foxn1^{nu}). Treatment initiated at a mean tumor volume of approximately 175-300 mm³. Single agent Compound 69 reported statistically significant tumor growth inhibition (p=0.0323) versus vehicle control. FIG. 12 shows the in vivo efficacy of exemplary compound 68 in inhibiting PDX tumor after 30 day treatment.

### Example 21

### Pharmacokinetic Properties of Exemplary Compounds 21 and 69 were Assessed in Female Sprague Dawley Rats

Sprague Dawley rats (n=3), weighing between 350 and 400 g (Charles River Laboratories, Portage, Mich.) were given oral gavage containing compound 21 or compound 89 dissolved in propylene glycol (PG):solutol:40% HP-b-CD in DI water (20:5:75 v:v) at a single dose of 10 mg/kg. After drug administration, blood samples were collected from the tail vein of the rats at various time points into 1.5 mL microcentrifuge tubes containing 0.1 mL of 10% EDTA anticoagulant. Plasma was then separated from cell pellets by centrifugation in a refrigerated centrifuge at 4° C. and transferred to a separate tube. Plasma samples were frozen at −80° C. until analysis.

HPLC-MS/MS Analysis of Plasma Samples. Plasma samples were extracted with chloroform/methanol (2:1) using traditional Folch method for lipid extraction. Methanol (1 mL) and chloroform (2 mL) were added to each plasma sample followed by addition of 5 ng trans-Tamoxifen-13C2, 15N to each sample as the internal standard. The mixtures were stored at −20° C. overnight. Next the samples were sonicated for 5 min and centrifuged with a Thermo Scientific Heraeus Megafuge16 Centrifuge. The top layer was transferred to another test tube. The bottom layer was washed with 1 mL chloroform/methanol (2:1), centrifuged, and the solvent was transferred and combined with previous washings. Eight tenth of a milliliter HPLC grade water was added to the extracts. After vortexing, the mixture was centrifuged. The bottom layer was dried out with nitrogen and re-suspended in 100 μL HPLC grade acetonitrile. An aliquot of 10 μL sample was injected onto a Hypersil Gold column (50×2.1 mm; particle size 1.9 μm, Thermo Scientific) on a Dionex Ultimate 3000 UPLC system equipped with a TSQ Vantage triple quadrupole mass spectrometer for analysis. A binary mobile phase (A: water with 0.05% formic acid, B: acetonitrile with 0.05% formic acid) was used to achieve the gradient of initial 30% B for 1 min and then to 80% B at 8 min, to 100% B at 9 min, and returned to 30% B for 4 min. The flow rate was controlled at 0.6 mL/min. The settings of HESI source were as follows: spray voltage (3200 volt); vaporizer temperature (365° C.); sheath gas pressure (45 psi); auxiliary gas pressure (10 psi); capillary temperature (330° C.). Nitrogen was used as the sheath gas and axillary gas. Argon was used as the collision gas.

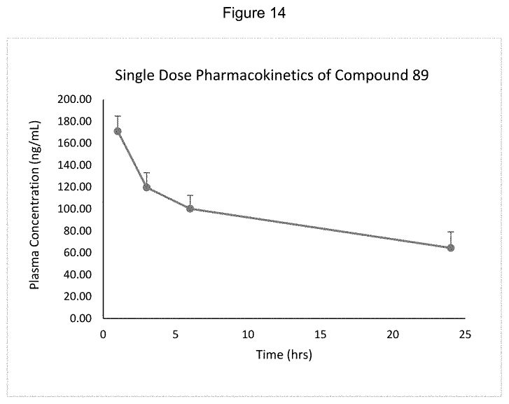

FIGS. 13 and 14 show the pharmacokinetic profile of exemplary compound 21 and 69, respectively.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or multiple elements together may also find a useful application in other types of methods differing from the type described above, as well as in other types of diseases differing from the type described herein. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is not intended to be limiting only by the following claims.

### REFERENCES CITED

1. Gustafsson J A. What pharmacologists can learn from recent advances in estrogen signalling. Trends Pharmacol Sci 2003; 24:479-485.
2. Barrios C, Forbes J F, Jonat W, Conte P, Gradishar W, Buzdar A, Gelmon K, Gnant M, Bonneterre J, Toi M, Hudis C, Robertson J F. The sequential use of endocrine treatment for advanced breast cancer: where are we? Ann Oncol. 2012, 23(6):1378-86.
3. Jasani B, Douglas-Jones A, Rhodes A, Wozniak S, Barrett-Lee P J, Gee J, Nicholson R. Measurement of estrogen receptor status by immunocytochemistry in paraffin wax sections. Methods Mol Med. 2006; 120:127-46.
4. Setiawan V W, Monroe K R, Wilkens L R, Kolonel L N, Pike M C, Henderson B E. Breast cancer risk factors defined by estrogen and progesterone receptor status: the multiethnic cohort study. Am J Epidemiol. 2009 May 15; 169(10):1251-9.
5. Nabholtz J M, Buzdar A, Pollak M et al. Anastrozole is superior to tamoxifen as first-line therapy for advanced breast cancer in postmenopausal women: results of a North American multicenter randomized trial. Arimidex Study Group. J Clin Oncol 2000; 18: 3758-3767.
6. Nabholtz J M, Bonneterre J, Buzdar A et al. Anastrozole (Arimidex) versus tamoxifen as first-line therapy for advanced breast cancer in postmenopausal women: survival analysis and updated safety results. Eur J Cancer 2003; 39: 1684-1689.
7. An S, Fu L. Small-molecule PROTACs: An emerging and promising approach for the development of targeted therapy drugs. EBioMedicine 2018; 36: 553-562.
8. Hammond et al., Immunohistochemical Testing of Estrogen and Progesterone Receptors in Breast Cancer, Journal of Clinical Oncology, 2010; 28, 2784-2795.

What is claimed is:

1. A compound represented by Formula (I), or a tautomer, stereoisomer, mixture of stereoisomers, pharmaceutically acceptable salt, or solvate thereof: 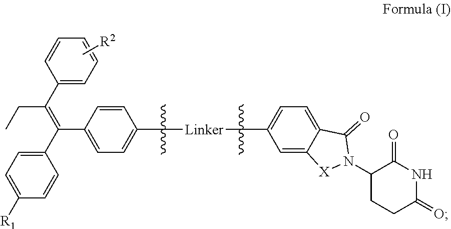
wherein R¹ and R² are each independently selected from the group consisting of hydrogen, deuterium, halogen, hydroxyl, alkyl, alkoxy, 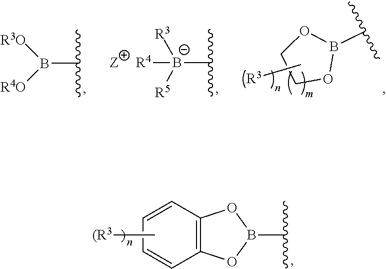
and any combination thereof;
X is selected from the group consisting of O, S, C(R³)(R⁴), and C═O;
wherein each occurrence of R³, R⁴, and R⁵ is independently selected from the group consisting of hydrogen, deuterium, halogen, hydroxyl, alkyl, cycloalkyl, heterocycloalkyl, alkenyl, cycloalkenyl, alkynyl, cycloalkynyl, aryl, aryl alkyl, heteroaryl, heteroaryl alkyl, and any combination thereof; and
each occurrence of Z is selected from the group consisting of Li, Na, and K; and
the linker is an optionally substituted linking moiety selected from the group consisting of a branched or unbranched, cyclized or uncyclized, saturated or unsaturated chain of 5 to 16 carbon atoms in length, and any combination thereof,
wherein 1 to 6 of the carbon atoms are optionally replaced with a heteroatom independently selected from O, N, and S.

2. The compound of claim 1, wherein R¹ is selected from the group consisting of 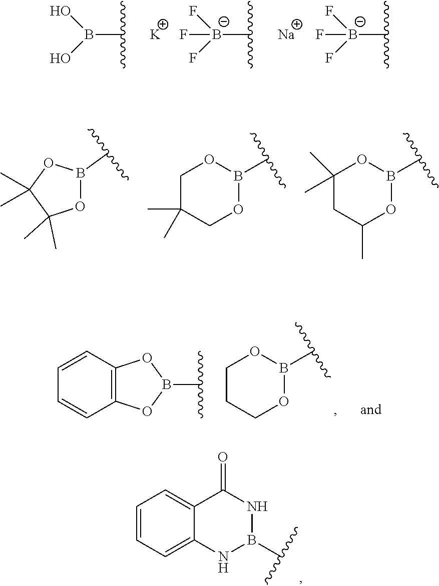
wherein the R¹ substituent point of attachment is on the substituent boron atom of R¹.

3. The compound of claim 1, wherein R² is selected from the group consisting of hydrogen, F, and Cl.

4. The compound of claim 1, wherein X is selected from the group consisting of CH₂ and C═O.

5. The compound of claim 1, wherein the linker is an optionally substituted linking moiety selected from the group consisting of a branched or linear C₅-C₁₆ alkyl, branched or linear amino-C₅-C₁₆ alkyl, branched or linear C₅-C₁₆ alkoxy, branched or linear thio-C₅-C₁₆ alkyl, C₅-C₁₆ cycloalkyl, amino-C₅-C₁₆ cycloalkyl, hydroxy-C₅-C₁₆ cycloalky, thio-C₅-C₁₆ cycloalkyl, and any combination thereof; wherein 1 to 6 of the carbon atoms are optionally replaced with a heteroatom.

6. The compound of claim 1, wherein the linker is selected from the group consisting of:

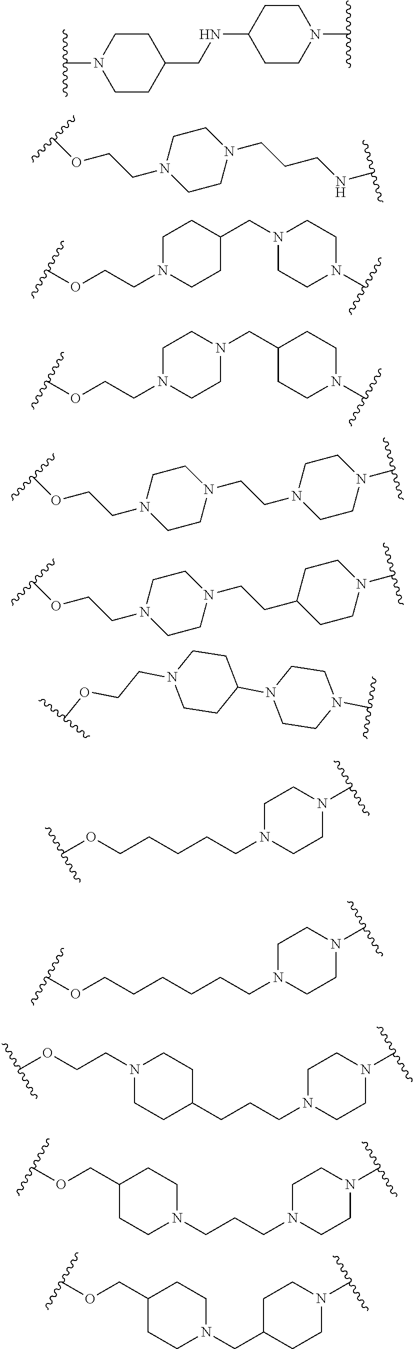

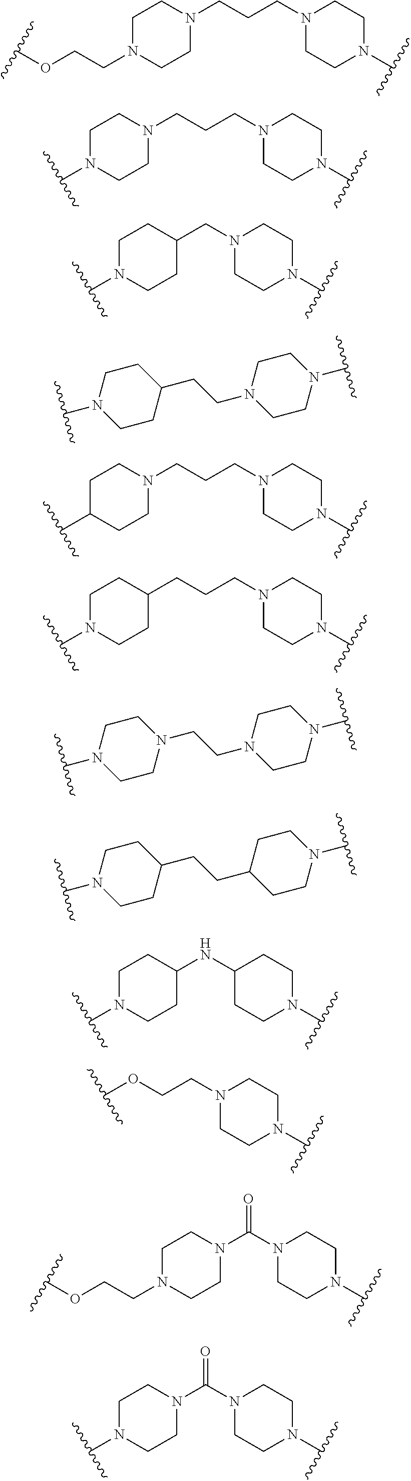

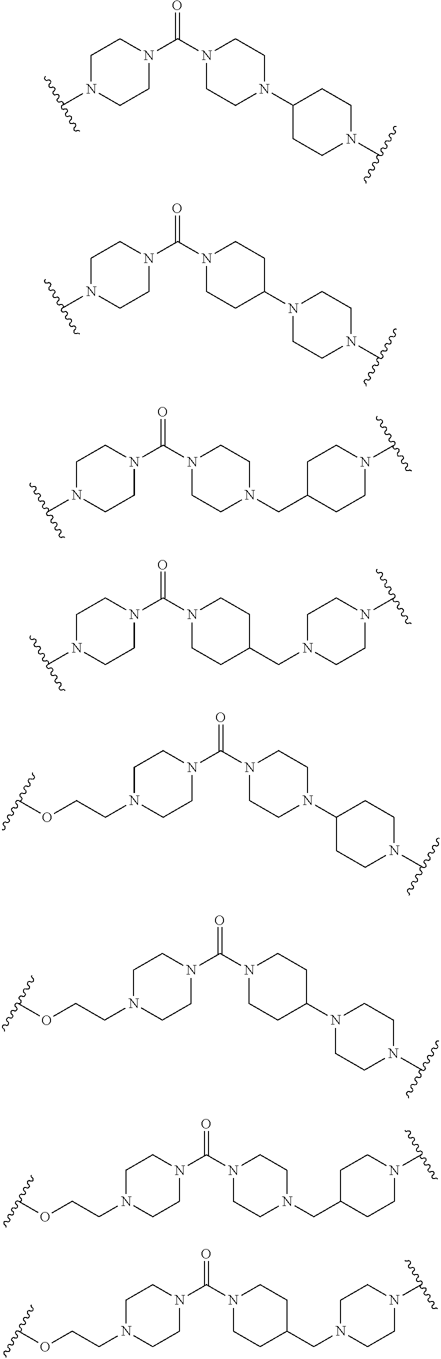

7. The compound of claim 1, wherein the compound represented by Formula (I) is selected from the group consisting of:
(Z)-(4-(1-(4-(4-((4-(2-(2,6-dioxopiperidin-3-yl)-1-oxoisoindolin-5-yl)piperazin-1-yl) methyl)piperidin-1-yl)phenyl)-2-phenylbut-1-en-1-yl)phenyl)boronic acid;
(E)-(4-(1-(4-(4-((4-(2-(2,6-dioxopiperidin-3-yl)-1-oxoisoindolin-5-yl)piperazin-1-yl) methyl)piperidin-1-yl)phenyl)-2-phenylbut-1-en-1-yl)phenyl)boronic acid;
(Z)-(4-(1-(4-(4-((4-(2-(2,6-dioxopiperidin-3-yl)-3-oxoisoindolin-5-yl)piperazin-1-yl) methyl)piperidin-1-yl)phenyl)-2-phenylbut-1-en-1-yl)phenyl)boronic acid;
(E)-(4-(1-(4-(4-((4-(2-(2,6-dioxopiperidin-3-yl)-3-oxoisoindolin-5-yl)piperazin-1-yl) methyl)piperidin-1-yl)phenyl)-2-phenylbut-1-en-1-yl)phenyl)boronic acid;
(Z)-(4-(1-(4-(4-((4-(2-(2,6-dioxopiperidin-3-yl)-1,3-dioxoisoindolin-5-yl)piperazin-1-yl) methyl)piperidin-1-yl)phenyl)-2-phenylbut-1-en-1-yl)phenyl)boronic acid;
(E)-(4-(1-(4-(4-((4-(2-(2,6-dioxopiperidin-3-yl)-1,3-dioxoisoindolin-5-yl)piperazin-1-yl) methyl)piperidin-1-yl)phenyl)-2-phenylbut-1-en-1-yl)phenyl)boronic acid;
(Z)-(4-(1-(4-(4-(2-(4-(2-(2,6-dioxopiperidin-3-yl)-1-oxoisoindolin-5-yl)piperazin-1-yl) ethyl)piperidin-1-yl)phenyl)-2-phenylbut-1-en-1-yl)phenyl)boronic acid;
(E)-(4-(1-(4-(4-(2-(4-(2-(2,6-dioxopiperidin-3-yl)-1-oxoisoindolin-5-yl)piperazin-1-yl) ethyl)piperidin-1-yl)phenyl)-2-phenylbut-1-en-1-yl)phenyl)boronic acid;
(Z)-3-(1-oxo-5-(4-(2-(1-(4-(2-phenyl-1-(4-(4,4,5,5-tetramethyl-1,3,2-dioxaborolan-2-yl)phenyl)but-1-en-1-yl)phenyl)piperidin-4-yl)ethyl)piperazin-1-yl)isoindolin-2-yl)piperidine-2,6-dione;
(E)-3-(1-oxo-5-(4-(2-(1-(4-(2-phenyl-1-(4-(4,4,5,5-tetramethyl-1,3,2-dioxaborolan-2-yl) phenyl)but-1-en-1-yl)phenyl)piperidin-4-yl)ethyl)piperazin-1-yl)isoindolin-2-yl)piperidine-2,6-dione;
(Z)-(4-(1-(4-(4-(((1-(2-(2,6-dioxopiperidin-3-yl)-1-oxoisoindolin-5-yl)piperidin-4-yl) amino)methyl)piperidin-1-yl)phenyl)-2-phenylbut-1-en-1-yl)phenyl)boronic acid;
(E)-(4-(1-(4-(4-(((1-(2-(2,6-dioxopiperidin-3-yl)-1-oxoisoindolin-5-yl)piperidin-4-yl) amino)methyl)piperidin-1-yl)phenyl)-2-phenylbut-1-en-1-yl)phenyl)boronic acid;
(Z)-(4-(1-(4-(4-(((1-(2-(2,6-dioxopiperidin-3-yl)-3-oxoisoindolin-5-yl)piperidin-4-yl) amino)methyl)piperidin-1-yl)phenyl)-2-phenylbut-1-en-1-yl)phenyl)boronic acid;
(E)-(4-(1-(4-(4-(((1-(2-(2,6-dioxopiperidin-3-yl)-3-oxoisoindolin-5-yl)piperidin-4-yl) amino)methyl)piperidin-1-yl)phenyl)-2-phenylbut-1-en-1-yl)phenyl)boronic acid;
(Z)-(4-(1-(4-(2-(4-(3-((2-(2,6-dioxopiperidin-3-yl)-1,3-dioxoisoindolin-5-yl)amino) propyl)piperazin-1-yl)ethoxy)phenyl)-2-phenylbut-1-en-1-yl)phenyl)boronic acid;
(E)-(4-(1-(4-(2-(4-(3-((2-(2,6-dioxopiperidin-3-yl)-1,3-dioxoisoindolin-5-yl)amino) propyl)piperazin-1-yl)ethoxy)phenyl)-2-phenylbut-1-en-1-yl)phenyl)boronic acid;
(Z)-(4-(1-(4-(2-(4-((1-(2-(2,6-dioxopiperidin-3-yl)-1,3-dioxoisoindolin-5-yl)piperidin-4-yl) methyl)piperazin-1-yl)ethoxy)phenyl)-2-phenylbut-1-en-1-yl)phenyl)boronic acid;
(E)-(4-(1-(4-(2-(4-((1-(2-(2,6-dioxopiperidin-3-yl)-1,3-dioxoisoindolin-5-yl)piperidin-4-yl) methyl)piperazin-1-yl)ethoxy)phenyl)-2-phenylbut-1-en-1-yl)phenyl)boronic acid;
(Z)-(4-(1-(4-(2-(4-((4-(2-(2,6-dioxopiperidin-3-yl)-1,3-dioxoisoindolin-5-yl)piperazin-1-yl) methyl)piperidin-1-yl)ethoxy)phenyl)-2-phenylbut-1-en-1-yl)phenyl)boronic acid;
(E)-(4-(1-(4-(2-(4-((4-(2-(2,6-dioxopiperidin-3-yl)-1,3-dioxoisoindolin-5-yl)piperazin-1-yl) methyl)piperidin-1-yl)ethoxy)phenyl)-2-phenylbut-1-en-1-yl)phenyl)boronic acid;
(Z)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid;
(E)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid;
(Z)-(4-(1-(4-(2-(4-(2-(4-(2-(2,6-dioxopiperidin-3-yl)-1,3-dioxoisoindolin-5-yl)piperazin-1-yl) ethyl)piperazin-1-yl)ethoxy)phenyl)-2-phenylbut-1-en-1-yl)phenyl)boronic acid;
(E)-(4-(1-(4-(2-(4-(2-(4-(2-(2,6-dioxopiperidin-3-yl)-1,3-dioxoisoindolin-5-yl)piperazin-1-yl) ethyl)piperazin-1-yl)ethoxy)phenyl)-2-phenylbut-1-en-1-yl)phenyl)boronic acid;
(Z)-(4-(1-(4-(2-(4-(2-(1-(2-(2,6-dioxopiperidin-3-yl)-1,3-dioxoisoindolin-5-yl)piperidin-4-yl) ethyl)piperazin-1-yl)ethoxy)phenyl)-2-phenylbut-1-en-1-yl)phenyl)boronic acid;
(E)-(4-(1-(4-(2-(4-(2-(1-(2-(2,6-dioxopiperidin-3-yl)-1,3-dioxoisoindolin-5-yl)piperidin-4-yl) ethyl)piperazin-1-yl)ethoxy)phenyl)-2-phenylbut-1-en-1-yl)phenyl)boronic acid;
(Z)-(4-(1-(4-(2-(4-(2-(4-(2-(2,6-dioxopiperidin-3-yl)-1,3-dioxoisoindolin-5-yl)piperazin-1-yl) ethyl)piperidin-1-yl)ethoxy)phenyl)-2-phenylbut-1-en-1-yl)phenyl)boronic acid;
(E)-(4-(1-(4-(2-(4-(2-(4-(2-(2,6-dioxopiperidin-3-yl)-1,3-dioxoisoindolin-5-yl)piperazin-1-yl) ethyl)piperidin-1-yl)ethoxy)phenyl)-2-phenylbut-1-en-1-yl)phenyl)boronic acid;
(Z)-(4-(1-(4-(2-(4-(2-(4-(2-(2,6-dioxopiperidin-3-yl)-1-oxoisoindolin-5-yl)piperazin-1-yl) ethyl)piperidin-1-yl)ethoxy)phenyl)-2-phenylbut-1-en-1-yl)phenyl)boronic acid;
(E)-(4-(1-(4-(2-(4-(2-(4-(2-(2,6-dioxopiperidin-3-yl)-1-oxoisoindolin-5-yl)piperazin-1-yl) ethyl)piperidin-1-yl)ethoxy)phenyl)-2-phenylbut-1-en-1-yl)phenyl)boronic acid;
(Z)-(4-(1-(4-(2-(4-(4-(2-(2,6-dioxopiperidin-3-yl)-1,3-dioxoisoindolin-5-yl)piperazin-1-yl)piperidin-1-yl)ethoxy)phenyl)-2-phenylbut-1-en-1-yl)phenyl)boronic acid;
(E)-(4-(1-(4-(2-(4-(4-(2-(2,6-dioxopiperidin-3-yl)-1,3-dioxoisoindolin-5-yl)piperazin-1-yl)piperidin-1-yl)ethoxy)phenyl)-2-phenylbut-1-en-1-yl)phenyl)boronic acid;
(Z)-(4-(1-(4-(2-(1′-(2-(2,6-dioxopiperidin-3-yl)-1,3-dioxoisoindolin-5-yl)-[4,4′-bipiperidin]-1-yl) ethoxy)phenyl)-2-phenylbut-1-en-1-yl)phenyl)boronic acid;
(E)-(4-(1-(4-(2-(1′-(2-(2,6-dioxopiperidin-3-yl)-1,3-dioxoisoindolin-5-yl)-[4,4′-bipiperidin]-1-yl) ethoxy)phenyl)-2-phenylbut-1-en-1-yl)phenyl)boronic acid;
(Z)-(4-(1-(4-((5-(4-(2-(2,6-dioxopiperidin-3-yl)-1-oxoisoindolin-5-yl)piperazin-1-yl) pentyl)oxy)phenyl)-2-phenylbut-1-en-1-yl)phenyl)boronic acid;
(E)-(4-(1-(4-((5-(4-(2-(2,6-dioxopiperidin-3-yl)-1-oxoisoindolin-5-yl)piperazin-1-yl) pentyl)oxy)phenyl)-2-phenylbut-1-en-1-yl)phenyl)boronic acid;
(Z)-(4-(1-(4-(2-(4-(3-(4-(2-(2,6-dioxopiperidin-3-yl)-1,3-dioxoisoindolin-5-yl)piperazin-1-yl) propyl)piperazin-1-yl)ethoxy)phenyl)-2-phenylbut-1-en-1-yl)phenyl)boronic acid;
(E)-(4-(1-(4-(2-(4-(3-(4-(2-(2,6-dioxopiperidin-3-yl)-1,3-dioxoisoindolin-5-yl)piperazin-1-yl) propyl)piperazin-1-yl)ethoxy)phenyl)-2-phenylbut-1-en-1-yl)phenyl)boronic acid;
(Z)-(4-(1-(4-(4-(((1-(2-(2,6-dioxopiperidin-3-yl)-1,3-dioxoisoindolin-5-yl)piperidin-4-yl) amino)methyl)piperidin-1-yl)phenyl)-2-phenylbut-1-en-1-yl)phenyl)boronic acid;
(E)-(4-(1-(4-(4-(((1-(2-(2,6-dioxopiperidin-3-yl)-1,3-dioxoisoindolin-5-yl)piperidin-4-yl) amino)methyl)piperidin-1-yl)phenyl)-2-phenylbut-1-en-1-yl)phenyl)boronic acid;
(Z)-(4-(1-(4-(2-(4-(2-(2,6-dioxopiperidin-3-yl)-1,3-dioxoisoindolin-5-yl)piperazin-1-yl) ethoxy)phenyl)-2-phenylbut-1-en-1-yl)phenyl)boronic acid;
(E)-(4-(1-(4-(2-(4-(2-(2,6-dioxopiperidin-3-yl)-1,3-dioxoisoindolin-5-yl)piperazin-1-yl) ethoxy)phenyl)-2-phenylbut-1-en-1-yl)phenyl)boronic acid;
(Z)-(4-(1-(4-(4-(3-(4-(2-(2,6-dioxopiperidin-3-yl)-1,3-dioxoisoindolin-5-yl)piperazin-1-yl) propyl)piperazin-1-yl)phenyl)-2-phenylbut-1-en-1-yl)phenyl)boronic acid;
(E)-(4-(1-(4-(4-(3-(4-(2-(2,6-dioxopiperidin-3-yl)-1,3-dioxoisoindolin-5-yl)piperazin-1-yl) propyl)piperazin-1-yl)phenyl)-2-phenylbut-1-en-1-yl)phenyl)boronic acid;
(Z)-(4-(1-(4-(4-(3-(4-(2-(2,6-dioxopiperidin-3-yl)-1-oxoisoindolin-5-yl)piperazin-1-yl) propyl)piperazin-1-yl)phenyl)-2-phenylbut-1-en-1-yl)phenyl)boronic acid;
(E)-(4-(1-(4-(4-(3-(4-(2-(2,6-dioxopiperidin-3-yl)-1-oxoisoindolin-5-yl)piperazin-1-yl) propyl)piperazin-1-yl)phenyl)-2-phenylbut-1-en-1-yl)phenyl)boronic acid;
(Z)-(4-(1-(4-(1-(3-(4-(2-(2,6-dioxopiperidin-3-yl)-1,3-dioxoisoindolin-5-yl)piperazin-1-yl) propyl)piperidin-4-yl)phenyl)-2-phenylbut-1-en-1-yl)phenyl)boronic acid;
(E)-(4-(1-(4-(1-(3-(4-(2-(2,6-dioxopiperidin-3-yl)-1,3-dioxoisoindolin-5-yl)piperazin-1-yl) propyl)piperidin-4-yl)phenyl)-2-phenylbut-1-en-1-yl)phenyl)boronic acid;
(Z)-(4-(1-(4-(1-(3-(4-(2-(2,6-dioxopiperidin-3-yl)-1-oxoisoindolin-5-yl)piperazin-1-yl) propyl)piperidin-4-yl)phenyl)-2-phenylbut-1-en-1-yl)phenyl)boronic acid;
(E)-(4-(1-(4-(1-(3-(4-(2-(2,6-dioxopiperidin-3-yl)-1-oxoisoindolin-5-yl)piperazin-1-yl) propyl)piperidin-4-yl)phenyl)-2-phenylbut-1-en-1-yl)phenyl)boronic acid;
(Z)-(4-(1-(4-(4-(3-(4-(2-(2,6-dioxopiperidin-3-yl)-1,3-dioxoisoindolin-5-yl)piperazin-1-yl) propyl)piperidin-1-yl)phenyl)-2-phenylbut-1-en-1-yl)phenyl)boronic acid;
(E)-(4-(1-(4-(4-(3-(4-(2-(2,6-dioxopiperidin-3-yl)-1,3-dioxoisoindolin-5-yl)piperazin-1-yl) propyl)piperidin-1-yl)phenyl)-2-phenylbut-1-en-1-yl)phenyl)boronic acid;
(Z)-(4-(1-(4-(4-(3-(4-(2-(2,6-dioxopiperidin-3-yl)-1-oxoisoindolin-5-yl)piperazin-1-yl) propyl)piperidin-1-yl)phenyl)-2-phenylbut-1-en-1-yl)phenyl)boronic acid;
(E)-(4-(1-(4-(4-(3-(4-(2-(2,6-dioxopiperidin-3-yl)-1-oxoisoindolin-5-yl)piperazin-1-yl) propyl)piperidin-1-yl)phenyl)-2-phenylbut-1-en-1-yl)phenyl)boronic acid;
(Z)-(4-(1-(4-(4-(3-(1-(2-(2,6-dioxopiperidin-3-yl)-1,3-dioxoisoindolin-5-yl)piperidin-4-yl) propyl)piperazin-1-yl)phenyl)-2-phenylbut-1-en-1-yl)phenyl)boronic acid;
(E)-(4-(1-(4-(4-(3-(1-(2-(2,6-dioxopiperidin-3-yl)-1,3-dioxoisoindolin-5-yl)piperidin-4-yl) propyl)piperazin-1-yl)phenyl)-2-phenlbut-1-en-1-yl)phenyl)boronic acid;
(Z)-(4-(1-(4-(4-(3-(1-(2-(2,6-dioxopiperidin-3-yl)-1-oxoisoindolin-5-yl)piperidin-4-yl) propyl)piperazin-1-yl)phenyl)-2-phenylbut-1-en-1-yl)phenyl)boronic acid;
(E)-(4-(1-(4-(4-(3-(1-(2-(2,6-dioxopiperidin-3-yl)-1-oxoisoindolin-5-yl)piperidin-4-yl) propyl)piperazin-1-yl)phenyl)-2-phenylbut-1-en-1-yl)phenyl)boronic acid;
(Z)-(4-(1-(4-(4-(2-(4-(2-(2,6-dioxopiperidin-3-yl)-1,3-dioxoisoindolin-5-yl)piperazin-1-yl) ethyl)piperazin-1-yl)phenyl)-2-phenylbut-1-en-1-yl)phenyl)boronic acid;
(E)-(4-(1-(4-(4-(2-(4-(2-(2,6-dioxopiperidin-3-yl)-1,3-dioxoisoindolin-5-yl)piperazin-1-yl) ethyl)piperazin-1-yl)phenyl)-2-phenylbut-1-en-1-yl)phenyl)boronic acid;
(Z)-(4-(1-(4-(4-(2-(4-(2-(2,6-dioxopiperidin-3-yl)-1-oxoisoindolin-5-yl)piperazin-1-yl) ethyl)piperazin-1-yl)phenyl)-2-phenylbut-1-en-1-yl)phenyl)boronic acid;
(E)-(4-(1-(4-(4-(2-(4-(2-(2,6-dioxopiperidin-3-yl)-1-oxoisoindolin-5-yl)piperazin-1-yl) ethyl)piperazin-1-yl)phenyl)-2-phenylbut-1-en-1-yl)phenyl)boronic acid;
(Z)-(4-(1-(4-(4-(2-(4-(2-(2,6-dioxopiperidin-3-yl)-1,3-dioxoisoindolin-5-yl)piperazin-1-yl) ethyl)piperidin-1-yl)phenyl)-2-phenylbut-1-en-1-yl)phenyl)boronic acid;
(E)-(4-(1-(4-(4-(2-(4-(2-(2,6-dioxopiperidin-3-yl)-1,3-dioxoisoindolin-5-yl)piperazin-1-yl) ethyl)piperidin-1-yl)phenyl)-2-phenylbut-1-en-1-yl)phenyl)boronic acid;
(Z)-(4-(1-(4-(4-(2-(1-(2-(2,6-dioxopiperidin-3-yl)-1,3-dioxoisoindolin-5-yl)piperidin-4-yl) ethyl)piperazin-1-yl)phenyl)-2-phenylbut-1-en-1-yl)phenyl)boronic acid;
(E)-(4-(1-(4-(4-(2-(1-(2-(2,6-dioxopiperidin-3-yl)-1,3-dioxoisoindolin-5-yl)piperidin-4-yl) ethyl)piperazin-1-yl)phenyl)-2-phenylbut-1-en-1-yl)phenyl)boronic acid;
(Z)-(4-(1-(4-(4-(2-(1-(2-(2,6-dioxopiperidin-3-yl)-1-oxoisoindolin-5-yl)piperidin-4-yl) ethyl)piperazin-1-yl)phenyl)-2-phenylbut-1-en-1-yl)phenyl)boronic acid;
(E)-(4-(1-(4-(4-(2-(1-(2-(2,6-dioxopiperidin-3-yl)-1-oxoisoindolin-5-yl)piperidin-4-yl) ethyl)piperazin-1-yl)phenyl)-2-phenylbut-1-en-1-yl)phenyl)boronic acid;
(S,Z)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid;
(R,Z)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid;
(S,E)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid;
(R,E)-(4-(1-(4-(2-(4-((1-(2-(2,6-dioxopiperidin-3-yl)-1-oxoisoindolin-5-yl)piperidin-4-yl) methyl)piperazin-1-yl)ethoxy)phenyl)-2-phenylbut-1-en-1-yl)phenyl)boronic acid;
(S,Z)-(4-(1-(4-(2-(4-((4-(2-(2,6-dioxopiperidin-3-yl)-1-oxoisoindolin-5-yl)piperazin-1-yl) methyl)piperidin-1-yl)ethoxy)phenyl)-2-phenylbut-1-en-1-yl)phenyl)boronic acid;
(R,Z)-(4-(1-(4-(2-(4-((4-(2-(2,6-dioxopiperidin-3-yl)-1-oxoisoindolin-5-yl)piperazin-1-yl) methyl)piperidin-1-yl)ethoxy)phenyl)-2-phenylbut-1-en-1-yl)phenyl)boronic acid;
(S,E)-(4-(1-(4-(2-(4-((4-(2-(2,6-dioxopiperidin-3-yl)-1-oxoisoindolin-5-yl)piperazin-1-yl) methyl)piperidin-1-yl)ethoxy)phenyl)-2-phenylbut-1-en-1-yl)phenyl)boronic acid;
(R,E)-(4-(1-(4-(2-(4-((4-(2-(2,6-dioxopiperidin-3-yl)-1-oxoisoindolin-5-yl)piperazin-1-yl) methyl)piperidin-1-yl)ethoxy)phenyl)-2-phenylbut-1-en-1-yl)phenyl)boronic acid;
(Z)-3-(5-(4-(4-(2-(4-(1-(4-hydroxyphenyl)-2-phenylbut-1-en-1-yl)phenoxy)ethyl)piperazine-1-carbonyl) piperazin-1-yl)-1-oxoisoindolin-2-yl)piperidine-2,6-dione;
(S,Z)-3-(5-(4-(4-(2-(4-(1-(4-hydroxyphenyl)-2-phenylbut-1-en-1-yl)phenoxy)ethyl)piperazine-1-carbonyl) piperazin-1-yl)-1-oxoisoindolin-2-yl)piperidine-2,6-dione;
(R,Z)-3-(5-(4-(4-(2-(4-(1-(4-hydroxyphenyl)-2-phenylbut-1-en-1-yl)phenoxy)ethyl)piperazine-1-carbonyl) piperazin-1-yl)-1-oxoisoindolin-2-yl)piperidine-2,6-dione;
(E)-3-(5-(4-(4-(2-(4-(1-(4-hydroxyphenyl)-2-phenylbut-1-en-1-yl)phenoxy)ethyl)piperazine-1-carbonyl) piperazin-1-yl)-1-oxoisoindolin-2-yl)piperidine-2,6-dione;
(S,E)-3-(5-(4-(4-(2-(4-(1-(4-hydroxyphenyl)-2-phenylbut-1-en-1-yl)phenoxy)ethyl)piperazine-1-carbonyl) piperazin-1-yl)-1-oxoisoindolin-2-yl)piperidine-2,6-dione;
(R,E)-3-(5-(4-(4-(2-(4-(1-(4-hydroxyphenyl)-2-phenylbut-1-en-1-yl)phenoxy)ethyl)piperazine-1-carbonyl) piperazin-1-yl)-1-oxoisoindolin-2-yl)piperidine-2,6-dione;
(Z)-(4-(1-(4-(2-(4-(4-(2-(2,6-dioxopiperidin-3-yl)-1-oxoisoindolin-5-yl)piperazine-1-carbonyl) piperazin-1-yl)ethoxy)phenyl)-2-phenylbut-1-en-1-yl)phenyl)boronic acid;
(S,Z)-(4-(1-(4-(2-(4-(4-(2-(2,6-dioxopiperidin-3-yl)-1-oxoisoindolin-5-yl)piperazine-1-carbonyl) piperazin-1-yl)ethoxy)phenyl)-2-phenylbut-1-en-1-yl)phenyl)boronic acid;
(R,Z)-(4-(1-(4-(2-(4-(4-(2-(2,6-dioxopiperidin-3-yl)-1-oxoisoindolin-5-yl)piperazine-1-carbonyl) piperazin-1-yl)ethoxy)phenyl)-2-phenylbut-1-en-1-yl)phenyl)boronic acid;
(E)-(4-(1-(4-(2-(4-(4-(2-(2,6-dioxopiperidin-3-yl)-1-oxoisoindolin-5-yl)piperazine-1-carbonyl) piperazin-1-yl)ethoxy)phenyl)-2-phenylbut-1-en-1-yl)phenyl)boronic acid;
(S,E)-(4-(1-(4-(2-(4-(4-(2-(2,6-dioxopiperidin-3-yl)-1-oxoisoindolin-5-yl)piperazine-1-carbonyl) piperazin-1-yl)ethoxy)phenyl)-2-phenylbut-1-en-1-yl)phenyl)boronic acid;
(R,E)-(4-(1-(4-(2-(4-(4-(2-(2,6-dioxopiperidin-3-yl)-1-oxoisoindolin-5-yl)piperazine-1-carbonyl) piperazin-1-yl)ethoxy)phenyl)-2-phenylbut-1-en-1-yl)phenyl)boronic acid;
(Z)-3-(5-(4-((1-(4-(2-(4-(1-(4-hydroxyphenyl)-2-phenylbut-1-en-1-yl)phenoxy)ethyl)piperazine-1-carbonyl) piperidin-4-yl)methyl)piperazin-1-yl)-1-oxoisoindolin-2-yl)piperidine-2,6-dione;
(E)-3-(5-(4-((1-(4-(2-(4-(1-(4-hydroxyphenyl)-2-phenylbut-1-en-1-yl)phenoxy)ethyl)piperazine-1-carbonyl) piperidin-4-yl)methyl)piperazin-1-yl)-1-oxoisoindolin-2-yl)piperidine-2,6-dione;
(Z)-(4-(1-(4-(2-(4-(4-((4-(2-(2,6-dioxopiperidin-3-yl)-1-oxoisoindolin-5-yl)piperazin-1-yl) methyl)piperidine-1-carbonyl)piperazin-1-yl)ethoxy)phenyl)-2-phenylbut-1-en-1-yl) phenyl)boronic acid;
(E)-(4-(1-(4-(2-(4-(4-((4-(2-(2,6-dioxopiperidin-3-yl)-1-oxoisoindolin-5-yl)piperazin-1-yl) methyl)piperidine-1-carbonyl)piperazin-1-yl)ethoxy)phenyl)-2-phenylbut-1-en-1-yl) phenyl)boronic acid;
(Z)-3-(5-(4-(1-(4-(2-(4-(1-(4-hydroxyphenyl)-2-phenylbut-1-en-1-yl)phenoxy)ethyl)piperazine-1-carbonyl) piperidin-4-yl)piperazin-1-yl)-1-oxoisoindolin-2-yl)piperidine-2,6-dione;
(E)-3-(5-(4-(1-(4-(2-(4-(1-(4-hydroxyphenyl)-2-phenylbut-1-en-1-yl)phenoxy)ethyl)piperazine-1-carbonyl) piperidin-4-yl)piperazin-1-yl)-1-oxoisoindolin-2-yl)piperidine-2,6-dione;
(Z)-(4-(1-(4-(2-(4-(4-(4-(2-(2,6-dioxopiperidin-3-yl)-1-oxoisoindolin-5-yl)piperazin-1-yl)piperidine-1-carbonyl)piperazin-1-yl)ethoxy)phenyl)-2-phenylbut-1-en-1-yl)phenyl)boronic acid;
(E)-(4-(1-(4-(2-(4-(4-(4-(2-(2,6-dioxopiperidin-3-yl)-1-oxoisoindolin-5-yl)piperazin-1-yl)piperidine-1-carbonyl)piperazin-1-yl)ethoxy)phenyl)-2-phenylbut-1-en-1-yl)phenyl)boronic acid;
(E)-3-(5-(4-(4-(4-(1-(4-hydroxyphenyl)-2-phenylbut-1-en-1-yl)phenyl)piperazine-1-carbonyl) piperazin-1-yl)-1-oxoisoindolin-2-yl)piperidine-2,6-dione;
(S,E)-3-(5-(4-(4-(4-(1-(4-hydroxyphenyl)-2-phenylbut-1-en-1-yl)phenyl)piperazine-1-carbonyl) piperazin-1-yl)-1-oxoisoindolin-2-yl)piperidine-2,6-dione;
(R,E)-3-(5-(4-(4-(4-(1-(4-hydroxyphenyl)-2-phenylbut-1-en-1-yl)phenyl)piperazine-1-carbonyl) piperazin-1-yl)-1-oxoisoindolin-2-yl)piperidine-2,6-dione;
(Z)-3-(5-(4-(4-(4-(1-(4-hydroxyphenyl)-2-phenylbut-1-en-1-yl)phenyl)piperazine-1-carbonyl) piperazin-1-yl)-1-oxoisoindolin-2-yl)piperidine-2,6-dione;
(S,Z)-3-(5-(4-(4-(4-(1-(4-hydroxyphenyl)-2-phenylbut-1-en-1-yl)phenyl)piperazine-1-carbonyl) piperazin-1-yl)-1-oxoisoindolin-2-yl)piperidine-2,6-dione;
(R,Z)-3-(5-(4-(4-(4-(1-(4-hydroxyphenyl)-2-phenylbut-1-en-1-yl)phenyl)piperazine-1-carbonyl) piperazin-1-yl)-1-oxoisoindolin-2-yl)piperidine-2,6-dione;
(Z)-(4-(1-(4-(4-(4-(2-(2,6-dioxopiperidin-3-yl)-1-oxoisoindolin-5-yl)piperazine-1-carbonyl) piperazin-1-yl)phenyl)-2-phenylbut-1-en-1-yl)phenyl)boronic acid;
(S,Z)-(4-(1-(4-(4-(4-(2-(2,6-dioxopiperidin-3-yl)-1-oxoisoindolin-5-yl)piperazine-1-carbonyl) piperazin-1-yl)phenyl)-2-phenylbut-1-en-1-yl)phenyl)boronic acid;
(R,Z)-(4-(1-(4-(4-(4-(2-(2,6-dioxopiperidin-3-yl)-1-oxoisoindolin-5-yl)piperazine-1-carbonyl) piperazin-1-yl)phenyl)-2-phenylbut-1-en-1-yl)phenyl)boronic acid;
(E)-(4-(1-(4-(4-(4-(2-(2,6-dioxopiperidin-3-yl)-1-oxoisoindolin-5-yl)piperazine-1-carbonyl) piperazin-1-yl)phenyl)-2-phenylbut-1-en-1-yl)phenyl)boronic acid;
(S,E)-(4-(1-(4-(4-(4-(2-(2,6-dioxopiperidin-3-yl)-1-oxoisoindolin-5-yl)piperazine-1-carbonyl) piperazin-1-yl)phenyl)-2-phenylbut-1-en-1-yl)phenyl)boronic acid;
(R,E)-(4-(1-(4-(4-(4-(2-(2,6-dioxopiperidin-3-yl)-1-oxoisoindolin-5-yl)piperazine-1-carbonyl) piperazin-1-yl)phenyl)-2-phenylbut-1-en-1-yl)phenyl)boronic acid;
(E)-3-(5-(4-(1-(4-(4-(1-(4-hydroxyphenyl)-2-phenylbut-1-en-1-yl)phenyl)piperazine-1-carbonyl) piperidin-4-yl)piperazin-1-yl)-1-oxoisoindolin-2-yl)piperidine-2,6-dione;
(Z)-(4-(1-(4-(4-(4-(4-(2-(2,6-dioxopiperidin-3-yl)-1-oxoisoindolin-5-yl)piperazin-1-yl)piperidine-1-carbonyl) piperazin-1-yl)phenyl)-2-phenylbut-1-en-1-yl)phenyl)boronic acid;
(Z)-3-(5-(4-(1-(4-(4-(1-(4-hydroxyphenyl)-2-phenylbut-1-en-1-yl)phenyl)piperazine-1-carbonyl) piperidin-4-yl)piperazin-1-yl)-1-oxoisoindolin-2-yl)piperidine-2,6-dione;
(E)-(4-(1-(4-(4-(4-(4-(2-(2,6-dioxopiperidin-3-yl)-1-oxoisoindolin-5-yl)piperazin-1-yl)piperidine-1-carbonyl) piperazin-1-yl)phenyl)-2-phenylbut-1-en-1-yl)phenyl)boronic acid;
(E)-3-(5-(4-((1-(4-(4-(1-(4-hydroxyphenyl)-2-phenylbut-1-en-1-yl)phenyl)piperazine-1-carbonyl) piperidin-4-yl)methyl)piperazin-1-yl)-1-oxoisoindolin-2-yl)piperidine-2,6-dione;
(Z)-(4-(1-(4-(4-(4-((4-(2-(2,6-dioxopiperidin-3-yl)-1-oxoisoindolin-5-yl)piperazin-1-yl) methyl)piperidine-1-carbonyl)piperazin-1-yl)phenyl)-2-phenylbut-1-en-1-yl)phenyl)boronic acid;
(Z)-3-(5-(4-((1-(4-(4-(1-(4-hydroxyphenyl)-2-phenylbut-1-en-1-yl)phenyl)piperazine-1-carbonyl) piperidin-4-yl)methyl)piperazin-1-yl)-1-oxoisoindolin-2-yl)piperidine-2,6-dione,
(E)-(4-(1-(4-(4-(4-((4-(2-(2,6-dioxopiperidin-3-yl)-1-oxoisoindolin-5-yl)piperazin-1-yl) methyl)piperidine-1-carbonyl)piperazin-1-yl)phenyl)-2-phenylbut-1-en-1-yl)phenyl)boronic acid; and any combination thereof.

8. A composition comprising at least one compound of claim 1.

9. A pharmaceutical composition comprising at least one compound of claim 1 and a pharmaceutically acceptable carrier.

10. A pharmaceutical formulation comprising at least one compound of claim 1 and a pharmaceutically acceptable carrier.

11. The pharmaceutical composition of claim 9, wherein the pharmaceutical composition is suitable for enteral administration, oral administration, or parenteral administration.

12. The pharmaceutical formulation of claim 10, wherein the pharmaceutical formulation is suitable for enteral administration, oral administration, or parenteral administration.

13. The composition of claim 8, wherein the composition is a pharmaceutical composition or a pharmaceutical formulation.

14. A method of preparing at least one compound of claim 1 or a composition thereof.

15. A method for treating a disease or disorder in a subject in need thereof, the method comprising administering to the subject an effective amount of at least one compound of claim 1 or a composition thereof.

16. The method of claim 15, wherein the disease or disorder is a disease or disorder associated with at least one estrogen receptor (ER).

17. The method of claim 15, wherein the disease or disorder is selected from the group consisting of a breast cancer, all stages of breast cancer, estrogen receptor (ER)-positive breast cancer, invasive breast cancer, and any combination thereof.

18. The method of claim 15, wherein the subject expresses a mutant estrogen receptor (ER)-α protein.

19. A method of reducing the level or activity of at least one estrogen receptor, the method comprising administering an effective amount of at least one compound of claim 1 or a composition thereof.

20. A method of inhibiting at least one estrogen receptor, the method comprising an effective amount of at least one compound of claim 1 or a composition thereof.
